SEQUENCE LISTING (967 sequences)

SEQ ID NO: 1 - DNA, 379 bp, Homo sapiens
  source 1..379: mol_type = other DNA
gaggtgcagctggtgcagtctggggctgaggtgaagaagcctgggtcctcggtgaaggtctcctgcaaggcttctggaggcaccttcagcaactatgctatcagctgggtgcgacaggcccctggacaagggcttgagtggatgggagggatcatccctatctttggtacagcaaactacgcacagaacttccagggcagagtcacgattaccgcggacgaatccatgagcacagcctacatggagctgagcagcctgagatctgaggacacggccgtatattactgtgcgggaggtgggaggtattgtagtggtggtaggtgccactctgcctactctgcctactggggccagggaaccctggtcaccgtctcctcag

SEQ ID NO: 2 - protein, 126 aa, Homo sapiens
EVQLVQSGAEVKKPGSSVKVSCKASGGTFSNYAISWVRQAPGQGLEWMGGIIPIFGTANYAQNFQGRVTITADESMSTAYMELSSLRSEDTAVYYCAGGGRYCSGGRCHSAYSAYWGQGTLVTVSS

SEQ ID NO: 3 - DNA, 322 bp, Homo sapiens
  source 1..322: mol_type = other DNA
gccatccagttgacccagtctccaggcaccctgtctttgcctccaggggaaagagccaccctctcctgcagggccagtcagagtgttagcagcagctacttagcctggtaccagcagaaacctggccaggctcccaggctcctcatctatggtgcatccagcagggccactggcatcccagacaggttcagtggcagtgggtctgggacagacttcactctcaccatcagcagactggaccctgaagattttgcagtgtattactgtcagcaatatggtagctcactcactttcggcggagggaccaaagtggatatcaaac

SEQ ID NO: 4 - protein, 107 aa, Homo sapiens
AIQLTQSPGTLSLPPGERATLSCRASQSVSSSYLAWYQQKPGQAPRLLIYGASSRATGIPDRFSGSGSGTDFTLTISRLDPEDFAVYYCQQYGSSLTFGGGTKVDIK

SEQ ID NO: 5 - protein, 8 aa, Homo sapiens
GGTFSNYA

SEQ ID NO: 6 - protein, 8 aa, Homo sapiens
IIPIFGTA

SEQ ID NO: 7 - protein, 19 aa, Homo sapiens
AGGGRYCSGGRCHSAYSAY

SEQ ID NO: 8 - protein, 7 aa, Homo sapiens
QSVSSSY

SEQ ID NO: 9 - skipped

SEQ ID NO: 10 - protein, 8 aa, Homo sapiens
QQYGSSLT

SEQ ID NO: 11 - DNA, 364 bp, Homo sapiens
  source 1..364: mol_type = other DNA
caggtgcagctggtggagtctgggggaggcttggtccaccctggggggtccctgagactctcctgttcagcctctggattcaccttcagtaactatgctatgcactgggtccgccaggctccagggaagggactggaatatgtttcagctattagtagtagtggggatatcacatactacgcggactccgtaaagggcagattcaccatctccagagacaattccaagaactcactgtatcttcaaatgaacagtctgagagctgaggacacggctgtttattactgtgtgaaagatgtaacgaggacctactacgtagtctttgactactggggccagggaaccctggtcaccgtctcctcag

SEQ ID NO: 12 - protein, 121 aa, Homo sapiens
QVQLVESGGGLVHPGGSLRLSCSASGFTFSNYAMHWVRQAPGKGLEYVSAISSSGDITYYADSVKGRFTISRDNSKNSLYLQMNSLRAEDTAVYYCVKDVTRTYYVVFDYWGQGTLVTVSS

SEQ ID NO: 13 - DNA, 322 bp, Homo sapiens
  source 1..322: mol_type = other DNA
gccatccagttgacccagtctccatcctccctgtctgcatctgtgggagacagagtcaccatcacttgccgggcaagtcagagcattagcagttatttaaattggtatcagcaggaaccagggaaagcccctaaactcctgatctatgctgcatccagtttgcaaggtggggtcccatcaaggttcagtggcagtggatctgggacagatttcactctcaccatcagcagtctgcaacctgaagattttgcaacttactactgtcaacagagttacactaccccgtacacttttggccaggggaccaaagtggatatcaaac

SEQ ID NO: 14 - protein, 107 aa, Homo sapiens
AIQLTQSPSSLSASVGDRVTITCRASQSISSYLNWYQQEPGKAPKLLIYAASSLQGGVPSRFSGSGSGTDFTLTISSLQPEDFATYYCQQSYTTPYTFGQGTKVDIK

SEQ ID NO: 15 - protein, 8 aa, Homo sapiens
GFTFSNYA

SEQ ID NO: 16 - protein, 8 aa, Homo sapiens
ISSSGDIT

SEQ ID NO: 17 - protein, 14 aa, Homo sapiens
VKDVTRTYYVVFDY

SEQ ID NO: 18 - protein, 6 aa, Homo sapiens
QSISSY

SEQ ID NO: 19 - skipped

SEQ ID NO: 20 - protein, 9 aa, Homo sapiens
QQSYTTPYT

SEQ ID NO: 21 - DNA, 358 bp, Homo sapiens
  source 1..358: mol_type = other DNA
caggtgcagctggtggagtctgggggaggcttggtccagcctggggggtccctgagactctcctgtgcagtctctggattcaccgtcagtaggaactacatgagctgggtccgccaggctccagggaaggggctggagtgggtctcacttatttatagcggtggtagcacattctacgcagactccgtgaagggcagattcaccatctccagagacaattccaagaacacgctgtatcttcaaatgaacagcctgagagccgaggacacggctgtgtattactgtgcgagagatctgtttcataggagtggttatcacgactactggggccagggaaccctggtcaccgtctcctcag

SEQ ID NO: 22 - protein, 119 aa, Homo sapiens
QVQLVESGGGLVQPGGSLRLSCAVSGFTVSRNYMSWVRQAPGKGLEWVSLIYSGGSTFYADSVKGRFTISRDNSKNTLYLQMNSLRAEDTAVYYCARDLFHRSGYHDYWGQGTLVTVSS

SEQ ID NO: 23 - DNA, 319 bp, Homo sapiens
  source 1..319: mol_type = other DNA
gtcatctggatgacccagtctccatcctccctgtctgcatctgtaggagacagagtcaccatcacttgccaggcgagtcaggacattaacaactatttaaattggtatcagcagaaaccagggaaagcccctaagctcctgatcttcgatgcctccaatttggaaacaggggtcccatcaaggttcagtggcagtggatctgggacagattttactttcaccatcagcagcctacagcctgaagatattgcaacatattactgtcaacagtatgataatctccctgccttcggcggagggaccaaagtggatatcaaac

SEQ ID NO: 24 - protein, 106 aa, Homo sapiens
VIWMTQSPSSLSASVGDRVTITCQASQDINNYLNWYQQKPGKAPKLLIFDASNLETGVPSRFSGSGSGTDFTFTISSLQPEDIATYYCQQYDNLPAFGGGTKVDIK

SEQ ID NO: 25 - protein, 8 aa, Homo sapiens
GFTVSRNY

SEQ ID NO: 26 - protein, 7 aa, Homo sapiens
IYSGGST

SEQ ID NO: 27 - protein, 13 aa, Homo sapiens
ARDLFHRSGYHDY

SEQ ID NO: 28 - protein, 6 aa, Homo sapiens
QDINNY

SEQ ID NO: 29 - skipped

SEQ ID NO: 30 - protein, 8 aa, Homo sapiens
QQYDNLPA

SEQ ID NO: 31 - DNA, 361 bp, Homo sapiens
  source 1..361: mol_type = other DNA
gaagtgcagctggtggagtctgggggaggcgtggtccagcctgggaggtccctgagactctcctgtgcagcgtctggattcaccttcagtaactatggcatgcactgggtccgccaggctccaggcaaggggctggagtgggtggcggttgtatggtatgatggaagcaagaaatactatgcagactccgtgaagggccgattcaccatctccagagacaattccaagaacaccctgtatctgcaaatgaacagcctgagagtcgaggacacggctgtgtattactgcgcgagagattttgcggtgggggaggagatcgctgactcctggggccagggaaccctggtcaccgtctcctcag

SEQ ID NO: 32 - protein, 120 aa, Homo sapiens
EVQLVESGGGVVQPGRSLRLSCAASGFTFSNYGMHWVRQAPGKGLEWVAVVWYDGSKKYYADSVKGRFTISRDNSKNTLYLQMNSLRVEDTAVYYCARDFAVGEEIADSWGQGTLVTVSS

SEQ ID NO: 33 - DNA, 325 bp, Homo sapiens
  source 1..325: mol_type = other DNA
tcctatgagctgactcagccaccctcggtgtcagtgtccccaggacaaacggccaggatcacctgctctggagatgcattgccaaaaaaatatgcttattggtaccagcagaagtcaggccaggcccctgtactggtcatctatgaggacagcaaacgaccctccgggatccctgagagattctctgggtccagctcagggacaatggccaccttgactatcagtggggcccaggtggaggatgaaggtgactactactgttactcaagagacagcagtggtgatcattgggtgttcggcgcagggaccaagctgaccgtcctag

SEQ ID NO: 34 - protein, 108 aa, Homo sapiens
SYELTQPPSVSVSPGQTARITCSGDALPKKYAYWYQQKSGQAPVLVIYEDSKRPSGIPERFSGSSSGTMATLTISGAQVEDEGDYYCYSRDSSGDHWVFGAGTKLTVL

SEQ ID NO: 35 - protein, 8 aa, Homo sapiens
GFTFSNYG

SEQ ID NO: 36 - protein, 8 aa, Homo sapiens
VWYDGSKK

SEQ ID NO: 37 - protein, 13 aa, Homo sapiens
ARDFAVGEEIADS

SEQ ID NO: 38 - protein, 6 aa, Homo sapiens
ALPKKY

SEQ ID NO: 39 - skipped

SEQ ID NO: 40 - protein, 11 aa, Homo sapiens
YSRDSSGDHWV

SEQ ID NO: 41 - DNA, 363 bp, Homo sapiens
  source 1..363: mol_type = other DNA
caggtgcagctggtggagtctgggggaggcgtggtccagcctgggaggtccctgagactctcctgtgcagcctctggattcaccttcagtacctatgctatgcactgggtccgccaggctccaggcaaggggctggagtgggtggctgttctttcatatgatggaagcaataaatactacgcagactccgtgaagggccgattcaccatctccagagacaattccaagaacacgctgtatctgcaaatgaacagcctgagagctgaggacacggctgtgtattactgtgcgaaagggggctcgtacgcgtactactactacatggacgtctggggcaaagggaccacggtcaccgtctcctca

SEQ ID NO: 42 - protein, 121 aa, Homo sapiens
QVQLVESGGGVVQPGRSLRLSCAASGFTFSTYAMHWVRQAPGKGLEWVAVLSYDGSNKYYADSVKGRFTISRDNSKNTLYLQMNSLRAEDTAVYYCAKGGSYAYYYYMDVWGKGTTVTVSS

SEQ ID NO: 43 - DNA, 322 bp, Homo sapiens
  source 1..322: mol_type = other DNA
gacatccagttgacccagtctccatcctccctgtctgcatctgtaggagacagagtcaccatcacttgccaggcgagtcaggacattagcaactatttaaattggtatcagcagaaaccagggaaagcccctaagctcctgatctacgatgcatccaatttggaaacaggggtcccatcaaggttcagtggaggtggatctgggacagattttactttcaccatcaccagcctgcagcctgaagatattgcaacatattactgtcaacagtatgataatctcccgctcactttcggcggagggaccaaagtggatatcaaac

SEQ ID NO: 44 - protein, 107 aa, Homo sapiens
DIQLTQSPSSLSASVGDRVTITCQASQDISNYLNWYQQKPGKAPKLLIYDASNLETGVPSRFSGGGSGTDFTFTITSLQPEDIATYYCQQYDNLPLTFGGGTKVDIK

SEQ ID NO: 45 - protein, 8 aa, Homo sapiens
GFTFSTYA

SEQ ID NO: 46 - protein, 8 aa, Homo sapiens
LSYDGSNK

SEQ ID NO: 47 - protein, 14 aa, Homo sapiens
AKGGSYAYYYYMDV

SEQ ID NO: 48 - protein, 6 aa, Homo sapiens
QDISNY

SEQ ID NO: 49 - skipped

SEQ ID NO: 50 - protein, 9 aa, Homo sapiens
QQYDNLPLT

SEQ ID NO: 51 - DNA, 376 bp, Homo sapiens
  source 1..376: mol_type = other DNA
gttcagctggtgcaggagtcgggcccaggactggtgaagccttcggagaccctgtccctcacctgcactgtctctggtggctccgtcagtagtggtagttactactggagctggatccggcagcccccagggaagggactggagtggattgggtatatgtatttcagtgggagcaccaactataatccctccctcaagagtcgagtcaccatatcattagccacgtccaagaaccagttctccctgaagctgagctctgtgaccgctgcggacacggccgtctattactgtgcgagaggggattacgatttttggagtggtccccccggtcgggtggacgtctggggcaaagggaccacggtcaccgtctcctcag

SEQ ID NO: 52 - protein, 125 aa, Homo sapiens
VQLVQESGPGLVKPSETLSLTCTVSGGSVSSGSYYWSWIRQPPGKGLEWIGYMYFSGSTNYNPSLKSRVTISLATSKNQFSLKLSSVTAADTAVYYCARGDYDFWSGPPGRVDVWGKGTTVTVSS

SEQ ID NO: 53 - DNA, 325 bp, Homo sapiens
  source 1..325: mol_type = other DNA
gaaatagtgatgacgcagtctccaggcaccctgtctttgtctccaggggaaagagccaccctctcctgcagggccagtcagagtgttagcagcagctacttagcctggtaccagcagaaacctggccaggctcccaggctcctcatctatggtgcatccagcagggccactggcatcccagacaggttcagtggcagtgggtctgggacagacttcactctcaccatcagcagactggagcctgaagattttgcagtgtattactgtcagcactatggtagttcacccgtaacttttggccaggggaccaaagtggatatcaaac

SEQ ID NO: 54 - protein, 108 aa, Homo sapiens
EIVMTQSPGTLSLSPGERATLSCRASQSVSSSYLAWYQQKPGQAPRLLIYGASSRATGIPDRFSGSGSGTDFTLTISRLEPEDFAVYYCQHYGSSPVTFGQGTKVDIK

SEQ ID NO: 55 - protein, 10 aa, Homo sapiens
GGSVSSGSYY

SEQ ID NO: 56 - protein, 7 aa, Homo sapiens
MYFSGST

SEQ ID NO: 57 - protein, 17 aa, Homo sapiens
ARGDYDFWSGPPGRVDV

SEQ ID NO: 58 - protein, 7 aa, Homo sapiens
QSVSSSY

SEQ ID NO: 59 - skipped

SEQ ID NO: 60 - protein, 9 aa, Homo sapiens
QHYGSSPVT

SEQ ID NO: 61 - DNA, 367 bp, Homo sapiens
  source 1..367: mol_type = other DNA
caggtgcagctggtgcagtctgggcctgaggtgaagaagcctgggacctcagtgaaggtctcctgcaaggcttctggattcacctttactagctctgctgtgcagtgggtgcgacaggctcgtggacaacgccttgagtggataggatggatcgtcgttggcagtggtaacacaaactacgcacagaagttccaggaaagagtcaccattaccagggacatgtccacaagcacagcctacatggagatgagcagcctgagatccgaggacacggccgtgtattactgtgcggcaccggcctgtggtaccagctgctctgatgcctttgatatctggggccaagggacaatggtcaccgtctcttcag

SEQ ID NO: 62 - protein, 122 aa, Homo sapiens
QVQLVQSGPEVKKPGTSVKVSCKASGFTFTSSAVQWVRQARGQRLEWIGWIVVGSGNTNYAQKFQERVTITRDMSTSTAYMEMSSLRSEDTAVYYCAAPACGTSCSDAFDIWGQGTMVTVSS

SEQ ID NO: 63 - DNA, 325 bp, Homo sapiens
  source 1..325: mol_type = other DNA
gacatccagatgacccagtctccaggcaccctgtctttgtctccaggggaaagagccaccctctcctgcagggccagtcagagtgttagcagcagctacttagcctggtaccagcagaaacctggccaggctcccaggctcctcatctatggtgcatccagcagggccactggcatcccagacaggttcagtggcagtgggtctgggacagacttcactctcaccatcagcagactggagcctgaagattttggagtgtattactgtcagcagtatggtagctcaccgtggacgttcggccaagggaccaaggtggaaatcaaac

SEQ ID NO: 64 - protein, 108 aa, Homo sapiens
DIQMTQSPGTLSLSPGERATLSCRASQSVSSSYLAWYQQKPGQAPRLLIYGASSRATGIPDRFSGSGSGTDFTLTISRLEPEDFGVYYCQQYGSSPWTFGQGTKVEIK

SEQ ID NO: 65 - protein, 8 aa, Homo sapiens
GFTFTSSA

SEQ ID NO: 66 - protein, 8 aa, Homo sapiens
IVVGSGNT

SEQ ID NO: 67 - protein, 15 aa, Homo sapiens
AAPACGTSCSDAFDI

SEQ ID NO: 68 - protein, 7 aa, Homo sapiens
QSVSSSY

SEQ ID NO: 69 - skipped

SEQ ID NO: 70 - protein, 9 aa, Homo sapiens
QQYGSSPWT

SEQ ID NO: 71 - DNA, 382 bp, Homo sapiens
  source 1..382: mol_type = other DNA
caggtgcagctggtggagtctgggggaggcttggtacagcctggcaggtccctgagactctcctgtgcagcctctggattcacctttgatgattatgccatgcactgggtccggcaacctccagggaagggcctggagtgggtctcaggtgtcagttggaacagtggtaccataggctatgcggactctgtgaagggccgattcatcatctccagagacaacgccaagaactccctgtatctgcaaatgaacagtctgaaagctgaggacacggccttgtattactgtgcaagagaagtgggggggacttttggagtccttatttcacgcgaggggggacttgattactggggccagggaaccctggtcaccgtctcctcag

SEQ ID NO: 72 - protein, 127 aa, Homo sapiens
QVQLVESGGGLVQPGRSLRLSCAASGFTFDDYAMHWVRQPPGKGLEWVSGVSWNSGTIGYADSVKGRFIISRDNAKNSLYLQMNSLKAEDTALYYCAREVGGTFGVLISREGGLDYWGQGTLVTVSS

SEQ ID NO: 73 - DNA, 325 bp, Homo sapiens
  source 1..325: mol_type = other DNA
tcctatgagctgacacagccaccctcggtgtcagtggccccaggacagacggccagaattacctgtgggggaaacaccattggaagtaaaagtgtgcactggtaccagcagagaccaggccaggcccctgtgctggtcgtctatgatgatagcgaccggccctcagggatccctgagcgattctctggctccaactctgggaacacggccaccctgaccatcagcagggtcgaagccggggatgaggccgactattactgtcaggtgtgggatagtagtagtgatcgggtggtattcggcggagggaccaagctgaccgtcctag

SEQ ID NO: 74 - protein, 108 aa, Homo sapiens
SYELTQPPSVSVAPGQTARITCGGNTIGSKSVHWYQQRPGQAPVLVVYDDSDRPSGIPERFSGSNSGNTATLTISRVEAGDEADYYCQVWDSSSDRVVFGGGTKLTVL

SEQ ID NO: 75 - protein, 8 aa, Homo sapiens
GFTFDDYA

SEQ ID NO: 76 - protein, 8 aa, Homo sapiens
VSWNSGTI

SEQ ID NO: 77 - protein, 20 aa, Homo sapiens
AREVGGTFGVLISREGGLDY

SEQ ID NO: 78 - protein, 6 aa, Homo sapiens
TIGSKS

SEQ ID NO: 79 - skipped

SEQ ID NO: 80 - protein, 11 aa, Homo sapiens
QVWDSSSDRVV

SEQ ID NO: 81 - DNA, 385 bp, Homo sapiens
  source 1..385: mol_type = other DNA
caggtgcagctgcaggagtcgggcccaggactggtgaagccttcggagaccctgtccctcatctgcactgtctctggtggctccgtcagcagtggtaatttctactggagctggatccggcagcccccagggaagggactggagtggattggatctatctattacactgggagccccaactacaacccctccctcaagagtcgagtcaccatatccctagacacgtccaagaaccagttctccctgaagctgagctctgtgaccgctgcggacacggccgtgtattactgtgcgagagagatctattattatgatagaagtggttcttacaactctgatgcttttgatatctggggccaagggacaatggtcaccgtctcttcag

SEQ ID NO: 82 - protein, 128 aa, Homo sapiens
QVQLQESGPGLVKPSETLSLICTVSGGSVSSGNFYWSWIRQPPGKGLEWIGSIYYTGSPNYNPSLKSRVTISLDTSKNQFSLKLSSVTAADTAVYYCAREIYYYDRSGSYNSDAFDIWGQGTMVTVSS

SEQ ID NO: 83 - DNA, 325 bp, Homo sapiens
  source 1..325: mol_type = other DNA
gatatcgtgatgactcagtctccagccaccctgtctgtgtctccaggggaaagaggcaccctctcctgcagggccagtcagagtgttagcagcaacttagcctggtaccagcagaaaccgggccaggctcccaggctcctcatctatggtgcatccacgagggccactggtatcccagccaggttcagtggcagtgggtctgggacagagttcactctcaccatcagcagcctgcagtctgaagattttgcagtttattactgccagcagtataataactggcctccgctcactttcggcggagggaccaaagtggatatcaaac

SEQ ID NO: 84 - protein, 108 aa, Homo sapiens
DIVMTQSPATLSVSPGERGTLSCRASQSVSSNLAWYQQKPGQAPRLLIYGASTRATGIPARFSGSGSGTEFTLTISSLQSEDFAVYYCQQYNNWPPLTFGGGTKVDIK

SEQ ID NO: 85 - protein, 10 aa, Homo sapiens
GGSVSSGNFY

SEQ ID NO: 86 - protein, 7 aa, Homo sapiens
IYYTGSP

SEQ ID NO: 87 - protein, 20 aa, Homo sapiens
AREIYYYDRSGSYNSDAFDI

SEQ ID NO: 88 - protein, 6 aa, Homo sapiens
QSVSSN

SEQ ID NO: 89 - skipped

SEQ ID NO: 90 - protein, 10 aa, Homo sapiens
QQYNNWPPLT

SEQ ID NO: 91 - DNA, 381 bp, Homo sapiens
  source 1..381: mol_type = other DNA
caggtgcagctggtggagtctgggggaggcgtggttcagcctgggaggtccctgagactctcctgtgcagcctctggattcaccttcaataactatcctttgcactgggtccgccaggctccaggcaaggggccggagtgggtggcagttatttcacaggatggaggcaataaatactacgtagactccgtgaagggccgattcaccatctccagagacaattccaagaacaccctgtatctgcaaatgaacaacctgagagctgaggacacggctctgtattactgtgcgagagatgttgtagtggtggtagctgctaggaaccactactacaacggtatggacgtctggggccaagggaccacggtcaccgtctcctca

SEQ ID NO: 92 - protein, 127 aa, Homo sapiens
QVQLVESGGGVVQPGRSLRLSCAASGFTFNNYPLHWVRQAPGKGPEWVAVISQDGGNKYYVDSVKGRFTISRDNSKNTLYLQMNNLRAEDTALYYCARDVVVVVAARNHYYNGMDVWGQGTTVTVSS

SEQ ID NO: 93 - DNA, 322 bp, Homo sapiens
  source 1..322: mol_type = other DNA
gacatccagttgacccagtctccatcttccgtgtctgcatctgtaggagacagagtcaccatcacttgtcgggcgagtcagggtattagcagctggttagcctggtatcagcagaaaccagggaaagcccctaagctcctgatctatgctgtatccagtttgcaaagtggggtcccatcaaggttcagcggcagtggatctgggacagatttcactctcaccatcagcagcctgcagcctgaagattttgcaacttactattgtcaacaggctaagagtttccctttcactttcggccctgggaccaaggtggagattaaac

SEQ ID NO: 94 - protein, 107 aa, Homo sapiens
DIQLTQSPSSVSASVGDRVTITCRASQGISSWLAWYQQKPGKAPKLLIYAVSSLQSGVPSRFSGSGSGTDFTLTISSLQPEDFATYYCQQAKSFPFTFGPGTKVEIK

SEQ ID NO: 95 - protein, 8 aa, Homo sapiens
GFTFNNYP

SEQ ID NO: 96 - protein, 8 aa, Homo sapiens
ISQDGGNK

SEQ ID NO: 97 - protein, 20 aa, Homo sapiens
ARDVVVVVAARNHYYNGMDV

SEQ ID NO: 98 - protein, 6 aa, Homo sapiens
QGISSW

SEQ ID NO: 99 - skipped

SEQ ID NO: 100 - protein, 9 aa, Homo sapiens
QQAKSFPFT

SEQ ID NO: 101 - DNA, 381 bp, Homo sapiens
  source 1..381: mol_type = other DNA
cagctgcagctgcaggagtcgggcccaggactggtgaagccttcacagaccctgtccctcacctgcactgtctctggtggctccatcagtagtggtagttataattggacctggatccggcagcccgccgggaagggactggagtggattgggcgtatatataatagtgggagcaccaactacaacccctccctcaagagtcgagtcaccatatcagtagacacgtccaagaaccagttgtccctgaaggtgaggtctgtgaccgccgcagacacggccgtgtattactgtgcgagacattgcagtggtggtacctgctacccgaagtactactacggtatggacgtctggggccaagggaccacggtcaccgtctcctca

SEQ ID NO: 102 - protein, 127 aa, Homo sapiens
QLQLQESGPGLVKPSQTLSLTCTVSGGSISSGSYNWTWIRQPAGKGLEWIGRIYNSGSTNYNPSLKSRVTISVDTSKNQLSLKVRSVTAADTAVYYCARHCSGGTCYPKYYYGMDVWGQGTTVTVSS

SEQ ID NO: 103 - DNA, 331 bp, Homo sapiens
  source 1..331: mol_type = other DNA
caatctgccctgactcagccaccctcggtgtctgaagcccccaggcagagggtcaccatctcctgttctggaagcagctccaacatcggaaataatgctgtaaactggtaccagcagttcccaggaaaggctcccaaactcctcatctattatgatgatctgctgccctcaggggtctctgaccgattctctggctccaagtctggcacctcagcctccctggccatcagtggggtccagtctgaggatgaggctgattattactgtgcagcatgggatgacagcctgaatgtcgtggtattcggcggagggaccaagctgaccgtcctag

SEQ ID NO: 104 - protein, 110 aa, Homo sapiens
QSALTQPPSVSEAPRQRVTISCSGSSSNIGNNAVNWYQQFPGKAPKLLIYYDDLLPSGVSDRFSGSKSGTSASLAISGVQSEDEADYYCAAWDDSLNVVVFGGGTKLTVL

SEQ ID NO: 105 - protein, 10 aa, Homo sapiens
GGSISSGSYN

SEQ ID NO: 106 - protein, 7 aa, Homo sapiens
IYNSGST

SEQ ID NO: 107 - protein, 19 aa, Homo sapiens
ARHCSGGTCYPKYYYGMDV

SEQ ID NO: 108 - protein, 8 aa, Homo sapiens
SSNIGNNA

SEQ ID NO: 109 - skipped

SEQ ID NO: 110 - protein, 11 aa, Homo sapiens
AAWDDSLNVVV

SEQ ID NO: 111 - DNA, 370 bp, Homo sapiens
  source 1..370: mol_type = other DNA
caggtgcagctggtggagtcgggcccaggactggtgaagccttcggagaccctgtccctcacctgcactgtctctggtggctccatcagcagtaatagttacttctggggctggatccgccagcccccagggacggggctggagtggattgggaatatctattatactgggagcacctactacaacccgtcgttcgagagtcgagtcaccatgtccgtagacacgtcgaagaaccagttctccctgaggctgagctctgtgaccgccgcagacacggctgtgtattactgtgcgagacatgtcagggcctacgactatgatgccccttttgatatctggggccaagggacaatggtcaccgtctcttcag

SEQ ID NO: 112 - protein, 123 aa, Homo sapiens
QVQLVESGPGLVKPSETLSLTCTVSGGSISSNSYFWGWIRQPPGTGLEWIGNIYYTGSTYYNPSFESRVTMSVDTSKNQFSLRLSSVTAADTAVYYCARHVRAYDYDAPFDIWGQGTMVTVSS

SEQ ID NO: 113 - DNA, 322 bp, Homo sapiens
  source 1..322: mol_type = other DNA
gtcatctggatgacccagtctccatcctccctgtctgcatctgtaggagacagagtcaccatcacttgccgggcaagtcagggcattagaaatgatttaggctggtatcagcagaaaccagggaaagcccctaagcgcctgatctatgctgcatccagtttgcaaagtggggtcccatcaaggttcagcggcagtggatctgggacacaattcactctcacaatcagcagcctgcagcctgaagattttgcaacttattactgtctacagattaatagttatccgctcactttcggcggagggaccaaggtggaaatcaaac

SEQ ID NO: 114 - protein, 107 aa, Homo sapiens
VIWMTQSPSSLSASVGDRVTITCRASQGIRNDLGWYQQKPGKAPKRLIYAASSLQSGVPSRFSGSGSGTQFTLTISSLQPEDFATYYCLQINSYPLTFGGGTKVEIK

SEQ ID NO: 115 - protein, 10 aa, Homo sapiens
GGSISSNSYF

SEQ ID NO: 116 - protein, 7 aa, Homo sapiens
IYYTGST

SEQ ID NO: 117 - protein, 15 aa, Homo sapiens
ARHVRAYDYDAPFDI

SEQ ID NO: 118 - protein, 6 aa, Homo sapiens
QGIRND

SEQ ID NO: 119 - skipped

SEQ ID NO: 120 - protein, 9 aa, Homo sapiens
LQINSYPLT

SEQ ID NO: 121 - DNA, 349 bp, Homo sapiens
  source 1..349: mol_type = other DNA
caggtacagctgcagcagtggggcgcaggactgttgaagccttcggagaccctgtccctcacctgcgctgtctatggtgggtccttcagtggttactactggagctggatccgccagcccccagggaaggggctggagtggattggggaaatcaatcatagtggaagcaccaactacaacccgtccctcaagagtcgagtcaccatatcagtagacacgtccaagaaccagttctccctgaagctgagttctgtgaccgccgcggacacggctgtgtattactgtgcgagaactgattactatgatagtatagactggggccagggaaccctggtcaccgtctcctcag

SEQ ID NO: 122 - protein, 116 aa, Homo sapiens
QVQLQQWGAGLLKPSETLSLTCAVYGGSFSGYYWSWIRQPPGKGLEWIGEINHSGSTNYNPSLKSRVTISVDTSKNQFSLKLSSVTAADTAVYYCARTDYYDSIDWGQGTLVTVSS

SEQ ID NO: 123 - DNA, 328 bp, Homo sapiens
  source 1..328: mol_type = other DNA
cagtctgtgctgactcaggagccctcactgactgtgtccccaggagggacagtcactctcacctgtggctccagcactggagctgtcaccagtggtcattatccctactggttccagcagaagcctggccaagtccccaggacactgatttatgatacaaggaacaaacactcctggacccctgcccggttctcaggctccctccttgggggcaaagctgccctgaccctttcgggtgcgcagcctgaggatgaggctgaatattactgcttgctctcctctagtggtgctcgggtgttcggcggagggaccaagctgaccgtcctag

SEQ ID NO: 124 - protein, 109 aa, Homo sapiens
QSVLTQEPSLTVSPGGTVTLTCGSSTGAVTSGHYPYWFQQKPGQVPRTLIYDTRNKHSWTPARFSGSLLGGKAALTLSGAQPEDEAEYYCLLSSSGARVFGGGTKLTVL

SEQ ID NO: 125 - protein, 8 aa, Homo sapiens
GGSFSGYY

SEQ ID NO: 126 - protein, 7 aa, Homo sapiens
INHSGST

SEQ ID NO: 127 - protein, 10 aa, Homo sapiens
ARTDYYDSID

SEQ ID NO: 128 - protein, 9 aa, Homo sapiens
TGAVTSGHY

SEQ ID NO: 129 - skipped

SEQ ID NO: 130 - protein, 9 aa, Homo sapiens
LLSSSGARV

SEQ ID NO: 131 - DNA, 358 bp, Homo sapiens
  source 1..358: mol_type = other DNA
gaggtgcagctggtggagtctgggggaggcttggtacagcctggggggtccctgagactctcctgtgcagcctctggattcaccttcagtacctacgacatccactgggtccgccaagctacaggaaaaggtctggagtgggtctcagctattggtactgctggtgacacatactattcaggctccgtgaagggccgattcaccatctccagagaaaatgccaagaactccttgtatcttcaaatgaacagcctgagagccggggacacggctgtgtattactgtgcaaggggtagtgggacctacttctactactttgactactggggccagggaaccctggtcaccgtctcctcag

SEQ ID NO: 132 - protein, 119 aa, Homo sapiens
EVQLVESGGGLVQPGGSLRLSCAASGFTFSTYDIHWVRQATGKGLEWVSAIGTAGDTYYSGSVKGRFTISRENAKNSLYLQMNSLRAGDTAVYYCARGSGTYFYYFDYWGQGTLVTVSS

SEQ ID NO: 133 - DNA, 325 bp, Homo sapiens
  source 1..325: mol_type = other DNA
gacatcgtgatgactcagtctccatcctccctgtctgcatctgtaggagacagaatcaccatcacttgccgggcaagtcagagcattaacaactatttaaattggtatcagcagaaaccagggaaagcccctaagctcctgatctatgctgcatcccgtttgcaaactggggtcccatcaaggttcagtggcagtggatctgggacagattccactctcaccatcaacactctgcaacctgaagattttgcaacttactactgtcaacagagttacagtgcccctccgtggacgttcggccaagggaccaaagtggatatcaaac

SEQ ID NO: 134 - protein, 108 aa, Homo sapiens
DIVMTQSPSSLSASVGDRITITCRASQSINNYLNWYQQKPGKAPKLLIYAASRLQTGVPSRFSGSGSGTDSTLTINTLQPEDFATYYCQQSYSAPPWTFGQGTKVDIK

SEQ ID NO: 135 - protein, 8 aa, Homo sapiens
GFTFSTYD

SEQ ID NO: 136 - protein, 7 aa, Homo sapiens
IGTAGDT

SEQ ID NO: 137 - protein, 13 aa, Homo sapiens
ARGSGTYFYYFDY

SEQ ID NO: 138 - protein, 6 aa, Homo sapiens
QSINNY

SEQ ID NO: 139 - skipped

SEQ ID NO: 140 - protein, 10 aa, Homo sapiens
QQSYSAPPWT

SEQ ID NO: 141 - DNA, 366 bp, Homo sapiens
  source 1..366: mol_type = other DNA
caggtgcagctggtggagtcgggcccaggactggtgaagccttcggagaccctgtccctcacctgcactgtctctggtggctcgatcagcagttcttactactggggctggatccgccagcccccagggaaggggctggagtggattgggagtgtctattatagtgggagcacctactacaacccgtccctcaagagtcgagtcaccatatccgtggacacgtccaagaaccagttctccctgaggctgagctctgtgaccgccgcagacacggctgtgtattattgtgcgaggctgatgaccacggaagactactactccggtatggacgtctggggccaagggaccacggtcaccgtctcctca

SEQ ID NO: 142 - protein, 122 aa, Homo sapiens
QVQLVESGPGLVKPSETLSLTCTVSGGSISSSYYWGWIRQPPGKGLEWIGSVYYSGSTYYNPSLKSRVTISVDTSKNQFSLRLSSVTAADTAVYYCARLMTTEDYYSGMDVWGQGTTVTVSS

SEQ ID NO: 143 - DNA, 322 bp, Homo sapiens
  source 1..322: mol_type = other DNA
gccatccagatgacccagtctccatcctcactgtctgcatctgtaggagacagagtcaccatcacttgtcgggcgagtcagggcattagcgattatttagcctggtttcagcagaaaccagggaaagcccctaagtccctgatctatgctgcatccagtttgcaaagtggggtcccatcaaagttcagcggcggtggatctgggacagatttcactctcaccatcagcagcctgcagcctgaagattttgcaacttattactgccaacagtatcatagttacccgatcaccttcggccaagggacacgactggagattaaac

SEQ ID NO: 144 - protein, 107 aa, Homo sapiens
AIQMTQSPSSLSASVGDRVTITCRASQGISDYLAWFQQKPGKAPKSLIYAASSLQSGVPSKFSGGGSGTDFTLTISSLQPEDFATYYCQQYHSYPITFGQGTRLEIK

SEQ ID NO: 145 - protein, 9 aa, Homo sapiens
GGSISSSYY

SEQ ID NO: 146 - protein, 7 aa, Homo sapiens
VYYSGST

SEQ ID NO: 147 - protein, 15 aa, Homo sapiens
ARLMTTEDYYSGMDV

SEQ ID NO: 148 - protein, 6 aa, Homo sapiens
QGISDY

SEQ ID NO: 149 - skipped

SEQ ID NO: 150 - protein, 9 aa, Homo sapiens
QQYHSYPIT

SEQ ID NO: 151 - DNA, 351 bp, Homo sapiens
  source 1..351: mol_type = other DNA
caggtgcagctggtggagtctggaggaggcttgatccagcctggggggtccctgagactctcctgtgcagcctctggggtcaccgtcagtagcaactacatgagttgggtccgccaggctccagggaaggggctggagtgggtctcaattatttatagtggtggtaccacatactacgcagactccgtgaagggccgattcaccatctccagagactcttccatgaacacgctgtatcttcaaatgaacagcctgagagccgaggacacggccgtgtattactgtgcgagagatctgatggtgtacggtatagacgtctggggccaagggaccacggtcaccgtctcctca

SEQ ID NO: 152 - protein, 117 aa, Homo sapiens
QVQLVESGGGLIQPGGSLRLSCAASGVTVSSNYMSWVRQAPGKGLEWVSIIYSGGTTYYADSVKGRFTISRDSSMNTLYLQMNSLRAEDTAVYYCARDLMVYGIDVWGQGTTVTVSS

SEQ ID NO: 153 - DNA, 328 bp, Homo sapiens
  source 1..328: mol_type = other DNA
gaaatagtgatgacgcagtctccatcctccctgtctgcatctgtaggagacagagtcaccatcacttgccgggccagtcagggcattagcagttatttagcctggtatcagcaaaaaccagggaaagcccctaagctcctgatctatgctgcatccactttgcaaagtggggtcccatcaaggttcagcggcagtggatctgggacagatttcactctcaccatcagcagcctgcagcctgaagattttgcaacttattactgtcaacagcttgatagttacccccccgggtacacttttggccaggggaccaaagtggatatcaaac

SEQ ID NO: 154 - protein, 109 aa, Homo sapiens
EIVMTQSPSSLSASVGDRVTITCRASQGISSYLAWYQQKPGKAPKLLIYAASTLQSGVPSRFSGSGSGTDFTLTISSLQPEDFATYYCQQLDSYPPGYTFGQGTKVDIK

SEQ ID NO: 155 - protein, 8 aa, Homo sapiens
GVTVSSNY

SEQ ID NO: 156 - protein, 7 aa, Homo sapiens
IYSGGTT

SEQ ID NO: 157 - protein, 11 aa, Homo sapiens
ARDLMVYGIDV

SEQ ID NO: 158 - protein, 6 aa, Homo sapiens
QGISSY

SEQ ID NO: 159 - skipped

SEQ ID NO: 160 - protein, 11 aa, Homo sapiens
QQLDSYPPGYT

SEQ ID NO: 161 - DNA, 351 bp, Homo sapiens
  source 1..351: mol_type = other DNA
gaggtgcagctgttggagtctggaggagacttgatccagcctggggggtccctgagactctcctgtgcagcctctggggtcaccgtcagtagcaactacatgagctgggtccgccaggctccagggaaggggctggagtgggtctcaattatttatcccggtgggagcacattctacgcagactccgtgaagggccgattcaccatctccagagacaattccaagaacacgctgtatcttcaaatgcacagcctgagagccgaggacacggccgtgtattactgtgcgagagatcttggctcaggggacatggacgtctggggcaaagggaccacggtcaccgtctcctca

SEQ ID NO: 162 - protein, 117 aa, Homo sapiens
EVQLLESGGDLIQPGGSLRLSCAASGVTVSSNYMSWVRQAPGKGLEWVSIIYPGGSTFYADSVKGRFTISRDNSKNTLYLQMHSLRAEDTAVYYCARDLGSGDMDVWGKGTTVTVSS

SEQ ID NO: 163 - DNA, 322 bp, Homo sapiens
  source 1..322: mol_type = other DNA
gacatcgtgatgactcagtctccatccttcctgtctgcatctgtaggagacagagtcaccatcacttgccgggccagtcagggcattagcagttatttagcctggtatcagcaaaaaccggggaaagcccctaagctcctgatccaagctgcatccactttgcaaagtggggtcccatcaaggttcagcggcagtggatctgggacagaattcactctcacaatcagcagcctgcagcctgaagattttgcaacttattactgtcaacagcttaatagttaccggtacacttttggccaggggaccaaggtggagatcaaac

SEQ ID NO: 164 - protein, 107 aa, Homo sapiens
DIVMTQSPSFLSASVGDRVTITCRASQGISSYLAWYQQKPGKAPKLLIQAASTLQSGVPSRFSGSGSGTEFTLTISSLQPEDFATYYCQQLNSYRYTFGQGTKVEIK

SEQ ID NO: 165 - protein, 8 aa, Homo sapiens
GVTVSSNY

SEQ ID NO: 166 - protein, 7 aa, Homo sapiens
IYPGGST

SEQ ID NO: 167 - protein, 11 aa, Homo sapiens
ARDLGSGDMDV

SEQ ID NO: 168 - protein, 6 aa, Homo sapiens
QGISSY

SEQ ID NO: 169 - skipped

SEQ ID NO: 170 - protein, 9 aa, Homo sapiens
QQLNSYRYT

SEQ ID NO: 171 - DNA, 369 bp, Homo sapiens
  source 1..369: mol_type = other DNA
gaggtgcagctggtggagtctgggggaggcgtggtccagcctgggaggtccctgagactctcctgtgcagcctctggattcaccttcagtagctatggcatgcactgggtccgccaggctccaggcaaggggctggagtgggtggcacttatatcatatgatggaggtaatagatactatgcagactccgtgaagggccgattcaccatctccagagacaattccaagaacacgctgtatctgcaaatgaacagactgagagctgaagacacggctatgtattactgtgcgaaagatcgtgatgatgggtgggattggtactacttcatggacgtctggggcaaagggaccacggtcaccgtctcctca

SEQ ID NO: 172 - protein, 123 aa, Homo sapiens
EVQLVESGGGVVQPGRSLRLSCAASGFTFSSYGMHWVRQAPGKGLEWVALISYDGGNRYYADSVKGRFTISRDNSKNTLYLQMNRLRAEDTAMYYCAKDRDDGWDWYYFMDVWGKGTTVTVSS

SEQ ID NO: 173 - DNA, 322 bp, Homo sapiens
  source 1..322: mol_type = other DNA
gacatccagttgacccagtctccaggcaccctgtctttgtctccaggggaaagagccaccctctcctgcagggccagtcagagtattagcggcaactacttagcctggtaccagcataaacctggccaggctcccagactcctcatctatggtgcatccaccagggccactggcatcccagacaggttcagtggcagtgggtctgggacagacttcactctcaccatcagcagactggagcctgaagattttgcagtgtattactgtcagcagtatggtagctcgtacacttttggccaggggaccaaggtggagatcaaac

SEQ ID NO: 174 - protein, 107 aa, Homo sapiens
DIQLTQSPGTLSLSPGERATLSCRASQSISGNYLAWYQHKPGQAPRLLIYGASTRATGIPDRFSGSGSGTDFTLTISRLEPEDFAVYYCQQYGSSYTFGQGTKVEIK

SEQ ID NO: 175 - protein, 8 aa, Homo sapiens
GFTFSSYG

SEQ ID NO: 176 - protein, 8 aa, Homo sapiens
ISYDGGNR

SEQ ID NO: 177 - protein, 16 aa, Homo sapiens
AKDRDDGWDWYYFMDV

SEQ ID NO: 178 - protein, 7 aa, Homo sapiens
QSISGNY

SEQ ID NO: 179 - skipped

SEQ ID NO: 180 - protein, 8 aa, Homo sapiens
QQYGSSYT

SEQ ID NO: 181 - DNA, 370 bp, Homo sapiens
  source 1..370: mol_type = other DNA
caggttcagctggtgcagtctgggcctgaggtgaagaagcctgggacctcagtgaaggtctcctgcaaggcttctggattcacctttactagctctgctgtgcagtgggtgcgacaggctcgtggacagcgccttgagtggataggatggatcgtcgttggcagtggtaacacaaactacgcacagaagttccaggaaagcgtcaccattaccagggacatgtccacaagcacagcctacatggagctgagcagcctgagatccgaggacacggccgtgtattactgtgcggccccacattgtattggtggtagctgccatgatgcttttgatatctggggccaagggacaatggtcaccgtctcttcag

SEQ ID NO: 182 - protein, 123 aa, Homo sapiens
QVQLVQSGPEVKKPGTSVKVSCKASGFTFTSSAVQWVRQARGQRLEWIGWIVVGSGNTNYAQKFQESVTITRDMSTSTAYMELSSLRSEDTAVYYCAAPHCIGGSCHDAFDIWGQGTMVTVSS

SEQ ID NO: 183 - DNA, 325 bp, Homo sapiens
  source 1..325: mol_type = other DNA
gatatcgtgatgacccagtctccaggcaccctatctttgtctccaggggaaagagccaccctctcctgcagggccagtcagagtgttagaagcagctacttagcctggtaccagcagaaacctggccaggctcccaggctcctcatctatggtgcatccaggaggggcactggcatcccagacaggttcagtggcagtgggtctgggacagacttcactctcaccatcagcagactggagcctgaagattttgcagtgtattactgtcagcagtatggtagctcaccctggacgttcggccaagggaccaaggtggaaatcaaac

SEQ ID NO: 184 - protein, 108 aa, Homo sapiens
DIVMTQSPGTLSLSPGERATLSCRASQSVRSSYLAWYQQKPGQAPRLLIYGASRRGTGIPDRFSGSGSGTDFTLTISRLEPEDFAVYYCQQYGSSPWTFGQGTKVEIK

SEQ ID NO: 185 - protein, 8 aa, Homo sapiens
GFTFTSSA

SEQ ID NO: 186 - protein, 8 aa, Homo sapiens
IVVGSGNT

SEQ ID NO: 187 - protein, 16 aa, Homo sapiens
AAPHCIGGSCHDAFDI

SEQ ID NO: 188 - protein, 7 aa, Homo sapiens
QSVRSSY

SEQ ID NO: 189 - skipped

SEQ ID NO: 190 - protein, 9 aa, Homo sapiens
QQYGSSPWT

SEQ ID NO: 191 - DNA, 352 bp, Homo sapiens
  source 1..352: mol_type = other DNA
caggtgcagctggtggagtcaggagcagaggtgaaaaagcccggggagtctctgaagatctcctgtaagggttctggatacagctttaccagctactggatcgtctgggtgcgccagatgcccgggaaaggcctggagtggatggggatcatctatcctggtgactctgataccaaatacagtccgtccttccaaggccaggtcagcatctcagccgacaagcccatcagcaccgcctacctgcagtggagcaggctgaaggcctcggacaccgccatgtattactgtgcgagactagggaattggctggtggactactggggccagggaaccctggtcaccgtctcctcag

SEQ ID NO: 192 - protein, 117 aa, Homo sapiens
QVQLVESGAEVKKPGESLKISCKGSGYSFTSYWIVWVRQMPGKGLEWMGIIYPGDSDTKYSPSFQGQVSISADKPISTAYLQWSRLKASDTAMYYCARLGNWLVDYWGQGTLVTVSS

SEQ ID NO: 193 - DNA, 343 bp, Homo sapiens
  source 1..343: mol_type = other DNA
gatattgtgatgactcagtctcctctctctctgtccgtcacccctggacagccggcctccatctcctgcaagtctagtcagagcctcctgcatagtgatggaaagacctatttgtattggtacctgcagaagccaggccagcctccacagctcctgatgtatgaagtttccaaccggttctctggagtgccagataggttcagtggcagcgggtcagggacagacttcacacttaaaatcagccgggtggagtctgaggatgttggggtttattactgcatgcaaagtatacagcttcctcgcgggatcaccttcggccaagggacacgactggagattaaac

SEQ ID NO: 194 - protein, 114 aa, Homo sapiens
DIVMTQSPLSLSVTPGQPASISCKSSQSLLHSDGKTYLYWYLQKPGQPPQLLMYEVSNRFSGVPDRFSGSGSGTDFTLKISRVESEDVGVYYCMQSIQLPRGITFGQGTRLEIK

SEQ ID NO: 195 - protein, 8 aa, Homo sapiens
GYSFTSYW

SEQ ID NO: 196 - protein, 8 aa, Homo sapiens
IYPGDSDT

SEQ ID NO: 197 - protein, 10 aa, Homo sapiens
ARLGNWLVDY

SEQ ID NO: 198 - protein, 11 aa, Homo sapiens
QSLLHSDGKTY

SEQ ID NO: 199 - skipped

SEQ ID NO: 200 - protein, 11 aa, Homo sapiens
MQSIQLPRGIT

SEQ ID NO: 201 - DNA, 343 bp, Homo sapiens
  source 1..343: mol_type = other DNA
gaggtgcagctggtggagtctggaggaggcttgatccagcctggggggtccctgagactctcctgtgcagcctctgggctcaccgtcagtcgcaattacatgagctgggtccgccaggctccagggaaggggctggagtgggtctcacttatttatagcggtggtagcacatactacgcagactccgtgaagggccgattcaccatctccagagacaattccaagaacacgctgtatcttcaaatgaacagcctgagagccgaggacacggccgtgtattactgtgcgagagatctacgcggagaagtctggggccaagggacaatggtcaccgtctcttcag

SEQ ID NO: 202 - protein, 114 aa, Homo sapiens
EVQLVESGGGLIQPGGSLRLSCAASGLTVSRNYMSWVRQAPGKGLEWVSLIYSGGSTYYADSVKGRFTISRDNSKNTLYLQMNSLRAEDTAVYYCARDLRGEVWGQGTMVTVSS

SEQ ID NO: 203 - DNA, 322 bp, Homo sapiens
  source 1..322: mol_type = other DNA
gccatccagatgacccagtctccatcctccctgtctgcatctgtaggagacagagtcaccatcacttgccaggcgagtcaggacattagcaactttttaaattggtatcagcagaaaccagggaaagcccctaagctcctgatctacgatgcatccaatttggaaacaggggtcccatcaaggttcagtggaagtggatctgggacagattttactttcaccatcagtagcctgcagcctgaagatattgcaacatattactgtcaccagtatgataatctccctcgaacgttcggccaagggaccaaagtggatatcaaac

SEQ ID NO: 204 - protein, 107 aa, Homo sapiens
AIQMTQSPSSLSASVGDRVTITCQASQDISNFLNWYQQKPGKAPKLLIYDASNLETGVPSRFSGSGSGTDFTFTISSLQPEDIATYYCHQYDNLPRTFGQGTKVDIK

SEQ ID NO: 205 - protein, 8 aa, Homo sapiens
GLTVSRNY

SEQ ID NO: 206 - protein, 7 aa, Homo sapiens
IYSGGST

SEQ ID NO: 207 - protein, 8 aa, Homo sapiens
ARDLRGEV

SEQ ID NO: 208 - protein, 6 aa, Homo sapiens
QDISNF

SEQ ID NO: 209 - skipped

SEQ ID NO: 210 - protein, 9 aa, Homo sapiens
HQYDNLPRT

SEQ ID NO: 211 - DNA, 372 bp, Homo sapiens
  source 1..372: mol_type = other DNA
gaggtgcagctggtggagtctgggggaggcttggtacagcctggggggtccctgagactctcctgtgcagcctctggattcaccttcagtaactacgacatgcactgggtccgccaagctacaggaaaaggtctggagtgggtctcacttattggtactgctggtgacacatactatccagactccgtgaagggccgattcaccatctccagagaaaatgccaagaactccttgtatcttcaaatgaacagcctgagagccggggacacggctgtgtattactgtgcaagagggcaacacactcaaatcggtcactactactactactacatggacgtctggggcaaagggaccacggtcaccgtctcctca

SEQ ID NO: 212 - protein, 124 aa, Homo sapiens
EVQLVESGGGLVQPGGSLRLSCAASGFTFSNYDMHWVRQATGKGLEWVSLIGTAGDTYYPDSVKGRFTISRENAKNSLYLQMNSLRAGDTAVYYCARGQHTQIGHYYYYYMDVWGKGTTVTVSS

SEQ ID NO: 213 - DNA, 328 bp, Homo sapiens
  source 1..328: mol_type = other DNA
gccatccggatgacccagtctccatcgtccctgtctgcatctgtaggagacagagtcaccatcacttgccgggcaagtcagagcattagcagctatttaaattggtatcagcagaaaccagggaaagcccctaagctcctgatctttgctgcatccagtttgcaaagtggggtcccatcaaggttcagtggcagtggatctgggacagattccactctaaccatcagcagtctgcaacctgaagattttgcaacttactactgtcaacagagttacagtaaccctccggagggcagttttggccaggggaccaaagtggagattaaac

SEQ ID NO: 214 - protein, 109 aa, Homo sapiens
AIRMTQSPSSLSASVGDRVTITCRASQSISSYLNWYQQKPGKAPKLLIFAASSLQSGVPSRFSGSGSGTDSTLTISSLQPEDFATYYCQQSYSNPPEGSFGQGTKVEIK

SEQ ID NO: 215 - protein, 8 aa, Homo sapiens
GFTFSNYD

SEQ ID NO: 216 - protein, 7 aa, Homo sapiens
IGTAGDT

SEQ ID NO: 217 - protein, 18 aa, Homo sapiens
ARGQHTQIGHYYYYYMDV

SEQ ID NO: 218 - protein, 6 aa, Homo sapiens
QSISSY

SEQ ID NO: 219 - skipped

SEQ ID NO: 220 - protein, 11 aa, Homo sapiens
QQSYSNPPEGS

SEQ ID NO: 221 - DNA, 361 bp, Homo sapiens
  source 1..361: mol_type = other DNA
gaagtgcagctggtggagactggaggaggcttgatccagcctggggggtccctgagactctcctgtgcagcctctgggttcaccgtcagtagcaactacatgagctgggtccgccaggctccagggaaggggctggagtgggtctcagttgtttatggcggtggtaccacatactacgcagactccgtgaagggccgattcaccatctccagagacaattccaagaacacgctgtatcttcaaatgaacagcctgagagccgaggacacggccgtatattactgtgcgactgacaatggatacagctatggtttttcatttgactactggggccagggaaccctggtcatcgtctcctcag

SEQ ID NO: 222 - protein, 120 aa, Homo sapiens
EVQLVETGGGLIQPGGSLRLSCAASGFTVSSNYMSWVRQAPGKGLEWVSVVYGGGTTYYADSVKGRFTISRDNSKNTLYLQMNSLRAEDTAVYYCATDNGYSYGFSFDYWGQGTLVIVSS

SEQ ID NO: 223 - DNA, 331 bp, Homo sapiens
  source 1..331: mol_type = other DNA
cagtctgtgctgactcagcctgcctccatgtctgggtctcctggacagtcgatcaccatctcctgcactggaaccagcagtgatgttgggggttataaccttgtctcctggtaccaacagcacccaggcaaagcccccaaactcatgatttatgagggcagtaagcggccctcaggggtttctaatcgcttctctggctccaagtctggcaacacggcctccctgacaatctctgggctccaggctgaggacgaggctgattattactgctgctcatatgcaggtagtagtaattgggtgttcggcggagggaccaagctgaccgtcctag

SEQ ID NO: 224 - protein, 110 aa, Homo sapiens
QSVLTQPASMSGSPGQSITISCTGTSSDVGGYNLVSWYQQHPGKAPKLMIYEGSKRPSGVSNRFSGSKSGNTASLTISGLQAEDEADYYCCSYAGSSNWVFGGGTKLTVL

SEQ ID NO: 225 - protein, 8 aa, Homo sapiens
GFTVSSNY

SEQ ID NO: 226 - protein, 7 aa, Homo sapiens
VYGGGTT

SEQ ID NO: 227 - protein, 14 aa, Homo sapiens
ATDNGYSYGFSFDY

SEQ ID NO: 228 - protein, 9 aa, Homo sapiens
SSDVGGYNL

SEQ ID NO: 229 - skipped

SEQ ID NO: 230 - protein, 10 aa, Homo sapiens
CSYAGSSNWV

SEQ ID NO: 231 - DNA, 373 bp, Homo sapiens
  source 1..373: mol_type = other DNA
caggtgcagctggtggagtctggggctgaggtggagaagcctggggcctcagtgaaggtctcctgcaaggcttctggatacaccttcaccggctactatatgcactgggtgcgacaggcccctggacaagggcttgagtggatgggatggatcaaccctatcagtggtggcacaaactatgcacagaagtttcagggcagggtcaccatgaccagggacacgtccatcagcacagcctacatggacctgagcaggctgagatctgacgacacggccgtgtattactgtgcgagaggaacgtattactatgatagtagtggttacatcccatttgactactggggccagggaaccctggtcaccgtctcctcag

SEQ ID NO: 232 - protein, 124 aa, Homo sapiens
QVQLVESGAEVEKPGASVKVSCKASGYTFTGYYMHWVRQAPGQGLEWMGWINPISGGTNYAQKFQGRVTMTRDTSISTAYMDLSRLRSDDTAVYYCARGTYYYDSSGYIPFDYWGQGTLVTVSS

SEQ ID NO: 233 - DNA, 331 bp, Homo sapiens
  source 1..331: mol_type = other DNA
cagtctgtgctgactcagcctgcctccgtatctgggtctcctggacagtcgatcaccatctcctgcactggaaccagcagtgatgttgggagttataaccttgtctcctggtaccaacagcacccaggcaaagcccccaaactcatgatttatgagggcagtaagcggccctcaggggtttctaatcgcttctctggctccaagtctggcaacacggcctccctgacaatctctgggctccaggctgaggacgaggctgattattactgctgctcatatgcaggtagtagcactttggtattcggcggagggaccaagctgaccgtcctag

SEQ ID NO: 234 - protein, 110 aa, Homo sapiens
QSVLTQPASVSGSPGQSITISCTGTSSDVGSYNLVSWYQQHPGKAPKLMIYEGSKRPSGVSNRFSGSKSGNTASLTISGLQAEDEADYYCCSYAGSSTLVFGGGTKLTVL

SEQ ID NO: 235 - protein, 8 aa, Homo sapiens
GYTFTGYY

SEQ ID NO: 236 - protein, 8 aa, Homo sapiens
INPISGGT

SEQ ID NO: 237 - protein, 17 aa, Homo sapiens
ARGTYYYDSSGYIPFDY

SEQ ID NO: 238 - protein, 9 aa, Homo sapiens
SSDVGSYNL

SEQ ID NO: 239 - skipped

SEQ ID NO: 240 - protein, 10 aa, Homo sapiens
CSYAGSSTLV

SEQ ID NO: 241 - DNA, 385 bp, Homo sapiens
  source 1..385: mol_type = other DNA
caggttcagctggtgcagtctgggtctgagttgaagaagcctggggcctcagtgaaggtttcctgcaaggcttctggatacaccttcagtagctatgctatgacttgggtgcgacaggcccctggacaagggcttgagtggatgggatggatcaacaccaacactgggaacccaacgtatgcccagggcttcacaggacggtttgtcttctccttggacacctctgtcagcacggcatatctgcagatcagcagcctaaaggctgaggacactgccgtgtattactgtgcgagagctctgggatattgtagtagtaccagctgctatcccgcttgggctgcttttgatatctggggccaagggacaatggtcaccgtctcttcag

SEQ ID NO: 242 - protein, 128 aa, Homo sapiens
QVQLVQSGSELKKPGASVKVSCKASGYTFSSYAMTWVRQAPGQGLEWMGWINTNTGNPTYAQGFTGRFVFSLDTSVSTAYLQISSLKAEDTAVYYCARALGYCSSTSCYPAWAAFDIWGQGTMVTVSS

SEQ ID NO: 243 - DNA, 316 bp, Homo sapiens
  source 1..316: mol_type = other DNA
tcctatgagctgactcagccactctcagtgtcagtggccctgggacagacggccagtattacctgtgggggaaacaacattggaagtaaaaatgtgcactggtaccagcagaagccaggccaggcccctgtgctggtcatctatagggatagcaaccggccctctgggatccctgagcgattctctggctccaactcggggaacacggccaccctgaccatcagcagagcccaagccggggatgaggctgactataactgtcaggtgtgggacagcagcgtggtattcggcggagggaccaagctgaccgtcctag

SEQ ID NO: 244 - protein, 105 aa, Homo sapiens
SYELTQPLSVSVALGQTASITCGGNNIGSKNVHWYQQKPGQAPVLVIYRDSNRPSGIPERFSGSNSGNTATLTISRAQAGDEADYNCQVWDSSVVFGGGTKLTVL

SEQ ID NO: 245 - protein, 8 aa, Homo sapiens
GYTFSSYA

SEQ ID NO: 246 - protein, 8 aa, Homo sapiens
INTNTGNP

SEQ ID NO: 247 - protein, 21 aa, Homo sapiens
ARALGYCSSTSCYPAWAAFDI

SEQ ID NO: 248 - protein, 6 aa, Homo sapiens
NIGSKN

SEQ ID NO: 249 - skipped

SEQ ID NO: 250 - protein, 8 aa, Homo sapiens
QVWDSSVV

SEQ ID NO: 251 - DNA, 358 bp, Homo sapiens
  source 1..358: mol_type = other DNA
gaggtgcagctggtggagtctggaggaggcttgatccagccgggggggtccctgagactctcctgtgcagcctctgggctcaccgtcagtagcaactacatgagttgggtccgccaggctccagggaaggggctggagtgggtctcagttatttatagtggtggtagcacgttctacgcagactccgtgaagggccgattcaccatctccagagacaattccaagaacacgctgtatcttcaaatgaacagcctgggagccgaggacacggccgtgtattactgtgcgagaggagaaggtagtcctggaaactggttcgacccctggggccagggaaccctggtcaccgtctcctcag

SEQ ID NO: 252 - protein, 119 aa, Homo sapiens
EVQLVESGGGLIQPGGSLRLSCAASGLTVSSNYMSWVRQAPGKGLEWVSVIYSGGSTFYADSVKGRFTISRDNSKNTLYLQMNSLGAEDTAVYYCARGEGSPGNWFDPWGQGTLVTVSS

SEQ ID NO: 253 - DNA, 322 bp, Homo sapiens
  source 1..322: mol_type = other DNA
gatgttgtgatgactcagtctccaggcaccctgtctttgtctccaggggaaagagccaccctctcctgcagggccagtcagagtgttcccagcagctacttagcctggtaccagcagaaacctggccaggctcccaggctcctcatctatggtgcatccaccagggccactggcatcccagacaggttcagtggcagtgggtctgggacagacttcactctcaccatcagcagactggagcctgaggattttgcagtgtattactgtcagcactatgatacctcaccccgtttcggcggagggaccaaagtggatatcaaac

SEQ ID NO: 254 - protein, 107 aa, Homo sapiens
DVVMTQSPGTLSLSPGERATLSCRASQSVPSSYLAWYQQKPGQAPRLLIYGASTRATGIPDRFSGSGSGTDFTLTISRLEPEDFAVYYCQHYDTSPRFGGGTKVDIK

SEQ ID NO: 255 - protein, 8 aa, Homo sapiens
GLTVSSNY

SEQ ID NO: 256 - protein, 7 aa, Homo sapiens
IYSGGST

SEQ ID NO: 257 - protein, 13 aa, Homo sapiens
ARGEGSPGNWFDP

SEQ ID NO: 258 - protein, 7 aa, Homo sapiens
QSVPSSY

SEQ ID NO: 259 - skipped

SEQ ID NO: 260 - protein, 8 aa, Homo sapiens
QHYDTSPR

SEQ ID NO: 261 - DNA, 370 bp, Homo sapiens
  source 1..370: mol_type = other DNA
caggtccagctggtacagtctgggcctgaggtgaagaagcctgggacctcagtgaaggtctcctgcaaggcttctggattcacctttactacctctgctgtgcagtgggtgcgacaggctcgtggacaacgccttgagtggataggatggatcgtcgttggcagtggtaacacaaactacgcacagaagttccaggaaagagtcaccattaccagggacatgtccacaaccacagcctacatggagctgagcagcctgagatccgaggacacggccgtgtatttctgtgcggcgcctcattgtaatagtaccagctgctatgacgcttttgatatctggggccaagggacaatggtcaccgtctcttcag

SEQ ID NO: 262 - protein, 123 aa, Homo sapiens
QVQLVQSGPEVKKPGTSVKVSCKASGFTFTTSAVQWVRQARGQRLEWIGWIVVGSGNTNYAQKFQERVTITRDMSTTTAYMELSSLRSEDTAVYFCAAPHCNSTSCYDAFDIWGQGTMVTVSS

SEQ ID NO: 263 - DNA, 325 bp, Homo sapiens
  source 1..325: mol_type = other DNA
gacatccagatgacccagtctccaggcaccctgtctttgtctccaggggaaggagccaccctctcctgcagggccagtcagagtgttagcagcagctacttagcctggtaccagcagaaacctggccaggctcccaggctcctcatctatggtgcatctagtggggccactggcatcccagacagattcagtggcagtgggtctgggacagacttcactctcaccatcagcagactggagcctgaagattttgcagtgtattactgtcagcagtatggtagctcaccttacacttttggccaggggaccaaggtggaaatcaaac

SEQ ID NO: 264 - protein, 108 aa, Homo sapiens
DIQMTQSPGTLSLSPGEGATLSCRASQSVSSSYLAWYQQKPGQAPRLLIYGASSGATGIPDRFSGSGSGTDFTLTISRLEPEDFAVYYCQQYGSSPYTFGQGTKVEIK

SEQ ID NO: 265 - protein, 8 aa, Homo sapiens
GFTFTTSA

SEQ ID NO: 266 - protein, 8 aa, Homo sapiens
IVVGSGNT

SEQ ID NO: 267 - protein, 16 aa, Homo sapiens
AAPHCNSTSCYDAFDI

SEQ ID NO: 268 - protein, 7 aa, Homo sapiens
QSVSSSY

SEQ ID NO: 269 - skipped

SEQ ID NO: 270 - protein, 9 aa, Homo sapiens
QQYGSSPYT

SEQ ID NO: 271 - DNA, 352 bp, Homo sapiens
  source 1..352: mol_type = other DNA
caggtgcagctggtggagtctggaggaggcttgatccagcctggggggtccctgagactctcctgtgcagcctctgggctcaccgtcaataggaactacatgagctggatccgccaggctccagggaaggggctggagtgggtctcagttatttatagcggtggtagtacattttacgcagactccgtgaagggccgattcaccatctccagagacaattccaagaacacactgtctcttcaaatgaacagcctgagagccgaggacacggccatttattactgtgcgagagacttctacgagggttcttttgatatctggggccaagggacaatggtcaccgtctcttcag

SEQ ID NO: 272 - protein, 117 aa, Homo sapiens
QVQLVESGGGLIQPGGSLRLSCAASGLTVNRNYMSWIRQAPGKGLEWVSVIYSGGSTFYADSVKGRFTISRDNSKNTLSLQMNSLRAEDTAIYYCARDFYEGSFDIWGQGTMVTVSS

SEQ ID NO: 273 - DNA, 325 bp, Homo sapiens
  source 1..325: mol_type = other DNA
gccatccagttgacccagtctccttccttcctgtctgcatctataggagacagagtcaccatcacttgccgggccagtcagggcattagcagttatttagcctggtatcagcaaaaaccagggaaagcccctaagctcctgatctatgctgcatccactttgcaaagtggggtcccatcaaggttcagcggcagtggatctgggacagaattcactctcacaatcagcagcctgcagcctgaagattttgcatcttattactgtcaacagcttaatagttaccccgctccggttttcggccctgggaccaaagtggatatcaaac

SEQ ID NO: 274 - protein, 108 aa, Homo sapiens
AIQLTQSPSFLSASIGDRVTITCRASQGISSYLAWYQQKPGKAPKLLIYAASTLQSGVPSRFSGSGSGTEFTLTISSLQPEDFASYYCQQLNSYPAPVFGPGTKVDIK

SEQ ID NO: 275 - protein, 8 aa, Homo sapiens
GLTVNRNY

SEQ ID NO: 276 - protein, 7 aa, Homo sapiens
IYSGGST

SEQ ID NO: 277 - protein, 11 aa, Homo sapiens
ARDFYEGSFDI

SEQ ID NO: 278 - protein, 6 aa, Homo sapiens
QGISSY

SEQ ID NO: 279 - skipped

SEQ ID NO: 280 - protein, 10 aa, Homo sapiens
QQLNSYPAPV

SEQ ID NO: 281 - DNA, 370 bp, Homo sapiens
  source 1..370: mol_type = other DNA
caggtacagctggtgcagtctggggctgaggtgaagaagcctggggcctcagtgaaggtctcctgcaaggcttctggttacatctttatcagatatggtattagctgggtgcgacaggcccctggacaagggcttgagtggatgggatggatcagcgctaacaatggttacacaaactatgcacagaagctccagggcagagtcaccatgaccacagacacatccacgagcacagcctacatggagctgaggagcctgagatctgacgacacggccgtgtattactgtgcgagagatgggggtattttgactggttatctcgactactttgaccactggggccagggaaccctggtcaccgtctcctcag

SEQ ID NO: 282 - protein, 123 aa, Homo sapiens
QVQLVQSGAEVKKPGASVKVSCKASGYIFIRYGISWVRQAPGQGLEWMGWISANNGYTNYAQKLQGRVTMTTDTSTSTAYMELRSLRSDDTAVYYCARDGGILTGYLDYFDHWGQGTLVTVSS

SEQ ID NO: 283 - DNA, 325 bp, Homo sapiens
  source 1..325: mol_type = other DNA
gacatccagatgacccagtctccatcctccctgtctgcatctgtaggagacagactcaccatcacttgccgggcaagtcagagcattgccagctatttaaattggtatcagcagaaaccagggaaagcccctaagctcctgatctatgctgcatccagtttgcaaagtggggtcccatcaaggttcagtggcagtggatctgggacagatttcactctcaccatcagcagtctgcaacctgaagattttgcaacttaccactgtcaacagagttacagtaccctcggaatcactttcggccctgggaccaaagtggatatcaaac

SEQ ID NO: 284 - protein, 108 aa, Homo sapiens
DIQMTQSPSSLSASVGDRLTITCRASQSIASYLNWYQQKPGKAPKLLIYAASSLQSGVPSRFSGSGSGTDFTLTISSLQPEDFATYHCQQSYSTLGITFGPGTKVDIK

SEQ ID NO: 285 - protein, 8 aa, Homo sapiens
GYIFIRYG

SEQ ID NO: 286 - protein, 8 aa, Homo sapiens
ISANNGYT

SEQ ID NO: 287 - protein, 16 aa, Homo sapiens
ARDGGILTGYLDYFDH

SEQ ID NO: 288 - protein, 6 aa, Homo sapiens
QSIASY

SEQ ID NO: 289 - skipped

SEQ ID NO: 290 - protein, 10 aa, Homo sapiens
QQSYSTLGIT

SEQ ID NO: 291 - DNA, 367 bp, Homo sapiens
  source 1..367: mol_type = other DNA
caggtgcagctggtggagtctgggggaggcttggtccagcctggggggtccctgagactctcctgtgcagcctctggattcccctttagtatctattggatgagctgggtccgccaggctccagggaaggggctggagtgggtggccaacataaagcaagatggaagtgagaaatactatgtggactctgtgaagggccgattcaccatctccagagacaacgccaagaactcactgtatctgcacatgaacagcctgagaggcgaggacacggctgtgtattactgtgcgagccgatattacgattttcgaccggaggcttggtttgactactggggccagggaaccctggtcaccgtctcctcag

SEQ ID NO: 292 - protein, 122 aa, Homo sapiens
QVQLVESGGGLVQPGGSLRLSCAASGFPFSIYWMSWVRQAPGKGLEWVANIKQDGSEKYYVDSVKGRFTISRDNAKNSLYLHMNSLRGEDTAVYYCASRYYDFRPEAWFDYWGQGTLVTVSS

SEQ ID NO: 293 - DNA, 343 bp, Homo sapiens
  source 1..343: mol_type = other DNA
gatattgtgatgacccagactccactctcctcacctgtcacccttggacagccggcctccatctcctgcaggtctagtcaaagcctcgtacacagggatggaaacacctacttgagctggcttcagcagaggccaggccagcctccaagactcctaatttataagatttctaaccggttctctggggtcccagacagattcagtggcagtggggcagggacagatttcacactgaaaatcagcagggtggaagctgaggatgtcggggtttattactgcatgcaagctacacaatttcctcatgggtacacttttggccaggggaccaaggtggagatcaaac

SEQ ID NO: 294 - protein, 114 aa, Homo sapiens
DIVMTQTPLSSPVTLGQPASISCRSSQSLVHRDGNTYLSWLQQRPGQPPRLLIYKISNRFSGVPDRFSGSGAGTDFTLKISRVEAEDVGVYYCMQATQFPHGYTFGQGTKVEIK

SEQ ID NO: 295 - protein, 8 aa, Homo sapiens
GFPFSIYW

SEQ ID NO: 296 - protein, 8 aa, Homo sapiens
IKQDGSEK

SEQ ID NO: 297 - protein, 15 aa, Homo sapiens
ASRYYDFRPEAWFDY

SEQ ID NO: 298 - protein, 11 aa, Homo sapiens
QSLVHRDGNTY

SEQ ID NO: 299 - skipped

SEQ ID NO: 300 - protein, 11 aa, Homo sapiens
MQATQFPHGYT

SEQ ID NO: 301 - DNA, 355 bp, Homo sapiens
  source 1..355: mol_type = other DNA
caggtgcagctgcaggagtctgggggaggcttggtccagcctggggggtccctgagactctcctgttcagcctctggattcaccgtcagtagcaactacatgacctgggtccgccaggctccagggaaggggctggagtgggtctcagttatttatagcggtggtagcacattctacgcagactccgtgaagggcagattcaccatctccagagacaattccaagaacacgctgtatcttcaaatgaacagcctgagagccgaggacaccgctgtgtattactgtgcgagagatctggaagaggccgggggatttgactactggggccagggaaccctggtcaccgtctcctcag

SEQ ID NO: 302 - protein, 118 aa, Homo sapiens
QVQLQESGGGLVQPGGSLRLSCSASGFTVSSNYMTWVRQAPGKGLEWVSVIYSGGSTFYADSVKGRFTISRDNSKNTLYLQMNSLRAEDTAVYYCARDLEEAGGFDYWGQGTLVTVSS

SEQ ID NO: 303 - DNA, 325 bp, Homo sapiens
  source 1..325: mol_type = other DNA
gaaattgtgttgacgcagtctccaggcaccctgtctttgtctccaggggaaaaagtcaccctctcctgcagggccagtcagagtgttagcagcacctacttagcctggtaccagcagaaacctggccaggctcccaggctcctcatctatggtgcatccagcagggccactggcgtcccagacaggttccgtggcagtgggtctgggacagacttcactctcaccatcagcagactggagcctgaagattttgcagtgtattactgtcagcagtatggtagctcgctgtacacttttggccaggggaccaaagtggatatcaaac

SEQ ID NO: 304 - protein, 108 aa, Homo sapiens
EIVLTQSPGTLSLSPGEKVTLSCRASQSVSSTYLAWYQQKPGQAPRLLIYGASSRATGVPDRFRGSGSGTDFTLTISRLEPEDFAVYYCQQYGSSLYTFGQGTKVDIK

SEQ ID NO: 305 - protein, 8 aa, Homo sapiens
GFTVSSNY

SEQ ID NO: 306 - protein, 7 aa, Homo sapiens
IYSGGST

SEQ ID NO: 307 - protein, 12 aa, Homo sapiens
ARDLEEAGGFDY

SEQ ID NO: 308 - protein, 7 aa, Homo sapiens
QSVSSTY

SEQ ID NO: 309 - skipped

SEQ ID NO: 310 - protein, 9 aa, Homo sapiens
QQYGSSLYT

SEQ ID NO: 311 - DNA, 385 bp, Homo sapiens
  source 1..385: mol_type = other DNA
cagctgcagctgcaggagtcgggcccaggactggtgaagccttcggagaccctgtccctcacctgcactgtctccggtgactccgtcagtaattactactggagctggatccggcagcccgccgggaagggactggagtggattgggcgtatctataccagtgggagcaccaactacaacccctccctcaagagtcgagtcaccatgtcagtagacacgtccaagaaccagttctccctgaagctgagctctgtgaccgccgcggacacggccgtgtattactgtgcgagagatcaccgggcttcccggtatagcagtggctggtacgaatggtggaactgcttcgacccctggggccagggaaccctggtcaccgtctcctcag

SEQ ID NO: 312 - protein, 128 aa, Homo sapiens
QLQLQESGPGLVKPSETLSLTCTVSGDSVSNYYWSWIRQPAGKGLEWIGRIYTSGSTNYNPSLKSRVTMSVDTSKNQFSLKLSSVTAADTAVYYCARDHRASRYSSGWYEWWNCFDPWGQGTLVTVSS

SEQ ID NO: 313 - DNA, 325 bp, Homo sapiens
  source 1..325: mol_type = other DNA
gacatccagatgacccagtctccatcctccctgtctgcatctgtaggagacagagtcaccatcacttgccgggcaagtcagagcattagcagctatttaaattggtatcagcagaaaccagggaaagcccctaagctcctgatctatgctgcatccagtttgcaaagtggggtcccgtcaaggttcagtggcagtggatctgggacagatttcactctcaccatcaacagtctgcaacctgaagattttgcaacttactactgtcaacagagttacagtacccccgcgctcactttcggcggagggaccaaagtggatatcaaac

SEQ ID NO: 314 - protein, 108 aa, Homo sapiens
DIQMTQSPSSLSASVGDRVTITCRASQSISSYLNWYQQKPGKAPKLLIYAASSLQSGVPSRFSGSGSGTDFTLTINSLQPEDFATYYCQQSYSTPALTFGGGTKVDIK

SEQ ID NO: 315 - protein, 8 aa, Homo sapiens
GDSVSNYY

SEQ ID NO: 316 - protein, 7 aa, Homo sapiens
IYTSGST

SEQ ID NO: 317 - protein, 22 aa, Homo sapiens
ARDHRASRYSSGWYEWWNCFDP

SEQ ID NO: 318 - protein, 6 aa, Homo sapiens
QSISSY

SEQ ID NO: 319 - skipped

SEQ ID NO: 320 - protein, 10 aa, Homo sapiens
QQSYSTPALT

SEQ ID NO: 321 - DNA, 367 bp, Homo sapiens
  source 1..367: mol_type = other DNA
caggttcagctggtgcagtctggggctgaggtgaagaagcctggggcctcagtgaaggtctcctgcaaggcttctggatacaccttcaccggctactatatgcactgggtgcgacaggcccctggacaagggcttgagtggatgggatggatcaaccctaacagtggtggcacaaactatacacagaagtttcagggcagggtcaccatgaccagggacacgtccatcagcacagcctacatggagctgagcaggctgagatctgacgacacggccgtgtattcctgtgcgagagatatggcgtttagtatggttcggggttcctttgactactggggccagggaaccctggtcaccgtctcctcag

SEQ ID NO: 322 - protein, 122 aa, Homo sapiens
QVQLVQSGAEVKKPGASVKVSCKASGYTFTGYYMHWVRQAPGQGLEWMGWINPNSGGTNYTQKFQGRVTMTRDTSISTAYMELSRLRSDDTAVYSCARDMAFSMVRGSFDYWGQGTLVTVSS

SEQ ID NO: 323 - DNA, 331 bp, Homo sapiens
  source 1..331: mol_type = other DNA
caggctgtgctgactcagcctccctccgcgtccgggtctcctggacagtcagtcaccatctcctgcactggaaccagcagtgacgttggtggttataactatgtctcctggtaccaacagcacccaggcaaagcccccaaactcatgatttatgaggtcagtaagcggccctcaggggtccctgatcgcttctctggctccaagtctggcaacacggcctccctgaccgtctctgggctccaggctgaggatgaggctgattattactgcagctcatatgcaggcagcaaccattgggtgttcggcggagggaccaagctgaccgtcctag

SEQ ID NO: 324 - protein, 110 aa, Homo sapiens
QAVLTQPPSASGSPGQSVTISCTGTSSDVGGYNYVSWYQQHPGKAPKLMIYEVSKRPSGVPDRFSGSKSGNTASLTVSGLQAEDEADYYCSSYAGSNHWVFGGGTKLTVL

SEQ ID NO: 325 - protein, 8 aa, Homo sapiens
GYTFTGYY

SEQ ID NO: 326 - protein, 8 aa, Homo sapiens
INPNSGGT

SEQ ID NO: 327 - protein, 15 aa, Homo sapiens
ARDMAFSMVRGSFDY

SEQ ID NO: 328 - protein, 9 aa, Homo sapiens
SSDVGGYNY

SEQ ID NO: 329 - skipped

SEQ ID NO: 330 - protein, 10 aa, Homo sapiens
SSYAGSNHWV

SEQ ID NO: 331 - DNA, 358 bp, Homo sapiens
  source 1..358: mol_type = other DNA
caggtgcagctggtgcagtctgggcctgaggtgaagaagcctgggacctcagtgaaggtctcctgcaaggcgtctggattcacccttactagctctgctatgcagtgggtgcgacaggctcgtggacaacgccttgagtggataggatggatcgtcgttggcagtggcaacacaaactacgcacagaagttccaggaaagagtcaccattaccagggacatgtccacaagcacagcctacatggagctgagcagcctgagatccgaggacacggccgtgtattattgtgcggccggccgtggctacaattcggactttgactactggggccagggaaccctggtcaccgtctcctcag

SEQ ID NO: 332 - protein, 119 aa, Homo sapiens
QVQLVQSGPEVKKPGTSVKVSCKASGFTLTSSAMQWVRQARGQRLEWIGWIVVGSGNTNYAQKFQERVTITRDMSTSTAYMELSSLRSEDTAVYYCAAGRGYNSDFDYWGQGTLVTVSS

SEQ ID NO: 333 - DNA, 325 bp, Homo sapiens
  source 1..325: mol_type = other DNA
gccatccggatgacccagtctccaggcaccctgtctttgtctccaggggaaagagccaccctctcctgcagggccagtcagagtgttagcagcagctacttagcctggtaccagcagagacctggccaggctcccaggctcctcatctatggtacatccagcagggccactggcatcccagacaggttcagtggcagtgggtctgggacagacttcactctcaccatcagcagactggagcctgaagattttgcagtgtattactgtcagcagtatggttactcagtgtacacttttggccaggggaccaaagtggatatcaaac

SEQ ID NO: 334 - protein, 108 aa, Homo sapiens
AIRMTQSPGTLSLSPGERATLSCRASQSVSSSYLAWYQQRPGQAPRLLIYGTSSRATGIPDRFSGSGSGTDFTLTISRLEPEDFAVYYCQQYGYSVYTFGQGTKVDIK

SEQ ID NO: 335 - protein, 8 aa, Homo sapiens
GFTLTSSA

SEQ ID NO: 336 - protein, 8 aa, Homo sapiens
IVVGSGNT

SEQ ID NO: 337 - protein, 12 aa, Homo sapiens
AAGRGYNSDFDY

SEQ ID NO: 338 - protein, 7 aa, Homo sapiens
QSVSSSY

SEQ ID NO: 339 - skipped

SEQ ID NO: 340 - protein, 9 aa, Homo sapiens
QQYGYSVYT

SEQ ID NO: 341 - DNA, 367 bp, Homo sapiens
  source 1..367: mol_type = other DNA
caggtgcagctggtggagtctgaggctgaggtgaagaagcctggggcctcagtgaaggtttcctgcaaggcatctggatacaccttcaccagctactatatgcactgggtgcgacaggcccctggacaagggcttcagtggatgggaataatcaaccctagtgctggtagcacaagctacgcacagaagttccagggcagagtcaccatgaccacggacacgtccacgaccacagtctacatggagctgagcagcctgagatctgaggacacggccgtgtattactgtgcgagagattctgtactagtaccagctgctaatgcttttgatatctggggccaagggacaatggtcaccgtctcttcag

SEQ ID NO: 342 - protein, 122 aa, Homo sapiens
QVQLVESEAEVKKPGASVKVSCKASGYTFTSYYMHWVRQAPGQGLQWMGIINPSAGSTSYAQKFQGRVTMTTDTSTTTVYMELSSLRSEDTAVYYCARDSVLVPAANAFDIWGQGTMVTVSS

SEQ ID NO: 343 - DNA, 322 bp, Homo sapiens
  source 1..322: mol_type = other DNA
gaaatagtgatgacgcagtctccagccaccctgtctttgtctccaggggaaagagccaccctctcctgcagggccagtcagagtgttagtagctacttagcctggtaccaacagaaacctggccaggctcccaggctcctcatctatgatgcatccaacagggccactggcatcccagccaggttcagtggcagtgggtctgggacagacttcactctcaccatcagcagcctagagcctgaagattttgcagtttattactgtcagcagcgtcgcaactggctattcactttcggccctgggaccaaagtggatatcaaac

SEQ ID NO: 344 - protein, 107 aa, Homo sapiens
EIVMTQSPATLSLSPGERATLSCRASQSVSSYLAWYQQKPGQAPRLLIYDASNRATGIPARFSGSGSGTDFTLTISSLEPEDFAVYYCQQRRNWLFTFGPGTKVDIK

SEQ ID NO: 345 - protein, 8 aa, Homo sapiens
GYTFTSYY

SEQ ID NO: 346 - protein, 8 aa, Homo sapiens
INPSAGST

SEQ ID NO: 347 - protein, 15 aa, Homo sapiens
ARDSVLVPAANAFDI

SEQ ID NO: 348 - protein, 6 aa, Homo sapiens
QSVSSY

SEQ ID NO: 349 - skipped

SEQ ID NO: 350 - protein, 9 aa, Homo sapiens
QQRRNWLFT

SEQ ID NO: 351 - DNA, 358 bp, Homo sapiens
  source 1..358: mol_type = other DNA
caggtgcagctggtgcagtctggggctgaggtgaagaagcctggggcctcagtgaaggtttcctgcaaggcttctggagacaccttcactagctatactctgcattgggtgcgccaggcccccggacaaaggcttgagtggatgggatggatcaacgctggcaatggttacacaaaatattcacagaagttccagggcagagtcaccattaccagggacacatccgcgagcacagcctacatggagctgagcagcctgagatctgaagacacggctgtgtattactgtgcgaaatgtactatgatagtagactactttgactactggggccagggaaccctggtcaccgtctcctcag

SEQ ID NO: 352 - protein, 119 aa, Homo sapiens
QVQLVQSGAEVKKPGASVKVSCKASGDTFTSYTLHWVRQAPGQRLEWMGWINAGNGYTKYSQKFQGRVTITRDTSASTAYMELSSLRSEDTAVYYCAKCTMIVDYFDYWGQGTLVTVSS

SEQ ID NO: 353 - DNA, 322 bp, Homo sapiens
  source 1..322: mol_type = other DNA
gccatccggatgacccagtctccttccaccctgtctgcatctgtaggagacagagtcaccatcacttgccgggccagtcagagtattagtggctggttggcctggtatcagcagaaaccagagaaagcccctaagctcctgatctatgatgcctccaatttggaaagtggggtcccatcaaggttcagcggcagtggatctgggacagaattcactctcaccatcaacagcctgcagcctgatgattttgcaacttattactgccaacagtataatagttacccgtggacgttcggccaagggaccaaagtggatatcaaac

SEQ ID NO: 354 - protein, 107 aa, Homo sapiens
AIRMTQSPSTLSASVGDRVTITCRASQSISGWLAWYQQKPEKAPKLLIYDASNLESGVPSRFSGSGSGTEFTLTINSLQPDDFATYYCQQYNSYPWTFGQGTKVDIK

SEQ ID NO: 355 - protein, 8 aa, Homo sapiens
GDTFTSYT

SEQ ID NO: 356 - protein, 8 aa, Homo sapiens
INAGNGYT

SEQ ID NO: 357 - protein, 12 aa, Homo sapiens
AKCTMIVDYFDY

SEQ ID NO: 358 - protein, 6 aa, Homo sapiens
QSISGW

SEQ ID NO: 359 - skipped

SEQ ID NO: 360 - protein, 9 aa, Homo sapiens
QQYNSYPWT

SEQ ID NO: 361 - DNA, 357 bp, Homo sapiens
  source 1..357: mol_type = other DNA
gaggtgcagctggtgcagtctggggctgaggtgaagaagcctggggcctcagtgaaggtctcctgcaaggcttctgggtacaccttcaccagttatgatatcaactgggtgcgacaggccactggacaagggcttgagtggatgggatggatgaaccctcacagtgataccacaggctatgcacagaagttccagggcagagtcaccatgaccaggaacacctccataaccacagcctacatggagctgagcagcctgagatctgaggacacggccgtgtattactgtgctcagggacccatagcagtgaactacatggacgtctggggcaaagggaccacggtcaccgtctcctca

SEQ ID NO: 362 - protein, 119 aa, Homo sapiens
EVQLVQSGAEVKKPGASVKVSCKASGYTFTSYDINWVRQATGQGLEWMGWMNPHSDTTGYAQKFQGRVTMTRNTSITTAYMELSSLRSEDTAVYYCAQGPIAVNYMDVWGKGTTVTVSS

SEQ ID NO: 363 - DNA, 325 bp, Homo sapiens
  source 1..325: mol_type = other DNA
cagcctgtgctgactcagccaccctcagtgtcagtggccccaggaaagacggccaggattacctgtgggggaagcaacattggaagtaaaagtgtgcactggtaccagcagaagccaggccaggcccctgtgctgatcatctattatgatagcgaccggccctcagggatccctgagcgattctctggctccaactctgggaacacggccaccctgaccatcagcagggtcgaagccggggatgaggccgacttttactgtcaggtgtgggatagtagtactgatcatgtggtattcggcggggggaccaagctgaccgtcctag

SEQ ID NO: 364 - protein, 108 aa, Homo sapiens
QPVLTQPPSVSVAPGKTARITCGGSNIGSKSVHWYQQKPGQAPVLIIYYDSDRPSGIPERFSGSNSGNTATLTISRVEAGDEADFYCQVWDSSTDHVVFGGGTKLTVL

SEQ ID NO: 365 - protein, 8 aa, Homo sapiens
GYTFTSYD

SEQ ID NO: 366 - protein, 8 aa, Homo sapiens
MNPHSDTT

SEQ ID NO: 367 - protein, 12 aa, Homo sapiens
AQGPIAVNYMDV

SEQ ID NO: 368 - protein, 6 aa, Homo sapiens
NIGSKS

SEQ ID NO: 369 - skipped

SEQ ID NO: 370 - protein, 11 aa, Homo sapiens
QVWDSSTDHVV

SEQ ID NO: 371 - DNA, 376 bp, Homo sapiens
  source 1..376: mol_type = other DNA
gaggtgcagctggtggagtctgggggaggcttggtcaagcctggagagtccctgagactctcctgtgcagcctctggattcaccttcagtgactactacatgacctggatccgccaggctccagggaaggggctggagtgggtttcatacattaggagtagtggtcatactatatactacgcagactctgtgaagggccgattcaccatctccagggacaacgccaagaactcactgtatctacaaatgaacagcctgagagtcgaggacacggccgtgtattactgtgcgagaggaggggttttacgatttttggagtggcctctcaatgcttttgatatctggggccaagggacaatggtcaccgtctcttcag

SEQ ID NO: 372 - protein, 125 aa, Homo sapiens
EVQLVESGGGLVKPGESLRLSCAASGFTFSDYYMTWIRQAPGKGLEWVSYIRSSGHTIYYADSVKGRFTISRDNAKNSLYLQMNSLRVEDTAVYYCARGGVLRFLEWPLNAFDIWGQGTMVTVSS

SEQ ID NO: 373 - DNA, 322 bp, Homo sapiens
  source 1..322: mol_type = other DNA
gacatccagttgacccagtctccatcctccctgtctgcatctgtaggagacagagtcaccatcacttgccgggcgagtcagggcattagcaattatttagcctggtatcagcagaaaccagggaaagttcctaagctcctgatctatgctgcatccactttgcaatcaggggtcccatctcggttcagtggcagtggatctgggacagatttcactctcaccatcagcagcctgcagcctgaagatgttgcaacttattactgtcaaaagtataacaatgccctcgggacgttcggccaagggaccaaggtggagatcaaac

SEQ ID NO: 374 - protein, 107 aa, Homo sapiens
DIQLTQSPSSLSASVGDRVTITCRASQGISNYLAWYQQKPGKVPKLLIYAASTLQSGVPSRFSGSGSGTDFTLTISSLQPEDVATYYCQKYNNALGTFGQGTKVEIK

SEQ ID NO: 375 - protein, 8 aa, Homo sapiens
GFTFSDYY

SEQ ID NO: 376 - protein, 8 aa, Homo sapiens
IRSSGHTI

SEQ ID NO: 377 - protein, 18 aa, Homo sapiens
ARGGVLRFLEWPLNAFDI

SEQ ID NO: 378 - protein, 6 aa, Homo sapiens
QGISNY

SEQ ID NO: 379 - skipped

SEQ ID NO: 380 - protein, 9 aa, Homo sapiens
QKYNNALGT

SEQ ID NO: 381 - DNA, 364 bp, Homo sapiens
  source 1..364: mol_type = other DNA
gaggtgcagctggtgcagtctggggctgaggtgaagaagcctggggcctcagtgaaggtctcctgcaaggcttctggatacaccttcaccggctactatatgcactgggtgcgacaggcccctggacaagggcttgagtggatgggatggatcagccctaacagtggtggcacaaactatgcacagaagtttcagggcagggtcaccatgaccagggacacgtccatcaccacagcctacatggacctgagcaggctgagatctgacgacacggccgtgtattactgtgcgagaggttattactatgaagccctcgatgcttttgatatctggggccaagggacaatggtcaccgtctcttcag

SEQ ID NO: 382 - protein, 121 aa, Homo sapiens
EVQLVQSGAEVKKPGASVKVSCKASGYTFTGYYMHWVRQAPGQGLEWMGWISPNSGGTNYAQKFQGRVTMTRDTSITTAYMDLSRLRSDDTAVYYCARGYYYEALDAFDIWGQGTMVTVSS

SEQ ID NO: 383 - DNA, 331 bp, Homo sapiens
  source 1..331: mol_type = other DNA
cagtctgtcgtgacgcagcctgcctccgtgtctgggtctcctggacagtcgatcaccatctcctgcactggaaccagcagtgacgttggtggttataactttgtctcctggtaccaacagcacccaggcaaagcccccaaactcatgatttatgaggtcagtaatcggccctcaggggtttctaatcgcttctctggctccaagtctggcatcacggcctccctgaccatctctgggctccaggctgaggacgaggctgattattactgcaactcatatacaagcaacagtactcgggtattcggcggagggaccaagctgaccgtcctag

SEQ ID NO: 384 - protein, 110 aa, Homo sapiens
QSVVTQPASVSGSPGQSITISCTGTSSDVGGYNFVSWYQQHPGKAPKLMIYEVSNRPSGVSNRFSGSKSGITASLTISGLQAEDEADYYCNSYTSNSTRVFGGGTKLTVL

SEQ ID NO: 385 - protein, 8 aa, Homo sapiens
GYTFTGYY

SEQ ID NO: 386 - protein, 8 aa, Homo sapiens
ISPNSGGT

SEQ ID NO: 387 - protein, 14 aa, Homo sapiens
ARGYYYEALDAFDI

SEQ ID NO: 388 - protein, 9 aa, Homo sapiens
SSDVGGYNF

SEQ ID NO: 389 - skipped

SEQ ID NO: 390 - protein, 10 aa, Homo sapiens
NSYTSNSTRV

SEQ ID NO: 391 - DNA, 382 bp, Homo sapiens
  source 1..382: mol_type = other DNA
caggtgcagctggtggagtctgggggaggcttggtccagcctggggggtccctgagactctcctgtgcagcctctggattcaccgtcagtagcaactacatgacctgggtccgccaggctccagggaaggggctggagtgggtctcagttatttatagcggtggtagcacatactacgcagactccgtgaagggcagattcaccatctccagagacaattccaagaacacgctatatcttcaaatgaacagcctgagagccgacgacacggctgtatattactgtgcgagagactctacagccgattacgatttttggagtggttattatgtaggtgcttttcatatctggggccaagggacaatggtcaccgtctcttcag

SEQ ID NO: 392 - protein, 127 aa, Homo sapiens
QVQLVESGGGLVQPGGSLRLSCAASGFTVSSNYMTWVRQAPGKGLEWVSVIYSGGSTYYADSVKGRFTISRDNSKNTLYLQMNSLRADDTAVYYCARDSTADYDFWSGYYVGAFHIWGQGTMVTVSS

SEQ ID NO: 393 - DNA, 331 bp, Homo sapiens
  source 1..331: mol_type = other DNA
cagactgtgctgactcagcctgcctccgtgtctgggtctcctggacagtcgatcaccatctcctgcactggaaccagcagtgacgttggtggttacaactatgtctcctggtaccaacagcacccaggcaaagcccccaaactcatgatttatgaggtcactaagcggccctcaggggtccctgatcgcttctctggctccaagtctggcaacacggcctccctgaccgtctctgggctccaggctgaggatgaggctgattattactgcagctcatatgcaggcagcaacaattgggtgttcggcggagggaccaagctgaccgtcctag

SEQ ID NO: 394 - protein, 110 aa, Homo sapiens
QTVLTQPASVSGSPGQSITISCTGTSSDVGGYNYVSWYQQHPGKAPKLMIYEVTKRPSGVPDRFSGSKSGNTASLTVSGLQAEDEADYYCSSYAGSNNWVFGGGTKLTVL

SEQ ID NO: 395 - protein, 8 aa, Homo sapiens
GFTVSSNY

SEQ ID NO: 396 - protein, 7 aa, Homo sapiens
IYSGGST

SEQ ID NO: 397 - protein, 21 aa, Homo sapiens
ARDSTADYDFWSGYYVGAFHI

SEQ ID NO: 398 - protein, 9 aa, Homo sapiens
SSDVGGYNY

SEQ ID NO: 399 - skipped

SEQ ID NO: 400 - protein, 10 aa, Homo sapiens
SSYAGSNNWV

SEQ ID NO: 401 - DNA, 370 bp, Homo sapiens
  source 1..370: mol_type = other DNA
gaggtgcagctggtggagtcgggcccaggactggtgaagccttcggagaccctgtccctcacctgcactgtctctggtgggtccatcagcagtagtagtcactactggggctggatccgccagcccccagggaaggggctggagtggattgggagtatttattatagtgagagtgcctactacaacccgtccctcaagagtcgagtcaccatgtcaatagacacgtccaagaaccagttctccctgaagctgaactctgtgaccgccgcggacacggccgtgtattactgtgcgagagtcactgagcctcggtggacttcttgttactttgactactggggccagggaaccctggtcaccgtctcctcag

SEQ ID NO: 402 - protein, 123 aa, Homo sapiens
EVQLVESGPGLVKPSETLSLTCTVSGGSISSSSHYWGWIRQPPGKGLEWIGSIYYSESAYYNPSLKSRVTMSIDTSKNQFSLKLNSVTAADTAVYYCARVTEPRWTSCYFDYWGQGTLVTVSS

SEQ ID NO: 403 - DNA, 325 bp, Homo sapiens
  source 1..325: mol_type = other DNA
gaaatagtgatgacgcagtctccagccaccctgtctttgtctccaggggaaagagccaccctctcctgcaggaccagtcagagtgttaccagctacttagcctggtaccaacagagacctggccaggctcccaggctcctcatctatgatgcatccgacagggccactggcatcccagccaggttcagtggcagtgggtctgggacagacttcactctcaccatcagcaacctagagcctgaagattttgcagtttattactgtcagctgcgtagcaactggcctccgatcaccttcggccaagggacacgactggagactaaac

SEQ ID NO: 404 - protein, 108 aa, Homo sapiens
EIVMTQSPATLSLSPGERATLSCRTSQSVTSYLAWYQQRPGQAPRLLIYDASDRATGIPARFSGSGSGTDFTLTISNLEPEDFAVYYCQLRSNWPPITFGQGTRLETK

SEQ ID NO: 405 - protein, 10 aa, Homo sapiens
GGSISSSSHY

SEQ ID NO: 406 - protein, 7 aa, Homo sapiens
IYYSESA

SEQ ID NO: 407 - protein, 15 aa, Homo sapiens
ARVTEPRWTSCYFDY

SEQ ID NO: 408 - protein, 6 aa, Homo sapiens
QSVTSY

SEQ ID NO: 409 - skipped

SEQ ID NO: 410 - protein, 10 aa, Homo sapiens
QLRSNWPPIT

SEQ ID NO: 411 - DNA, 370 bp, Homo sapiens
  source 1..370: mol_type = other DNA
gaagtgcagctggtggagtcgggcccaggactggtgaagccttcggagaccctgtccctcacctgcactgtctctgggggctccatcagcagtagtagttactactggggctggatccgccagcccccagggaaggggctggagtggattgggagtatctattatagtgggagcacctactacaacccgtccctcaagagtcgagtcaccatatcagtagacacgtccaagaaccagttctccctgaagctgagctctgtgaccgccgcggacacggccgtgtttttctgtgcgagagagaggagcgctcctctcgcgggcaactggttcgacccctggggccagggaaccctggtcaccgtctcttcag

SEQ ID NO: 412 - protein, 123 aa, Homo sapiens
EVQLVESGPGLVKPSETLSLTCTVSGGSISSSSYYWGWIRQPPGKGLEWIGSIYYSGSTYYNPSLKSRVTISVDTSKNQFSLKLSSVTAADTAVFFCARERSAPLAGNWFDPWGQGTLVTVSS

SEQ ID NO: 413 - DNA, 325 bp, Homo sapiens
  source 1..325: mol_type = other DNA
gacatccagatgacccagtctccatccttcctgtctgcatctgtaggagacagagtcaccatcacttgccgggccagtcagggcattagcagttatttagcctggtatcagcaaaaaccagggaaagcccctaagctcctgatctatgctgcatccactttgcaaagtggggtcccatcaaggttcagcggcagtggatctgggacagaattcactctcacaatcagcagcctgcagcctgaagattttgcaacttattactgtcaacagcttaatacttacccttcgatcaccttcggccaagggacacgactggagattaaac

SEQ ID NO: 414 - protein, 108 aa, Homo sapiens
DIQMTQSPSFLSASVGDRVTITCRASQGISSYLAWYQQKPGKAPKLLIYAASTLQSGVPSRFSGSGSGTEFTLTISSLQPEDFATYYCQQLNTYPSITFGQGTRLEIK

SEQ ID NO: 415 - protein, 10 aa, Homo sapiens
GGSISSSSYY

SEQ ID NO: 416 - protein, 7 aa, Homo sapiens
IYYSGST

SEQ ID NO: 417 - protein, 15 aa, Homo sapiens
ARERSAPLAGNWFDP

SEQ ID NO: 418 - protein, 6 aa, Homo sapiens
QGISSY

SEQ ID NO: 419 - skipped

SEQ ID NO: 420 - protein, 10 aa, Homo sapiens
QQLNTYPSIT

SEQ ID NO: 421 - DNA, 369 bp, Homo sapiens
  source 1..369: mol_type = other DNA
gaagtgcagctggtggagtctgggggaggcgtggtccagcctgggaggcccctgagactctcctgtgcagcctctggattccccttcagtaactatggcatgcactgggtccgccaggctccaggcaaggggctggaatgggtggcagttatatggtatgatggaagtaataaatactatgcagactccgtgaagggccgattcaccatctccagagacaattccaagaacacgctgtatctgcaaatgaacaacctgagagctgaggacacggctatatattactgtgcgaaagatgggtacacggcccactactactactactacatggacgtctggggcaaagggaccacggtcaccgtctcctca

SEQ ID NO: 422 - protein, 123 aa, Homo sapiens
EVQLVESGGGVVQPGRPLRLSCAASGFPFSNYGMHWVRQAPGKGLEWVAVIWYDGSNKYYADSVKGRFTISRDNSKNTLYLQMNNLRAEDTAIYYCAKDGYTAHYYYYYMDVWGKGTTVTVSS

SEQ ID NO: 423 - DNA, 328 bp, Homo sapiens
  source 1..328: mol_type = other DNA
gccatccagttgacccagtctccaggcaccctgtctttgtctccaggggaaagagccaccctctcctgcagggccagtcagagtgttagcagcagctacttagcctggtaccagcagaaacctggccaggctcccaggctcctaatctatggtgcatccagcagggccactggcatcccaggcaggttcagtggcagtgggtctgggacagacttcactctcaccatcagcagactggagcctgaagattttgcagtgtattactgtcagcagtatggtagctcacctgggatcactttcggcggagggaccaaagtggatatcaaac

SEQ ID NO: 424 - protein, 109 aa, Homo sapiens
AIQLTQSPGTLSLSPGERATLSCRASQSVSSSYLAWYQQKPGQAPRLLIYGASSRATGIPGRFSGSGSGTDFTLTISRLEPEDFAVYYCQQYGSSPGITFGGGTKVDIK

SEQ ID NO: 425 - protein, 8 aa, Homo sapiens
GFPFSNYG

SEQ ID NO: 426 - protein, 8 aa, Homo sapiens
IWYDGSNK

SEQ ID NO: 427 - protein, 16 aa, Homo sapiens
AKDGYTAHYYYYYMDV

SEQ ID NO: 428 - protein, 7 aa, Homo sapiens
QSVSSSY

SEQ ID NO: 429 - skipped

SEQ ID NO: 430 - protein, 10 aa, Homo sapiens
QQYGSSPGIT

SEQ ID NO: 431 - DNA, 372 bp, Homo sapiens
  source 1..372: mol_type = other DNA
gaagtgcagctggtggagtctgggggaggcgtggtccagcctgggaggtccctgagactctcctgtgcagcctctggattcaccttcagtaactatggcatacactgggtccgccaggctccaggcaaggggctggagtgggtggcagttatttcatatgatggaagtcataaatattatgcagactctgtgaagggccgattcaccatctccagagacaattccaagaacacgctatatctgcaaatgaacagcctgaaaactgaggacacggctgtgtattactgtgcgaaagatagttcagctgctattccctactactactacggtatggacgtctggggccaagggaccacggtcaccgtctcttca

SEQ ID NO: 432 - protein, 124 aa, Homo sapiens
EVQLVESGGGVVQPGRSLRLSCAASGFTFSNYGIHWVRQAPGKGLEWVAVISYDGSHKYYADSVKGRFTISRDNSKNTLYLQMNSLKTEDTAVYYCAKDSSAAIPYYYYGMDVWGQGTTVTVSS

SEQ ID NO: 433 - DNA, 340 bp, Homo sapiens
  source 1..340: mol_type = other DNA
gccatccagatgacccagtctccagactccctggctgtgtctctgggcgagagggccaccatcaactgcaagtccagccagagtattttatacaactccaacaataagacctacttagcttggtaccagcagaaaccaggacagcctcctaagctgctcattttctgggcatctacccgggaatccggggtccctgaccgattcagtggcagcgggtctgggacagatttcactctcaccatcagcagcctgcaggctgaagatgtggcagtttattactgtcagcaatattatagtattccccttattttcggccctgggaccaaagtggatatcaaac

SEQ ID NO: 434 - protein, 113 aa, Homo sapiens
AIQMTQSPDSLAVSLGERATINCKSSQSILYNSNNKTYLAWYQQKPGQPPKLLIFWASTRESGVPDRFSGSGSGTDFTLTISSLQAEDVAVYYCQQYYSIPLIFGPGTKVDIK

SEQ ID NO: 435 - protein, 8 aa, Homo sapiens
GFTFSNYG

SEQ ID NO: 436 - protein, 8 aa, Homo sapiens
ISYDGSHK

SEQ ID NO: 437 - protein, 17 aa, Homo sapiens
AKDSSAAIPYYYYGMDV

SEQ ID NO: 438 - protein, 12 aa, Homo sapiens
QSILYNSNNKTY

SEQ ID NO: 439 - skipped

SEQ ID NO: 440 - protein, 9 aa, Homo sapiens
QQYYSIPLI

SEQ ID NO: 441 - DNA, 370 bp, Homo sapiens
  source 1..370: mol_type = other DNA
cagctgcagctgcaggagtcgggcccaggactggtgaagccttcggagaccctgtccctcacctgcactgtctctggtggctccatcagcagtagtagttactactggggctggatccgccagcccccagggaaggggctggagtggattgggaatgtctactatagtgggggcacctactgcaacccgtccctcaagagtcgagtcaccatatcagtagacacgtccaagaatcagttctccctgaacctgagctccgtgaccgccgcggacacggccgtgtattactgtgcgagaatatggttcggggagcctgcgggtgggtactttgactactggggccagggaaccctggtcaccgtctcctcag

SEQ ID NO: 442 - protein, 123 aa, Homo sapiens
QLQLQESGPGLVKPSETLSLTCTVSGGSISSSSYYWGWIRQPPGKGLEWIGNVYYSGGTYCNPSLKSRVTISVDTSKNQFSLNLSSVTAADTAVYYCARIWFGEPAGGYFDYWGQGTLVTVSS

SEQ ID NO: 443 - DNA, 319 bp, Homo sapiens
  source 1..319: mol_type = other DNA
tcctatgagctgactcagccaccctcagtgtccgtgtccccaggacagacagccagcatcacctgctctggacataagttgggggataaaaatgcttgctggtatcagcagaagccaggccagtcccctgtgctggtcatctatgaatataacaagcggccctcagggatccctgagcgattctctggctccaactctgggaacacagccactctgaccatcagcgggacccaggctatggatgaggctgactattactgtcaggcgtgggacaccggcactcatgtcttcggaactgggaccaaggtcaccgtcctag

SEQ ID NO: 444 - protein, 106 aa, Homo sapiens
SYELTQPPSVSVSPGQTASITCSGHKLGDKNACWYQQKPGQSPVLVIYEYNKRPSGIPERFSGSNSGNTATLTISGTQAMDEADYYCQAWDTGTHVFGTGTKVTVL

SEQ ID NO: 445 - protein, 10 aa, Homo sapiens
GGSISSSSYY

SEQ ID NO: 446 - protein, 7 aa, Homo sapiens
VYYSGGT

SEQ ID NO: 447 - protein, 15 aa, Homo sapiens
ARIWFGEPAGGYFDY

SEQ ID NO: 448 - protein, 6 aa, Homo sapiens
KLGDKN

SEQ ID NO: 449 - skipped

SEQ ID NO: 450 - protein, 9 aa, Homo sapiens
QAWDTGTHV

SEQ ID NO: 451 - DNA, 370 bp, Homo sapiens
  source 1..370: mol_type = other DNA
caggtccagctggtacagtcgggcccaggactggtgaagccttcacagaccctgtccctcacctgctctgtctctgatggctccatcagcagtagtgattactactggagctggatccgccagccccccgggaagggcctggagtggattgggtacatctattacactgggagcacctactacaacccgtccctcaagagtcgagtttccatatcagtagacaggtccaagaaccaattctccctgaagctgagttctgtgactgccgcagacacggccgtttactattgtgccagactcgtagtaccatctccgaagggctcctggttcgacccctggggccagggaaccctggtcaccgtctcctcaa

SEQ ID NO: 452 - protein, 123 aa, Homo sapiens
QVQLVQSGPGLVKPSQTLSLTCSVSDGSISSSDYYWSWIRQPPGKGLEWIGYIYYTGSTYYNPSLKSRVSISVDRSKNQFSLKLSSVTAADTAVYYCARLVVPSPKGSWFDPWGQGTLVTVSS

SEQ ID NO: 453 - DNA, 331 bp, Homo sapiens
  source 1..331: mol_type = other DNA
tcctatgagctgactcagcctgcctccgtgtctgggtctcctggacagtcgatcaccatctcctgcactgggaccagcattgatgttgggaattataaccttgcctcctggtaccaacagcacccaggcaaagcccccaaactcatcatttatgagggcagtaggcggccctcaggggtttctaatcgcttctctggcgccaagtctggcaacacggcctccctgacaatctctgggctccaggctgaggacgaggctgattattactgctgctcatatgtaggtagtagcacttatgtcttcggatctgggaccaaggtcaccgtcctag

SEQ ID NO: 454 - protein, 110 aa, Homo sapiens
SYELTQPASVSGSPGQSITISCTGTSIDVGNYNLASWYQQHPGKAPKLIIYEGSRRPSGVSNRFSGAKSGNTASLTISGLQAEDEADYYCCSYVGSSTYVFGSGTKVTVL

SEQ ID NO: 455 - protein, 10 aa, Homo sapiens
DGSISSSDYY

SEQ ID NO: 456 - protein, 7 aa, Homo sapiens
IYYTGST

SEQ ID NO: 457 - protein, 15 aa, Homo sapiens
ARLVVPSPKGSWFDP

SEQ ID NO: 458 - protein, 9 aa, Homo sapiens
SIDVGNYNL

SEQ ID NO: 459 - skipped

SEQ ID NO: 460 - protein, 10 aa, Homo sapiens
CSYVGSSTYV

SEQ ID NO: 461 - DNA, 370 bp, Homo sapiens
  source 1..370: mol_type = other DNA
caggtccagctggtacagtctgggcctgaggtgaagaagcctgggacctcagtgaaggtctcctgcaaggcttctggattcacctttactagctctgctatgcagtgggtgcgacaggctcgtggacaacgccttgagtggataggatggatcgtcgttggcagtggtaacacaaactacgcacagaagttccaggaaagagtcaccattaccagggacatgtccacaagcacagcctacatggagctgagcagcctgagatccgaggacacggccgtgtattactgtgcggcagtttattgtagtggtggtagctgtaatgatgcttttgatatctggggccaagggacaatggtcaccgtctcttcag

SEQ ID NO: 462 - protein, 123 aa, Homo sapiens
QVQLVQSGPEVKKPGTSVKVSCKASGFTFTSSAMQWVRQARGQRLEWIGWIVVGSGNTNYAQKFQERVTITRDMSTSTAYMELSSLRSEDTAVYYCAAVYCSGGSCNDAFDIWGQGTMVTVSS

SEQ ID NO: 463 - DNA, 325 bp, Homo sapiens
  source 1..325: mol_type = other DNA
gaaatagtgatgacgcagtctccaggcaccctgtctttgtctccaggggaaagagccaccctctcctgcagggccagtcagagtgttagcagcagctacttagcctggtaccagcagaaacctggccaggctcccaggctcctcatctatggtgcatccagcagggccactggcatcccagacaggttcagtggcagtgggtctgggacagacttcactctcaccatcagcagactggagcctgaagattttgcagtgtattactgtcagcagtatggtagctcacccttcactttcggcggagggaccaaggtggaaatcaaac

SEQ ID NO: 464 - protein, 108 aa, Homo sapiens
EIVMTQSPGTLSLSPGERATLSCRASQSVSSSYLAWYQQKPGQAPRLLIYGASSRATGIPDRFSGSGSGTDFTLTISRLEPEDFAVYYCQQYGSSPFTFGGGTKVEIK

SEQ ID NO: 465 - protein, 8 aa, Homo sapiens
GFTFTSSA

SEQ ID NO: 466 - protein, 8 aa, Homo sapiens
IVVGSGNT

SEQ ID NO: 467 - protein, 16 aa, Homo sapiens
AAVYCSGGSCNDAFDI

SEQ ID NO: 468 - protein, 7 aa, Homo sapiens
QSVSSSY

SEQ ID NO: 469 - skipped

SEQ ID NO: 470 - protein, 9 aa, Homo sapiens
QQYGSSPFT

SEQ ID NO: 471 - DNA, 367 bp, Homo sapiens
  source 1..367: mol_type = other DNA
caggtacagctgcagcagtcgggcccaggactggtgaagccttcggagaccctgtccctcacctgcactgtctctggtgcctccattagtaattattactggagttggatccggcagcccccagggaagggactggagtgggttggatatatctattacactgggagcaccaaccacaacccctccctcaagagtcgagtcaccatatcactagacacgtccaagaatcagttctccctgaggctgagctctgtgaccgctgcggacacggccgtctattactgtgcgcgagcctattgtagtggtggtagctgcttcgatacttttgatatctggggccaagggacaatggtcaccgtctcttcag

SEQ ID NO: 472 - protein, 122 aa, Homo sapiens
QVQLQQSGPGLVKPSETLSLTCTVSGASISNYYWSWIRQPPGKGLEWVGYIYYTGSTNHNPSLKSRVTISLDTSKNQFSLRLSSVTAADTAVYYCARAYCSGGSCFDTFDIWGQGTMVTVSS

SEQ ID NO: 473 - DNA, 325 bp, Homo sapiens
  source 1..325: mol_type = other DNA
tcctatgagctgacacagccaccctcggtgtcagtggccccaggacagacggccagaattacctgtgggggaaacaacattggaagtaaaagtgtgcactggttccagcagaagccaggccaggcccctgtgctggtcgtctatgatgatagcgaccggccctcagggatccctgagcgattctctggctccaactctgggaacacggcctccctgaccatcagcagggtcgaagccggggatgaggccgactattactgtcaggtgtgggatagtgctagtgattcaggtgtcttcggaactgggaccaagctcaccgtcccag

SEQ ID NO: 474 - protein, 108 aa, Homo sapiens
SYELTQPPSVSVAPGQTARITCGGNNIGSKSVHWFQQKPGQAPVLVVYDDSDRPSGIPERFSGSNSGNTASLTISRVEAGDEADYYCQVWDSASDSGVFGTGTKLTVP

SEQ ID NO: 475 - protein, 8 aa, Homo sapiens
GASISNYY

SEQ ID NO: 476 - protein, 7 aa, Homo sapiens
IYYTGST

SEQ ID NO: 477 - protein, 16 aa, Homo sapiens
ARAYCSGGSCFDTFDI

SEQ ID NO: 478 - protein, 6 aa, Homo sapiens
NIGSKS

SEQ ID NO: 479 - skipped

SEQ ID NO: 480 - protein, 11 aa, Homo sapiens
QVWDSASDSGV

SEQ ID NO: 481 - DNA, 351 bp, Homo sapiens
  source 1..351: mol_type = other DNA
gaagtgcagctgttggagtctggaggaggcttggtccagcctggggggtccctgagactctcctgtgcagcctctgggttaaccgtcagaagcaactacatgaactgggtccgccaggctccagggaaggggctggagtgggtctcacttatttatagcggtggtagtacattctacgcagactccgtgaagggccgattcaccatctccagacacgattccaagaacacactgtatcttcaaatgaacagcctgagagctgaggacacggccgtgtattactgtgcgcgagatttggtagtctacggaatggacgtctggggccaagggaccacggtcaccgtctcctca

SEQ ID NO: 482 - protein, 117 aa, Homo sapiens
EVQLLESGGGLVQPGGSLRLSCAASGLTVRSNYMNWVRQAPGKGLEWVSLIYSGGSTFYADSVKGRFTISRHDSKNTLYLQMNSLRAEDTAVYYCARDLVVYGMDVWGQGTTVTVSS

SEQ ID NO: 483 - DNA, 322 bp, Homo sapiens
  source 1..322: mol_type = other DNA
gacatccagatgacccagtctccaggcaccctgtctttgtctccaggggaaagagccaccctctcctgcagggccagtcagagtgttagcagcagctccttagcctggtaccagcagaaacatggccaggctcccaggctcctcatctatggtacatccagcagggccactggcatcccagacaggttcagtggcagtgggtctgggacagacttcactctcaccatcagtggactggagcctgaagattttgcagtgtattactgtcagcagtatggtagctcaccccttttcggcggggggaccaaggtggaaatcaaac

SEQ ID NO: 484 - protein, 107 aa, Homo sapiens
DIQMTQSPGTLSLSPGERATLSCRASQSVSSSSLAWYQQKHGQAPRLLIYGTSSRATGIPDRFSGSGSGTDFTLTISGLEPEDFAVYYCQQYGSSPLFGGGTKVEIK

SEQ ID NO: 485 - protein, 8 aa, Homo sapiens
GLTVRSNY

SEQ ID NO: 486 - protein, 7 aa, Homo sapiens
IYSGGST

SEQ ID NO: 487 - protein, 11 aa, Homo sapiens
ARDLVVYGMDV

SEQ ID NO: 488 - protein, 7 aa, Homo sapiens
QSVSSSS

SEQ ID NO: 489 - skipped

SEQ ID NO: 490 - protein, 8 aa, Homo sapiens
QQYGSSPL

SEQ ID NO: 491 - DNA, 382 bp, Homo sapiens
  source 1..382: mol_type = other DNA
gaggtgcagctggtggagtctgggggaggcgtggtccagcctgggaggtccctgagactctcctgtgcagcctctggattcaccttcagtaattatggcatgcaccgggtccgccaggctccaggcaaggggctggagtgggtggcacttatttcatatgaagaaagtaatagatattatggagactccgtgaggggccgattcaccatctccagagacaattccaagaacactctgtatctgcaaatgaacagcctgagacctgaggacacggctgtgtattactgtgcgaaagatcaaggcccggctactgtgatggtgactgctattcggggcgctatggacgtctggggccaagggaccacggtcaccgtctcctcag

SEQ ID NO: 492 - protein, 127 aa, Homo sapiens
EVQLVESGGGVVQPGRSLRLSCAASGFTFSNYGMHRVRQAPGKGLEWVALISYEESNRYYGDSVRGRFTISRDNSKNTLYLQMNSLRPEDTAVYYCAKDQGPATVMVTAIRGAMDVWGQGTTVTVSS

SEQ ID NO: 493 - DNA, 343 bp, Homo sapiens
  source 1..343: mol_type = other DNA
gacatccagttgacccagtctccagattccctggctgtgtctctgggcgagagggccaccatcaactgcaagtccagccagagtgttttatacagctccaacaataagaactacttagcttggtaccagcagaaaccaggccagcctcctaaactcctcatttactgggcgtctacccgggaatccggggtccctgaccgattcagtggcagcgggtctgggacagatttcactctcaccatcagcagcctgcaggctgaagatgtggcagtatattactgtcagcaatattttggttctccttcgatcaccttcggccaagggacacgactggagattaaac

SEQ ID NO: 494 - protein, 114 aa, Homo sapiens
DIQLTQSPDSLAVSLGERATINCKSSQSVLYSSNNKNYLAWYQQKPGQPPKLLIYWASTRESGVPDRFSGSGSGTDFTLTISSLQAEDVAVYYCQQYFGSPSITFGQGTRLEIK

SEQ ID NO: 495 - protein, 8 aa, Homo sapiens
GFTFSNYG

SEQ ID NO: 496 - protein, 8 aa, Homo sapiens
ISYEESNR

SEQ ID NO: 497 - protein, 20 aa, Homo sapiens
AKDQGPATVMVTAIRGAMDV

SEQ ID NO: 498 - protein, 12 aa, Homo sapiens
QSVLYSSNNKNY

SEQ ID NO: 499 - skipped

SEQ ID NO: 500 - protein, 10 aa, Homo sapiens
QQYFGSPSIT

SEQ ID NO: 501 - DNA, 373 bp, Homo sapiens
  source 1..373: mol_type = other DNA
caggtgcagctggtgcagtctggggctgaggtgaagaagcctggggcctcagtgaaggtctcctgcaaggcttctggatacaccttcaccgactactatatgcactgggtgcgacaggcccctggacaagggcttgagtggatgggatggatcaactctaaagatggtggcgcgaactatgcacagaagtttcagggcagggtcaccctgaccagggacacgtcaatcgacacagcctacatagaactgagcaggctcagatctgacgacacggccgtgtattactgtgcgagatccgcctctacagtaaccgaaccaccgacaaactggttcgacccctggggccagggaaccctggtcaccgtctcctcag

SEQ ID NO: 502 - protein, 124 aa, Homo sapiens
QVQLVQSGAEVKKPGASVKVSCKASGYTFTDYYMHWVRQAPGQGLEWMGWINSKDGGANYAQKFQGRVTLTRDTSIDTAYIELSRLRSDDTAVYYCARSASTVTEPPTNWFDPWGQGTLVTVSS

SEQ ID NO: 503 - DNA, 325 bp, Homo sapiens
  source 1..325: mol_type = other DNA
gatgttgtgatgactcagtctccatcctccctgtctgcatctgtaggcgacagagtcaccgtcacttgccgggcaagtcagggcattagaaatgatttaggctggtatcagcagaaaccagggaaagctcctaagcgcctgatctatgctgcatccagtttgcaaagtggtgtcccatcaaggttcagcggcagtggatctgggacagacttcactctcacaatcagcagcctgcagcctgaagattttgcaacttattactgtctacagcataatagttacctccgtttcactttcggccctgggaccaaagtggatatcaaac

SEQ ID NO: 504 - protein, 108 aa, Homo sapiens
DVVMTQSPSSLSASVGDRVTVTCRASQGIRNDLGWYQQKPGKAPKRLIYAASSLQSGVPSRFSGSGSGTDFTLTISSLQPEDFATYYCLQHNSYLRFTFGPGTKVDIK

SEQ ID NO: 505 - protein, 8 aa, Homo sapiens
GYTFTDYY

SEQ ID NO: 506 - protein, 8 aa, Homo sapiens
INSKDGGA

SEQ ID NO: 507 - protein, 17 aa, Homo sapiens
ARSASTVTEPPTNWFDP

SEQ ID NO: 508 - protein, 6 aa, Homo sapiens
QGIRND

SEQ ID NO: 509 - skipped

SEQ ID NO: 510 - protein, 10 aa, Homo sapiens
LQHNSYLRFT

SEQ ID NO: 511 - DNA, 376 bp, Homo sapiens
  source 1..376: mol_type = other DNA
gaagtgcagctggtgcagtctggggctgaggtgaagaagcctggggcctcagtgaaggtctcctgcaaggcttctggatacaccttcaccggctactatatgcactgggtgcgacaggcccctggacaagggcttgagtggatgggatggatcaaccctaacagtggtggcacaaactatgcacagaagtttcagggcagggtcaccatgaccagggacacgtccatcaccacaggctacatggagctgagcagcctgagatctgacgacacggccctgtattactgtgcgagagttggggctcacgattactatgatagtagtgacaactggttcgacccctggggccagggaaccctggtcaccgtcttctcag

SEQ ID NO: 512 - protein, 125 aa, Homo sapiens
EVQLVQSGAEVKKPGASVKVSCKASGYTFTGYYMHWVRQAPGQGLEWMGWINPNSGGTNYAQKFQGRVTMTRDTSITTGYMELSSLRSDDTALYYCARVGAHDYYDSSDNWFDPWGQGTLVTVFS

SEQ ID NO: 513 - DNA, 322 bp, Homo sapiens
  source 1..322: mol_type = other DNA
gacatccagatgacccagtctccatcttccgtgtctgcatctgtaggagacagactcaccatcacttgtcgggcgagtcagggtattagcagctggttagcctggtatcagcagaaaccagggaaagcccctaagctcctgatctatgctgcatccagtttgcaaagtggggtcccatcaaggttcagcggcagtggatctgggacagatttcactctcaccatcagcagcctgcagcctgaagattttgcaacttactattgtcaacaggctaacagtttcccgtggacgttcggccaagggaccaaggtggagatcaaac

SEQ ID NO: 514 - protein, 107 aa, Homo sapiens
DIQMTQSPSSVSASVGDRLTITCRASQGISSWLAWYQQKPGKAPKLLIYAASSLQSGVPSRFSGSGSGTDFTLTISSLQPEDFATYYCQQANSFPWTFGQGTKVEIK

SEQ ID NO: 515 - protein, 8 aa, Homo sapiens
GYTFTGYY

SEQ ID NO: 516 - protein, 8 aa, Homo sapiens
INPNSGGT

SEQ ID NO: 517 - protein, 18 aa, Homo sapiens
ARVGAHDYYDSSDNWFDP

SEQ ID NO: 518 - protein, 6 aa, Homo sapiens
QGISSW

SEQ ID NO: 519 - skipped

SEQ ID NO: 520 - protein, 9 aa, Homo sapiens
QQANSFPWT

SEQ ID NO: 521 - DNA, 375 bp, Homo sapiens
  source 1..375: mol_type = other DNA
caggtgcagctggtgcagtctggggctgaggtgaagaagcctggggcctcagtgaaggtctcctgcaaggcttctggataccccctcaccggctactatatacactgggtgcgacaggcccctggacaaggacttgagtggatgggatggctcaaccctaacagtggtggcacaaagtatgcacagaagtttcagggcagggtcaccatgaccagggacacgtccatcagcacaggctacatggagctgagcaggctgagatctgacgacacggccgtgtactactgtgcgagagatggggggggaatagatgattacgttcaggaggacggtatggacgtctggggccaagggcccatggtcaccgtctcttca

SEQ ID NO: 522 - protein, 125 aa, Homo sapiens
QVQLVQSGAEVKKPGASVKVSCKASGYPLTGYYIHWVRQAPGQGLEWMGWLNPNSGGTKYAQKFQGRVTMTRDTSISTGYMELSRLRSDDTAVYYCARDGGGIDDYVQEDGMDVWGQGPMVTVSS

SEQ ID NO: 523 - DNA, 325 bp, Homo sapiens
  source 1..325: mol_type = other DNA
caatctgccctgactcagccaccctcggtgtcagtgtccccaggacagacggccaggatcacctgctctggagatgcattgtcaaagcaacatgcttattggtaccagcagaagccaggccaggcccctgtattggtgatatataaagacagtgagaggccctcagggatccctgagcgattctctggctccagctcagggacaatagtcacgttgaccatcagtggagtccaggcagaagacgaggctgactattactgtcaatcagcagacaacagtggtagtagatatgtcttcggaactgggaccaaggtcaccgtcctag

SEQ ID NO: 524 - protein, 108 aa, Homo sapiens
QSALTQPPSVSVSPGQTARITCSGDALSKQHAYWYQQKPGQAPVLVIYKDSERPSGIPERFSGSSSGTIVTLTISGVQAEDEADYYCQSADNSGSRYVFGTGTKVTVL

SEQ ID NO: 525 - protein, 8 aa, Homo sapiens
GYPLTGYY

SEQ ID NO: 526 - protein, 8 aa, Homo sapiens
LNPNSGGT

SEQ ID NO: 527 - protein, 18 aa, Homo sapiens
ARDGGGIDDYVQEDGMDV

SEQ ID NO: 528 - protein, 6 aa, Homo sapiens
ALSKQH

SEQ ID NO: 529 - skipped

SEQ ID NO: 530 - protein, 11 aa, Homo sapiens
QSADNSGSRYV

SEQ ID NO: 531 - DNA, 355 bp, Homo sapiens
  source 1..355: mol_type = other DNA
gaagtgcagctggtggagtctgggggaggcttggtacagcctggggggtccctgagactctcctgtgcagcctctggattcaccttcagtagctatagcatgaactgggtccgccaggctccagggaaggggctggagtgggtctcatacattagtggcattaatagtgccatatattacgcagactctgtgaagggccgcttcaccatctccagagacaacgccaagaactcactgtatctgcaaatgaacagcctgagagtcgaggacacggctgtgtattactgtgcgagagataaatacttaggtataaaagatatgtggggccaagggacaatggtcaccgtctcttcag

SEQ ID NO: 532 - protein, 118 aa, Homo sapiens
EVQLVESGGGLVQPGGSLRLSCAASGFTFSSYSMNWVRQAPGKGLEWVSYISGINSAIYYADSVKGRFTISRDNAKNSLYLQMNSLRVEDTAVYYCARDKYLGIKDMWGQGTMVTVSS

SEQ ID NO: 533 - DNA, 322 bp, Homo sapiens
  source 1..322: mol_type = other DNA
gacatccagatgacccagtctccagccaccctgtctttgtctccaggggaaagagccaccctctcctgcagggccagtcagagtgttagcacctacttagcctggtaccaacagaaacctggccaggctcccaggctcgtcatctatgatgcatccaacagggccactggcatcccagccaggttcagtggcggtgggtctgggacagacttcactctcaccatcagcagcctagagcctgaagattttgcagtttattactgtcaacagcgtctcaactggcctctcactttcggcggagggaccaaagtggatatcaaac

SEQ ID NO: 534 - protein, 107 aa, Homo sapiens
DIQMTQSPATLSLSPGERATLSCRASQSVSTYLAWYQQKPGQAPRLVIYDASNRATGIPARFSGGGSGTDFTLTISSLEPEDFAVYYCQQRLNWPLTFGGGTKVDIK

SEQ ID NO: 535 - protein, 8 aa, Homo sapiens
GFTFSSYS

SEQ ID NO: 536 - protein, 8 aa, Homo sapiens
ISGINSAI

SEQ ID NO: 537 - protein, 11 aa, Homo sapiens
ARDKYLGIKDM

SEQ ID NO: 538 - protein, 6 aa, Homo sapiens
QSVSTY

SEQ ID NO: 539 - skipped

SEQ ID NO: 540 - protein, 9 aa, Homo sapiens
QQRLNWPLT

SEQ ID NO: 541 - DNA, 375 bp, Homo sapiens
  source 1..375: mol_type = other DNA
gaggtgcagctggtacagtctggagcagaggtgaaaaagccgggggagtctctgaagatctcctgtaagggctctggatacagctttaccaactactggatcggctgggtgcgccagatgcccgggaaaggcctggagtggatggggatcatctatcctggtgactctggtaccagatacagcccgtccttccaaggccaggtcaccatctcagccgacaagtccatcagaaccgcctacctgcagtggagcagcctgaaggcctcggacagcgccatgtattactgtgcgaggtctagagtgggagctactgggggctattatgactactatatggacgtctggggccaagggaccacggtcaccgtctcctca

SEQ ID NO: 542 - protein, 125 aa, Homo sapiens
EVQLVQSGAEVKKPGESLKISCKGSGYSFTNYWIGWVRQMPGKGLEWMGIIYPGDSGTRYSPSFQGQVTISADKSIRTAYLQWSSLKASDSAMYYCARSRVGATGGYYDYYMDVWGQGTTVTVSS

SEQ ID NO: 543 - DNA, 331 bp, Homo sapiens
  source 1..331: mol_type = other DNA
cagtctgtgttgacgcagccaccctcagcgtctgggacccccgggcagagggtcaccatctcttgttctggaagcagctccaacctcggaggtaatactgtaaactggtaccagcagctcccaggaacggcccccaaactcctcatctatagtaataatcagcggccctcaggggtccctgaccgattctctggctccaagtctggcacctcagcctccctggccatcagtgggctccagtctgaggatgaggctgattattactgtgcagcatgggatgacagcctgaatggtcccgtcttcggaactgggaccaaggtcaccgtcctag

SEQ ID NO: 544 - protein, 110 aa, Homo sapiens
QSVLTQPPSASGTPGQRVTISCSGSSSNLGGNTVNWYQQLPGTAPKLLIYSNNQRPSGVPDRFSGSKSGTSASLAISGLQSEDEADYYCAAWDDSLNGPVFGTGTKVTVL

SEQ ID NO: 545 - protein, 8 aa, Homo sapiens
GYSFTNYW

SEQ ID NO: 546 - protein, 8 aa, Homo sapiens
IYPGDSGT

SEQ ID NO: 547 - protein, 18 aa, Homo sapiens
ARSRVGATGGYYDYYMDV

SEQ ID NO: 548 - protein, 8 aa, Homo sapiens
SSNLGGNT

SEQ ID NO: 549 - skipped

SEQ ID NO: 550 - protein, 11 aa, Homo sapiens
AAWDDSLNGPV

SEQ ID NO: 551 - DNA, 370 bp, Homo sapiens
  source 1..370: mol_type = other DNA
caggtgcagctggtggagtcgggcccaggacaggtgaagccttcggagaccctgtccctcacctgcactgtctctggtggctccatcagcagtagtagttactactggggctggatccgccagcccccagggaagggactggagtggattgggagtatctattatagtgggagcgcctactataacccgtccctcaagagtcgagtcaccatatccgtagacacgtccaagaaccagttctccctgaagctgaactctgtgaccgccgcagacacggctgtcttttactgtgcgagacacgcagctcccagtccgggggacaactggttcgacccctggggccagggaaccctggtcaccgtctcctcag

SEQ ID NO: 552 - protein, 123 aa, Homo sapiens
QVQLVESGPGQVKPSETLSLTCTVSGGSISSSSYYWGWIRQPPGKGLEWIGSIYYSGSAYYNPSLKSRVTISVDTSKNQFSLKLNSVTAADTAVFYCARHAAPSPGDNWFDPWGQGTLVTVSS

SEQ ID NO: 553 - DNA, 322 bp, Homo sapiens
  source 1..322: mol_type = other DNA
cagtctgtgttgactcagccaccctcggtgtcagtgtccccaggacagacggcccggatcacctgctctggagatgcattgtcaacgcaaaatggtaattggtaccagcagaagccaggccaggcccctgtgatggtgatatgtaaagacagtgagaggccctcagggatccctgagcgattctctggctccaggtcagggacaacagtcacgttgaccatcagtggagtccaggcagaagacgaagctgactatcactgtcaatcagcagacaacagggcacatgtagtattcggcggagggaccaagctgaccgtcctag

SEQ ID NO: 554 - protein, 107 aa, Homo sapiens
QSVLTQPPSVSVSPGQTARITCSGDALSTQNGNWYQQKPGQAPVMVICKDSERPSGIPERFSGSRSGTTVTLTISGVQAEDEADYHCQSADNRAHVVFGGGTKLTVL

SEQ ID NO: 555 - protein, 10 aa, Homo sapiens
GGSISSSSYY

SEQ ID NO: 556 - protein, 7 aa, Homo sapiens
IYYSGSA

SEQ ID NO: 557 - protein, 15 aa, Homo sapiens
ARHAAPSPGDNWFDP

SEQ ID NO: 558 - protein, 6 aa, Homo sapiens
ALSTQN

SEQ ID NO: 559 - skipped

SEQ ID NO: 560 - protein, 10 aa, Homo sapiens
QSADNRAHVV

SEQ ID NO: 561 - DNA, 378 bp, Homo sapiens
  source 1..378: mol_type = other DNA
gaggtgcagctggtggagtctgggggaggcgtggtccagcctgggaggtccctgagactctcctgtgcagcgtctggattcacctttagtagttatggcatgcactgggtccgccaggctccaggcaaggggctggagtgggtggcagttatatggtatgatggaagtaataacttctatgcagactccgtgaagggccgattcaccatctccagagacaatttcaagaacacgctgtatttgcaaatgaacagcctgagagccgaggacacggctgtgtattactgtgcgagatcatattgtagtggtggtttctgcttcggctactactatggtttggacgtgtggggccaagggaccacggtcaccgtctcctca

SEQ ID NO: 562 - protein, 126 aa, Homo sapiens
EVQLVESGGGVVQPGRSLRLSCAASGFTFSSYGMHWVRQAPGKGLEWVAVIWYDGSNNFYADSVKGRFTISRDNFKNTLYLQMNSLRAEDTAVYYCARSYCSGGFCFGYYYGLDVWGQGTTVTVSS

SEQ ID NO: 563 - DNA, 325 bp, Homo sapiens
  source 1..325: mol_type = other DNA
tcctatgagctgactcagccaccctcagtgtcagtggccccaggaaagacggccacgattacctgtgggggaaacaacattggaactaaaagtgtgcactggtaccagcagaagccaggccaggcccctgtgttggtcatctattataatagcgaccggccctccgggatccctgagcgattctctggctccaactctgggaacacggtcaccctgaccatcagcagggtcgaagccggggatgaggccgactattactgtcaggtgtgggatagtggtagtgatcattatgtcttcggaactgggaccaaggtcaccgtcgtag

SEQ ID NO: 564 - protein, 108 aa, Homo sapiens
SYELTQPPSVSVAPGKTATITCGGNNIGTKSVHWYQQKPGQAPVLVIYYNSDRPSGIPERFSGSNSGNTVTLTISRVEAGDEADYYCQVWDSGSDHYVFGTGTKVTVV

SEQ ID NO: 565 - protein, 8 aa, Homo sapiens
GFTFSSYG

SEQ ID NO: 566 - protein, 8 aa, Homo sapiens
IWYDGSNN

SEQ ID NO: 567 - protein, 19 aa, Homo sapiens
ARSYCSGGFCFGYYYGLDV

SEQ ID NO: 568 - protein, 6 aa, Homo sapiens
NIGTKS

SEQ ID NO: 569 - skipped

SEQ ID NO: 570 - protein, 11 aa, Homo sapiens
QVWDSGSDHYV

SEQ ID NO: 571 - DNA, 358 bp, Homo sapiens
  source 1..358: mol_type = other DNA
caggtgcagctggtgcagtctggagctgaggtgaagaagcctggggcctcagtgaaggtctcctgcaaggcttctggttacacctttaccagctatggtatcagctgggtgcgacaggcccctggacaagggcttgagtggatgggatggatcagcccttacaatggtaacacacactatgcacagaagctccagggcagagtcaccatgaccacagacacatccacgagcacagcctacatggagctgaggagcctgagatctgacgacacggccgtatattactgtgcgagagatggggagttattgggctggttcgacccctggggccagggaaccctggtcaccgtctcctcag

SEQ ID NO: 572 - protein, 119 aa, Homo sapiens
QVQLVQSGAEVKKPGASVKVSCKASGYTFTSYGISWVRQAPGQGLEWMGWISPYNGNTHYAQKLQGRVTMTTDTSTSTAYMELRSLRSDDTAVYYCARDGELLGWFDPWGQGTLVTVSS

SEQ ID NO: 573 - DNA, 331 bp, Homo sapiens
  source 1..331: mol_type = other DNA
cagtctgtcgtgacgcagcctgcctccgtgtctgggtctcctggacagtcgatcaccatctcctgcactggaaccagcagtgatgttgggagttataaccttgtctcctggtaccaacagcacccaggcaaagcccccaaactcatgatttatgcgggcagtaagcggccctcaggggtttctaatcgcttctctggctccaagtctggcaacacggcctccctgacaatctctgggctccaggctgaggacgaggctgattattactgctgctcatatgcaggtagtagcacttgggtgttcggcggagggaccaagctgaccgtcctag

SEQ ID NO: 574 - protein, 110 aa, Homo sapiens
QSVVTQPASVSGSPGQSITISCTGTSSDVGSYNLVSWYQQHPGKAPKLMIYAGSKRPSGVSNRFSGSKSGNTASLTISGLQAEDEADYYCCSYAGSSTWVFGGGTKLTVL

SEQ ID NO: 575 - protein, 8 aa, Homo sapiens
GYTFTSYG

SEQ ID NO: 576 - protein, 8 aa, Homo sapiens
ISPYNGNT

SEQ ID NO: 577 - protein, 12 aa, Homo sapiens
ARDGELLGWFDP

SEQ ID NO: 578 - protein, 9 aa, Homo sapiens
SSDVGSYNL

SEQ ID NO: 579 - skipped

SEQ ID NO: 580 - protein, 10 aa, Homo sapiens
CSYAGSSTWV

SEQ ID NO: 581 - DNA, 378 bp, Homo sapiens
  source 1..378: mol_type = other DNA
ccaggtcagctggtggaatctgggggaagcttggtacagcctgggggggccctgagactctcctgtgaagcctctggattcacctttagcgactatgccatgagctgggtccgccaggctccagggaaggggctggagtgggtctcagttattaatagtagtggtggtatcacaaactacgcagactccgtgaagggccggttcaccatctccagaaacaattccaagaacacgctctatctgcaaatgaacagcctgagaggcgacgacacggccatatattactgtgcgaagggacccccgagaattaacaccttctacaggcactactacggtatggacgtctggggccaagggatcacggtcaccgtctcctca

SEQ ID NO: 582 - protein, 126 aa, Homo sapiens
PGQLVESGGSLVQPGGALRLSCEASGFTFSDYAMSWVRQAPGKGLEWVSVINSSGGITNYADSVKGRFTISRNNSKNTLYLQMNSLRGDDTAIYYCAKGPPRINTFYRHYYGMDVWGQGITVTVSS

SEQ ID NO: 583 - DNA, 322 bp, Homo sapiens
  source 1..322: mol_type = other DNA
gccatccagttgacccagtctccatcctccctgtctgcatctgtaggagacagagtcaccatcacttgccaggcgagtcaggacattaggaactatttaaattggtatcagcagaaaccagggaaagcccctaagctcctgatctacgatgcatccaatttggaaacaggggtcccatcaaggttcagtggaagtggatctgggacagattttactttcaccatcggcagcctgcagcctgaagatattgcaacatattactgtcaacaatatgataatctccgggccactttcggcggagggaccaaggtggagatcaaac

SEQ ID NO: 584 - protein, 107 aa, Homo sapiens
AIQLTQSPSSLSASVGDRVTITCQASQDIRNYLNWYQQKPGKAPKLLIYDASNLETGVPSRFSGSGSGTDFTFTIGSLQPEDIATYYCQQYDNLRATFGGGTKVEIK

SEQ ID NO: 585 - protein, 8 aa, Homo sapiens
GFTFSDYA

SEQ ID NO: 586 - protein, 8 aa, Homo sapiens
INSSGGIT

SEQ ID NO: 587 - protein, 19 aa, Homo sapiens
AKGPPRINTFYRHYYGMDV

SEQ ID NO: 588 - protein, 6 aa, Homo sapiens
QDIRNY

SEQ ID NO: 589 - skipped

SEQ ID NO: 590 - protein, 9 aa, Homo sapiens
QQYDNLRAT

SEQ ID NO: 591 - DNA, 370 bp, Homo sapiens
  source 1..370: mol_type = other DNA
caggtgcagctggtggagtctgggcctgaaatgaagaagcctgggacctcagtgaaggtctcctgcaaggcttctggattcacctttattacgtctgctgttcagtgggtgcgacaggctcgtggacaacgccttgagtggatgggatggatcgccgttggcagtggtaacacaaactacgcacagaaattccaggacagagtcaccattaacagggacatgtccacaagcacagcctacatggagctgagcagcctgagatccgaggacacggccgtgtattactgtgcggccccgcattgtaatcgtaccagctgccatgatggttttgatatctggggccaagggacaatggtcaccgtctcttcag

SEQ ID NO: 592 - protein, 123 aa, Homo sapiens
QVQLVESGPEMKKPGTSVKVSCKASGFTFITSAVQWVRQARGQRLEWMGWIAVGSGNTNYAQKFQDRVTINRDMSTSTAYMELSSLRSEDTAVYYCAAPHCNRTSCHDGFDIWGQGTMVTVSS

SEQ ID NO: 593 - DNA, 325 bp, Homo sapiens
  source 1..325: mol_type = other DNA
gaaattgtgttgacgcagtctccaggcaccctgtctttgtctccaggggaaagagccaccctctcctgcagggccagtcagagtgttagcagaaactacttagcctggtaccagcagaaacctggccaggttcccaggctcctcatctatggtgcatccagcagggccactggcatcccagacaggttcagaggtagtgggtctgggacagacttcactctcaccatcaacagactggagtctgaagattttgcagtgtattactgtcagcagtatggtagctccctattcactttcggccctgggaccaaagtggatatcaaac

SEQ ID NO: 594 - protein, 108 aa, Homo sapiens
EIVLTQSPGTLSLSPGERATLSCRASQSVSRNYLAWYQQKPGQVPRLLIYGASSRATGIPDRFRGSGSGTDFTLTINRLESEDFAVYYCQQYGSSLFTFGPGTKVDIK

SEQ ID NO: 595 - protein, 8 aa, Homo sapiens
GFTFITSA

SEQ ID NO: 596 - protein, 8 aa, Homo sapiens
IAVGSGNT

SEQ ID NO: 597 - protein, 16 aa, Homo sapiens
AAPHCNRTSCHDGFDI

SEQ ID NO: 598 - protein, 7 aa, Homo sapiens
QSVSRNY

SEQ ID NO: 599 - skipped

SEQ ID NO: 600 - protein, 9 aa, Homo sapiens
QQYGSSLFT

SEQ ID NO: 601 - DNA, 361 bp, Homo sapiens
  source 1..361: mol_type = other DNA
gaagtgcagctggtggagtcggggggaggcctggtcaagcctggggagtccctgagactctcctgtgcagcctctggattcaccttcagtagctatgccatgaactgggtccgccaggctccagggaaggggctggagtgggtctcatccattagtactggtagttatttcatatactactcagactcagtgaagggccgattcaccatttccagagacaacgccaagaactcactgtatctgcaaatgaacagcctgagagccgcggacacggctatctattactgtgcgagaggaaaggaagatacaagcgctgcttttgatatctggggccaagggacaatggtcaccgtctcttcag

SEQ ID NO: 602 - protein, 120 aa, Homo sapiens
EVQLVESGGGLVKPGESLRLSCAASGFTFSSYAMNWVRQAPGKGLEWVSSISTGSYFIYYSDSVKGRFTISRDNAKNSLYLQMNSLRAADTAIYYCARGKEDTSAAFDIWGQGTMVTVSS

SEQ ID NO: 603 - DNA, 325 bp, Homo sapiens
  source 1..325: mol_type = other DNA
gccatccagatgacccagtctcttcctcctgcgactctggctccaggggaaagagccaccctctcctgcagggccagtcagagtgttagcaacaacttagcctggtaccagcagaaacctggccaggctcccaggctcctcatctatggtgcatccaccagggccactggtatcccagccaggttcagtggcagtgggtctgggacagagttcactctcaccatcagcagcctgcagtctgaagattttgcagtttattactgtcagcagtataataactggcctccgtggacgttcggccaagggaccaaagtggatatcaaac

SEQ ID NO: 604 - protein, 108 aa, Homo sapiens
AIQMTQSLPPATLAPGERATLSCRASQSVSNNLAWYQQKPGQAPRLLIYGASTRATGIPARFSGSGSGTEFTLTISSLQSEDFAVYYCQQYNNWPPWTFGQGTKVDIK

SEQ ID NO: 605 - protein, 8 aa, Homo sapiens
GFTFSSYA

SEQ ID NO: 606 - protein, 8 aa, Homo sapiens
ISTGSYFI

SEQ ID NO: 607 - protein, 13 aa, Homo sapiens
ARGKEDTSAAFDI

SEQ ID NO: 608 - protein, 6 aa, Homo sapiens
QSVSNN

SEQ ID NO: 609 - skipped

SEQ ID NO: 610 - protein, 10 aa, Homo sapiens [sequence omitted]

SEQ ID NO: 611 - DNA, 355 bp, Homo sapiens
  source 1..355: mol_type = other DNA
gaggtgcagctggtgcagtctggggctgaggtgaagaagcctgggtcctcggtgaaggtctcctgcaaggcttctggaggcaccttcagcagctctgttatcagctgggtgcgacaggcccctggacaaggccttgagtggatgggagggatcatccctctctttggttcagcaaactacgcacagaagttccagggcagagtcacgattaccgcggacgaatccacgagcacagcctacatggagatgactagcctgagatctgaagacacggccgtgtattactgtgcgaaagtttcccagtgggcgttaatactcttctggggccagggaaccctggtcaccgtctcctcag

SEQ ID NO: 612 - protein, 118 aa, Homo sapiens
EVQLVQSGAEVKKPGSSVKVSCKASGGTFSSSVISWVRQAPGQGLEWMGGIIPLFGSANYAQKFQGRVTITADESTSTAYMEMTSLRSEDTAVYYCAKVSQWALILFWGQGTLVTVSS

SEQ ID NO: 613 - DNA, 328 bp, Homo sapiens
  source 1..328: mol_type = other DNA
gccatccggatgacccagtctccaggcaccctgtctttgtctccaggggaaagagccaccctctcctgcagggccagtcagagtgttagcagcagctacttagcctggtaccagcagaaacctggccaggctcccaggctcctcatctatggtgcatccagcagggccactggcatcccagacaggttcagtggcagtgggtctgggacagacttcactctcaccatcagcagactggagcctgaagattttgcagtgtattactgtcagcagtatggtacctcaccttcgtggacgttcggccaagggaccaaggtggagatcaaac

SEQ ID NO: 614 - protein, 109 aa, Homo sapiens
AIRMTQSPGTLSLSPGERATLSCRASQSVSSSYLAWYQQKPGQAPRLLIYGASSRATGIPDRFSGSGSGTDFTLTISRLEPEDFAVYYCQQYGTSPSWTFGQGTKVEIK

SEQ ID NO: 615 - protein, 8 aa, Homo sapiens
GGTFSSSV

SEQ ID NO: 616 - protein, 8 aa, Homo sapiens
IIPLFGSA

SEQ ID NO: 617 - protein, 11 aa, Homo sapiens
AKVSQWALILF

SEQ ID NO: 618 - protein, 7 aa, Homo sapiens
QSVSSSY

SEQ ID NO: 619 - skipped

SEQ ID NO: 620 - protein, 10 aa, Homo sapiens
QQYGTSPSWT

SEQ ID NO: 621 - DNA, 355 bp, Homo sapiens
  source 1..355: mol_type = other DNA
gaggtgcagctggtgcagtctggggctgaggtgaagaagcctgggtcctcggtgaaggtctcctgcaaggcgtctagaggcaccttcaacacctatgttttcacctgggtgcgacaggcccctggacaagggcttgagtggatgggagggatcatccctttctttggtacagcagactacgcacagaagttccagggcagagtcacgattaccgcggacgactccacgagcacagcctacatggagctgagcagcctgagatctgaggacacggccgtgtattactgttcgaggctcagccagtgggacctactacccatgtggggccagggaaccctggtcaccgtctcctcag

SEQ ID NO: 622 - protein, 118 aa, Homo sapiens
EVQLVQSGAEVKKPGSSVKVSCKASRGTFNTYVFTWVRQAPGQGLEWMGGIIPFFGTADYAQKFQGRVTITADDSTSTAYMELSSLRSEDTAVYYCSRLSQWDLLPMWGQGTLVTVSS

SEQ ID NO: 623 - DNA, 331 bp, Homo sapiens
  source 1..331: mol_type = other DNA
gatattgtgatgactcagtctccaggcaccctgtctttgtctccaggggaaagagccaccctctcctgcagggccagtcagagttttaccagcagctacttagcctggtaccagcagaaacctggccaggctcccaggctcctcatctatggtgcatccagcagggccactggcatcccagacaggttcagtggcactgggtctgggacagacttcactctcaccatcagcagactggagcctgaagattttgcagtatattactgtcagcagtatggtacgtcacctcgcatgtacacttttggccaggggaccaaagtggatatcaaac

SEQ ID NO: 624 - protein, 110 aa, Homo sapiens
DIVMTQSPGTLSLSPGERATLSCRASQSFTSSYLAWYQQKPGQAPRLLIYGASSRATGIPDRFSGTGSGTDFTLTISRLEPEDFAVYYCQQYGTSPRMYTFGQGTKVDIK

SEQ ID NO: 625 - protein, 8 aa, Homo sapiens
RGTFNTYV

SEQ ID NO: 626 - protein, 8 aa, Homo sapiens
IIPFFGTA

SEQ ID NO: 627 - protein, 11 aa, Homo sapiens
SRLSQWDLLPM

SEQ ID NO: 628 - protein, 7 aa, Homo sapiens
QSFTSSY

SEQ ID NO: 629 - skipped

SEQ ID NO: 630 - protein, 11 aa, Homo sapiens
QQYGTSPRMYT

SEQ ID NO: 631 - DNA, 361 bp, Homo sapiens
  source 1..361: mol_type = other DNA
cagctgcagctggtggagtctggggctgaggtgaagaagcctggggcttcagtgaaggtctcctgcaaggtttccggatacaccctcactgaattatccatgcactgggtgcgacaggctcctggaaaagggcttgagtggatggggggttttgatcctgaagatggtgagacaatctacgcacagaagttccagggcagagtcaccatgaccgaggacacatctatagacacagtgtacatggagctgagcagcctgagatctgaggacacggccgtgtattactgtgcaatagatcgcaagcactggctggtaggtcttgactactggggccagggaaccctggtcaccgtctcctcag

SEQ ID NO: 632 - protein, 120 aa, Homo sapiens
QLQLVESGAEVKKPGASVKVSCKVSGYTLTELSMHWVRQAPGKGLEWMGGFDPEDGETIYAQKFQGRVTMTEDTSIDTVYMELSSLRSEDTAVYYCAIDRKHWLVGLDYWGQGTLVTVSS

SEQ ID NO: 633 - DNA, 322 bp, Homo sapiens
  source 1..322: mol_type = other DNA
gccatccggatgacccagtctccatcctcactgtctgcatctgtaggagacagagtcaccatcacttgtcgggcgagtcagggcattaggaattatttagcctggtttcagcagaaaccagggaaagcccctaagtccctgatctatgctgcatccagtttgcaaagtggggtcccatcaaagttcagcggcagtggatctgggacagatttcactctcaccatcagcagcctgcagcctgaagattttgcaacttattactgccaacagtataatagttaccccctcaccttcggccaagggacacgactggagattaaac

SEQ ID NO: 634 - protein, 107 aa, Homo sapiens
AIRMTQSPSSLSASVGDRVTITCRASQGIRNYLAWFQQKPGKAPKSLIYAASSLQSGVPSKFSGSGSGTDFTLTISSLQPEDFATYYCQQYNSYPLTFGQGTRLEIK

SEQ ID NO: 635 - protein, 8 aa, Homo sapiens
GYTLTELS

SEQ ID NO: 636 - protein, 8 aa, Homo sapiens
FDPEDGET

SEQ ID NO: 637 - protein, 13 aa, Homo sapiens
AIDRKHWLVGLDY

SEQ ID NO: 638 - protein, 6 aa, Homo sapiens
QGIRNY

SEQ ID NO: 639 - skipped

SEQ ID NO: 640 - protein, 9 aa, Homo sapiens
QQYNSYPLT

SEQ ID NO: 641 - DNA, 364 bp, Homo sapiens
  source 1..364: mol_type = other DNA
caggtgcagctggtgcagtccggagcagaggtgaaaaagcccggggagtctctgaggatctcctgtaagggttctggacacaactctcccagctactggattagctgggtgcgccagatgcccgggaaaggcctggagtggatggggagaattgatcctagtgactcttataccaactacagcccgtccttccaaggccatgtcaccatctcagctgacaagtccatcagtactgcctacctacagtggagcagcctgcaggcctcggacaccgccatttattactgtgcgagacacgtggttgcattgactcatttgtaccctgactactggggccagggaaccctggtcaccgtctcctcag

SEQ ID NO: 642 - protein, 121 aa, Homo sapiens
QVQLVQSGAEVKKPGESLRISCKGSGHNSPSYWISWVRQMPGKGLEWMGRIDPSDSYTNYSPSFQGHVTISADKSISTAYLQWSSLQASDTAIYYCARHVVALTHLYPDYWGQGTLVTVSS

SEQ ID NO: 643 - DNA, 325 bp, Homo sapiens
  source 1..325: mol_type = other DNA
gacatccagatgacccagtctccagccaccctgtctgtgtctccaggggaaagagccaccctctcctgcagggccagtcaaagtgttagcagcaccttagcctggtaccagcagaaacctggccaggctcccaggctcctcatctatggtgcatccaccagggccactggtatcccagccaggttcagtggcagtgggtctgggacagagttcactctcaccatcagcagcctgcagtctgaagattttgcagtttattactgtcagcaatataataactggtccacgtggacgttcggccaagggaccaaagtggatatcaaac

SEQ ID NO: 644 - protein, 108 aa, Homo sapiens
DIQMTQSPATLSVSPGERATLSCRASQSVSSTLAWYQQKPGQAPRLLIYGASTRATGIPARFSGSGSGTEFTLTISSLQSEDFAVYYCQQYNNWSTWTFGQGTKVDIK

SEQ ID NO: 645 - protein, 8 aa, Homo sapiens
GHNSPSYW

SEQ ID NO: 646 - protein, 8 aa, Homo sapiens
IDPSDSYT

SEQ ID NO: 647 - protein, 14 aa, Homo sapiens
ARHVVALTHLYPDY

SEQ ID NO: 648 - protein, 6 aa, Homo sapiens
QSVSST

SEQ ID NO: 649 - skipped

SEQ ID NO: 650 - protein, 10 aa, Homo sapiens
QQYNNWSTWT

SEQ ID NO: 651 - DNA, 379 bp, Homo sapiens
  source 1..379: mol_type = other DNA
caggtgcagctgcaggagtcgggcccaggactggtgaagccttcggagaccctgtccctcacctgcactgtctttggtggctccatcaccagtagtaatcactactgggtctggatccgccagcccccagggaaggggctggagtggattgggagtatgtattatagtgggagcaccgcctacaacccgtccctcacgaatcgagtcaccatatccgtagacacgtccaagaaccagttctccctgaagctgagctccgtgaccgccgcagacacggctgtgtattactgtgcgagacaaatcgggcccaagaggccctcgcaagtggctgactggttcgacccctggggccagggaaccctggtcaccgtctcctcag

SEQ ID NO: 652 - protein, 126 aa, Homo sapiens
QVQLQESGPGLVKPSETLSLTCTVFGGSITSSNHYWVWIRQPPGKGLEWIGSMYYSGSTAYNPSLTNRVTISVDTSKNQFSLKLSSVTAADTAVYYCARQIGPKRPSQVADWFDPWGQGTLVTVSS

SEQ ID NO: 653 - DNA, 322 bp, Homo sapiens
  source 1..322: mol_type = other DNA
gacatccagttgacccagtctccatccttcctgtctgcatctgtaggagacagagtcaccatcacttgccgggccagtcagggcattagcagttatttagcctggtatcagcaaaaacctgggaaagcccctaagctcctgatctatgctgcatccactttgcaaagtggggtcccatcaaggttcagcggcagtggatctgggacagaattcactctcacaatcagcagcctgcagcctgaagattttgcaacttattactgtcaacagcttaatagttacccgctcactttcggcggagggaccaaggtggaaatcaaac

SEQ ID NO: 654 - protein, 107 aa, Homo sapiens
DIQLTQSPSFLSASVGDRVTITCRASQGISSYLAWYQQKPGKAPKLLIYAASTLQSGVPSRFSGSGSGTEFTLTISSLQPEDFATYYCQQLNSYPLTFGGGTKVEIK

SEQ ID NO: 655 - protein, 10 aa, Homo sapiens
GGSITSSNHY

SEQ ID NO: 656 - protein, 7 aa, Homo sapiens
MYYSGST

SEQ ID NO: 657 - protein, 18 aa, Homo sapiens
ARQIGPKRPSQVADWFDP

SEQ ID NO: 658 - protein, 6 aa, Homo sapiens
QGISSY

SEQ ID NO: 659 - skipped

SEQ ID NO: 660 - protein, 9 aa, Homo sapiens
QQLNSYPLT

SEQ ID NO: 661 - DNA, 382 bp, Homo sapiens
  source 1..382: mol_type = other DNA
caggtgcagctgcaggagtcgggcccaggactggtgaagccttcggagaccctgtccctcacttgcactgtctctggtgactccatcagcagtagtcgttactactggggctggatccgccagcccccagggaaggggctggagtggattgggactttctattatagtgggatcacgtactacaacccgtccctcaagagtcgagtcaccatattcgtagacacgtccaagaaccagttctccctgaagctgagctctgtgaccgccgcagacacggctgtttattactgtgcgagaccccgaccccccgattactatgataatagtggtgcgctcctttttgatatctggggccaagggacaatggtcaccgtctcttcag

SEQ ID NO: 662 - protein, 127 aa, Homo sapiens
QVQLQESGPGLVKPSETLSLTCTVSGDSISSSRYYWGWIRQPPGKGLEWIGTFYYSGITYYNPSLKSRVTIFVDTSKNQFSLKLSSVTAADTAVYYCARPRPPDYYDNSGALLFDIWGQGTMVTVSS

SEQ ID NO: 663 - DNA, 325 bp, Homo sapiens
  source 1..325: mol_type = other DNA
gccatccggatgacccagtctccttccaccctgtctgcatctgtaggagacagagtcaccatcgcttgccgggccagtcagagtattagtgcctggttggcctggtatcagcagaaaccagggaaagcccctaagctcctgatctataaggcatctagtttagaaagtggggtcccatcaaggttcagcggcagtggatctgggacagaattcactctcaccatcaacagcctgcagcctgatgattttgccacttattactgccaacagtatattagttcttctccgtggacgttcggccaagggaccaaggtggaaatcaaac

SEQ ID NO: 664 - protein, 108 aa, Homo sapiens
AIRMTQSPSTLSASVGDRVTIACRASQSISAWLAWYQQKPGKAPKLLIYKASSLESGVPSRFSGSGSGTEFTLTINSLQPDDFATYYCQQYISSSPWTFGQGTKVEIK

SEQ ID NO: 665 - protein, 10 aa, Homo sapiens
GDSISSSRYY

SEQ ID NO: 666 - protein, 7 aa, Homo sapiens
FYYSGIT

SEQ ID NO: 667 - protein, 19 aa, Homo sapiens
ARPRPPDYYDNSGALLFDI

SEQ ID NO: 668 - protein, 6 aa, Homo sapiens
QSISAW

SEQ ID NO: 669 - skipped

SEQ ID NO: 670 - protein, 10 aa, Homo sapiens
QQYISSSPWT

SEQ ID NO: 671 - DNA, 370 bp, Homo sapiens
  source 1..370: mol_type = other DNA
cagctgcagctgcaggagtcgggcccaggactggtgaggccttcacagaccctgtccctctcctgcactgtctctggtggctccatcagcagtgccactcactactggagctggatccgccagcacccagggagaggcctggagtggattgggtacatctattacactgggggcaccttttacaatccgtccctcaagagtcgacttaccatatcagtggacacgtctaagaaccagttctccctgaagctgagcgctgtgactgccgcggacacggccgtgtattactgtgcgagagttatagcagctcgtccgggatctacctactttgacttctggggccggggaaccctggtcaccgtctcctcag

SEQ ID NO: 672 - protein, 123 aa, Homo sapiens
QLQLQESGPGLVRPSQTLSLSCTVSGGSISSATHYWSWIRQHPGRGLEWIGYIYYTGGTFYNPSLKSRLTISVDTSKNQFSLKLSAVTAADTAVYYCARVIAARPGSTYFDFWGRGTLVTVSS

SEQ ID NO: 673 - DNA, 337 bp, Homo sapiens
  source 1..337: mol_type = other DNA
caatctgccctgactcagcctgcctccgtgtctgggtctcctggacagtcgatcaccatctcctgcactggaaccagcagtgacgttagtggctataactatgtctcctggtaccaacaacacccagacaaagcccccaaactcttgatttatgatgtcactaatcggcccacaggggtttctaatcgcttctctgcctccaagtctggcaacacggcctccctgaccatctctgggctccaggctgaggacgaggctgattattactgcagctcagatacaaatagtattcctcggtatgtggtgttcggcggagggaccaagctgaccgtcctag

SEQ ID NO: 674 - protein, 112 aa, Homo sapiens
QSALTQPASVSGSPGQSITISCTGTSSDVSGYNYVSWYQQHPDKAPKLLIYDVTNRPTGVSNRFSASKSGNTASLTISGLQAEDEADYYCSSDTNSIPRYVVFGGGTKLTVL

SEQ ID NO: 675 - protein, 10 aa, Homo sapiens
GGSISSATHY

SEQ ID NO: 676 - protein, 7 aa, Homo sapiens
IYYTGGT

SEQ ID NO: 677 - protein, 15 aa, Homo sapiens
ARVIAARPGSTYFDF

SEQ ID NO: 678 - protein, 9 aa, Homo sapiens
SSDVSGYNY

SEQ ID NO: 679 - skipped

SEQ ID NO: 680 - protein, 9 aa, Homo sapiens
SSDTNSIPR

SEQ ID NO: 681 - DNA, 394 bp, Homo sapiens
  source 1..394: mol_type = other DNA
gaggtgcagctggtggagtctggggctgaggtgaagaagcctgggtcctcggtgaaggtctcctgcaaggcttctggaggcaccttcagcagctatgctatcaactgggtgcgacaggcccctggacaagggcttgagtggatgggagggatcatccctatctttcgtacgccgcactacgcacagaaattccagggcagagtcacgattaccgcggacgaatctacgggcacagcctacatggagctgagcagcctgcgatctgaagacacggccgtgtattactgtgcgagcccctcttgtggtggtgactgcccccagtacttaaaatcatccaaactagactggtacttcgatctctggggccgtggcaccctggtcaccgtctcctcag

SEQ ID NO: 682 - protein, 131 aa, Homo sapiens
EVQLVESGAEVKKPGSSVKVSCKASGGTFSSYAINWVRQAPGQGLEWMGGIIPIFRTPHYAQKFQGRVTITADESTGTAYMELSSLRSEDTAVYYCASPSCGGDCPQYLKSSKLDWYFDLWGRGTLVTVSS

SEQ ID NO: 683 - DNA, 325 bp, Homo sapiens
  source 1..325: mol_type = other DNA
gtcatctggatgacccagtctccaggcaccctgtctttgtctccaggggaaagagccaccctctcctgcagggccagtcagagtgttagcagcacctacttagcctggtaccagcagaaacctggccaggctcccaggctcctcatctatggtgcatccagcagggccactggcatcccagacaggttcagtggcagtgggtctgggacagacttcactctcaccatcagcagactggaacctgaagattttgcagtgtattactgtcagcactatggtagctcacctctcaccttcggccaagggacacgactggagattaaac

SEQ ID NO: 684 - protein, 108 aa, Homo sapiens
VIWMTQSPGTLSLSPGERATLSCRASQSVSSTYLAWYQQKPGQAPRLLIYGASSRATGIPDRFSGSGSGTDFTLTISRLEPEDFAVYYCQHYGSSPLTFGQGTRLEIK

SEQ ID NO: 685 - protein, 8 aa, Homo sapiens
GGTFSSYA

SEQ ID NO: 686 - protein, 8 aa, Homo sapiens
IIPIFRTP

SEQ ID NO: 687 - protein, 24 aa, Homo sapiens
ASPSCGGDCPQYLKSSKLDWYFDL

SEQ ID NO: 688 - protein, 7 aa, Homo sapiens
QSVSSTY

SEQ ID NO: 689 - skipped

SEQ ID NO: 690 - protein, 9 aa, Homo sapiens
QHYGSSPLT

SEQ ID NO: 691 - DNA, 355 bp, Homo sapiens
  source 1..355: mol_type = other DNA
gaggtgcagctggtggagtctggaggaggcttgatccagcctggggggtccctgagactctcctgtgcagcctctgagatcatcgtcagtaggaactacatgagctgggtccgccaggctccagggaaggggctggagtgggtctcagttatttatagcggtggtagcacgttctacgcagactccgtgaagggccgattcaccatctccagagacaattccaagaacacgctgtatcttcaaatgaacagcctgagagccgaggacacggccgtgtattactgtgcgagagacctcgacgtagtgggaggtactgactactggggccagggaaccctggtcaccgtctcctcag

SEQ ID NO: 692 - protein, 118 aa, Homo sapiens
EVQLVESGGGLIQPGGSLRLSCAASEIIVSRNYMSWVRQAPGKGLEWVSVIYSGGSTFYADSVKGRFTISRDNSKNTLYLQMNSLRAEDTAVYYCARDLDVVGGTDYWGQGTLVTVSS

SEQ ID NO: 693 - DNA, 328 bp, Homo sapiens
  source 1..328: mol_type = other DNA
gaaattgtgttgacacagtctccaggcaccctgtctttgtctccaggggaaagagccaccctctcctgcagggccagtcagagtgttagcagcagctacttagcctggtaccagcagaaacctggccaggctcccaggctcctcatctatggtgcatccagcagggccactggcatcccagacaggttcagtggcagtgggtctgggacagacttcactctcaccatcagcagactggagcctgaagattttgcagtgtattactgtcagcagtatggtagctcaccagggtacacttttggccaggggaccaaagtggatatcaaac

SEQ ID NO: 694 - protein, 109 aa, Homo sapiens
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSPGYTFGQGTKVDIK

SEQ ID NO: 695 - protein, 8 aa, Homo sapiens
EIIVSRNY

SEQ ID NO: 696 - protein, 7 aa, Homo sapiens
IYSGGST

SEQ ID NO: 697 - protein, 12 aa, Homo sapiens
ARDLDVVGGTDY

SEQ ID NO: 698 - protein, 7 aa, Homo sapiens
QSVSSSY

SEQ ID NO: 699 - skipped

SEQ ID NO: 700 - protein, 10 aa, Homo sapiens
QQYGSSPGYT

SEQ ID NO: 701 - DNA, 373 bp, Homo sapiens
  source 1..373: mol_type = other DNA
gaggtgcagctgttggagtcgggcccaggactggtgaagccttcggagaccctgtccctcacctgcaccgtctctggtggctccatcagcagatactcctggtcctggatccggcagcccgccgggaggggactggagtggatcgggcgtatgtatagcagtgggggcaccaactataacccctccctcgagagtcgagtcaccatgtcacttgacacgtccaagaagcagttctccctgaagctgagctctgtgaccgccgcggacacggccgtgtattactgtgcggcggcttcaattgatcaagtatgggggacttatcgtgatgcttttgatatctggggtcaagggacaatggtcaccgtctcttcag

SEQ ID NO: 702 - protein, 124 aa, Homo sapiens
EVQLLESGPGLVKPSETLSLTCTVSGGSISRYSWSWIRQPAGRGLEWIGRMYSSGGTNYNPSLESRVTMSLDTSKKQFSLKLSSVTAADTAVYYCAAASIDQVWGTYRDAFDIWGQGTMVTVSS

SEQ ID NO: 703 - DNA, 322 bp, Homo sapiens
  source 1..322: mol_type = other DNA
gccatccggatgacccagtctccatcctccctggctgcatctgtaggagacagagtcaccatctcttgccgggcaggtcagagcattagcagctttttacattggtatcagcagaaagtagggaaagcccctaagctcctgatctatgatgcgtccagtttgcaaagtggggtcccatcaaggttcagtggcagtggatctgggacagatttcactctcaccatcagcagtctgcaacctgaagattttgcagcttactactgtcaacagagttacgaaaacccgcttactttcggcggagggaccaaagtggatatcaaac

SEQ ID NO: 704 - protein, 107 aa, Homo sapiens
AIRMTQSPSSLAASVGDRVTISCRAGQSISSFLHWYQQKVGKAPKLLIYDASSLQSGVPSRFSGSGSGTDFTLTISSLQPEDFAAYYCQQSYENPLTFGGGTKVDIK

SEQ ID NO: 705 - protein, 8 aa, Homo sapiens
GGSISRYS

SEQ ID NO: 706 - protein, 7 aa, Homo sapiens
MYSSGGT

SEQ ID NO: 707 - protein, 18 aa, Homo sapiens
AAASIDQVWGTYRDAFDI

SEQ ID NO: 708 - protein, 6 aa, Homo sapiens
QSISSF

SEQ ID NO: 709 - skipped

SEQ ID NO: 710 - protein, 9 aa, Homo sapiens
QQSYENPLT

SEQ ID NO: 711 - DNA, 373 bp, Homo sapiens
  source 1..373: mol_type = other DNA
gaagtgcagctggtggagtctggggctgaggtgaagaggcctggggcctcagtgaaggtttcctgcaaggcatctggatacaccttcaccaactactttatgcactgggtgcgacaggcccctggacaagggcttgagtggatgggagttatcaaccctagtgatggtggcgcaagctacccacagaagttccagggcagagtcaccatgaccagggacacgtccacgagcacagtctacatggatctgagcagcctgagatctgaggacacggccgtctattcctgtgcgaggggggcttttgatgttagcggcagctggtacgtcccctttgactactggggccagggaactctggtcaccgtctcctcag

SEQ ID NO: 712 - protein, 124 aa, Homo sapiens
EVQLVESGAEVKRPGASVKVSCKASGYTFTNYFMHWVRQAPGQGLEWMGVINPSDGGASYPQKFQGRVTMTRDTSTSTVYMDLSSLRSEDTAVYSCARGAFDVSGSWYVPFDYWGQGTLVTVSS

SEQ ID NO: 713 - DNA, 340 bp, Homo sapiens
  source 1..340: mol_type = other DNA
cagtctgtcgtgacgcagccgccctcagtgtctggggccccagggcagagggtcaccatctcctgcactgggagcagctccaacatcggggcaggttatgatgtacactggtaccagcagcttccaggagcagcccccaaactcctcatctatggtaacaccaatcggccctcaggggtccctgaccgattctctggctccaagtctggcacctccgcctccctggccatcactgggctccaggctgaggatgaggctgattattactgccagtcctatgacatcaccctgagtggttcggggtatgtcttcggaactgggaccaaggtcaccgtcctag

SEQ ID NO: 714 - protein, 113 aa, Homo sapiens
QSVVTQPPSVSGAPGQRVTISCTGSSSNIGAGYDVHWYQQLPGAAPKLLIYGNTNRPSGVPDRFSGSKSGTSASLAITGLQAEDEADYYCQSYDITLSGSGYVFGTGTKVTVL

SEQ ID NO: 715 - protein, 8 aa, Homo sapiens
GYTFTNYF

SEQ ID NO: 716 - protein, 8 aa, Homo sapiens
INPSDGGA

SEQ ID NO: 717 - protein, 17 aa, Homo sapiens
ARGAFDVSGSWYVPFDY

SEQ ID NO: 718 - protein, 9 aa, Homo sapiens
SSNIGAGYD

SEQ ID NO: 719 - skipped

SEQ ID NO: 720 - protein, 13 aa, Homo sapiens
QSYDITLSGSGYV

SEQ ID NO: 721 - DNA, 367 bp, Homo sapiens
  source 1..367: mol_type = other DNA
caggtgcagctggtggagtctgggggaggcgtggtccagcctgggaggtccctgagactctcctgtgcagcctctggattcaccttcaggacctatgctgtgcactgggtccgccaggctccaggcaaggggccagagtgggtggcagttatatcatatgatggaagtaataaatactacgcagactccgttaagggccgattcaccctctccagagacacttccaagaacacgctgtatctgcaaatgaacagcctgagagctgaggacacggctgtgtattactgtgcgagcagaggggacacggtgactacaggtgacgcttttgatatctggggccaagggacaatggtcaccgtctcttcag

SEQ ID NO: 722 - protein, 122 aa, Homo sapiens
QVQLVESGGGVVQPGRSLRLSCAASGFTFRTYAVHWVRQAPGKGPEWVAVISYDGSNKYYADSVKGRFTLSRDTSKNTLYLQMNSLRAEDTAVYYCASRGDTVTTGDAFDIWGQGTMVTVSS

SEQ ID NO: 723 - DNA, 334 bp, Homo sapiens
  source 1..334: mol_type = other DNA
tcctatgagctgacacagccaccctcggtgtcagtgtccccaggacagacggccaggatcacctgctctggagatgcattgccaaagcaatatacttattggtaccagcagaagccaggccagccccctgtgctggtgatatataaagacagtgagaggccctcagggatccctgagcgattctctggctccagctcagggacaacagtcacgttgaccatcagtggagtccaggcagaagatgaggctgactattactgtcaatcaacagacagcagtgctacttatccgggaaatgtggttttcggcggagggaccaagttgaccgtcctag

SEQ ID NO: 724 - protein, 111 aa, Homo sapiens
SYELTQPPSVSVSPGQTARITCSGDALPKQYTYWYQQKPGQPPVLVIYKDSERPSGIPERFSGSSSGTTVTLTISGVQAEDEADYYCQSTDSSATYPGNVVFGGGTKLTVL

SEQ ID NO: 725 - protein, 8 aa, Homo sapiens
GFTFRTYA

SEQ ID NO: 726 - protein, 8 aa, Homo sapiens
ISYDGSNK

SEQ ID NO: 727 - protein, 15 aa, Homo sapiens
ASRGDTVTTGDAFDI

SEQ ID NO: 728 - protein, 6 aa, Homo sapiens
ALPKQY

SEQ ID NO: 729 - skipped

SEQ ID NO: 730 - protein, 14 aa, Homo sapiens
QSTDSSATYPGNVV

SEQ ID NO: 731 - DNA, 370 bp, Homo sapiens
  source 1..370: mol_type = other DNA
gaggtgcagctggtggagtctgggcctgaggtgaagaagcctgggacctcagtgaaggtctcctgcaaggcgtctggattcagttttagtatgtctgctatgcagtgggtgcgacgggctcgtggacaacgccttgagtggataggatggatcgtccctggcagtggtaacgcaaactacgcgcagaagtttcaggaaagagtcaccattactagggacgagtccacaaacacaggttatatggagttgagcagcctgagatccgaggacacggccgtgtattattgtgcggcccctcattgtaataagaccaactgctatgatgcttttgatatctggggccaagggacaatggtcaccgtctcttcag

SEQ ID NO: 732 - protein, 123 aa, Homo sapiens
EVQLVESGPEVKKPGTSVKVSCKASGFSFSMSAMQWVRRARGQRLEWIGWIVPGSGNANYAQKFQERVTITRDESTNTGYMELSSLRSEDTAVYYCAAPHCNKTNCYDAFDIWGQGTMVTVSS

SEQ ID NO: 733 - DNA, 325 bp, Homo sapiens
  source 1..325: mol_type = other DNA
gccatccggatgacccagtctccaggcaccctgtctttgtctccaggggaaagagccaccctctcctgcagggccagtcagagcgttaggagcagttacttagcctggtaccagcagaaacctggccaggctcccaggctcctcatctatggtgcatccaccagggccactggcatcccagacaggttcagtggcagtgggtctgggacagacttcattctcaccatcaacagactggagcctgaagatcttgcagtctattactgtcagcagtttggtagctcaccatggacgttcggccaagggaccaaagtggatatcaaac

SEQ ID NO: 734 - protein, 108 aa, Homo sapiens
AIRMTQSPGTLSLSPGERATLSCRASQSVRSSYLAWYQQKPGQAPRLLIYGASTRATGIPDRFSGSGSGTDFILTINRLEPEDLAVYYCQQFGSSPWTFGQGTKVDIK

SEQ ID NO: 735 - protein, 8 aa, Homo sapiens
GFSFSMSA

SEQ ID NO: 736 - protein, 8 aa, Homo sapiens
IVPGSGNA

SEQ ID NO: 737 - protein, 16 aa, Homo sapiens
AAPHCNKTNCYDAFDI

SEQ ID NO: 738 - protein, 7 aa, Homo sapiens
QSVRSSY

SEQ ID NO: 739 - skipped

SEQ ID NO: 740 - protein, 9 aa, Homo sapiens
QQFGSSPWT

SEQ ID NO: 741 - DNA, 355 bp, Homo sapiens
  source 1..355: mol_type = other DNA
gaggtgcagctggtggagtctgggggaggtgtggtccagcctggggggtccctgagactctcctgtgcagcctctggaatcatagtcagtgccaactacatgacctgggtccgccaggctccagggaaggggctggaatgggtctcagttatttatcccggtggtagcacattctacgcggactccgtgaagggccgattcaccatctccagagacaactccaagaacacactgtatcttcaaatgaacagcctgagagttgaggactcggctgtgtattactgtgcgagagatttggagctggctggtttcaatgacttctggggtcagggaaccctggtcaccgtctcctcag

SEQ ID NO: 742 - protein, 118 aa, Homo sapiens
EVQLVESGGGVVQPGGSLRLSCAASGIIVSANYMTWVRQAPGKGLEWVSVIYPGGSTFYADSVKGRFTISRDNSKNTLYLQMNSLRVEDSAVYYCARDLELAGFNDFWGQGTLVTVSS

SEQ ID NO: 743 - DNA, 328 bp, Homo sapiens
  source 1..328: mol_type = other DNA
gatgttgtgatgactcagtctccaggcaccctggctttgtctccaggggaaagagccaccctctcctgcaggaccagccagagtgttagcagcaactacttagcctggtaccagcagaaacctggccaggctcccaggctcctcatctatggtgcatccagcagggccactggcatcccagacaggttcagtggcagtgggtctgggacagacttcactctcaccatcagcagactggagcctgaagattttgcagtgtattactgtcagcagtttggtagttcacctcggtacacttttggccaggggaccaaggtggagatcaaac

SEQ ID NO: 744 - protein, 109 aa, Homo sapiens
DVVMTQSPGTLALSPGERATLSCRTSQSVSSNYLAWYQQKPGQAPRLLIYGASSRATGIPDRFSGSGSGTDFTLTISRLEPEDFAVYYCQQFGSSPRYTFGQGTKVEIK

SEQ ID NO: 745 - protein, 8 aa, Homo sapiens
GIIVSANY

SEQ ID NO: 746 - protein, 7 aa, Homo sapiens
IYPGGST

SEQ ID NO: 747 - protein, 12 aa, Homo sapiens
ARDLELAGFNDF

SEQ ID NO: 748 - protein, 7 aa, Homo sapiens
QSVSSNY

SEQ ID NO: 749 - skipped

SEQ ID NO: 750 - protein, 10 aa, Homo sapiens
QQFGSSPRYT

SEQ ID NO: 751 - DNA, 355 bp, Homo sapiens
  source 1..355: mol_type = other DNA
caggtgcagctggtggagtctgggggaggtgtggtccagcctggggggtccctgagactctcctgtgcagcctctggagtcaccgtcagtagcaactacatgagctgggtccgccaggctccagggaaggggctggagtgggtctcagttctttatgccggtggtagcacattctacgcagactccgtgaagggccgattcaccatctccagagacaattccaagaacacgctgtatcttcaaatgaacagcctgagagctgaggacacggctgcgtattactgtgcgagagatttggcagtggctggtttccttgactcctggggccagggaaccctggtcaccgtctcctcag

SEQ ID NO: 752 - protein, 118 aa, Homo sapiens
QVQLVESGGGVVQPGGSLRLSCAASGVTVSSNYMSWVRQAPGKGLEWVSVLYAGGSTFYADSVKGRFTISRDNSKNTLYLQMNSLRAEDTAAYYCARDLAVAGFLDSWGQGTLVTVSS

SEQ ID NO: 753 - DNA, 328 bp, Homo sapiens
  source 1..328: mol_type = other DNA
gatattgtgatgacccagtctccgggcaccctgtctttgtctccaggggaaagagccaccctctcctgcagggccagtcagggtgttagcagcatctacttagcctggtaccagcagaaacctggccaggctcccaggctcgtcctctatggcgcatccagtagggccactggcatcccagacaggttcagtggcagtgggtctgggacagacttcactctcaccatcagcagactggagcctgaagattttgcagtgtattactgtcagcagtatggtagctcacctcggtacacttttggccaggggaccaaggtggagatcaaac

SEQ ID NO: 754 - protein, 109 aa, Homo sapiens
DIVMTQSPGTLSLSPGERATLSCRASQGVSSIYLAWYQQKPGQAPRLVLYGASSRATGIPDRFSGSGSGTDFTLTISRLEPEDFAVYYCQQYGSSPRYTFGQGTKVEIK

SEQ ID NO: 755 - protein, 8 aa, Homo sapiens
GVTVSSNY

SEQ ID NO: 756 - protein, 7 aa, Homo sapiens
LYAGGST

SEQ ID NO: 757 - protein, 12 aa, Homo sapiens
ARDLAVAGFLDS

SEQ ID NO: 758 - protein, 7 aa, Homo sapiens
QGVSSIY

SEQ ID NO: 759 - skipped

SEQ ID NO: 760 - protein, 10 aa, Homo sapiens
QQYGSSPRYT

SEQ ID NO: 761 - DNA, 351 bp, Homo sapiens
  source 1..351: mol_type = other DNA
caggtgcagctggtggagtctggaggaggcttgatccagccgggggggtccctgagactctcctgtgcagcctctgggatcaccgtcagtagcaactacatgacctgggtccgccaggctccagggaaggggctggagtgggtctcacttctttatgccggtggtagcgcattctatgctgactccgtgaagggccgattcaccatctccagagacaattccaagaacacgctgtatcttctaatgaacagcctgagagtcggcgacacggccgtttattactgtgcgagagatctccaggtctacggtatggacgtctggggccaagggaccacggtcaccgtctcctca

SEQ ID NO: 762 - protein, 117 aa, Homo sapiens
QVQLVESGGGLIQPGGSLRLSCAASGITVSSNYMTWVRQAPGKGLEWVSLLYAGGSAFYADSVKGRFTISRDNSKNTLYLLMNSLRVGDTAVYYCARDLQVYGMDVWGQGTTVTVSS

SEQ ID NO: 763 - DNA, 322 bp, Homo sapiens
  source 1..322: mol_type = other DNA
tcctatgagctgactcagccaccctcggtgtcagtggccccaggacagacggccaggattacctgtgggggaaacaacgatggagctaaaagtgtgcactggtaccagcagaagccaggccaggcccctgtgctggtcgtctatgatgatagcgaccggccctcagggatccctgaacgattctctggctccaactctgggaacacggccaccctgaccatcaccaggatcgaagccggggatgaggccgactattactgtcaggtctgggatagtagtcgtgatcatgtcttcggaactgggaccaaggtcaccgtcctgg

SEQ ID NO: 764 - protein, 107 aa, Homo sapiens
SYELTQPPSVSVAPGQTARITCGGNNDGAKSVHWYQQKPGQAPVLVVYDDSDRPSGIPERFSGSNSGNTATLTITRIEAGDEADYYCQVWDSSRDHVFGTGTKVTVL

SEQ ID NO: 765 - protein, 8 aa, Homo sapiens
GITVSSNY

SEQ ID NO: 766 - protein, 7 aa, Homo sapiens
LYAGGSA

SEQ ID NO: 767 - protein, 11 aa, Homo sapiens
ARDLQVYGMDV

SEQ ID NO: 768 - protein, 6 aa, Homo sapiens
NDGAKS

SEQ ID NO: 769 - skipped

SEQ ID NO: 770 - protein, 10 aa, Homo sapiens
QVWDSSRDHV

SEQ ID NO: 771 - DNA, 351 bp, Homo sapiens
  source 1..351: mol_type = other DNA
gaggtgcagctggtggagtctgggggaggcttggtccagcctggggggtccctgaggctctcctgtgaagcctctgaaataaccgtcagtagcaactacatgaattgggtccgccaggctccagggaaggggctggagtgggtctcagttctttttgccggtggtactacatactacgcagactccgtgaagggccgattcaccatctccagagacaattccaagaacacactgtatcttcaaatgaacaccctgagaattgaggacacggctatttattactgtgcgagagatctcgtcgcatacggtgtggacgtctggggccaagggaccacggtcaccgtctcctca

SEQ ID NO: 772 - protein, 117 aa, Homo sapiens
EVQLVESGGGLVQPGGSLRLSCEASEITVSSNYMNWVRQAPGKGLEWVSVLFAGGTTYYADSVKGRFTISRDNSKNTLYLQMNTLRIEDTAIYYCARDLVAYGVDVWGQGTTVTVSS

SEQ ID NO: 773 - DNA, 322 bp, Homo sapiens
  source 1..322: mol_type = other DNA
gccatccagatgacccagtctccatccttcctgtctgcatctgtaggagacagagtcaccatcacttgccgggccagtcagggcattagcggtgatttagcctggtatcagcaaaaaccagggaaagcccctaagctcctgatctatgctgcatccactttgcaaagtggggtcccatcaaggttcagcggcagtggatctgggacagaattcactctcacaatcagcagcctgcagcctgaagattttgcaacttattactgtcaacaccttaatagttaccctctcacgttcggcggagggaccaaggtggaaatcaaac

SEQ ID NO: 774 - protein, 107 aa, Homo sapiens
AIQMTQSPSFLSASVGDRVTITCRASQGISGDLAWYQQKPGKAPKLLIYAASTLQSGVPSRFSGSGSGTEFTLTISSLQPEDFATYYCQHLNSYPLTFGGGTKVEIK

SEQ ID NO: 775 - protein, 8 aa, Homo sapiens
EITVSSNY

SEQ ID NO: 776 - protein, 7 aa, Homo sapiens
LFAGGTT

SEQ ID NO: 777 - protein, 11 aa, Homo sapiens
ARDLVAYGVDV

SEQ ID NO: 778 - protein, 6 aa, Homo sapiens
QGISGD

SEQ ID NO: 779 - skipped

SEQ ID NO: 780 - protein, 9 aa, Homo sapiens
QHLNSYPLT

SEQ ID NO: 781 - DNA, 352 bp, Homo sapiens
  source 1..352: mol_type = other DNA
caggtacagctgcaggagtcgggcccaggactggtgaagtcttcacagaccctgtccctcacgtgcactgtctctggtgactccatcagccgtggtggttactactggagctggatccgccagcacccagggaagggcctggagtggattgggtccatctattacagtgggagcacctactacaacccgtccctcaagagtcgatttaccatatcagtagacacgtctaagaaccagttctccctgaagctgagctctgtgactgccgcggacacggccgtgtatcactgtgcgagagaaattggtttccttgactactggggccagggaaccctggtcaccgtctcctcag

SEQ ID NO: 782 - protein, 117 aa, Homo sapiens
QVQLQESGPGLVKSSQTLSLTCTVSGDSISRGGYYWSWIRQHPGKGLEWIGSIYYSGSTYYNPSLKSRFTISVDTSKNQFSLKLSSVTAADTAVYHCAREIGFLDYWGQGTLVTVSS

SEQ ID NO: 783 - DNA, 325 bp, Homo sapiens
  source 1..325: mol_type = other DNA
gccatccagatgacccagtctccatcctccctgtctgcatctgtaggagacagagtcaccatcacttgccgggcgagtcaggccattagcaattctttagcctggtatcagcagaaaccagggaaagcccctaagctcctactctatgctgcatccacattggaaagtggggtcccatccaggttcagtggcagtggatctgggacggatttcactctcaccatcagcagcctgcagcctgaagattttgcaacttatttctgtcagcagtactatagtacccctccgaggacgttcggccaagggaccaaagtggatatcaaac

SEQ ID NO: 784 - protein, 108 aa, Homo sapiens
AIQMTQSPSSLSASVGDRVTITCRASQAISNSLAWYQQKPGKAPKLLLYAASTLESGVPSRFSGSGSGTDFTLTISSLQPEDFATYFCQQYYSTPPRTFGQGTKVDIK

SEQ ID NO: 785 - protein, 10 aa, Homo sapiens
GDSISRGGYY

SEQ ID NO: 786 - protein, 7 aa, Homo sapiens
IYYSGST

SEQ ID NO: 787 - protein, 9 aa, Homo sapiens
AREIGFLDY

SEQ ID NO: 788 - protein, 6 aa, Homo sapiens
QAISNS

SEQ ID NO: 789 - skipped

SEQ ID NO: 790 - protein, 10 aa, Homo sapiens
QQYYSTPPRT

SEQ ID NO: 791 - DNA, 382 bp, Homo sapiens
  source 1..382: mol_type = other DNA
caggtgcagctggtggagtctggggctgaggtgaagaagcctgggtcctcggtgaaggtctcctgcaaggcttctggaggcaccttcagcagccatggtgtcatctgggtgcgacaggcccctggacaagggcttgagtggatgggagggatcatccccatctttcccacagcaaactacgcacagaaattccagggcagagtcacaattaccgcggacaaaccctccaacacagcctacatggagctgagcagcctgagatctgaggacacggccgtatattactgtgcgagggcaaggggagaacatgattccgtttggggaagttttcattactattttgactactggggccagggaaccctggtcaccgtctcctcag

SEQ ID NO: 792 - protein, 127 aa, Homo sapiens
QVQLVESGAEVKKPGSSVKVSCKASGGTFSSHGVIWVRQAPGQGLEWMGGIIPIFPTANYAQKFQGRVTITADKPSNTAYMELSSLRSEDTAVYYCARARGEHDSVWGSFHYYFDYWGQGTLVTVSS

SEQ ID NO: 793 - DNA, 325 bp, Homo sapiens
  source 1..325: mol_type = other DNA
cgtcattggatgacccagtctccagccaccctgtctgtgtctccaggggaaagagccaccctctcctgcagggccagtcaaagtattggcagcaacttagcctggtaccagcagaaacctggtcaggctcccaggctcctcatctatggtgcagccaccagggccactggtatcccagccaggttcagtggcagtgggtctgggacagagttcactctcaccatcagcagcctgcagtctgaagattttgcagtttactactgtcagcagtataatgactggcctccgaggacgttcggccaagggaccaaggtggaaatcaaac

SEQ ID NO: 794 - protein, 108 aa, Homo sapiens
RHWMTQSPATLSVSPGERATLSCRASQSIGSNLAWYQQKPGQAPRLLIYGAATRATGIPARFSGSGSGTEFTLTISSLQSEDFAVYYCQQYNDWPPRTFGQGTKVEIK

SEQ ID NO: 795 - protein, 8 aa, Homo sapiens
GGTFSSHG

SEQ ID NO: 796 - protein, 8 aa, Homo sapiens
IIPIFPTA

SEQ ID NO: 797 - protein, 20 aa, Homo sapiens
ARARGEHDSVWGSFHYYFDY

SEQ ID NO: 798 - protein, 6 aa, Homo sapiens
QSIGSN

SEQ ID NO: 799 - skipped

SEQ ID NO: 800 - protein, 10 aa, Homo sapiens
QQYNDWPPRT

SEQ ID NO: 801 - DNA, 369 bp, Homo sapiens
  source 1..369: mol_type = other DNA
caggtgcagctggtggagtctgggggaggcttggtacagcctggcaggtccctgagactctcctgtgcagcctctggattcacgtttgatgattatgccatgcactgggtccggcaagttccagggaagggcctggagtgggtctcaggaattagttggaacagtggtagcatagtctatgcggactttgtgaagggccgattcaccatcgccagagacaacgccaagaactccctgtttctgcaaatgaacagtctgagagctgaggacacggccttgtattactgtgcaaaaagtacggctctacgtcatcagtactactacggtatggacgtctggggccaagggaccacggtcaccgtctcctca

SEQ ID NO: 802 - protein, 123 aa, Homo sapiens
QVQLVESGGGLVQPGRSLRLSCAASGFTFDDYAMHWVRQVPGKGLEWVSGISWNSGSIVYADFVKGRFTIARDNAKNSLFLQMNSLRAEDTALYYCAKSTALRHQYYYGMDVWGQGTTVTVSS

SEQ ID NO: 803 - DNA, 322 bp, Homo sapiens
  source 1..322: mol_type = other DNA
gccatccagatgacccagtctccatcctccctgtctgcatctgtaggagacagagtcaccatcacttgccggacaagtcagaccattagcagctatttaaattggtatcagcagaaaccagggaaagcccctaagctcctgatatatgacgcatccagtttgcaaagtggggtcccatcaaggttcagtggcagtggatatgggacagatttcactctcaccatcagcagtctgcaacctgaagattttgcaacttacttctgtcaacagagttacaataccccgtacgcttttggccaggggaccaaggtggagatcaaac

SEQ ID NO: 804 - protein, 107 aa, Homo sapiens
AIQMTQSPSSLSASVGDRVTITCRTSQTISSYLNWYQQKPGKAPKLLIYDASSLQSGVPSRFSGSGYGTDFTLTISSLQPEDFATYFCQQSYNTPYAFGQGTKVEIK

SEQ ID NO: 805 - protein, 8 aa, Homo sapiens
GFTFDDYA

SEQ ID NO: 806 - protein, 8 aa, Homo sapiens
ISWNSGSI

SEQ ID NO: 807 - protein, 16 aa, Homo sapiens
AKSTALRHQYYYGMDV

SEQ ID NO: 808 - protein, 6 aa, Homo sapiens
QTISSY

SEQ ID NO: 809 - skipped

SEQ ID NO: 810 - protein, 9 aa, Homo sapiens
QQSYNTPYA

SEQ ID NO: 811 - DNA, 358 bp, Homo sapiens
  source 1..358: mol_type = other DNA
caggttcagctggtgcagtctggcactgaggtgaagaagcctggggcctcagtgaaggtctcctgcaaggcttctgattacacctttaccaggtttggtatcatctgggtgcgacaggcccctggacaagggcttgagtggatgggacagatcaacccttacaatggtaacacagactatgcacagaagttccagggcagagtcaccttgaccacagacacatccacgaacacagcctacatggaactgaggagcctgagatctgacgacacggccgtgtattattgtgcgaggtccgctgggagccctaccggccttgactactggggccagggaaccctggtcaccgtctcctcag

SEQ ID NO: 812 - protein, 119 aa, Homo sapiens
QVQLVQSGTEVKKPGASVKVSCKASDYTFTRFGIIWVRQAPGQGLEWMGQINPYNGNTDYAQKFQGRVTLTTDTSTNTAYMELRSLRSDDTAVYYCARSAGSPTGLDYWGQGTLVTVSS

SEQ ID NO: 813 - DNA, 331 bp, Homo sapiens
  source 1..331: mol_type = other DNA
cagtctgtcgtgacgcagccaccctcggtgtctgaagcccccaggcagagggtcaccatctcctgttctggaagcaactccaacatcggaaataatgctgtaaactggtaccagcagctcccaggaaaggctcccaaactcctcgtctattatgatgatctgctgccctcaggggtctctgaccgattctctggctccaagtctggcacctcagcctccctggccatcagtgggctccagtctgaggataaggctgattattactgtgcagcatgggatgacagcctgaatgccttggtgttcggcggagggaccaagctgaccgtcctag

SEQ ID NO: 814 - protein, 110 aa, Homo sapiens
QSVVTQPPSVSEAPRQRVTISCSGSNSNIGNNAVNWYQQLPGKAPKLLVYYDDLLPSGVSDRFSGSKSGTSASLAISGLQSEDKADYYCAAWDDSLNALVFGGGTKLTVL

SEQ ID NO: 815 - protein, 8 aa, Homo sapiens
DYTFTRFG

SEQ ID NO: 816 - protein, 8 aa, Homo sapiens
INPYNGNT

SEQ ID NO: 817 - protein, 12 aa, Homo sapiens
ARSAGSPTGLDY

SEQ ID NO: 818 - protein, 8 aa, Homo sapiens
NSNIGNNA

SEQ ID NO: 819 - skipped

SEQ ID NO: 820 - protein, 11 aa, Homo sapiens
AAWDDSLNALV

SEQ ID NO: 821 - DNA, 354 bp, Homo sapiens
  source 1..354: mol_type = other DNA
gaagtgcagctgttggagtctgggggaggcttggtccagcctggggggtccctgagactctcctgtgtagcctctggactcaccgtcagtagcaactacatgagctgggtccgccaggctccagggaaggggctggagtgggtctcaattatttatcccggtggtaccacatactacgcagactccgtgaagggcagattcaccacctccagagacaaatccaagaacacgctgtatcttcaaatgaacagcctgagagccgaggacacggctgtgtattactgtgcgagagatctggcagtggctgggggtatggacgtctggggccaagggaccacggtcaccgtctcctca

SEQ ID NO: 822 - protein, 118 aa, Homo sapiens
EVQLLESGGGLVQPGGSLRLSCVASGLTVSSNYMSWVRQAPGKGLEWVSIIYPGGTTYYADSVKGRFTTSRDKSKNTLYLQMNSLRAEDTAVYYCARDLAVAGGMDVWGQGTTVTVSS

SEQ ID NO: 823 - DNA, 322 bp, Homo sapiens
  source 1..322: mol_type = other DNA
gaaatagtgatgacgcagtctccatcctccctgtctgcatctgtaggagacagagttaccatcacttgccgggcaagtcagggcattggaaatgatttagggtggtatcagcagaaaccagggaaagcccctaaagtcctgatttatgctgcatccaatttacaaagtggggtcccatcaaggttcagcggcagtggatctggcacagatttcactctcaccatcagcagcctgcagcctgaagattttgcaacttattactgtctacaagattccaattatccctacacttttggccaggggaccaaggtggagatcaaac

SEQ ID NO: 824 - protein, 107 aa, Homo sapiens
EIVMTQSPSSLSASVGDRVTITCRASQGIGNDLGWYQQKPGKAPKVLIYAASNLQSGVPSRFSGSGSGTDFTLTISSLQPEDFATYYCLQDSNYPYTFGQGTKVEIK

SEQ ID NO: 825 - protein, 8 aa, Homo sapiens
GLTVSSNY

SEQ ID NO: 826 - protein, 7 aa, Homo sapiens
IYPGGTT

SEQ ID NO: 827 - protein, 12 aa, Homo sapiens
ARDLAVAGGMDV

SEQ ID NO: 828 - protein, 6 aa, Homo sapiens
QGIGND

SEQ ID NO: 829 - skipped

SEQ ID NO: 830 - protein, 9 aa, Homo sapiens
LQDSNYPYT

SEQ ID NO: 831 - DNA, 355 bp, Homo sapiens
  source 1..355: mol_type = other DNA
gaagtgcagctggtggagtctgggggaggcttggtccagcctggggggtccctgagactctcctgtgcagcctctggagtcatcgtcagtagcaactacatgagctgggtccgccaggctccagggaaggggctgcaatgggtctcagttatttatagcggtggtagcactttctacgcagactccgtgaagggcagattcaccatctccagagacaattccaagaacacgttgtatcttcaaatgaacagcctgagagccgaggacacggctgtgtattactgtgcgagagatttgttagaggcaggcggaactgactactggggccagggaaccctggtcaccgtctcctcag

SEQ ID NO: 832 - protein, 118 aa, Homo sapiens
EVQLVESGGGLVQPGGSLRLSCAASGVIVSSNYMSWVRQAPGKGLQWVSVIYSGGSTFYADSVKGRFTISRDNSKNTLYLQMNSLRAEDTAVYYCARDLLEAGGTDYWGQGTLVTVSS

SEQ ID NO: 833 - DNA, 325 bp, Homo sapiens
  source 1..325: mol_type = other DNA
gatgttgtgatgactcagtctccaggcaccctgtctttgtctccaggggaaagagccaccctctcctgcagggccagtcagtttattggcagctcctacttagcctggtaccagcagaaacctggccaggctcccaggctcctcatctatggtgcatccaacagggccactggcgtcccagacaggttcagtggcagtgggtctgggacagacttcactctcaccatcagcagactggagcctgaagattttgcagtgtattactgtcagcagtatgggagtgcacctgggacgttcggccaagggaccaaggtggaaatcaaac

SEQ ID NO: 834 - protein, 108 aa, Homo sapiens
DVVMTQSPGTLSLSPGERATLSCRASQFIGSSYLAWYQQKPGQAPRLLIYGASNRATGVPDRFSGSGSGTDFTLTISRLEPEDFAVYYCQQYGSAPGTFGQGTKVEIK

SEQ ID NO: 835 - protein, 8 aa, Homo sapiens
GVIVSSNY

SEQ ID NO: 836 - protein, 7 aa, Homo sapiens
IYSGGST

SEQ ID NO: 837 - protein, 12 aa, Homo sapiens
ARDLLEAGGTDY

SEQ ID NO: 838 - protein, 7 aa, Homo sapiens
QFIGSSY

SEQ ID NO: 839 - skipped

SEQ ID NO: 840 - protein, 9 aa, Homo sapiens
QQYGSAPGT

SEQ ID NO: 841 - DNA, 351 bp, Homo sapiens
  source 1..351: mol_type = other DNA
caggtgcagctggtggagtctggaggaggcttggtccagcctggggggtccctgagactctcctgtgcagcctctggtttaatcgtcagtaggaactacatgagctgggtccgccaggctccagggaaggggctggagtgggtctcacttatttatgccggtggtagcacattctactcagactccgtgaagggccgattcaccatctccagacacagttccgagaacacgctgtttcttcaaatgaacagcctgagagctgaggacacggctgtgtattattgtgcgagagatctagtccactacggcatggacgtctggggccaagggaccacggtcaccgtctcctca

SEQ ID NO: 842 - protein, 117 aa, Homo sapiens
QVQLVESGGGLVQPGGSLRLSCAASGLIVSRNYMSWVRQAPGKGLEWVSLIYAGGSTFYSDSVKGRFTISRHSSENTLFLQMNSLRAEDTAVYYCARDLVHYGMDVWGQGTTVTVSS

SEQ ID NO: 843 - DNA, 331 bp, Homo sapiens
  source 1..331: mol_type = other DNA
aattttatgctgactcagcctgcctccgtgtctgggtctcctggacagtcgatcaccatctcctgcactggaaccagcagtgacgttggtggttataactatgtctcctggtaccaacagcacccaggcaaagcccccaaactcatgatttatgatgtcagtaatcggccctcaggggtttctaatcgcttctctggctccaactctggcaacacggcctccctcaccatctctgggctccaggctgaggacgaggctgattattactgcagctcatatacaagcggcagcacttgggtgttcggcggagggaccaagctgaccgtcctag

SEQ ID NO: 844 - protein, 110 aa, Homo sapiens
NFMLTQPASVSGSPGQSITISCTGTSSDVGGYNYVSWYQQHPGKAPKLMIYDVSNRPSGVSNRFSGSNSGNTASLTISGLQAEDEADYYCSSYTSGSTWVFGGGTKLTVL

SEQ ID NO: 845 - protein, 8 aa, Homo sapiens
GLIVSRNY

SEQ ID NO: 846 - protein, 7 aa, Homo sapiens
IYAGGST

SEQ ID NO: 847 - protein, 11 aa, Homo sapiens
ARDLVHYGMDV

SEQ ID NO: 848 - protein, 9 aa, Homo sapiens
SSDVGGYNY

SEQ ID NO: 849 - skipped

SEQ ID NO: 850 - protein, 10 aa, Homo sapiens
SSYTSGSTWV

SEQ ID NO: 851 - DNA, 390 bp, Homo sapiens
  source 1..390: mol_type = other DNA
gaagtgcagctggtggagtctggggctgaggtgaagaagcctgggtcctcagtgaaggtctcctgcaaggcttctggaggcaccttcagcaggtatgctatcagctgggtgcgacaggcccctggacaaggacttgagtggatgggagggatcatccctatctttgatgcaacaaactacgcacagaagttccatgacagagtcaccattaccgcggacaaatccgcgagcacagcctacatggaactgagcagcctgagatctgacgacacggccgtgtattactgtgcgagagaacggacatattgtagtggtggtacttgctacggaggatacttctactacggtatggacgtctggggccaaggaaccacggtcaccgtctcctca

SEQ ID NO: 852 - protein, 130 aa, Homo sapiens
EVQLVESGAEVKKPGSSVKVSCKASGGTFSRYAISWVRQAPGQGLEWMGGIIPIFDATNYAQKFHDRVTITADKSASTAYMELSSLRSDDTAVYYCARERTYCSGGTCYGGYFYYGMDVWGQGTTVTVSS

SEQ ID NO: 853 - DNA, 331 bp, Homo sapiens
  source 1..331: mol_type = other DNA
cagtctgtgctgactcagccaccctcagcgtctgggacccccgggcagagggtcaccatctcttgttctggaagcagctccaacatcggaggcgatattgtaaactggtacctccagctcccagggacggcccccaaactcctcatttatagtaataatcagcggccctcaggcgtccctgaccgattctctggctccaggtctggcacctcagcctccctggccatcagtgggctccagtctgaggatgagggttattattactgtgcagcatgggatgacagcctgaatggtcaagtgttcggcggagggaccaagctgaccgtcctag

SEQ ID NO: 854 - protein, 110 aa, Homo sapiens
QSVLTQPPSASGTPGQRVTISCSGSSSNIGGDIVNWYLQLPGTAPKLLIYSNNQRPSGVPDRFSGSRSGTSASLAISGLQSEDEGYYYCAAWDDSLNGQVFGGGTKLTVL

SEQ ID NO: 855 - protein, 8 aa, Homo sapiens
GGTFSRYA

SEQ ID NO: 856 - protein, 8 aa, Homo sapiens
IIPIFDAT

SEQ ID NO: 857 - protein, 23 aa, Homo sapiens
ARERTYCSGGTCYGGYFYYGMDV

SEQ ID NO: 858 - protein, 8 aa, Homo sapiens
SSNIGGDI

SEQ ID NO: 859 - skipped

SEQ ID NO: 860 - protein, 11 aa, Homo sapiens
AAWDDSLNGQV

SEQ ID NO: 861 - DNA, 384 bp, Homo sapiens
  source 1..384: mol_type = other DNA
gaggtgcagctggtgcagtctggggctgaggtgaagaagcctgggtcctcggtgaaggtctcctgcaaggcttctggaggcaccttcagtagctatggtatcagctgggtgcgacaggcccctggactagggcttgagtggatggggggggtcatccctatcctaagtgcaaaacactacgcgcagcggttccagggcagagtcacgatcaccgcggacaagtccacgggcacagcctacatggagctgagcagcctgagatctgaggacacggccgtatactactgtgcgagagatatccttcatcatgacgacctttgggggaggttctactacgacggtatggacgtctggggccaagggaccacggtcaccgtctcctca

SEQ ID NO: 862 - protein, 128 aa, Homo sapiens
EVQLVQSGAEVKKPGSSVKVSCKASGGTFSSYGISWVRQAPGLGLEWMGGVIPILSAKHYAQRFQGRVTITADKSTGTAYMELSSLRSEDTAVYYCARDILHHDDLWGRFYYDGMDVWGQGTTVTVSS

SEQ ID NO: 863 - DNA, 331 bp, Homo sapiens
  source 1..331: mol_type = other DNA
cagtctgtcgtgacgcagccaccctcagcgtctgggacccccgggcagagggtcaccatctcttgttctggaagcagctccgacatcggaagtaatactgtaaactggtaccagcagctcccaggaacggcccccaaactcctcatctatactaataatcagcggccctcaggggtccctgaccgattctctggctccaagtctggcacctcagcctccctggccatcactgggctccagtctgaggatgaggctgattatttctgtgcagcatgggatgaaagcctgaatggtcgagtgttcggcggagggaccaagctgaccgtcctag

SEQ ID NO: 864 - protein, 110 aa, Homo sapiens
QSVVTQPPSASGTPGQRVTISCSGSSSDIGSNTVNWYQQLPGTAPKLLIYTNNQRPSGVPDRFSGSKSGTSASLAITGLQSEDEADYFCAAWDESLNGRVFGGGTKLTVL

SEQ ID NO: 865 - protein, 8 aa, Homo sapiens
GGTFSSYG

SEQ ID NO: 866 - protein, 8 aa, Homo sapiens
VIPILSAK

SEQ ID NO: 867 - protein, 21 aa, Homo sapiens
ARDILHHDDLWGRFYYDGMDV

SEQ ID NO: 868 - protein, 8 aa, Homo sapiens
SSDIGSNT

SEQ ID NO: 869 - skipped

SEQ ID NO: 870 - protein, 11 aa, Homo sapiens
AAWDESLNGRV

SEQ ID NO: 871 - DNA, 349 bp, Homo sapiens
  source 1..349: mol_type = other DNA
gaagtgcaactggtggagtctgggggaggcgtggtccagcctgggaggtccctgagactctcctgtgcagcgtctggattcaccttcagtaactatggcatgcactgggtccgccaggctccaggcaagggactggagtgggtggcagtttattggtatgatggaggtaataaattctatgcagactccgtgaagggccgattcaccatctccagagacaattccaagaatacgttgtatctgcaaatgaacagcctgagagtcgaggacacggctgtttattactgtgcgagagatacggctcctccggactactggggccagggaaccctggtcaccgtctcctcag

SEQ ID NO: 872 - protein, 116 aa, Homo sapiens
EVQLVESGGGVVQPGRSLRLSCAASGFTFSNYGMHWVRQAPGKGLEWVAVYWYDGGNKFYADSVKGRFTISRDNSKNTLYLQMNSLRVEDTAVYYCARDTAPPDYWGQGTLVTVSS

SEQ ID NO: 873 - DNA, 328 bp, Homo sapiens
  source 1..328: mol_type = other DNA
gccatccggatgacccagtctccaggcaccctgtctttgtctccaggggaaagagccaccctctcctgcagggccagtcagagtattagtagcagcttcttagcctggtaccagcagaaacctggccaggctcccaggctcctcatctatggtgcatccagcagggccactggcatcccagacaggttcagtggcagtgggtctgggacagacttcactctcaccatcagcagactggagcctgaagattttgcagtgtattactgtcagcagtatggtacctcaccaaggctcactttcggcggagggaccaaagtggatatcaaac

SEQ ID NO: 874 - protein, 109 aa, Homo sapiens
AIRMTQSPGTLSLSPGERATLSCRASQSISSSFLAWYQQKPGQAPRLLIYGASSRATGIPDRFSGSGSGTDFTLTISRLEPEDFAVYYCQQYGTSPRLTFGGGTKVDIK

SEQ ID NO: 875 - protein, 8 aa, Homo sapiens
GFTFSNYG

SEQ ID NO: 876 - protein, 8 aa, Homo sapiens
YWYDGGNK

SEQ ID NO: 877 - protein, 9 aa, Homo sapiens
ARDTAPPDY

SEQ ID NO: 878 - protein, 7 aa, Homo sapiens
QSISSSF

SEQ ID NO: 879 - skipped

SEQ ID NO: 880 - protein, 10 aa, Homo sapiens
QQYGTSPRLT

SEQ ID NO: 881 - DNA, 349 bp, Homo sapiens
  source 1..349: mol_type = other DNA
gaggtgcagctgttggagtctgggggaggcgtggtccagcctggaaggtccctgagactctcctgtgcagcgtctggattcaaattcagtgactatggcatgcactgggtccgccaggctccaggcaaggggctggagtgggtggcagtttattggtatgatggaggtactaaattctatgcagactccgtgaagggccgattcaccatctccagagacaattccaagaatacgttgtatctgcaaatgagcagcctgagagtcgaggacacggctgtttattactgtgcgagagatacggctcctccggactactggggccagggaaccctggtcaccgtctcctcag

SEQ ID NO: 882 - protein, 116 aa, Homo sapiens
EVQLLESGGGVVQPGRSLRLSCAASGFKFSDYGMHWVRQAPGKGLEWVAVYWYDGGTKFYADSVKGRFTISRDNSKNTLYLQMSSLRVEDTAVYYCARDTAPPDYWGQGTLVTVSS

SEQ ID NO: 883 - DNA, 328 bp, Homo sapiens
  source 1..328: mol_type = other DNA
gaaattgtgttgacgcagtctccaggcaccctgtctttgtctccaggggaaagagccaccctctcctgcagggccagtcagagtattagtagcaacttcttagcctggtaccagcagaaacctggccaggctcccaggctcctcatctatggtgcatccagcagggccactggcatcccagacaggttcagtggcagtgggtctgggacagacttcactctcaccatcagcagactggagcctgaagattttgcagtgtattactgtcagcagtatggtacctcaccaaggctcactttcggcggagggaccaaagtggatatcaaac

SEQ ID NO: 884 - protein, 109 aa, Homo sapiens
EIVLTQSPGTLSLSPGERATLSCRASQSISSNFLAWYQQKPGQAPRLLIYGASSRATGIPDRFSGSGSGTDFTLTISRLEPEDFAVYYCQQYGTSPRLTFGGGTKVDIK

SEQ ID NO: 885 - protein, 8 aa, Homo sapiens
GFKFSDYG

SEQ ID NO: 886 - protein, 8 aa, Homo sapiens
YWYDGGTK

SEQ ID NO: 887 - protein, 9 aa, Homo sapiens
ARDTAPPDY

SEQ ID NO: 888 - protein, 7 aa, Homo sapiens
QSISSNF

SEQ ID NO: 889 - skipped

SEQ ID NO: 890 - protein, 10 aa, Homo sapiens
QQYGTSPRLT

SEQ ID NO: 891 - DNA, 369 bp, Homo sapiens
  source 1..369: mol_type = other DNA
caggttcagctggtgcagtctggggctgaggtgaagaagcctgggtcctcggtgaaggtctcctgcaaggcttctggaggcaccttcagcagttatggtatcaggtgggtgcgacaggcccctggacaagggcttgagtggatgggagggatcatccccgtgtttggtgcaacaaactacgcacagaagttccaggacagagtcacaattaccgcggacaaatccacggccacagcctacatggaattgagtagcctgaaatctgacgacacggccgtgtatttttgtgcgagagatgcccttagtgccagtggctggacgggcccctttgactcgtggggccagggaaccctggtcaccgtctcctca

SEQ ID NO: 892 - protein, 123 aa, Homo sapiens
QVQLVQSGAEVKKPGSSVKVSCKASGGTFSSYGIRWVRQAPGQGLEWMGGIIPVFGATNYAQKFQDRVTITADKSTATAYMELSSLKSDDTAVYFCARDALSASGWTGPFDSWGQGTLVTVSS

SEQ ID NO: 893 - DNA, 337 bp, Homo sapiens
  source 1..337: mol_type = other DNA
cagtctgtgttgacgcagccgccctcagtgtctggggccccggggcagagggtcaccatctcctgcactgggagcagctccaacatcggggcagattatgatgtacactggtaccagcaacttccaggagcagcccccaaactcctcatctatggtaacaacaaccggccctcaggggtccctgaccgattctccggctccaagtctggcacctcagcctccctggccatcactgggctccaggctgaggatgaggctgattattactgccagtcctatgacagcagccagaatgctttctatgtcttcggaactgggaccaaggtcaccgtcctag

SEQ ID NO: 894 - protein, 112 aa, Homo sapiens
QSVLTQPPSVSGAPGQRVTISCTGSSSNIGADYDVHWYQQLPGAAPKLLIYGNNNRPSGVPDRFSGSKSGTSASLAITGLQAEDEADYYCQSYDSSQNAFYVFGTGTKVTVL

SEQ ID NO: 895 - protein, 8 aa, Homo sapiens
GGTFSSYG

SEQ ID NO: 896 - protein, 8 aa, Homo sapiens
IIPVFGAT

SEQ ID NO: 897 - protein, 16 aa, Homo sapiens
ARDALSASGWTGPFDS

SEQ ID NO: 898 - protein, 9 aa, Homo sapiens
SSNIGADYD

SEQ ID NO: 899 - skipped

SEQ ID NO: 900 - protein, 12 aa, Homo sapiens
QSYDSSQNAFYV

SEQ ID NO: 901 - DNA, 390 bp, Homo sapiens
  source 1..390: mol_type = other DNA
caggtgcagctggtggagtctgggggaggcttggtacagcctggcaggtccctgagactctcctgtgcagcctctggattcacctttgatgattatgccatgcactgggtccggcaagctccagggaagggcctggagtgggtctcaggaagtacttggaatagtggtaccatagactatgcggactctgtgaagggccgattcaccatctccagagacaacgccaagaactccctgtatctgcaaatgaacagtctgagagctgaggacacggccttgtattactgtgcaaaagataggtttcgtaaaggttgtagtagtaccggctgctataaggagaactacggtatggacgtctggggccaagggaccacggtcaccgtctcctca

SEQ ID NO: 902 - protein, 130 aa, Homo sapiens
QVQLVESGGGLVQPGRSLRLSCAASGFTFDDYAMHWVRQAPGKGLEWVSGSTWNSGTIDYADSVKGRFTISRDNAKNSLYLQMNSLRAEDTALYYCAKDRFRKGCSSTGCYKENYGMDVWGQGTTVTVSS

SEQ ID NO: 903 - DNA, 325 bp, Homo sapiens
  source 1..325: mol_type = other DNA
cagtctgtggtgactcagccaccctcggtgtcagtggccccaggacagacggccaggattacctgtggaggaaccaacattggaagtaaaagtgtccactggtaccagcagaagccaggccaggcccctgtgctggtcgtctatgatgatagcgaccggccctcagggatccctgagcgattctctggctccaactctgggaacacggccaccctgaccatcacctgggtcgaagccggggatgaggccgactattactgtcaggtgtgggatagtagtagtgataatgtgctattcggcggagggaccaagctgaccgtcctag

SEQ ID NO: 904 - protein, 108 aa, Homo sapiens
QSVVTQPPSVSVAPGQTARITCGGTNIGSKSVHWYQQKPGQAPVLVVYDDSDRPSGIPERFSGSNSGNTATLTITWVEAGDEADYYCQVWDSSSDNVLFGGGTKLTVL

SEQ ID NO: 905 - protein, 8 aa, Homo sapiens
GFTFDDYA

SEQ ID NO: 906 - protein, 8 aa, Homo sapiens
STWNSGTI

SEQ ID NO: 907 - protein, 23 aa, Homo sapiens
AKDRFRKGCSSTGCYKENYGMDV

SEQ ID NO: 908 - protein, 6 aa, Homo sapiens
NIGSKS

SEQ ID NO: 909 - skipped

SEQ ID NO: 910 - protein, 11 aa, Homo sapiens
QVWDSSSDNVL

SEQ ID NO: 911 - DNA, 355 bp, Homo sapiens
  source 1..355: mol_type = other DNA
gaggtgcagctggtggagtctgggggaggtgtggtccagcctggggggtccctgagactctcctgtgcagcctctggaatcatagtcagtgccaactacatgacctgggtccgccaggctccagggaagggactggaatgggtctcagttatttaccccggtggtagcacattctacgcggactccgtgaagggccgattcaccatctccagagacaactccaagaacacgttgtatcttcaaatgaacagcctgagagttgaggactcggctgtgtattactgtgcgagagatttggagctggctggtttcaatgactactggggccagggaaccctggtcaccgtctcctcag

SEQ ID NO: 912 - protein, 118 aa, Homo sapiens
EVQLVESGGGVVQPGGSLRLSCAASGIIVSANYMTWVRQAPGKGLEWVSVIYPGGSTFYADSVKGRFTISRDNSKNTLYLQMNSLRVEDSAVYYCARDLELAGFNDYWGQGTLVTVSS

SEQ ID NO: 913 - DNA, 328 bp, Homo sapiens
  source 1..328: mol_type = other DNA
gatattgtgatgactcagtctccaggcaccctgtctttgtctccaggggaaagagccaccctctcctgcaggaccagtcagagtgttagcagcaactacttagcctggtaccagcagaaacctggccaggctcccaggctcctcatctatggtgcatccagcagggccactggcatcccagacaggttcagtggcagtgggtctgggacagacttcactctcaccatcagcagactggagcctgaagattttgcagtgtattactgtcagcagtttggtagttcacctcggtacacttttggccaggggaccaaggtggagatcaaac

SEQ ID NO: 914 - protein, 109 aa, Homo sapiens
DIVMTQSPGTLSLSPGERATLSCRTSQSVSSNYLAWYQQKPGQAPRLLIYGASSRATGIPDRFSGSGSGTDFTLTISRLEPEDFAVYYCQQFGSSPRYTFGQGTKVEIK

SEQ ID NO: 915 - protein, 8 aa, Homo sapiens
GIIVSANY

SEQ ID NO: 916 - protein, 7 aa, Homo sapiens
IYPGGST

SEQ ID NO: 917 - protein, 12 aa, Homo sapiens
ARDLELAGFNDY

SEQ ID NO: 918 - protein, 7 aa, Homo sapiens
QSVSSNY

SEQ ID NO: 919 - skipped

SEQ ID NO: 920 - protein, 10 aa, Homo sapiens
QQFGSSPRYT

SEQ ID NO: 921 - DNA, 355 bp, Homo sapiens
  source 1..355: mol_type = other DNA
caggtgcagctggtggagtctggggctgaggtgaagaagcctgggtcctcggtgaaggtctcctgcaaggcttctggaggaaacttcaacatgtatactatcagttgggtgcgacaggcccctggacgaggacttgagtggatgggaaggttcatccctatcgctaataaagcaaactacgcacagaactttccgggcagagtcaccattaccgcggacaaatccactagcacagtctacatggagctgagaagcctgacatctgacgacacggccgtgtattactgtgcgagaagtgggagctacgatgcttttgatgtgtggggccaagggacaatggtcaccgtctcttcag

SEQ ID NO: 922 - protein, 118 aa, Homo sapiens
QVQLVESGAEVKKPGSSVKVSCKASGGNFNMYTISWVRQAPGRGLEWMGRFIPIANKANYAQNFPGRVTITADKSTSTVYMELRSLTSDDTAVYYCARSGSYDAFDVWGQGTMVTVSS

SEQ ID NO: 923 - DNA, 325 bp, Homo sapiens
  source 1..325: mol_type = other DNA
gccatccggatgacccagtctccttccaccctgtctgcatctgtaggagacagagtcaccatcacttgccgggccagtcagactattaatagttggttggcctggtatcagcagaaacccgggaaagcccctaagctcctgatctatgatgcctccaatttggaaagtggggtcccatcaaggttcagcggcagtggatctgggacagaattcactctcaccatcagcagcctgcagcctgatgattttgcaacttattactgccaacagtatgaaagttattctccgatcaccttcggccaagggacacgactggagattaaac

SEQ ID NO: 924 - protein, 108 aa, Homo sapiens
AIRMTQSPSTLSASVGDRVTITCRASQTINSWLAWYQQKPGKAPKLLIYDASNLESGVPSRFSGSGSGTEFTLTISSLQPDDFATYYCQQYESYSPITFGQGTRLEIK

SEQ ID NO: 925 - protein, 8 aa, Homo sapiens
GGNFNMYT

SEQ ID NO: 926 - protein, 8 aa, Homo sapiens
FIPIANKA

SEQ ID NO: 927 - protein, 11 aa, Homo sapiens
ARSGSYDAFDV

SEQ ID NO: 928 - protein, 6 aa, Homo sapiens
QTINSW

SEQ ID NO: 929 - skipped

SEQ ID NO: 930 - protein, 10 aa, Homo sapiens
QQYESYSPIT

SEQ ID NO: 931 - DNA, 369 bp, Homo sapiens
  source 1..369: mol_type = other DNA
caggtgcagctggtggagtctgggggagtcgtggtacagcctggggggtccctgagactctcctgtgcagcctctggattcagctttgatgattatagcatgcactgggtccgtcaagctccggggaagggtctggagtgggtctctgtcatttactgggatggtgttagcaaatactatgcagactctgtgaagggccgattcaccatctccagagacaacagcaaaaactccctgtatttgcaaatgaacagtctgagaactgaggacaccgccgtatattactgtgcaaaagatagtgaggattgtagtagtaccagctgctacatggacgtctggggcaaagggaccacggtcaccgtctcctca

SEQ ID NO: 932 - protein, 123 aa, Homo sapiens
QVQLVESGGVVVQPGGSLRLSCAASGFSFDDYSMHWVRQAPGKGLEWVSVIYWDGVSKYYADSVKGRFTISRDNSKNSLYLQMNSLRTEDTAVYYCAKDSEDCSSTSCYMDVWGKGTTVTVSS

SEQ ID NO: 933 - DNA, 340 bp, Homo sapiens
  source 1..340: mol_type = other DNA
gaaattgtgttgacacagtctccagactccctggctgtgtctctgggcgagagggccaccatcaactgcaagtccagccagaatgttttatacagctccaacaataagaattacttagcttggtaccagcagaaaccaggacagcctcctcaactactcatttactgggcatctacccgggaatccggggtccctgaccgattcagtggcagcgggtctgggacagatttcactctcaccatcagcagcctgcaggctgaagatgtggcagtttattactgtcaccaatattatagtactccattcactttcggccctgggaccaaagtggatatcaaac

SEQ ID NO: 934 - protein, 113 aa, Homo sapiens
EIVLTQSPDSLAVSLGERATINCKSSQNVLYSSNNKNYLAWYQQKPGQPPQLLIYWASTRESGVPDRFSGSGSGTDFTLTISSLQAEDVAVYYCHQYYSTPFTFGPGTKVDIK

SEQ ID NO: 935 - protein, 8 aa, Homo sapiens
GFSFDDYS

SEQ ID NO: 936 - protein, 8 aa, Homo sapiens
IYWDGVSK

SEQ ID NO: 937 - protein, 16 aa, Homo sapiens
AKDSEDCSSTSCYMDV

SEQ ID NO: 938 - protein, 12 aa, Homo sapiens
QNVLYSSNNKNY

SEQ ID NO: 939 - skipped

SEQ ID NO: 940 - protein, 9 aa, Homo sapiens
HQYYSTPFT

SEQ ID NO: 941 - DNA, 370 bp, Homo sapiens
  source 1..370: mol_type = other DNA
caggttcagctggtgcagtctggagctgaggtgaagaagcctggggcctcagtgaaggtctcctgcaaggctgctggttacagctttatgaactacggtatcaactgggtgcgacaggcccctggacaagggcttgagtggatgggatggatcaacacttacaatggtaacgcaaagtatgcacagaagttccagggccgagtcaccatgaccacagacacatccacgagcacagcctacatggagctgaggagcctgagatcgggcgacacggccgtgtattactgtgcgagggaccctttcaccggttatgatgacgtttgggggggggactactggggccagggaaccctggtcaccgtctcctcag

SEQ ID NO: 942 - protein, 123 aa, Homo sapiens
QVQLVQSGAEVKKPGASVKVSCKAAGYSFMNYGINWVRQAPGQGLEWMGWINTYNGNAKYAQKFQGRVTMTTDTSTSTAYMELRSLRSGDTAVYYCARDPFTGYDDVWGGDYWGQGTLVTVSS

SEQ ID NO: 943 - DNA, 340 bp, Homo sapiens
  source 1..340: mol_type = other DNA
gccatccagatgacccagtctccagactccctggctgtgtctctgggcgagagggccaccatcaactgcaagtccagccagagtgttttatacagctccaacaataagaattacttagcttggtaccagcagaaaccaggacagcctcctaagctggtcatttactgggcatctacccgggaatccggggtccctgaccgattcagtggcagcgggtctgggacagatttcactctcaccatcagcagcctgcaggctgaagatgtggcagtttattactgtcaccaatattatagtagtcctcgcacttttggccaggggaccaaggtggaaatcaaac

SEQ ID NO: 944 - protein, 113 aa, Homo sapiens
AIQMTQSPDSLAVSLGERATINCKSSQSVLYSSNNKNYLAWYQQKPGQPPKLVIYWASTRESGVPDRFSGSGSGTDFTLTISSLQAEDVAVYYCHQYYSSPRTFGQGTKVEIK

SEQ ID NO: 945 - protein, 8 aa, Homo sapiens
GYSFMNYG

SEQ ID NO: 946 - protein, 8 aa, Homo sapiens
INTYNGNA

SEQ ID NO: 947 - protein, 16 aa, Homo sapiens
ARDPFTGYDDVWGGDY

SEQ ID NO: 948 - protein, 12 aa, Homo sapiens
QSVLYSSNNKNY

SEQ ID NO: 949 - skipped

SEQ ID NO: 950 - protein, 9 aa, Homo sapiens
HQYYSSPRT

SEQ ID NO: 951 - DNA, 375 bp, Homo sapiens
  source 1..375: mol_type = other DNA
gaggtgcagctgttggagactgggggaggcttggttcagcccggcaggtccctgagactctcctgtgcagcctcgggattcccctttgatgattatgccatccactgggtccggctagctccagggaagggcctggagtgggtctcaagtattagttgggatagtggtagcataggctatgcggactctgtgaagggccggttcaccatctccagagacaacgccaagaactccctgtatctgcaaatgaacagtctgagagctgaggacacggccttgtattactgtgcaaagggggcctttcccgggtatagcagtggctggtactacggtttggacgtctggggccaaggggccacggtcaccgtctcctca

SEQ ID NO: 952 - protein, 125 aa, Homo sapiens
EVQLLETGGGLVQPGRSLRLSCAASGFPFDDYAIHWVRLAPGKGLEWVSSISWDSGSIGYADSVKGRFTISRDNAKNSLYLQMNSLRAEDTALYYCAKGAFPGYSSGWYYGLDVWGQGATVTVSS

SEQ ID NO: 953 - DNA, 328 bp, Homo sapiens
  source 1..328: mol_type = other DNA
cagtctgtcgtgacgcagcctccctccgcgtcggggtctcttggacagtcagtcaccatctcctgcactggaaccagcagtgacgttggtggttacaactatgtctcttggtaccaacaacacccaggcaaagcccccaaactcatgatttttgaggtcagtaagcggccctcaggggtccctgatcgcttctctggctccaagtctggcaacacggcctccctgaccgtctctgggctccaggctgaggatgaggctgattattactgcagctcatatgcaggcaacaaaggggtcttcggcggagggaccaaattgaccgtcctcg

SEQ ID NO: 954 - protein, 109 aa, Homo sapiens
QSVVTQPPSASGSLGQSVTISCTGTSSDVGGYNYVSWYQQHPGKAPKLMIFEVSKRPSGVPDRFSGSKSGNTASLTVSGLQAEDEADYYCSSYAGNKGVFGGGTKLTVL

SEQ ID NO: 955 - protein, 8 aa, Homo sapiens
GFPFDDYA

SEQ ID NO: 956 - protein, 8 aa, Homo sapiens
ISWDSGSI

SEQ ID NO: 957 - protein, 18 aa, Homo sapiens
AKGAFPGYSSGWYYGLDV

SEQ ID NO: 958 - protein, 9 aa, Homo sapiens
SSDVGGYNY

SEQ ID NO: 959 - skipped

SEQ ID NO: 960 - protein, 9 aa, Homo sapiens
SSYAGNKGV

SEQ ID NO: 961 - protein, 97 aa, Homo sapiens
MQLVQSGPEVKKPGTSVKVSCKASGFTFTSSAVQWVRQARGQRLEWIGWIVVGSGNTNYAQKFQERVTITRDMSTSTAYMELSSLRSEDTAVYYCAA

SEQ ID NO: 962 - DNA, 369 bp, Homo sapiens
  source 1..369: mol_type = other DNA
caaatgcagctggtgcagtctgggcctgaggtgaagaagcctgggacctcagtgaaggtctcctgcaaggcttctggattcacctttatgagctctgctgtgcagtgggtgcgacaggctcgtggacaacgccttgagtggataggatggatcgtcattggcagtggtaacacaaactacgcacagaagttccaggaaagagtcaccattaccagggacatgtccacaagcacagcctacatggagctgagcagcctgagatccgaggacacggccgtgtattactgtgcggccccatattgtagtagtatcagctgcaatgatggttttgatatctggggccaagggacaatggtcaccgtctcttca

SEQ ID NO: 963 - protein, 123 aa, Homo sapiens
QMQLVQSGPEVKKPGTSVKVSCKASGFTFMSSAVQWVRQARGQRLEWIGWIVIGSGNTNYAQKFQERVTITRDMSTSTAYMELSSLRSEDTAVYYCAAPYCSSISCNDGFDIWGQGTMVTVSS

SEQ ID NO: 964 - DNA, 327 bp, Homo sapiens
  source 1..327: mol_type = other DNA
gaaattgtgttgacgcagtctccaggcaccctgtctttgtctccaggggagagagccaccctctcctgcagggccagtcagagtgttagcagcagctacttagcctggtaccagcagaaacctggccaggctcccaggctcctcatctatggtgcatccagcagggccactggcatcccagacaggttcagtggcagtgggtctgggacagacttcactctcaccatcagcagactggagcctgaagattttgcagtgtattactgtcagcactatggtagctcacggggttggacgttcggccaagggaccaaggtggaaatcaaa

SEQ ID NO: 965 - protein, 109 aa, Homo sapiens
EIVLTQSPGTLSLSPGERATLSCRASQSVSSSYLAWYQQKPGQAPRLLIYGASSRATGIPDRFSGSGSGTDFTLTISRLEPEDFAVYYCQHYGSSRGWTFGQGTKVEIK

SEQ ID NO: 966 - protein, 1273 aa, Homo sapiens
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 967 - protein, 96 aa, Homo sapiens
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSP

(19) United States
(12) Patent Application Publication
(10) Pub. No.: US 2025/0154231 A1
(43) Pub. Date: May 15, 2025

(54) ANTIBODIES CAPABLE OF BINDING TO THE SPIKE PROTEIN OF CONONAVIRUS SARS-COV-2

(71) Applicant: RQBIO COVID LIMITED, London (GB)

(72) Inventors: Juthathip MONGKOLSAPAYA, Oxford (GB); Gavin SCREATON, Oxford (GB)

(21) Appl. No.: 18/839,067

(22) PCT Filed: Feb. 17, 2023
(86) PCT No.: PCT/EP2023/054109

§ 371 Date: Aug. 16, 2024

(30) Foreign Application Priority Data
Feb. 18, 2022 (GB) 2202232.1
Mar. 11, 2022 (GB) 2203423.5
May 9, 2022 (GB) 2206777.1
Aug. 26, 2022 (GB) 2212470.5
Sep. 26, 2022 (GB) 2214036.2
Oct. 18, 2022 (GB) 2215418.1
Feb. 10, 2023 (GB) 2301959.9

(51) Int. Cl. C07K 16/10; A61K 45/06; A61P 31/14; G01N 33/569

(52) U.S. Cl. CPC C07K 16/1003; A61K 45/06; A61P 31/14; G01N 33/56983; C07K 2317/52; C07K 2317/565; C07K 2317/76; C07K 2317/94; G01N 2333/165

(57) ABSTRACT

The disclosure relates to antibodies useful for the prevention, treatment and/or diagnosis of coronavirus infections, and diseases and/or complications associated with coronavirus infections, including COVID-19. In particular, the disclosure relates to antibodies capable of binding to the spike protein of coronavirus SARS-CoV-2 and uses thereof.

### FIELD OF INVENTION

The invention relates to antibodies useful for the prevention, treatment and/or diagnosis of coronavirus infections, and diseases and/or complications associated with coronavirus infections, including COVID-19.

### BACKGROUND OF THE INVENTION

A severe viral acute respiratory syndrome named COVID-19 was first reported in Wuhan, China in December 2019. The virus rapidly disseminated globally leading to the pandemic with >200M confirmed infections and over 4.4M deaths in 12 months. The causative agent, SARS-CoV-2, is a beta coronavirus, related to SARS-CoV-1 and MERS coronaviruses, all of which cause severe respiratory syndromes.

Tremendous advances in our understanding of the disease and the virus have been made in the months since the identification of SARS-CoV-2 as the causative agent a of COVID-19. There are now a number of proven treatments including dexamethasone and Tocilizumab as well as monoclonal antibodies (mAbs), which have been shown to be effective when used in both prophylactic and therapeutic settings (Baum et al., 2020, Science 369, 1014-1018). Despite these advances, the pandemic is far from under control, leading to successive waves of infection.

Coronaviruses have four structural proteins: nucleocapsid, envelope, membrane and spike (S) proteins. The spike protein is the most prominent surface protein. It has an elongated trimeric structure and is responsible for engagement of target cells and triggering fusion of viral and host membranes. The spike protein from SARS-CoV-2 and SARS-CoV-1 both use angiotensin-converting enzyme 2 (ACE2) as a cell surface receptor. ACE2 is expressed in a number of tissues, including epithelial cells of the upper and lower respiratory tracts.

The S protein consists of two subunits, S1, which mediates receptor binding, and S2, responsible for viral and host cell membrane fusion. It is a dynamic structure capable of transitioning to a post-fusion state by cleavage between S1 and S2 following receptor binding or trypsin treatment. In some SARS-CoV-2 sequences a furin protease cleavage site is inserted between the S1 and S2 subunits, and a mutation of the cleavage site attenuates disease in animal models. The S1 fragment occupies the membrane distal tip of S and can be subdivided into an N-terminal domain (NTD) and receptor binding domain (RBD). While both regions are immunogenic, the RBD contains the interacting surface for ACE2 binding. Although usually packed down against the top of S2, RBDs can swing upwards to engage ACE2. Monoclonal antibodies (mAbs) recognize one or both of ‘up’ and ‘down’ conformations.

The S protein is relatively conserved between SARS-CoV-2 and SARS-CoV-1 (76%), but the RBD and NTD are less conserved (74% and 50% respectively) than the S2 domain (90%). Conservation with MERS-CoV and the seasonal human coronaviruses is much lower (19-21%). Overall, SARS-CoV-2 antibodies show limited cross-reactivity, even with SARS-CoV-1.

S is involved in viral attachment to target cells via the interaction of cell surface expressed ACE2 with the S receptor binding motif (otherwise known as the ACE-2 footprint), a 25 amino acid patch that lies at the tip of the receptor binding domain (RBD), in the S1 fragment of spike. Following attachment, cleavage of S releases S1, allowing a major conformational change in S2 exposing the hydrophobic fusion loop, to execute fusion of viral and host cell membranes, releasing the viral genome into the host cell cytoplasm to initiate viral replication.

Analysis of large panels of mAbs generated from SARS-CoV-2 infected individuals reveals mAbs binding to multiple epitopes across S1 and S2. The majority of mAbs generated against the original strains of SARS-CoV-2, although able to bind S with high affinity, show little or no neutralizing activity. Genomic surveillance of SARS-CoV-2 has identified many thousands of mutations in structural and non-structural proteins. However, towards the end of 2020, viral variants were described that rapidly became the dominant strains locally and led to global spread and their designation of variants of concern (VoC).

Alpha (B.1.1.7) was first identified in the UK, with increased transmission. B.1.1.7 harbours 9 amino-acid changes in the spike, including N501Y in the ACE2 interacting surface. Beta (501Y.V2 also known as B.1.351) was first reported in South Africa. Gamma (P.1, 501Y.V2) was first reported in Brazil, which have 10 and 12 amino-acid changes in the spike protein, respectively. Delta was first reported from India and has now spread globally, causing outbreaks in a number of countries. Omicron BA.1 was first reported in late November 2021 in Southern Africa and spread around the world, becoming the dominant variant in many countries and almost completely displaced Delta.

A succession of sub-lineages of Omicron have emerged, including BA.1.1, BA.2, BA.2.12.1, BA.2.75 and BA.4/5, which have outcompeted preceding strains to become regionally or globally dominant. Over 30 mutations are found in the Omicron S protein, including 15 substitutions in the RBD, leading to increased transmissibility (Suzuki et al., 2022 “Attenuated fusogenicity and pathogenicity of SARS-CoV-2 Omicron variant.” Nature 603, 700-705) and widespread large reductions in neutralizing antibody titres (Dejnirattisai et al., 2022 “SARS-CoV-2 Omicron-B.1.1.529 leads to widespread escape from neutralizing antibody responses.” Cell 185, 467-484 e415).

Omicron BA.2 was reported at nearly the same time as BA.1. The proportion of Omicron infections caused by BA.2 has been increasing in several countries and it became the dominant sub-lineage in Denmark and India.

BA.1.1, containing an additional R346K mutation in RBD, at one point accounted for about 40% of Omicron sequences globally, and about 35-60% in the UK and the USA (Iketani et al., 2022 “Antibody evasion properties of SARS-CoV-2 Omicron sublineages.” Nature 604, 553-556), but was soon outcompeted by BA.2. BA.2, which contains 8 unique substitutions in S, including 6 within the RBD, and lacks 13 mutations found in BA.1 (Nutalai et al., 2022), has become the dominant strain across the world as of August 2022. Recently, BA.2.12.1 has been identified in multiple countries, and caused a large regional outbreak in the North America (58% of the sequences as of May 25, 2022) (Del Rio and Malani, 2022, “COVID-19 in 2022—The Beginning of the End or the End of the Beginning?” JAMA 327, 2389-2390).

It is now becoming clear that BA.2 has a small transmission advantage against BA.1 although there is no evidence of increased disease severity. In early April 2022, two new Omicron lineages were reported from Gauteng in South Africa and designated BA.4 and BA.5. BA.4 and BA.5 (which have identical S sequences) became the dominant Omicron strains in Gauteng, fueling a new wave of infection in South Africa.

Since June 2022, BA.4/5, which has both higher receptor binding affinity and a markedly enhanced escape from antibody responses (Tuekprakhon et al., 2022 “Antibody escape of SARS-CoV-2 Omicron BA.4 and BA.5 from vaccine and BA.1 serum.” Cell 185, 2422-2433 e2413) quickly spread from South Africa across the world and has now become the new globally dominant strain, with BA.5 in the ascendency in many regions. These variants (particularly BA.5) now account for the majority of sequenced cases in many countries.

In early May 2022, a new Omicron sub-lineage designated as BA.2.75 emerged in India. This strain has since spread to many countries including the UK, US, Australia, Germany and Canada. However, the true prevalence of BA.2.75 is difficult to determine as sequencing in many countries is patchy and has been greatly scaled back.

All of these variants contain multiple mutations in S and include changes in the RBD, NTD and in some cases the furin cleavage site between S1 and S2. The RBD mutations found in Alpha (N501Y), Beta (K417N, E484K, N501Y), Gamma (K417T, E484K, N501Y) and Delta (L452R, T478K) are located in or closely adjacent to the ACE2 interacting surface where they have the potential to modulate ACE2 interaction and disrupt the binding of neutralizing antibodies. Increased affinity of ACE2 interaction has been dominated for Alpha, Beta, Gamma and Delta (7, 19, 19, 2-fold, respectively) and may play a role in increasing viral transmissibility. Omicron contains an unprecedented number of mutations concentrated in the Spike (S) gene which carries 30 substitutions plus the deletion of 6 and insertion of 3 residues. Omicron BA.1 (RBD mutations of G339D, S371L, S373P, S375F, K417N, N440K, G446S, S477N, T478K, E484A, Q493R, G496S, Q498R, N501Y, Y505H) contains unique mutations S371L, G446S and G496S and in some isolates R346K (BA.1.1), while BA.2 carries S371F, T376A, D405N and R408S. BA.3 contains no unique mutations relative to BA.1 and BA.2 and appears to be a fusion of the two, being BA.1-like at the N terminus and switching to become BA.2-like at the C-terminus from the mutation G496S.

BA.2.75 contains multiple mutational changes in the S protein compared to BA.2, including four substitutions in the NTD (W152R, F157L, I210V and G257S) and four in the RBD: D339H, G446S, N460K and R493Q.

Three new variants related to BA.2, namely BA.2.11, BA.2.12.1 and BA.2.13, have also been detected in multiple countries. These contain a single mutation of L452R, L452Q and L452M compared to the BA.2 Spike receptor-binding domain (RBD) respectively (FIG. 29). Among them, BA.2.12.1, first identified in New York, became dominant in the US, accounting for about 58% of SARS-CoV-2 isolates as of May 25, 2022. While L452R is found in Delta and Kappa variants, and L452Q in Lambda, L452M is novel.

Considering the physico-chemical properties of the side chain of residue 452, BA.2.13 would be expected to be a relatively modest change; L to M will increase the size of the side chain but it remains hydrophobic. L to Q in BA.2.12.1 introduces some polar character, whilst BA.2.11 is the most radical with L to R introducing a large basic amino acid.

Further variants, BA.4 and BA.5, which have identical S sequences, appear to have evolved from BA.2. The sequences of BA.4 and BA.5 are highly related to the sequence of BA.2, but contain additional mutations. In particular, residues 69 and 70 of the NTD have been deleted (also found in Alpha, BA.1 and BA.3) and they contain two additional substitutions in the RBD: L452R (also found in Delta) and F486V. Finally BA.4 and BA.5 lack the Q493R change seen in BA.1 and BA.2, reverting to Q493 as in the Victoria/Wuhan strain. When looking at the RBD, BA.4 and BA.5 have assembled mutations at all of the previously described positions in the VoC Alpha (N501Y), Beta (K417N, E484K, N501Y), Gamma (K417T, E484K, N501Y), Delta (L452, T478K), the only difference is E484A in BA.4 and BA.5 rather than E484K Beta and Gamma.

As of September 2022, a new variant related to BA.4/5, designated BA.4.6, has emerged and expanded in the United States where BA.5 dominates (87.5% prevalence as of 10 September 2022, tripling from less than 2% of sequences in early July 2022 to over 6% in mid-August 2022). Compared to BA.4/5, BA.4.6 contains two extra mutations in the Spike protein (S), R346T in the RBD and N658S in the C-terminal domain. The R346T mutation has raised concern for enhanced antibody evasion over BA.4/5, as the R346K mutation in BA.1.1 reduced serum neutralisation compared to BA.1 and impaired the activity of a number of monoclonal antibodies (mAbs) (Nutalai, et al., 2022). SARS-CoV-2 detection kits using monoclonal antibodies have also been developed. Examples include lateral flow tests by, e.g. Innova (SARS-CoV-2 Antigen Rapid Qualitative Test) and Quidel (Sofia 2 SARS Antigen FIA). However, these tests are reported to be highly inaccurate.

As of January 2023, further variants have emerged such as BQ.1 and XBB, which carry up to 8 additional RBD amino-acid substitutions compared to BA.2.

Structure function mapping of panels of monoclonal antibodies (mAbs)isolated from infected cases has led to considerable understanding of the antigenicity of S and mechanisms of neutralization. The majority of potent neutralizing antibodies bind at or in close proximity to the footprint of ACE2 and function by blocking ACE2 interaction, thereby preventing cellular attachment and infection. A second site of interaction of potent mAbs is in proximity to N-linked glycan at position N343, exemplified by S309, these antibodies do not block ACE2 interaction but may function to destabilize the S trimer. The third group of potent mAbs bind to the N-terminal domain in S1 and their mode of action is at present unclear. Another RBD epitope of potential interest lies outside of the ACE2 footprint and whilst mAbs binding here are not potent neutralizers they can nevertheless effectively protect in vivo (Huo et al., 2020; Sun et al., 2021; Yuan et al., 2020; Zhou et al., 2020).

Following BA.5 several new trends were observed in the evolution of Omicron: i) the emergence of ‘second generation’ BA.2 variants (including derivatives of BA.5)—variants with long phylogenetic branch lengths, multiple antigenic mutations and a lack of genetic intermediates, for example BA.2.75, BJ.1, BS.1, BA.2.10.4 and BA.2.3.20 (van der Straten et al. 2022. Immunity 55, 1725-1731) and ii) accelerated antigenic drift, seen both in BA.5 (Tuekprakhon et al., 2022) and within these second generation BA.2 lineages, notably BQ.1 and BA.2.75 (https://nextstrain.org/nextclade/sars-cov-2/21L). Finally, recombination between two of these second-generation variants (BJ.1 and BM.1.1.1) has produced XBB. Many of these variants show a large degree of convergent evolution in known antigenic RBD residues, and mutations lie in areas that may threaten the binding of neutralizing antibodies, leading to further escape from protection from infection afforded by vaccine or previous SARS-CoV-2 infection, including prior Omicron infection.

At present a number of lineages are growing rapidly from within both the BA.2 and BA.5 branches. Most striking is the large degree of convergent evolution, particularly at antigenic RBD positions such as 346, 444, 446, 452, 460, 486, 490, and 494. These lineages include examples from the BA.4/5 branch (which naturally contains L452R, F486V and the reversion R493Q), such as BA.4.6 and BF.7 (R346T), BA.4.7 (R346S), BQ.1 (K444T, N460K) and BQ.1.1 (R346T, K444T, N460K); from the BA.2.75 branch (which naturally contains G446S, N460K and the reversion R493Q), BA.2.75.2 (R346T and F486V), BN.1 (R346T, K356T, F490S). There are also examples of several other second generation BA.2 variant lines such as BJ.1 (aka BA.2.10.1.1; R346T, L368I, V445P, G446S, V483A and F490V), BA.2.10.4 (G446S, F486P, S494P and the R493Q reversion), BS.1 (BA.2.3.2.1; R346T, L452R, N460K, G476S), BA.2.3.20 (K444R, N450D, L452M, N460K, E484R and the Q493R reversion), and finally a BA.2.75×BJ.1 recombinant, XBB (which relative to BA.2 contains R346T, L368I, V445P, G446S, N460K, F486S, F490S).

These second-generation BA.2 variants have become dominant globally, with BQ.1 alone accounting for 50% of infections as of 27 Dec. 2022 (https://cov-spectrum.org/explore/World/AllSamples/Past6M) and XBB.1.5 (XBB.1+F486P) expanding rapidly in North America).

Outside the RBD the degree of convergent evolution is lesser but still present. Many of the second-generation BA.2 variant lineages contain deletions or mutations in the NTD, often similar to that seen in the VoCs, for example Δ˜144 in BJ.1 and BA.2.10.4 (previously seen in Alpha and BA.1) and NSP12 G671S in BJ.1, BA.2.75 and BA.2.10.4 (previously seen in Delta).

All currently approved SARS-CoV-2 vaccines are designed to induce antibody (and T-cell) responses to S and contain the S sequence found in the original Wuhan strain. There is therefore particular concern as to whether the S mutations in the VoCs could cause immune escape, leading to vaccine failure or susceptibility to repeat infections in previously infected individuals.

The extensive mutational burden in Omicron S disrupts the activity of the majority of mAb binding to the three sites of binding of potent antibodies described above, the ACE-2 footprint, around the N343 glycan and the NTD. This leads to severe knock down or complete loss of the neutralizing capacity of serum from natural infection or vaccination, which has contributed to the increased transmissibility and explosive spread of Omicron.

It is an object of the invention to identify further and improved antibodies useful for preventing, treating and/or diagnosing coronavirus infections, and diseases and/or complications associated with coronavirus infections, including COVID-19, especially the Omicron variants of concern (VoCs) and as-yet-unidentified variants having further mutations in the ACE-2 footprint, RBD and/or NTD in the spike protein of SARS-CoV-2.

### SUMMARY OF THE INVENTION

The inventors identified 28 human monoclonal antibodies (mAbs) recognizing the spike protein of SARS-CoV-2 (see Table 3). These antibodies showed potent neutralisation activity against SARS-CoV-2. Some of the Table 3 antibodies demonstrated potent neutralization effects that were broadly effective against the hCoV-19/Wuhan/WIV04/2019 strain, as well as SARS-CoV-2 strains from various lineages, such as Victoria (Wuhan+S247R), Alpha, Beta, Gamma, Delta, Omicron, including Omicron BA.2.11, Omicron BA.2.12.1, Omicron BA.2.13, Omicron Omicron BA.2.3.20, Omicron BA.2.10.4, Omicron BA.1, Omicron BA.1.1, Omicron BA.2, Omicron BA.2.75, BA.2.75.2, Omicron BA.3, Omicron BA.4.6, Omicron BA.4/5, Omicron BJ.1, Omicron BS.1, Omicron BN.1, Omicron XBB, and/or Omicron XBB.1 strains.

Many of the Table 3 mAbs used public V-genes (V-genes shared by the majority of the population). The inventors have previously shown that it is possible to generate further antibodies by swapping the light and heavy chains of the antibodies in Tables 1, 2 and 3 which are derived from the same public V-genes. Antibodies derived from the same public V-genes provided particularly useful mixed-chain antibodies.

In particular, the inventors found that antibodies Omi02, Omi03, Omi12, Omi18, Omi28, Omi39 and Omi42 were particularly effective at cross-neutralising SARS-CoV-2 strains Victoria, Alpha, Beta, Gamma, Delta and Omicron.

Accordingly, the invention provides an antibody capable of binding to the spike protein of coronavirus SARS-CoV-2, wherein the antibody comprises at least three CDRs of any one of the 28 antibodies in Table 3.

The invention provides an antibody capable of binding to the spike protein of coronavirus SARS-CoV-2, wherein the antibody comprises at least three CDRs of antibody Omi12, or of any one of the 27 antibodies in Table 3.

The invention also provides a combination of antibodies comprising two or more antibodies according to the invention.

The invention also provides a combination of antibodies comprising (a) an antibody of the invention; and (b) an antibody comprising at least three CDRs of an antibody in Table 1 or Table 2. For example, the antibody may comprise (i) at least four, five, or all six CDRs of an antibody in Table 1 or Table 2; (ii) a heavy chain variable domain comprising or consist of an amino acid sequence having at least 80% sequence identity to the heavy chain variable domain of an antibody in Table 1 or Table 2; (iii) a light chain variable domain comprising or consisting of an amino acid sequence having at least 80% sequence identity to the light chain variable domain of an antibody in Table 1 or Table 2; and/or (iv) a heavy chain variable domain and a light chain variable domain comprising or consisting of an amino acid sequence having at least 80% identity to the heavy chain variable domain and light chain domain, respectively, of an antibody in Table 1 or Table 2.

The invention also provides one or more polynucleotides encoding an antibody of the invention, one or more vectors comprising said polynucleotides, or a host cell comprising said vectors.

The invention also provides a method for producing an antibody that is capable of binding to the spike protein of coronavirus SARS-CoV-2, comprising culturing the host cell of the invention and isolating the antibody from said culture.

The invention also provides a pharmaceutical composition comprising: (a) an antibody or a combination of antibodies of the invention, and (b) at least one pharmaceutically acceptable diluent or carrier.

The invention also provides an antibody, a combination of antibodies or a pharmaceutical composition of the invention, for use in a method for treatment of the human or animal body by therapy.

The invention also provides an antibody, a combination of antibodies or a pharmaceutical composition of the invention, for use in a method of treating or preventing coronavirus infection, or a disease or complication associated with coronavirus infection.

The invention also provides a method of treating or preventing coronavirus infection, or a disease or complication associated with coronavirus infection in a subject, comprising administering a therapeutically effective amount of an antibody, a combination of antibodies or the pharmaceutical composition of the invention, to said subject.

The invention also provides a method of identifying the presence of coronavirus, or a protein fragment thereof, in a sample, comprising (i) contacting the sample with an antibody or combination of antibodies of the invention, and (ii) detecting the presence or absence of an antibody-antigen complex, wherein the presence of the antibody-antigen complex indicates the presence of coronavirus, or a fragment thereof, in the sample.

The invention also provides a method of treating or preventing coronavirus infection, or a disease or complication associated therewith, in a subject, comprising identifying the presence of coronavirus according to the method of the invention, and treating the subject with the antibody or combination according to the invention, an anti-viral or an anti-inflammatory agent.

The invention also provides the use of an antibody, a combination of antibodies or a pharmaceutical composition of the invention, for preventing, treating and/or diagnosing coronavirus infection, or a disease or complication associated therewith.

The invention also provides the use of an antibody, a combination of antibodies or a pharmaceutical composition of the invention, for the manufacture of a medicament for treating or preventing coronavirus infection, or a disease or complication associated therewith.

### BRIEF DESCRIPTION OF THE FIGURES

FIG. 1. The BA.2 sub-lineage of Omicron and generation of a panel of Omicron mAb. FIG. 1 relates only to the first 22 Omicron antibodies disclosed in Tables 13 and 14 (i.e. Omi02 to Omi35). (A) FRNT50 titres against Victoria and Omicron BA.1 from the donors for the production of Omicron mAb are shown. (B) FACS plots showing the sorting of B cells using full length Omicron S. (C) Proportion of RBD and NTD binding antibodies found in the Omicron mAb compared to early pandemic mAb. (D) Heavy and Light chain variable gene usage. (E) Somatic mutations found in the potent Omicron mAb (FRNT50<100 ng/ml) compared to the early pandemic set.

FIG. 2. Neutralization curves using Omicron mAb. (A) Victoria, Alpha, Beta, Gamma, Delta and Omicron BA.1 viruses. (B) neutralization of Victoria, BA.1, BA.1.1, BA.2 and BA.3 pseudoviruses by Omicron mAb. (C) neutralization of Victoria, BA.1, BA.1.1, BA.2 and BA.3 pseudoviruses by antibodies being developed for commercial use.

FIG. 3. Neutralization of Victoria, BA.1, BA.1.1, BA.2 and BA.3 pseudoviruses.

Neutralization of Victoria, BA.1, BA.1.1, BA.2 and BA.3 pseudoviruses 28 days following the second and third doses of (A) AZD1222 (n=41), (B) BNT162b2 (n=20). (C) Live virus neutralization assays with Victoria, Alpha, Beta, Gamma, Delta and Omicron viruses using sera obtained <14 days and >21 days following symptom onset (D) Neutralization of Victoria, BA.1, BA.1.1, BA.2 and BA.3 pseudoviruses by early and late sera. Geometric mean titres are shown above each column. The Wilcoxon matched-pairs signed rank test (A and B) and Mann-Whitney test (C and D) were used for the analysis and two-tailed P values were calculated.

FIG. 4. Pseudoviral neutralization curves. Pseudoviral neutralization curves for BA.1, BA.1.1, BA.2 and BA.3 on Early pandemic mAb (B) Beta mAb.

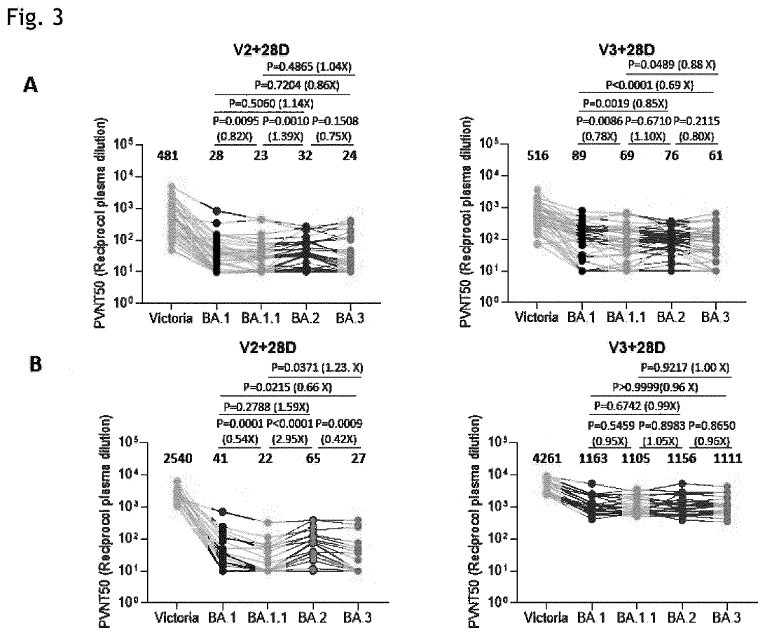

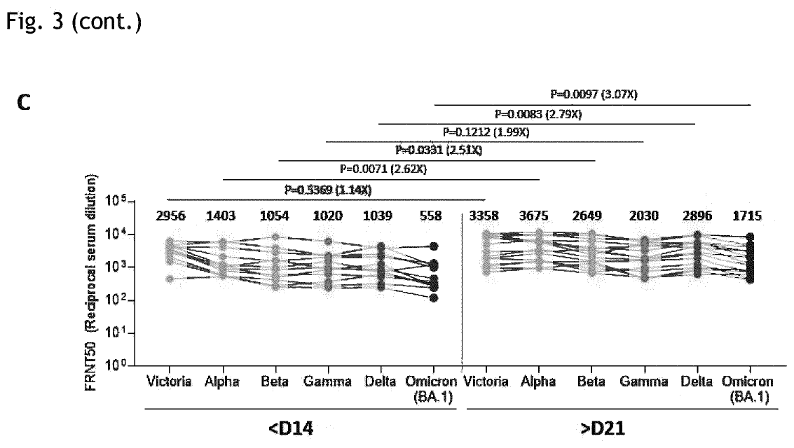

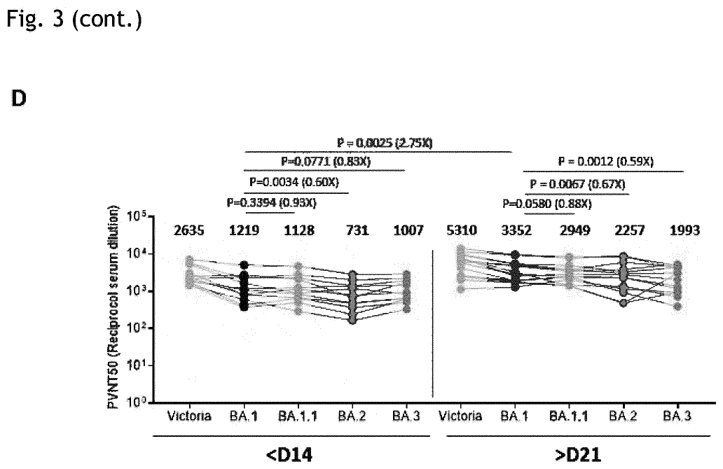

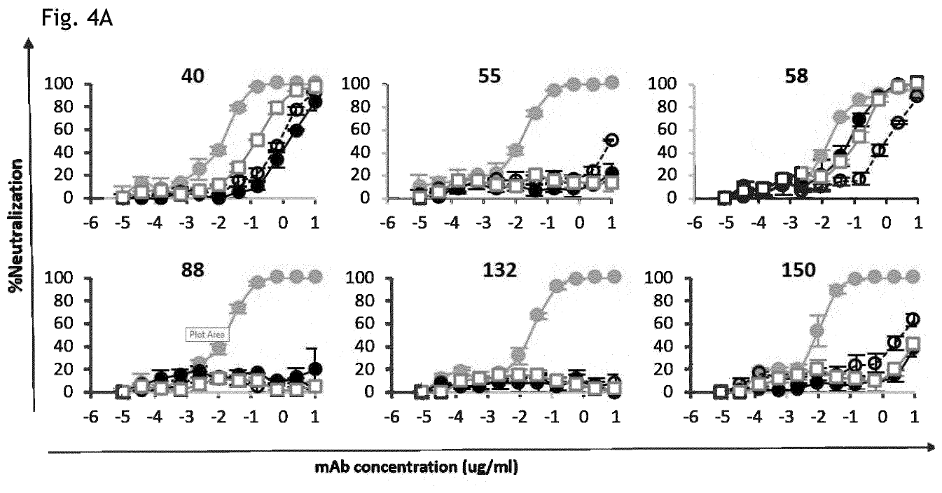

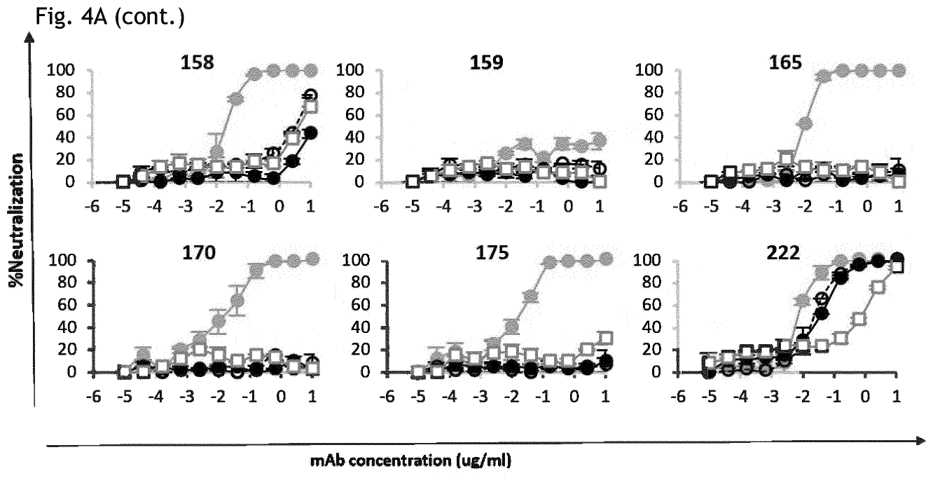

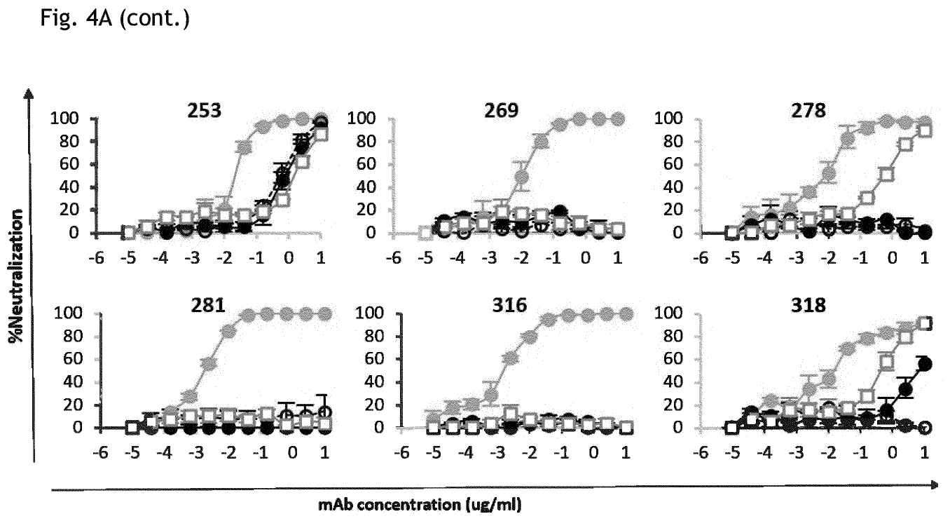

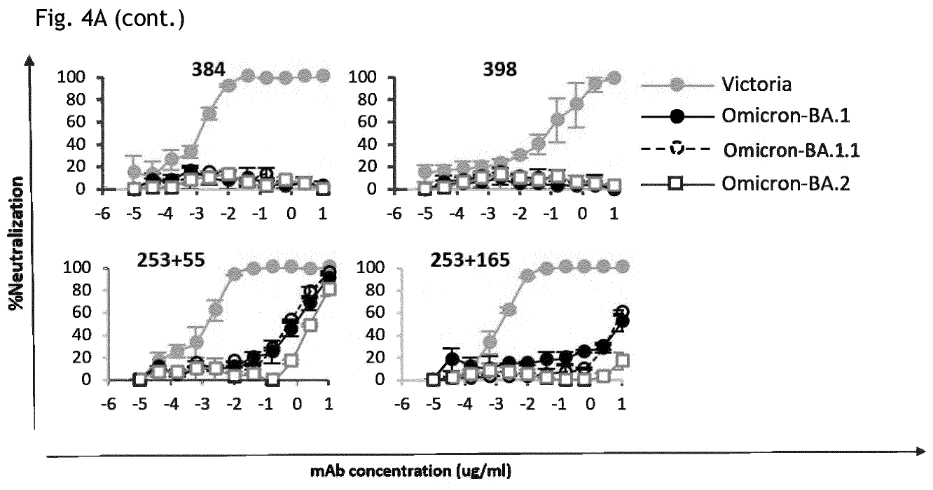

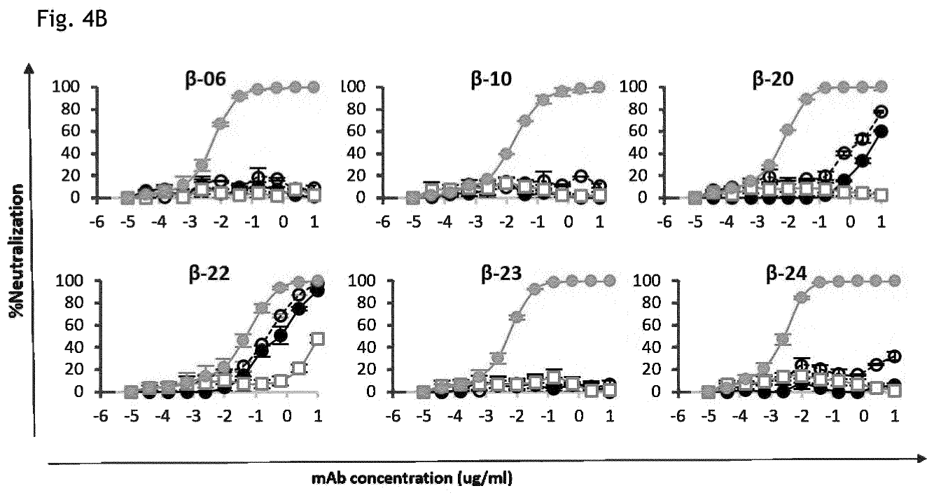

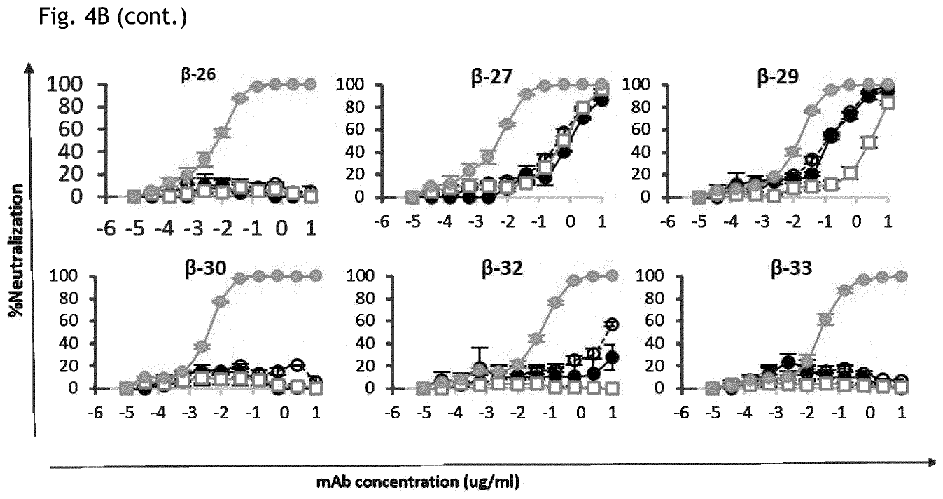

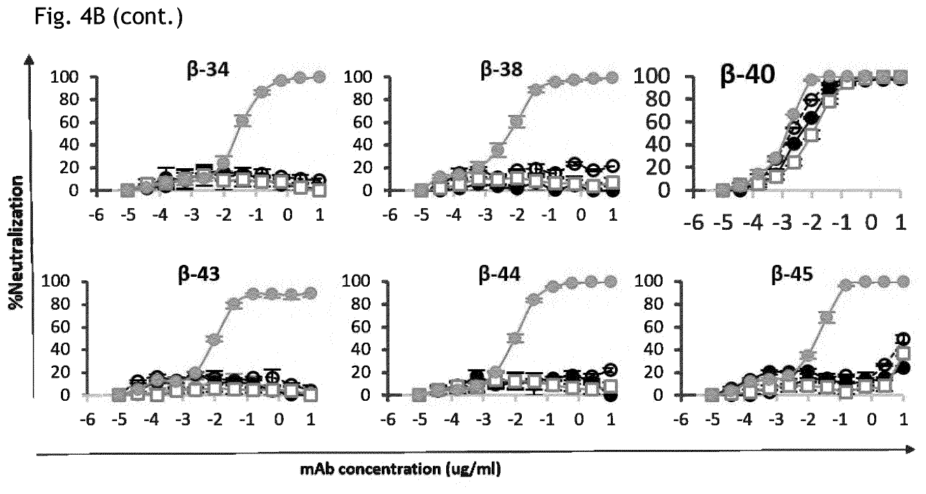

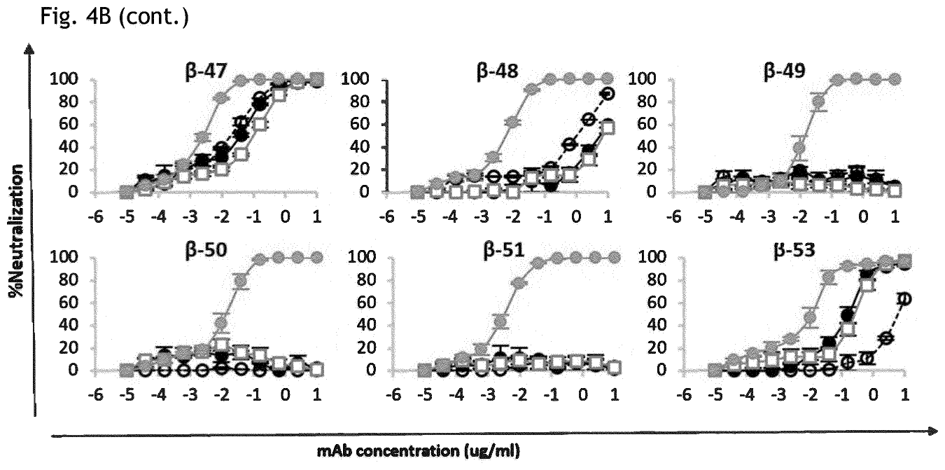

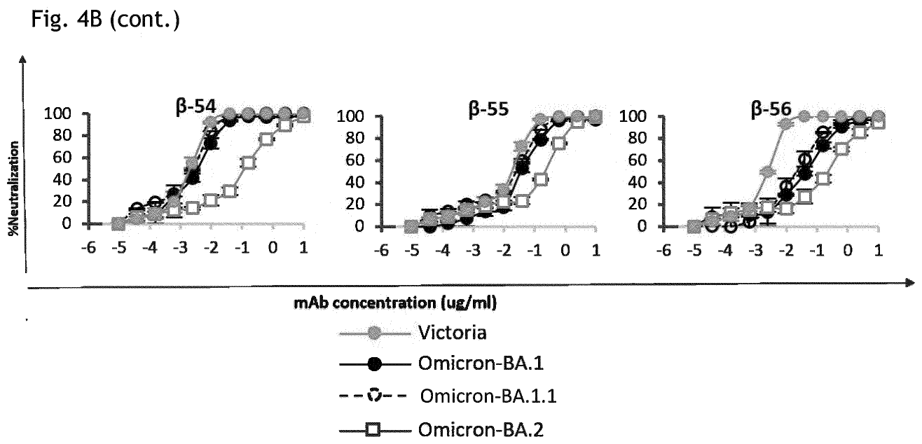

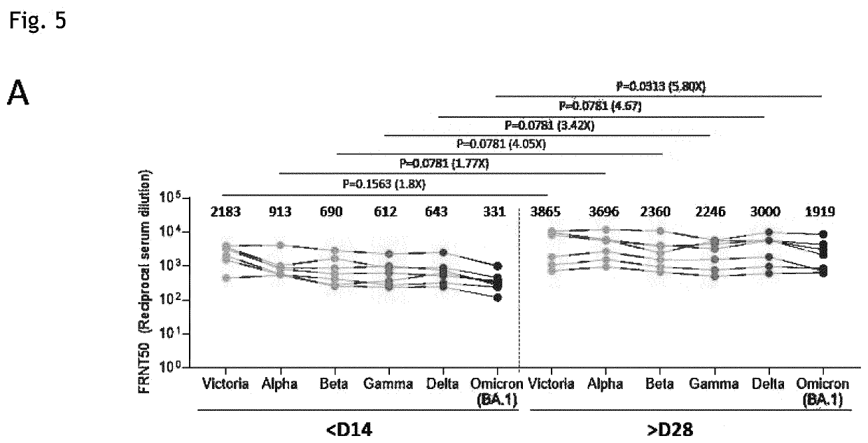

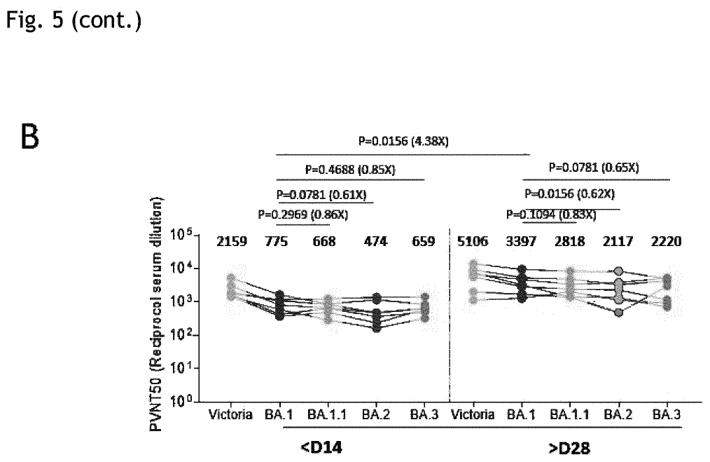

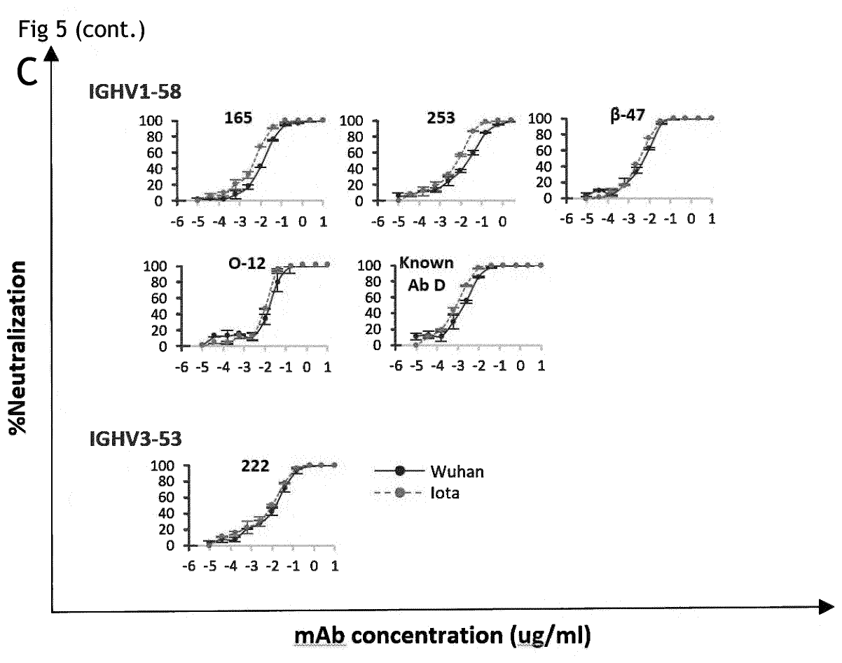

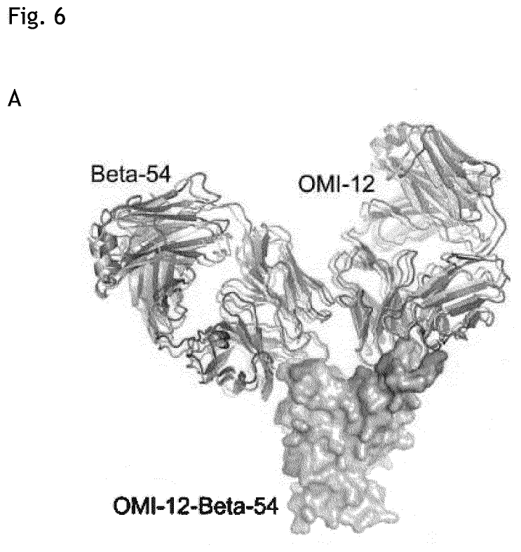

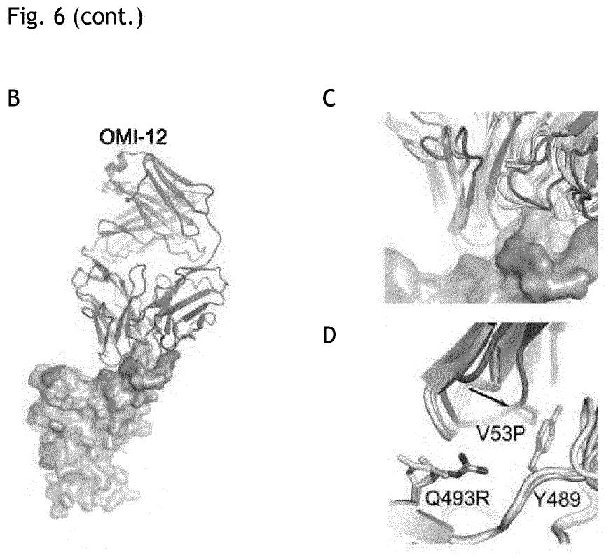

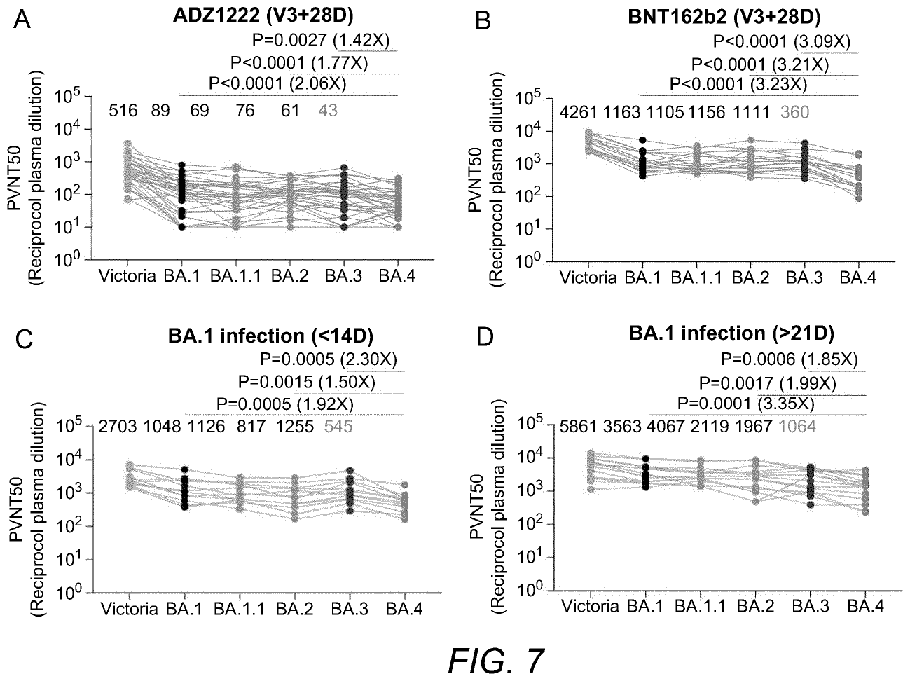

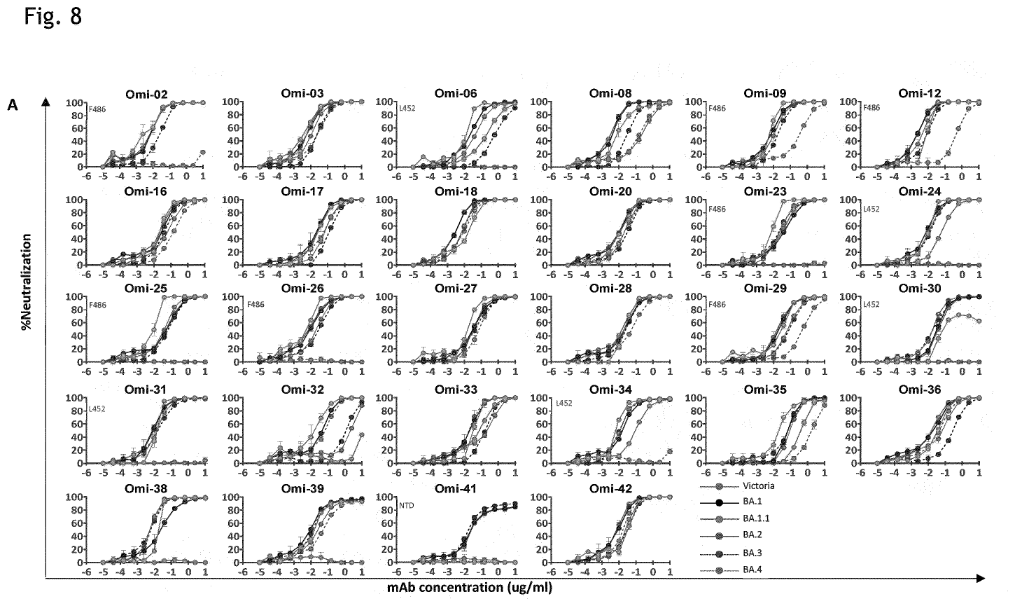

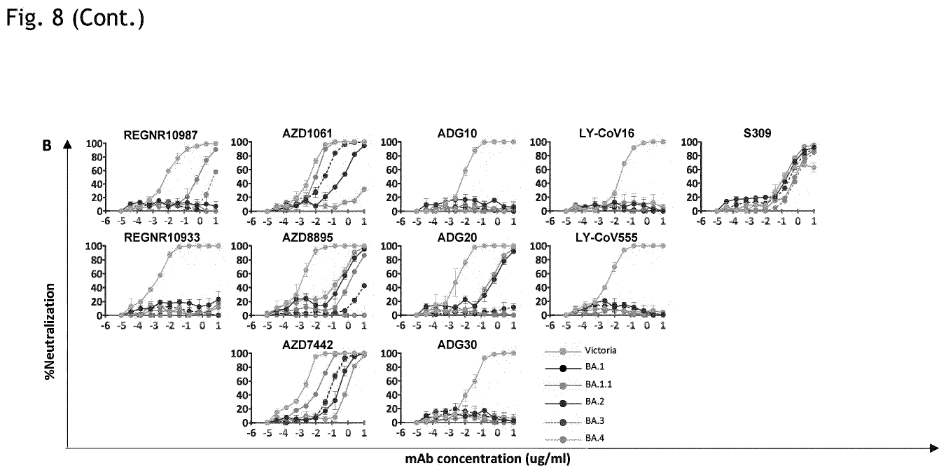

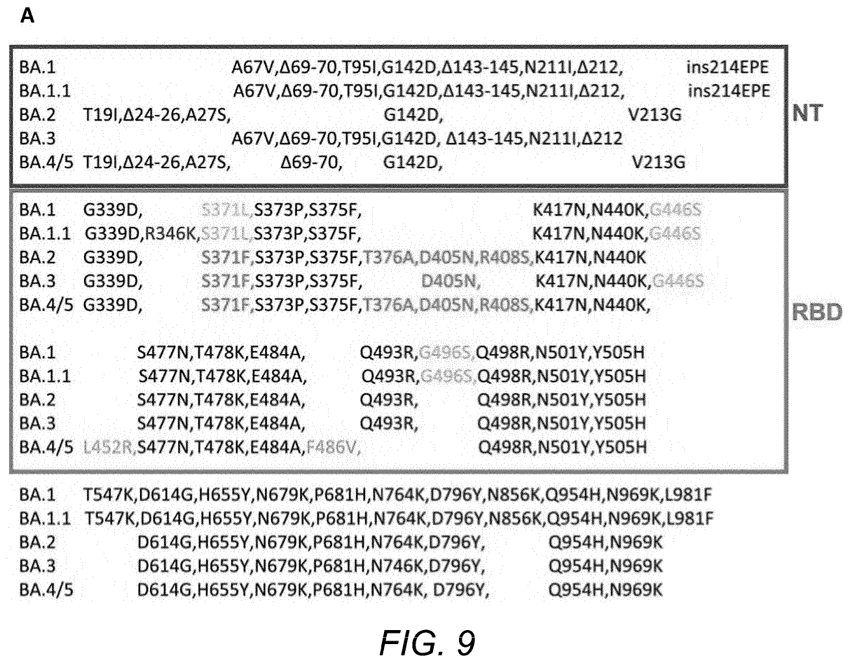

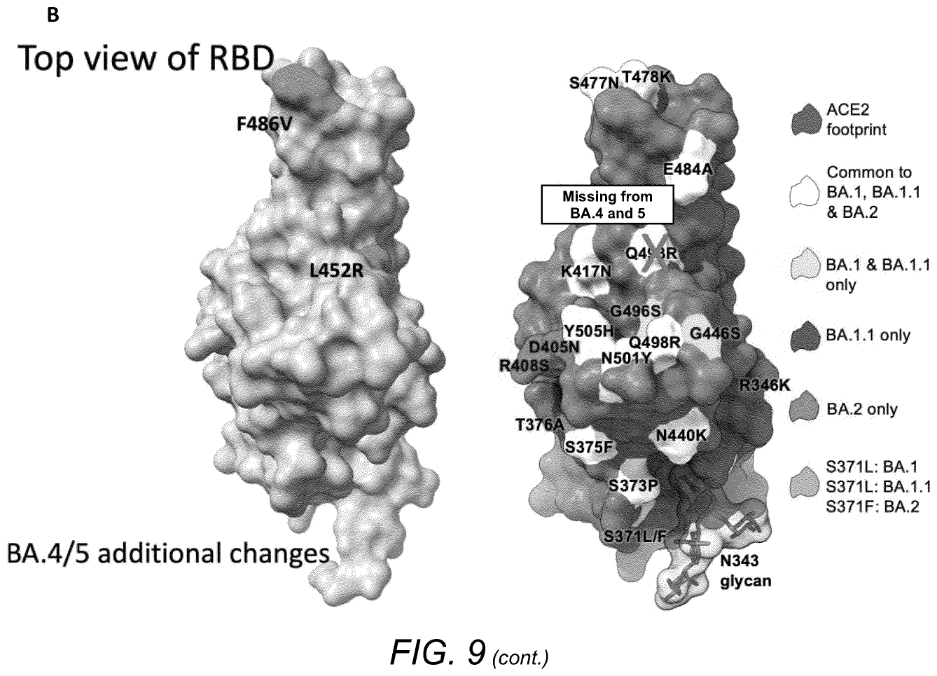

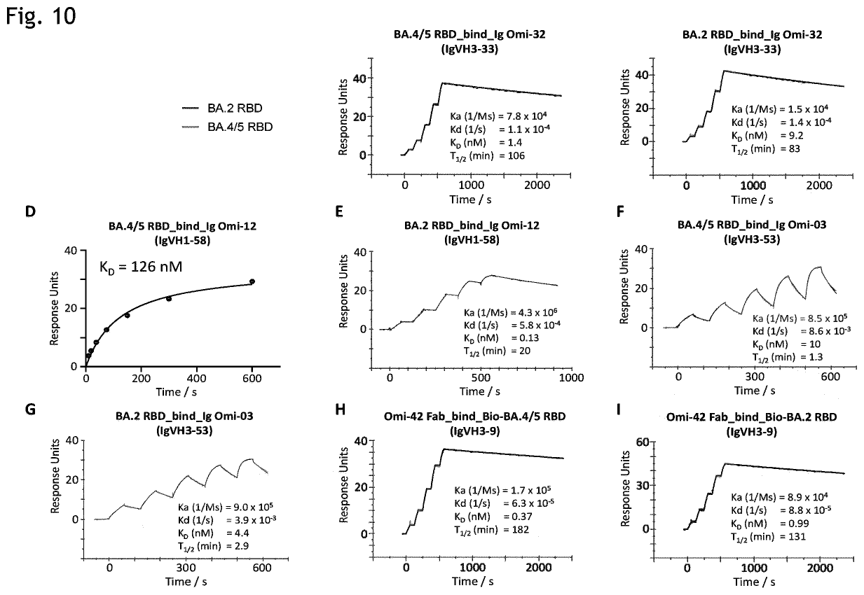

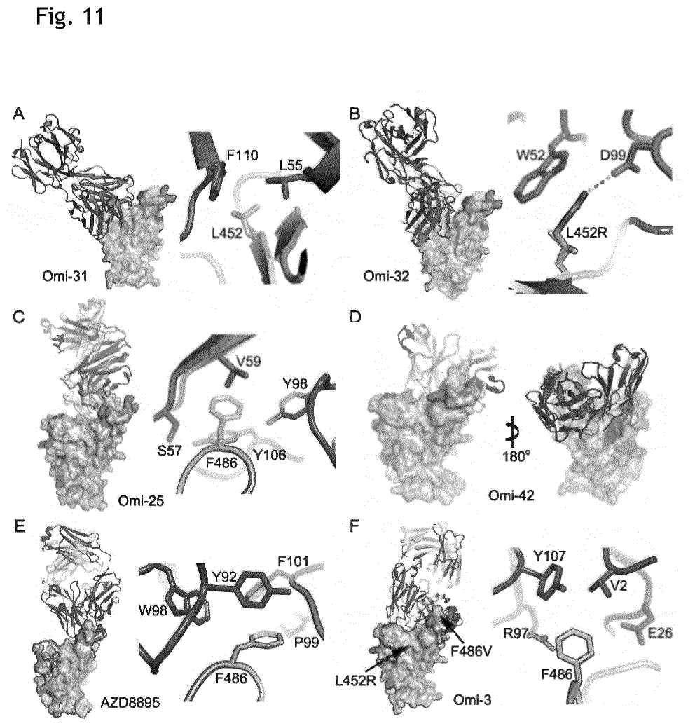

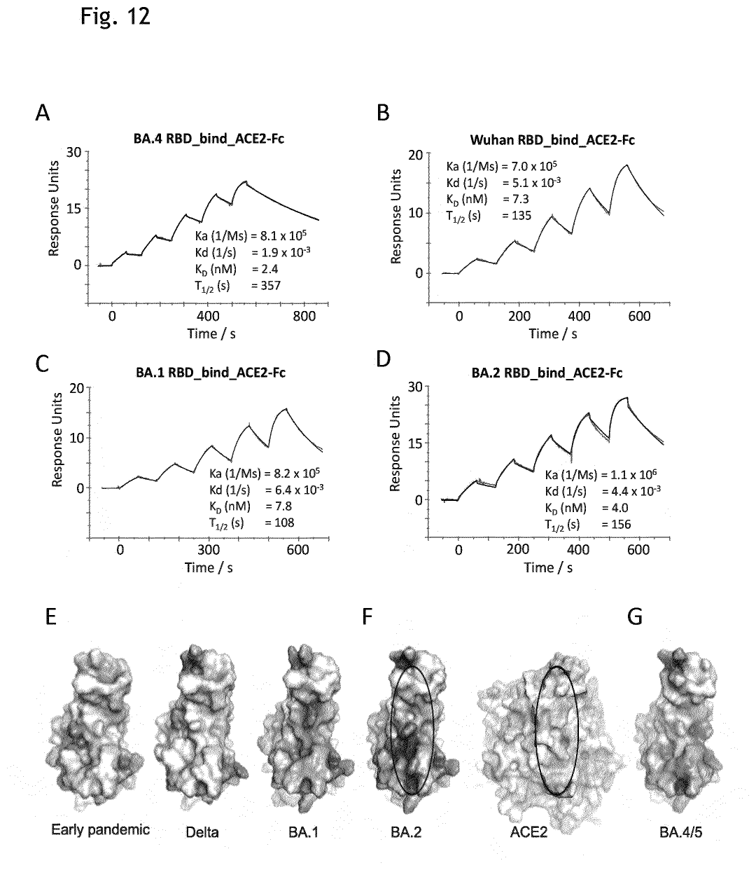

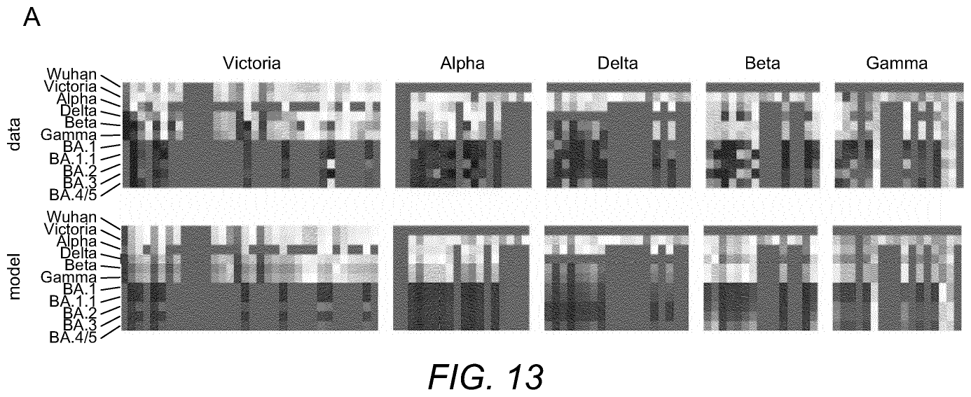

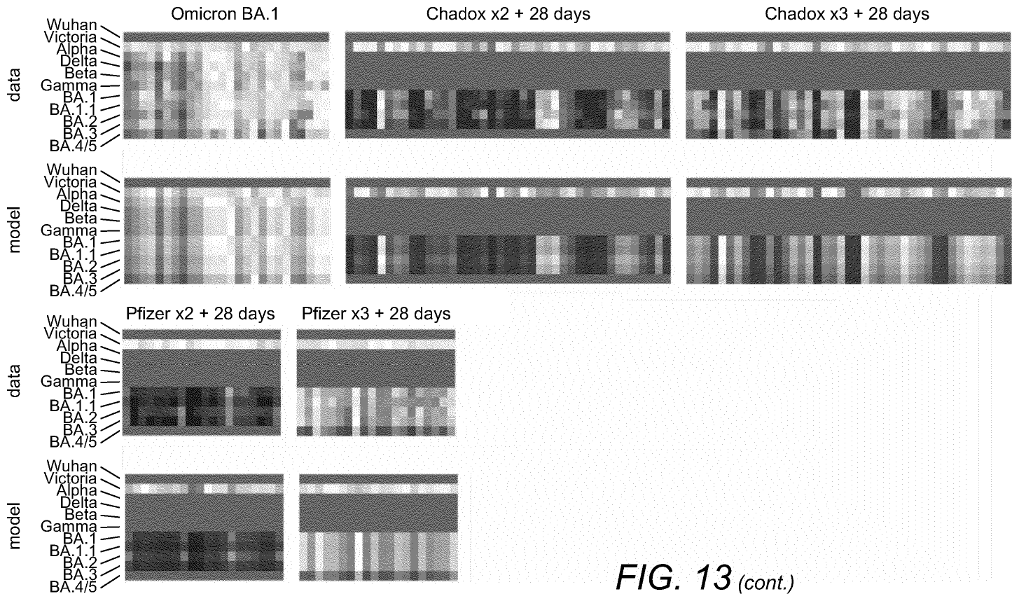

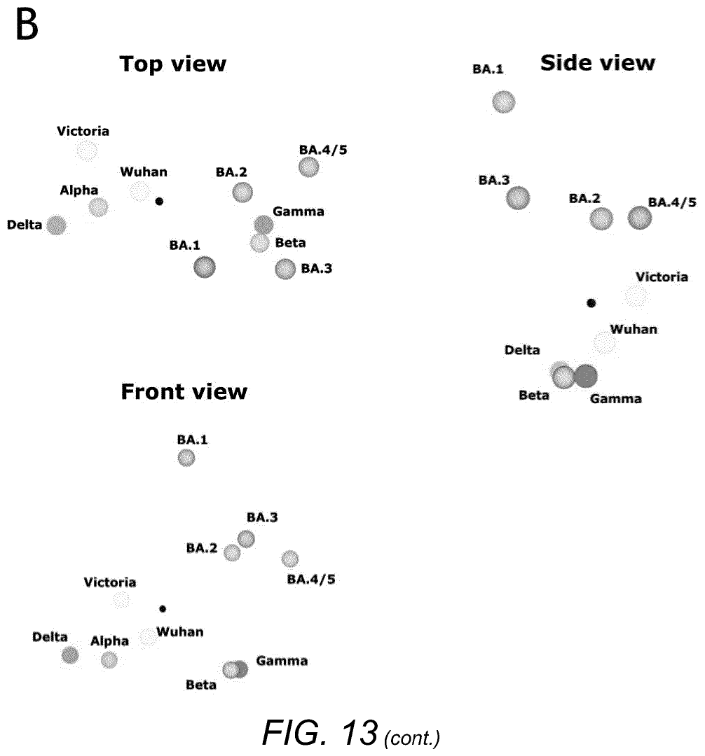

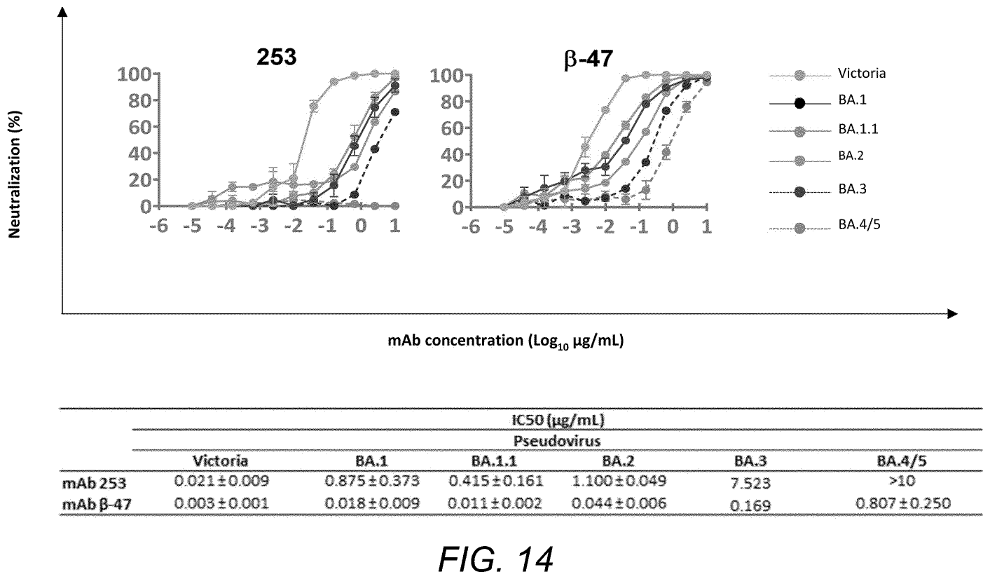

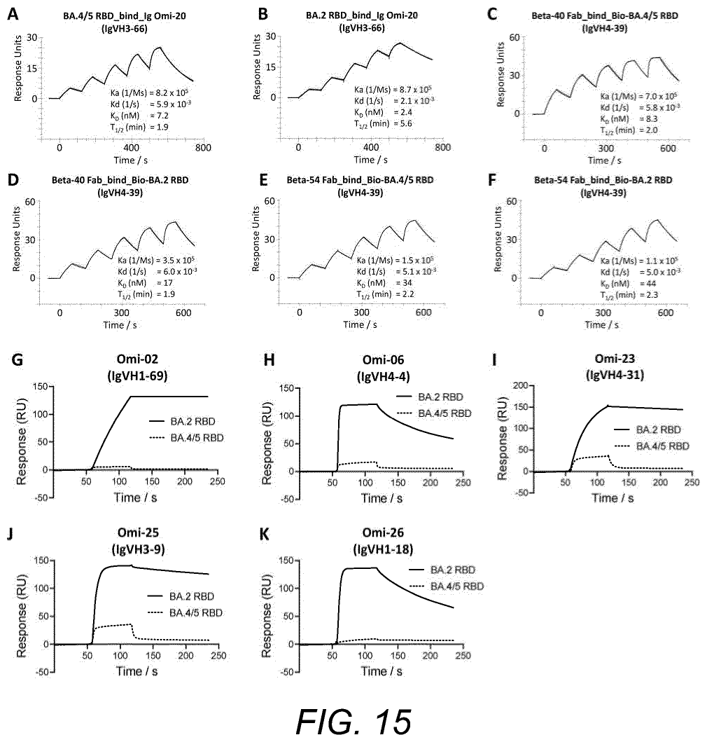

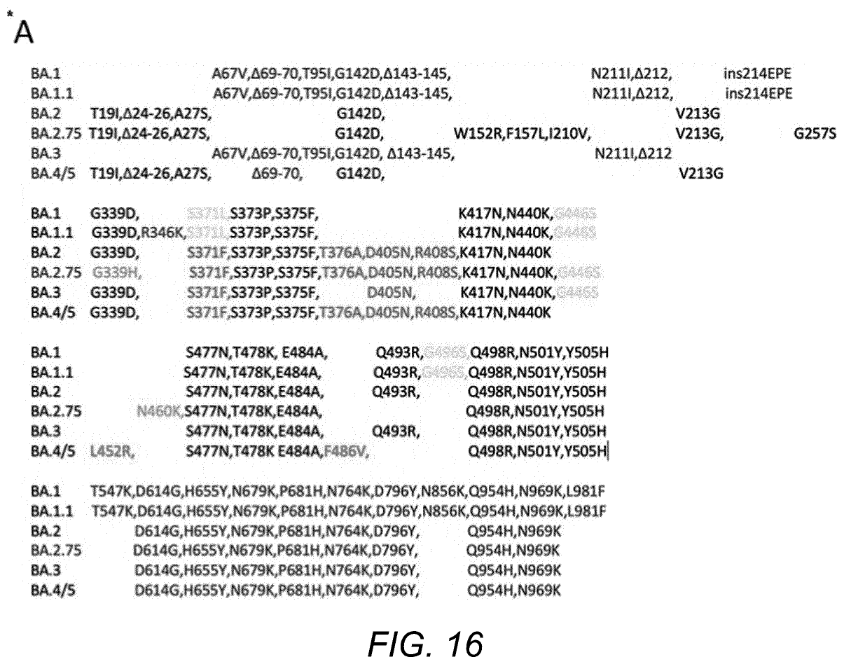

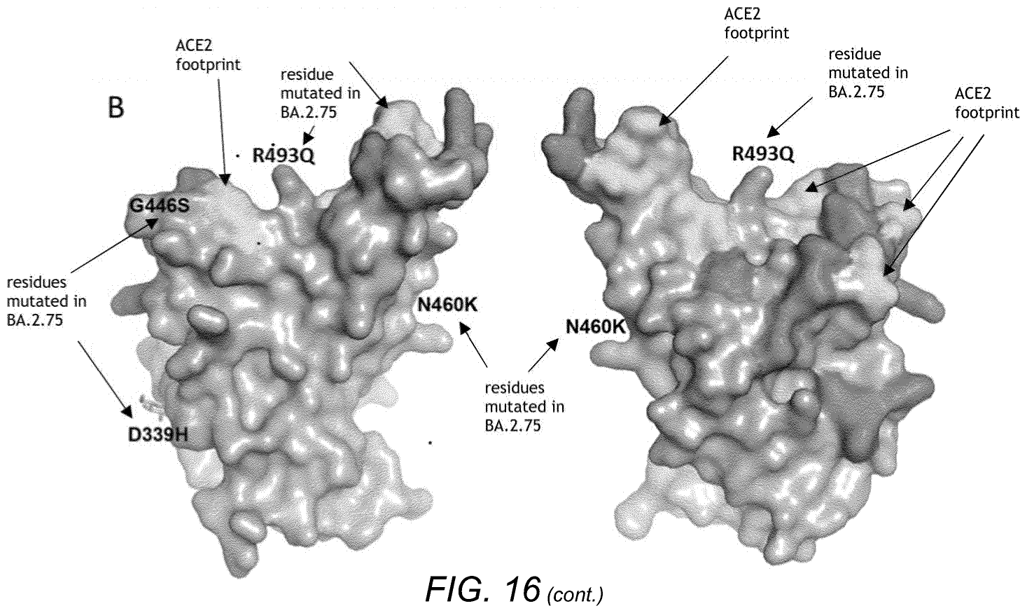

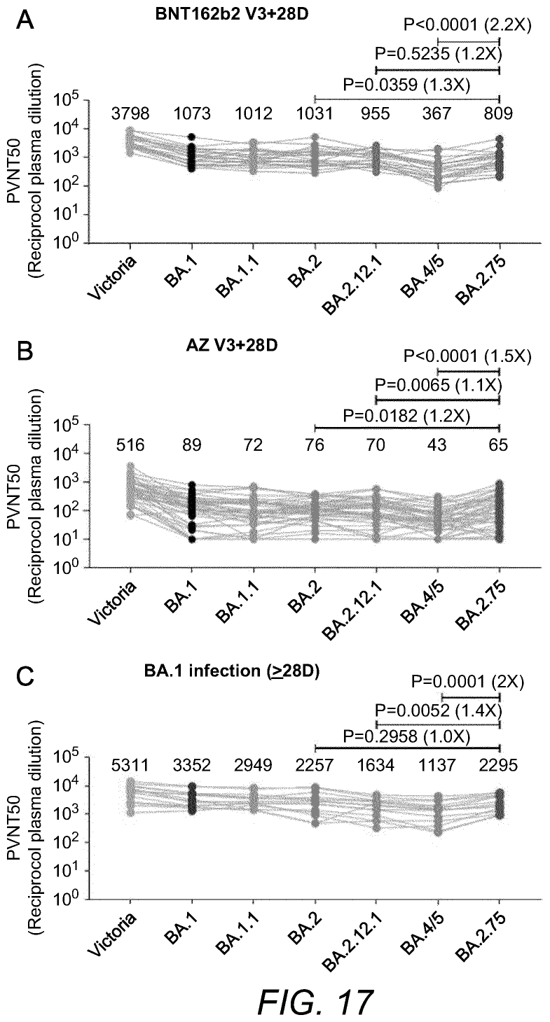

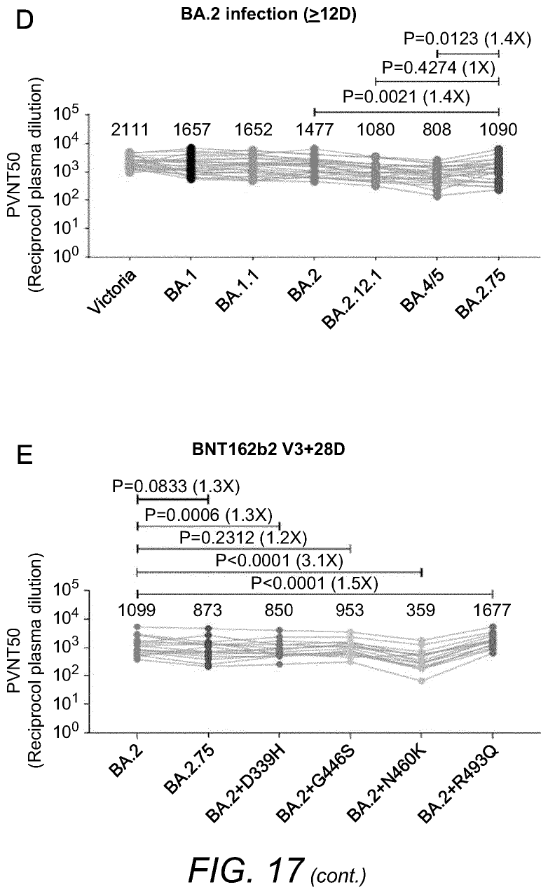

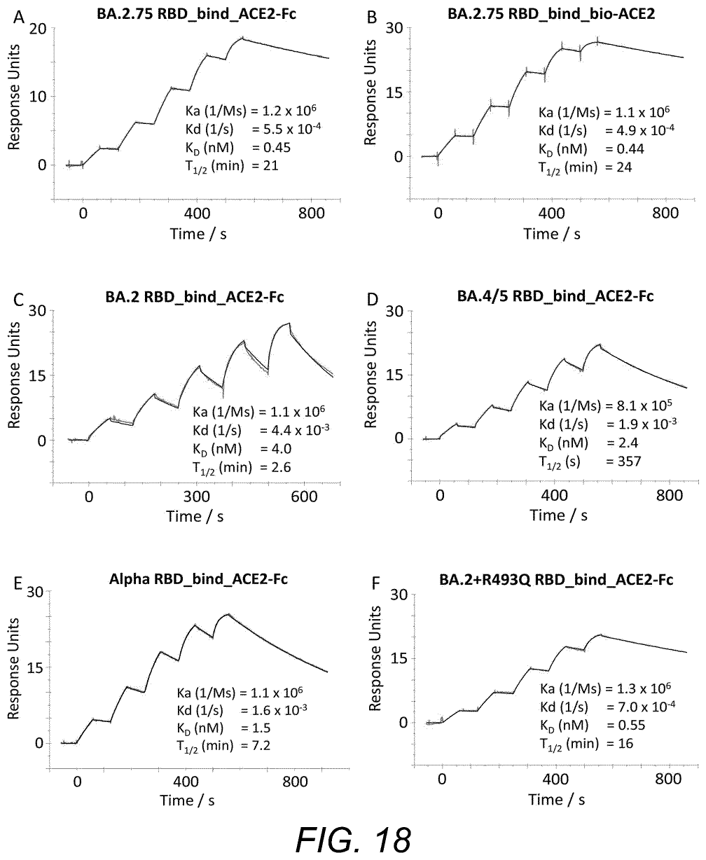

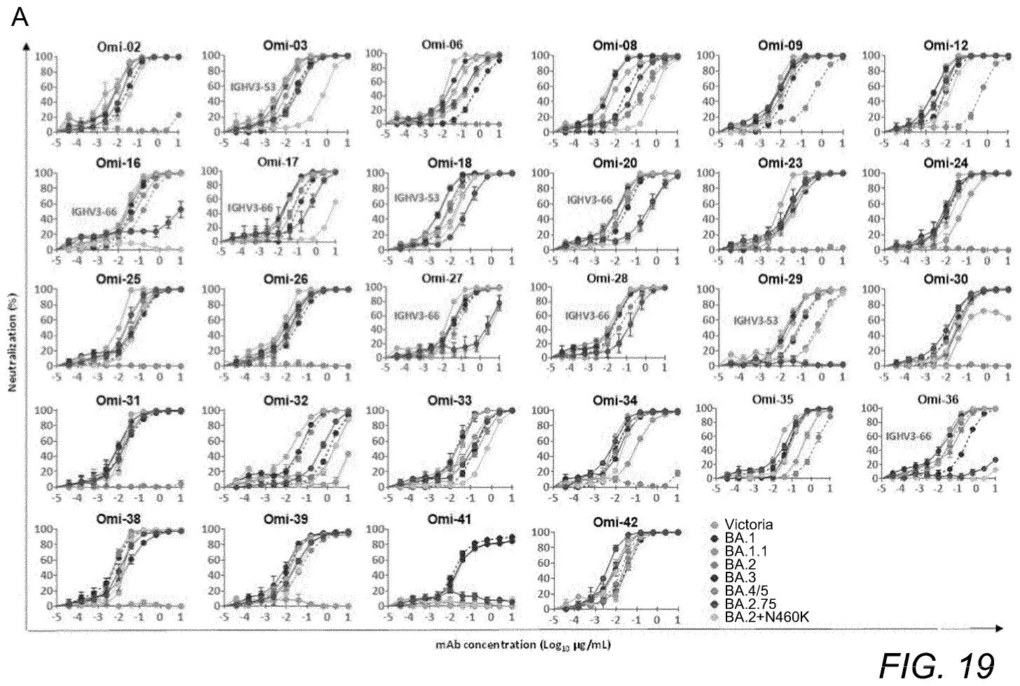

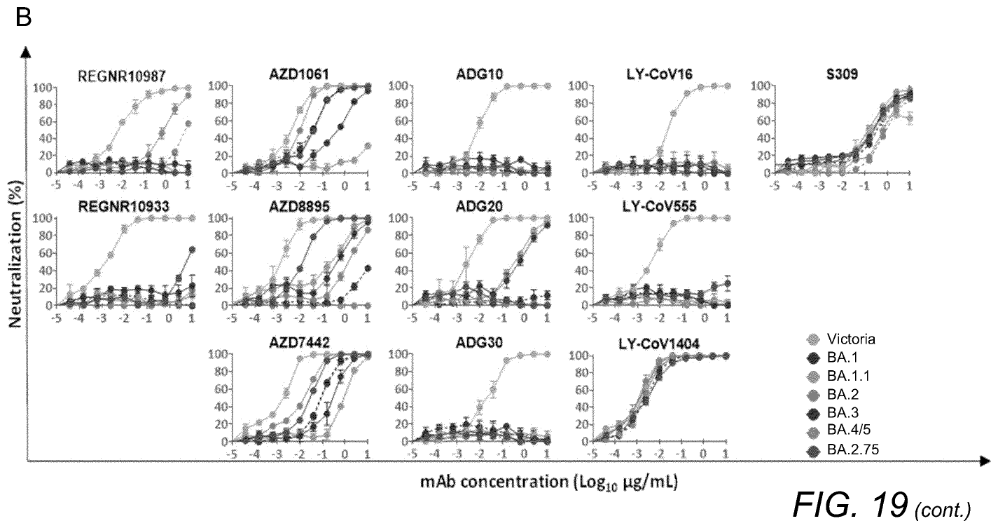

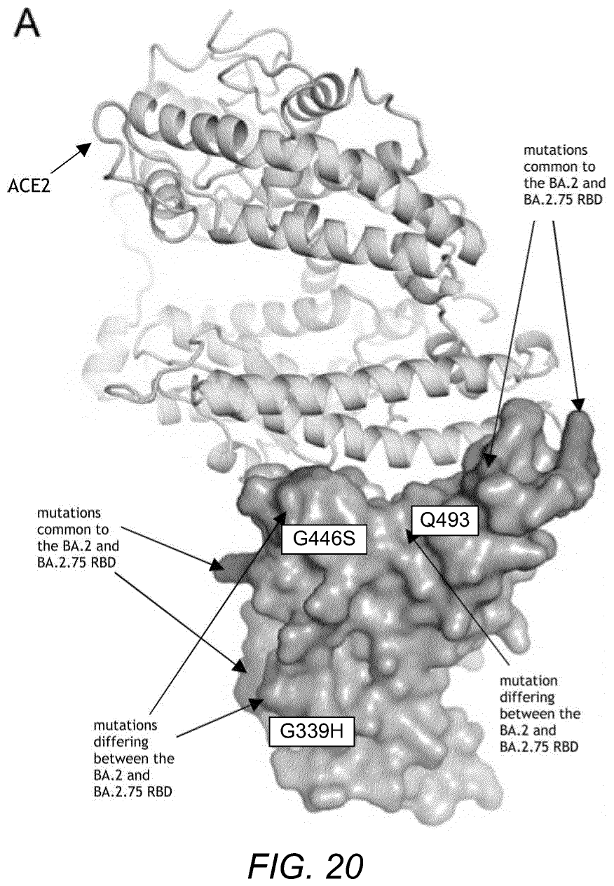

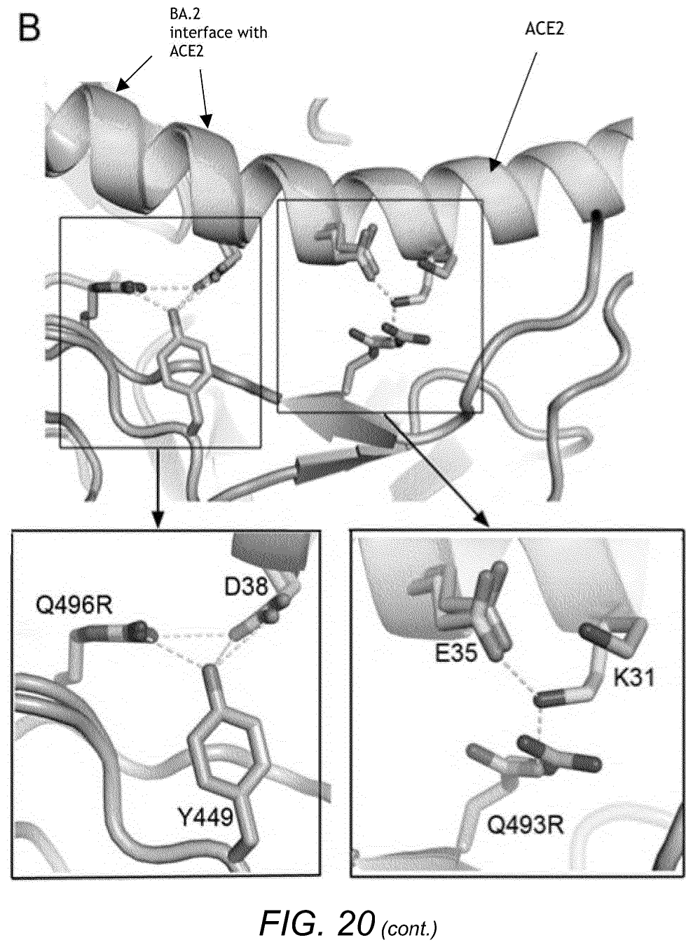

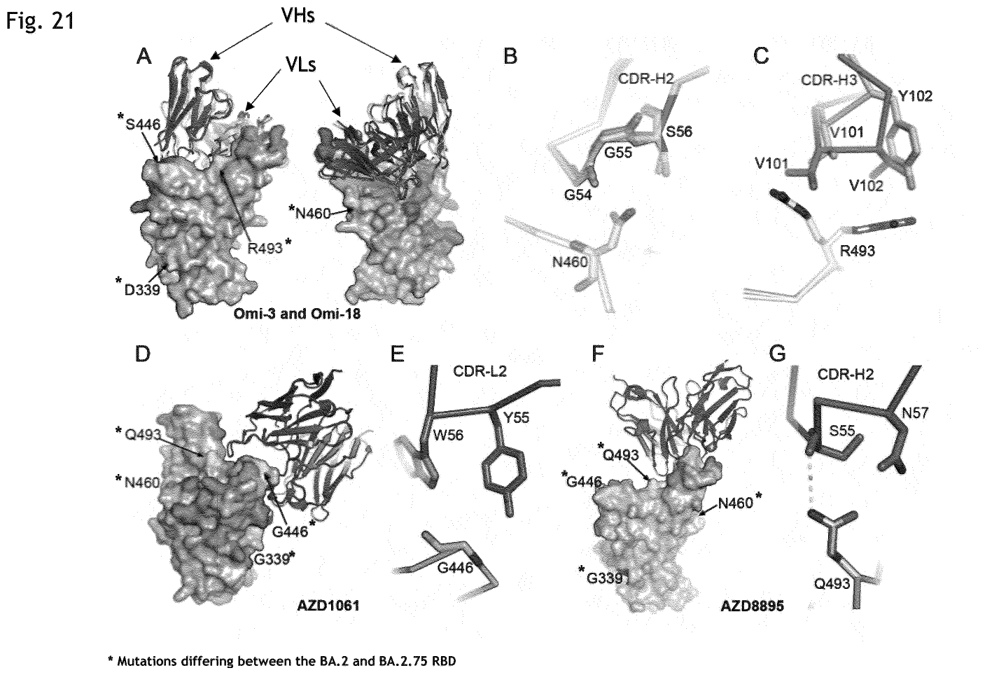

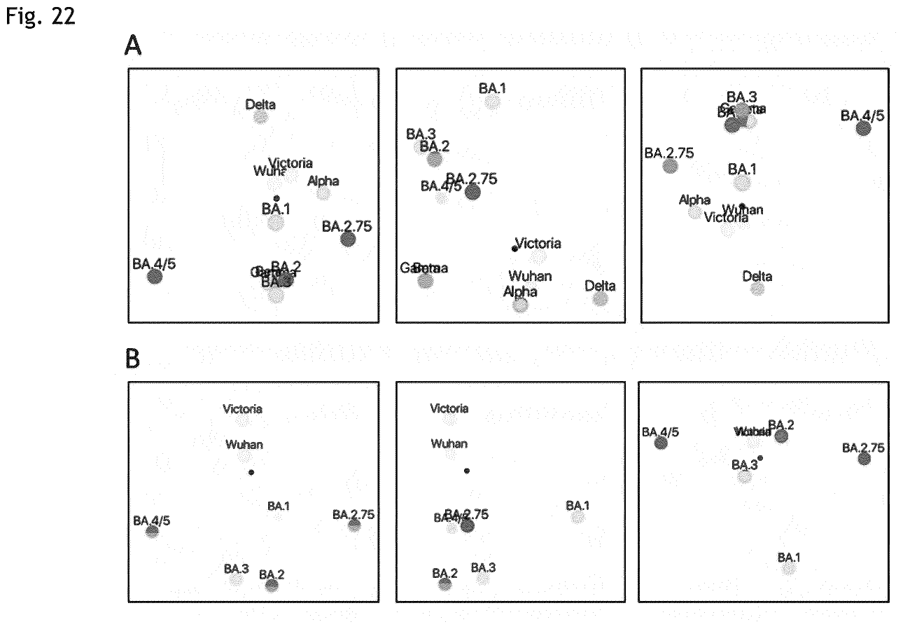

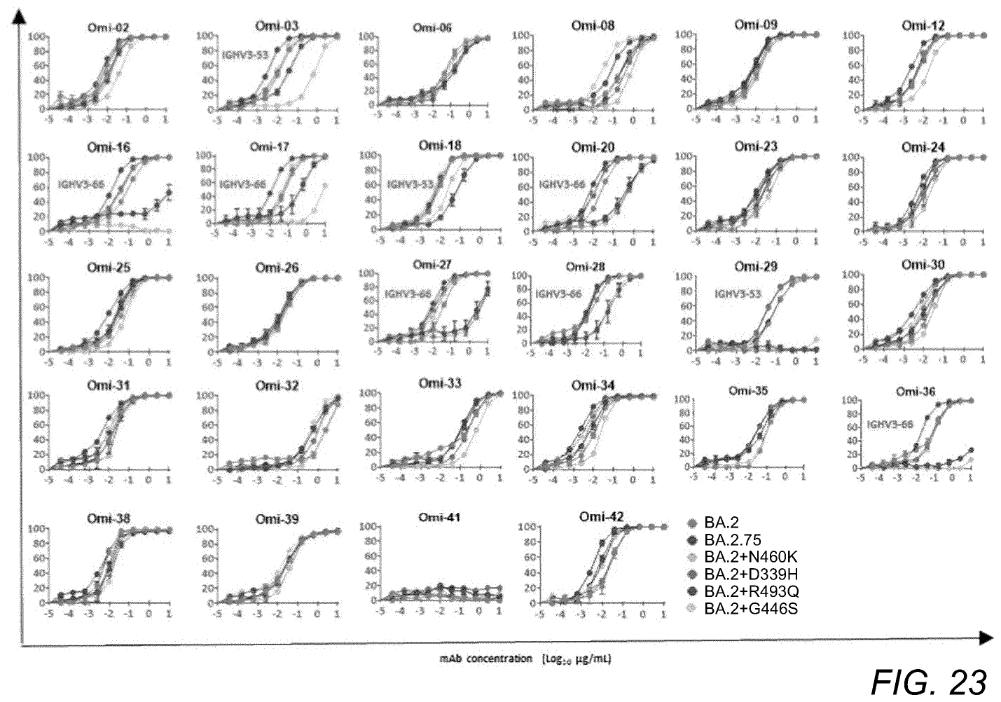

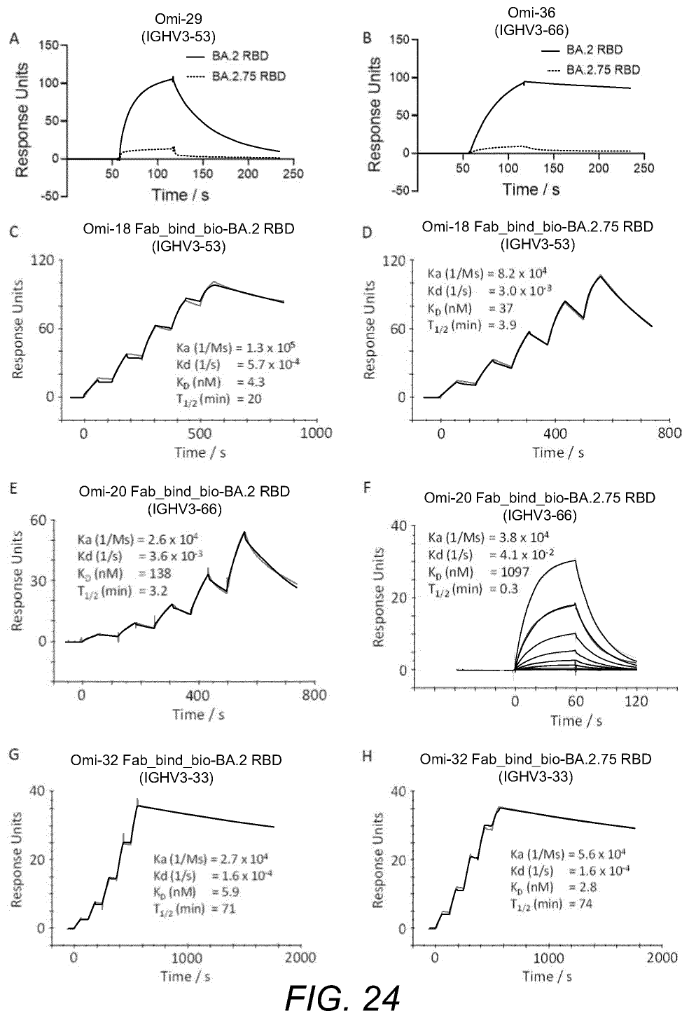

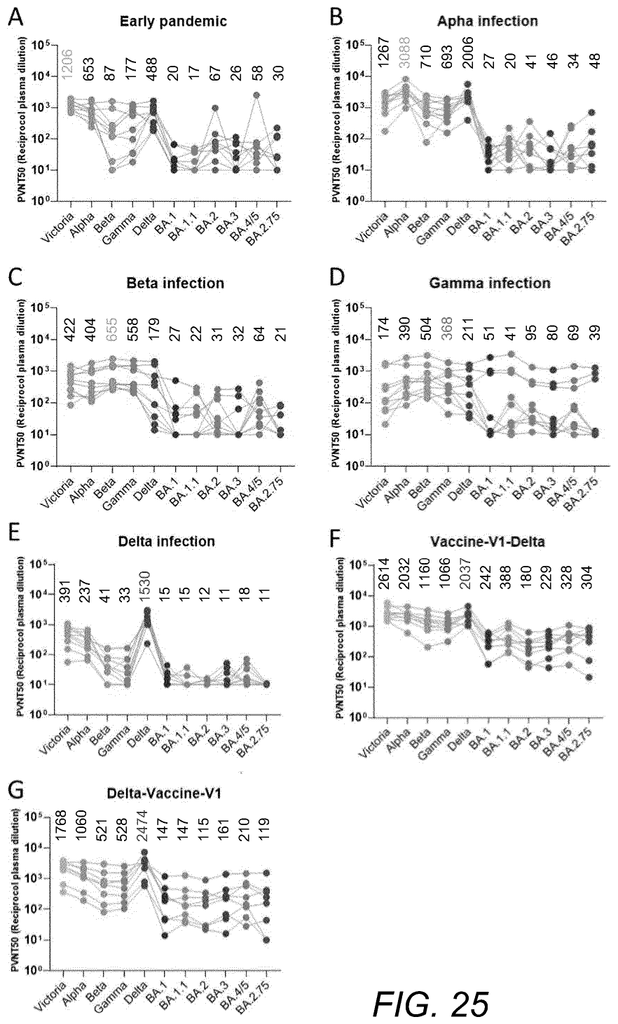

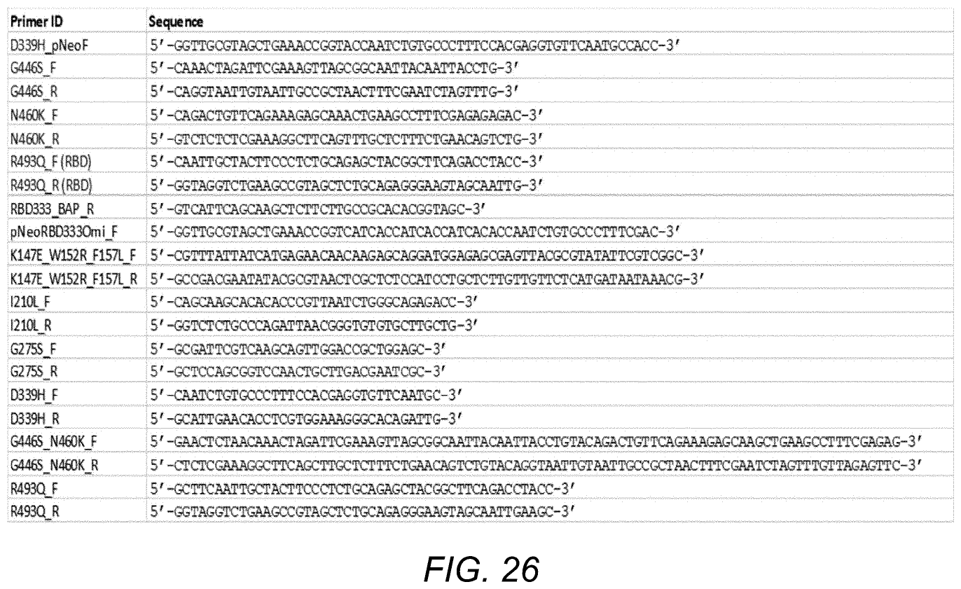

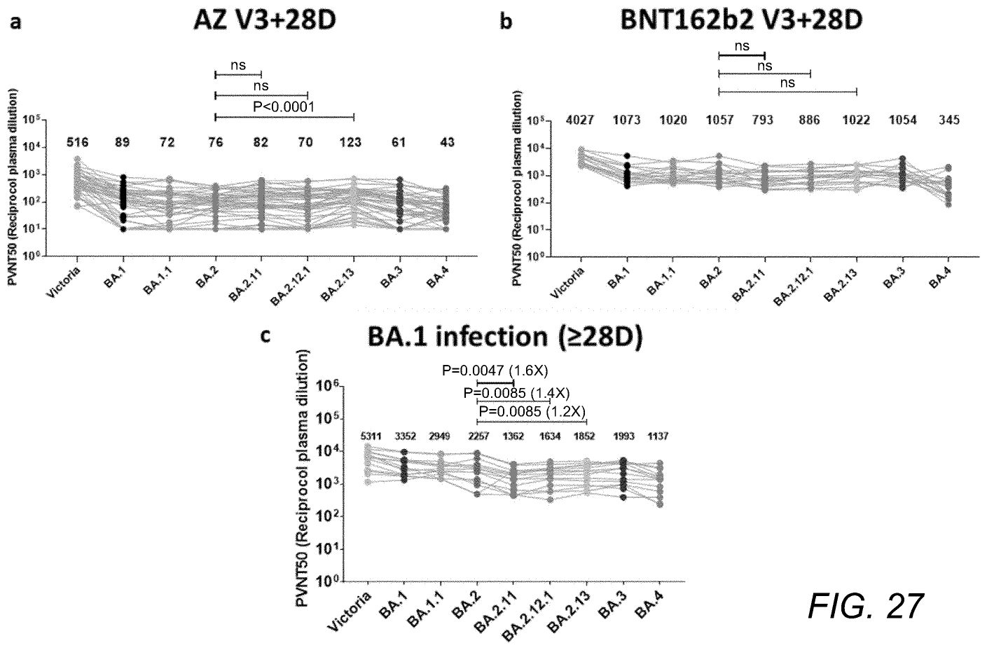

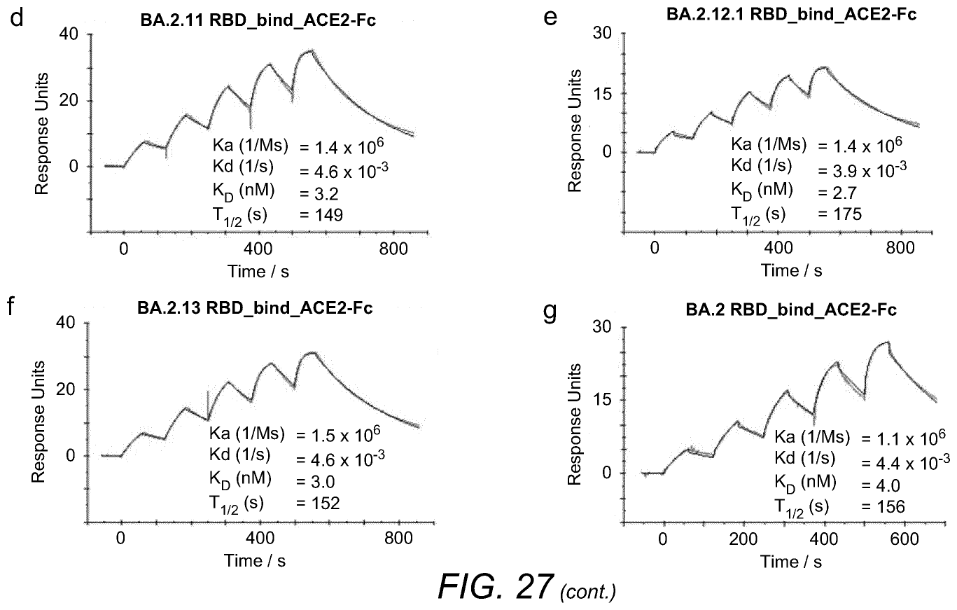

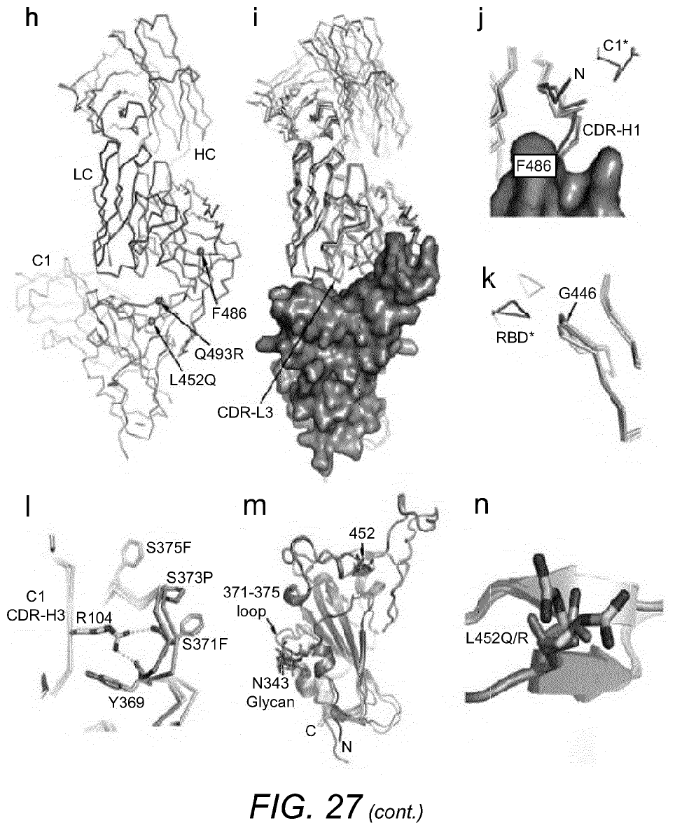

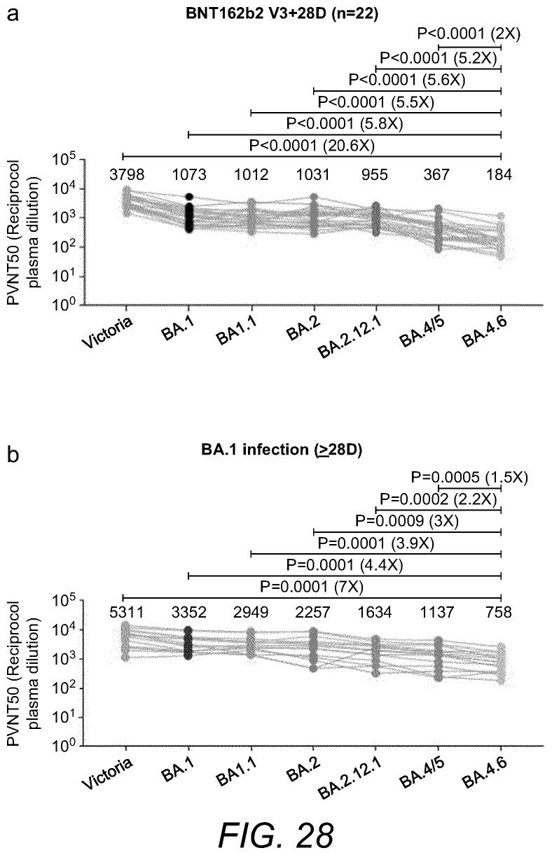

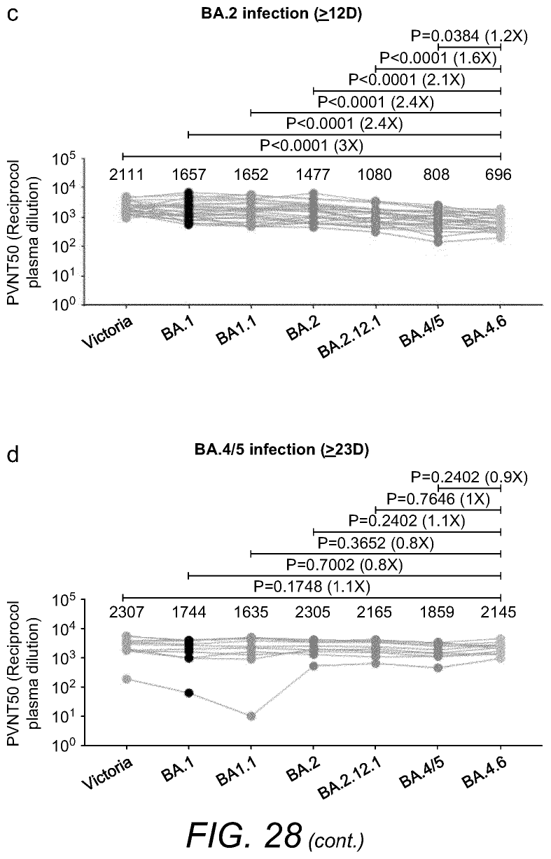

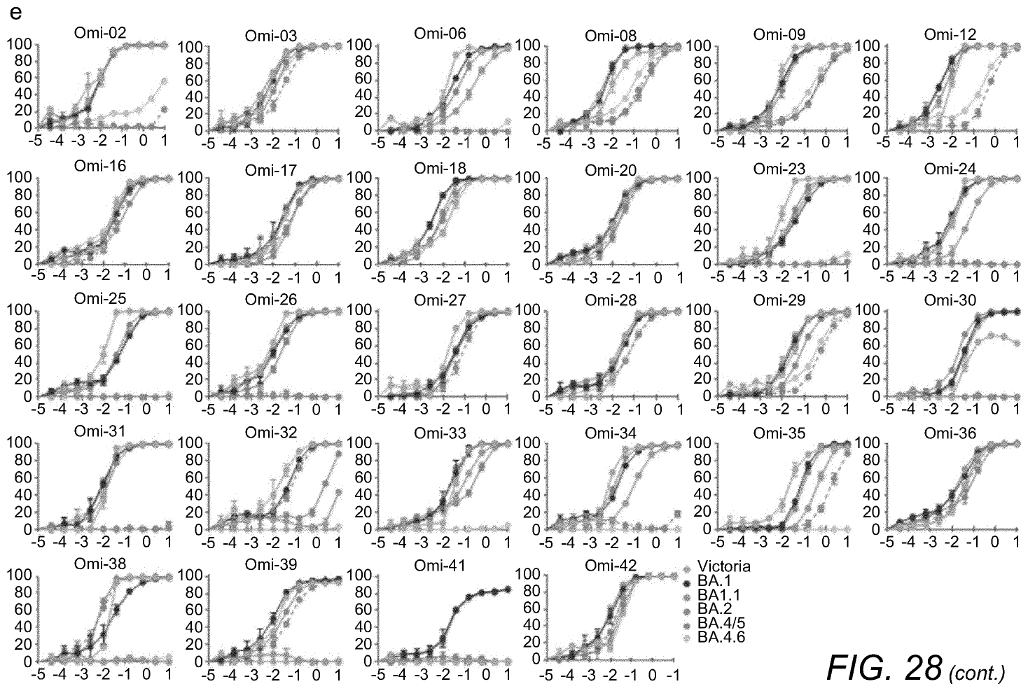

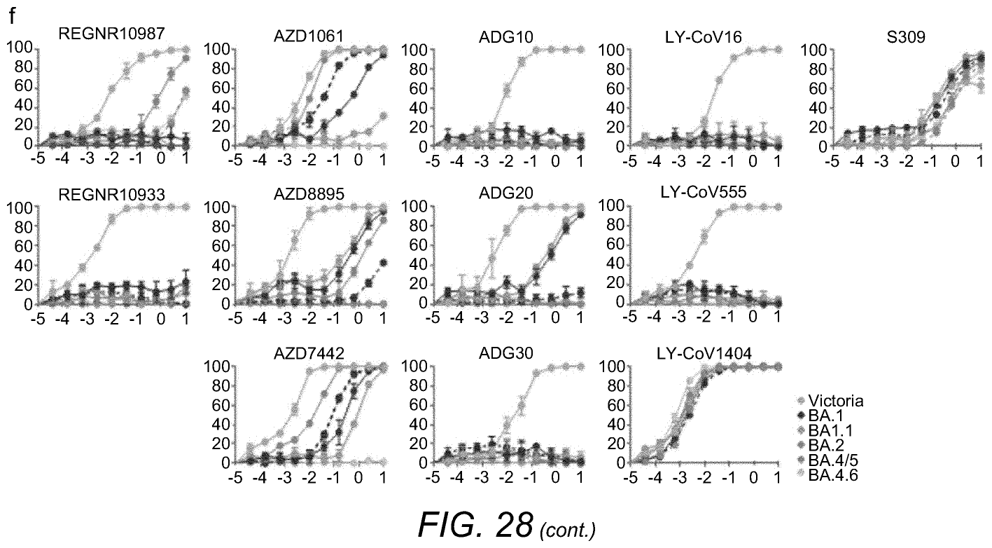

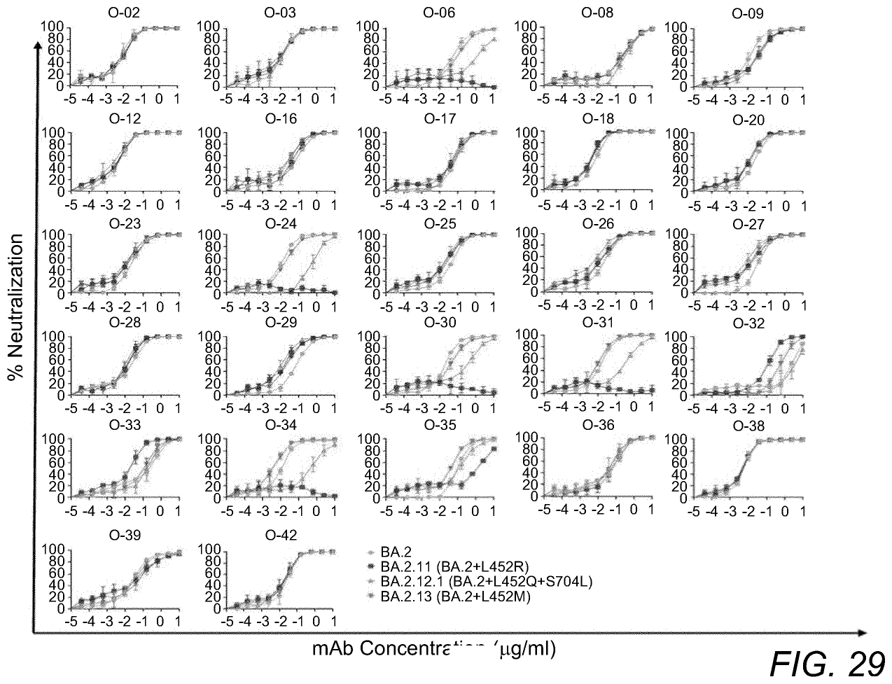

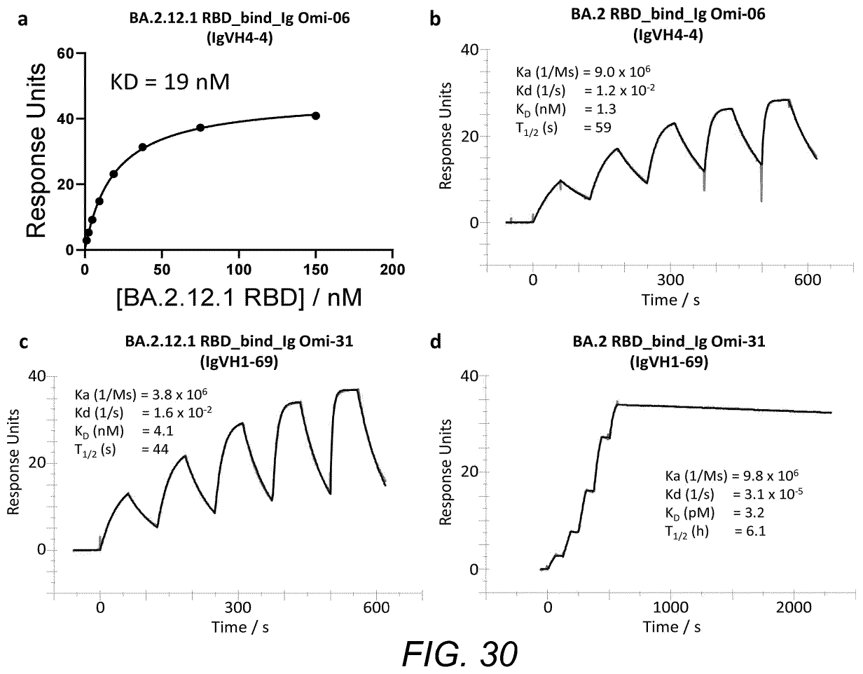

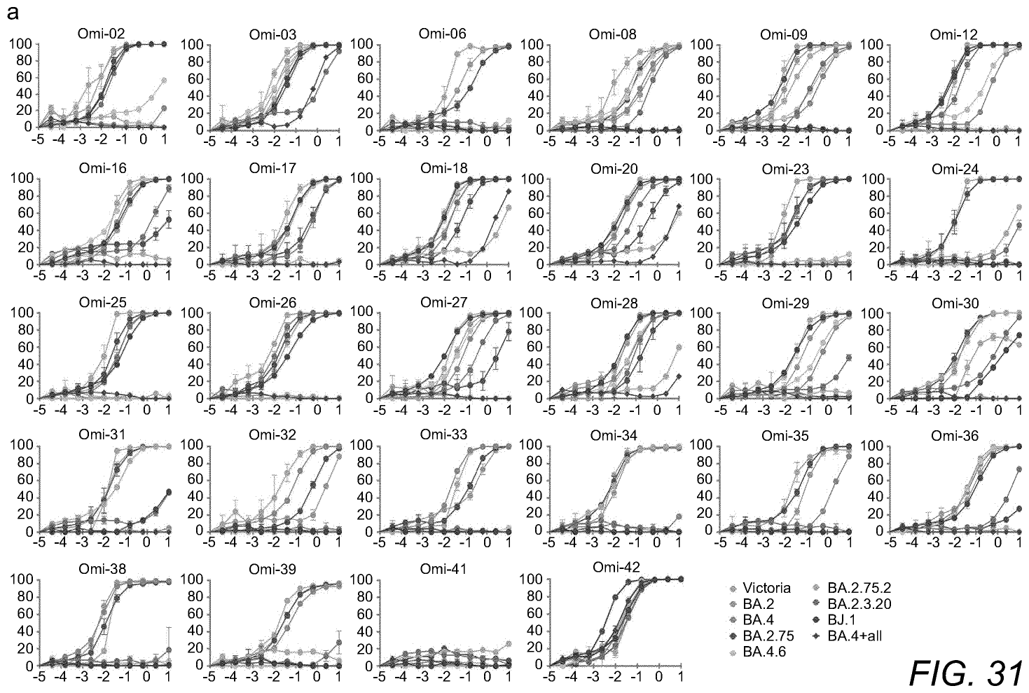

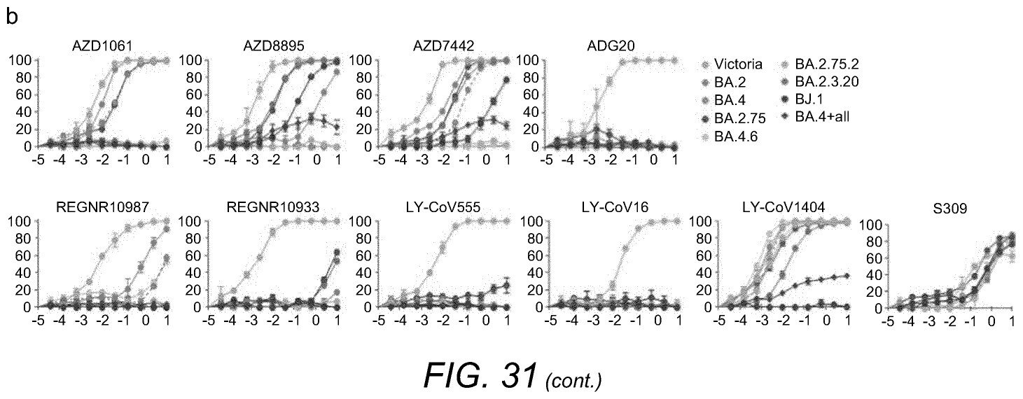

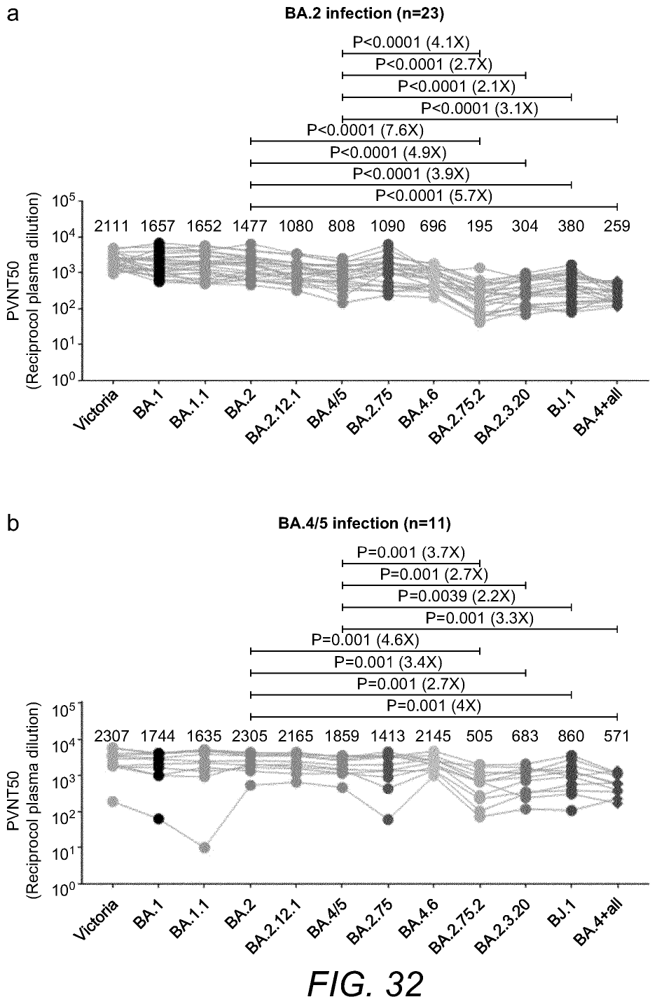

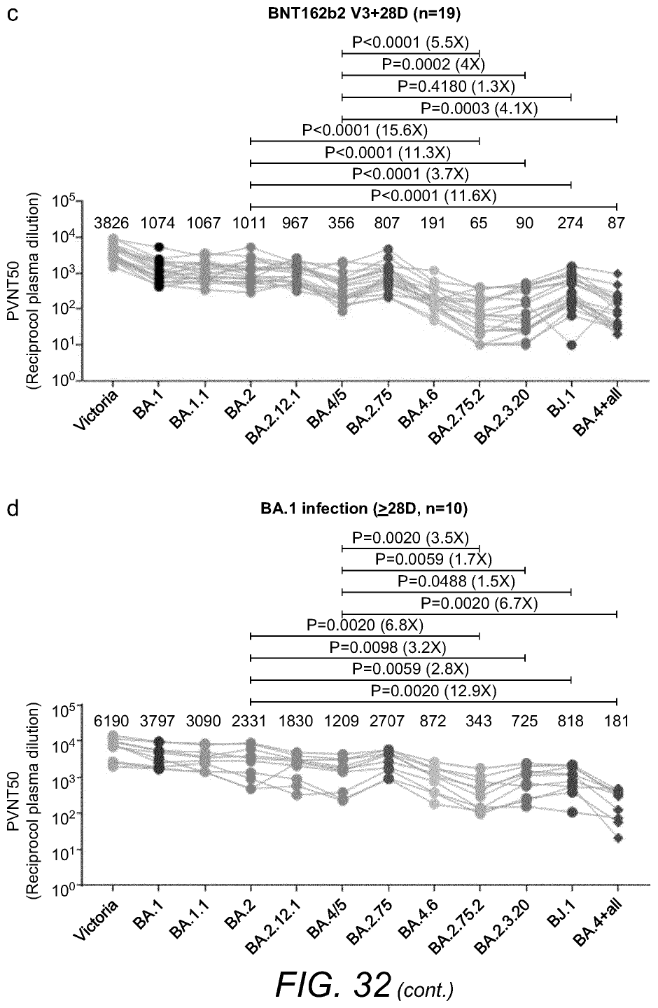

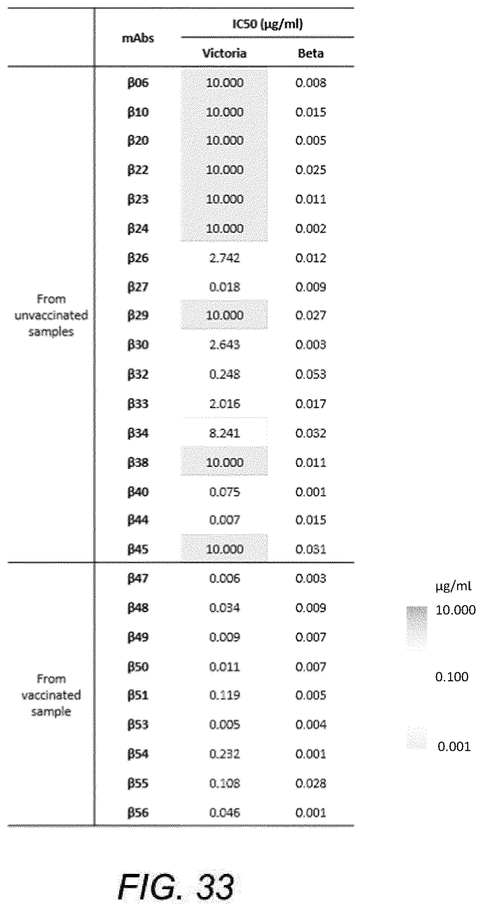

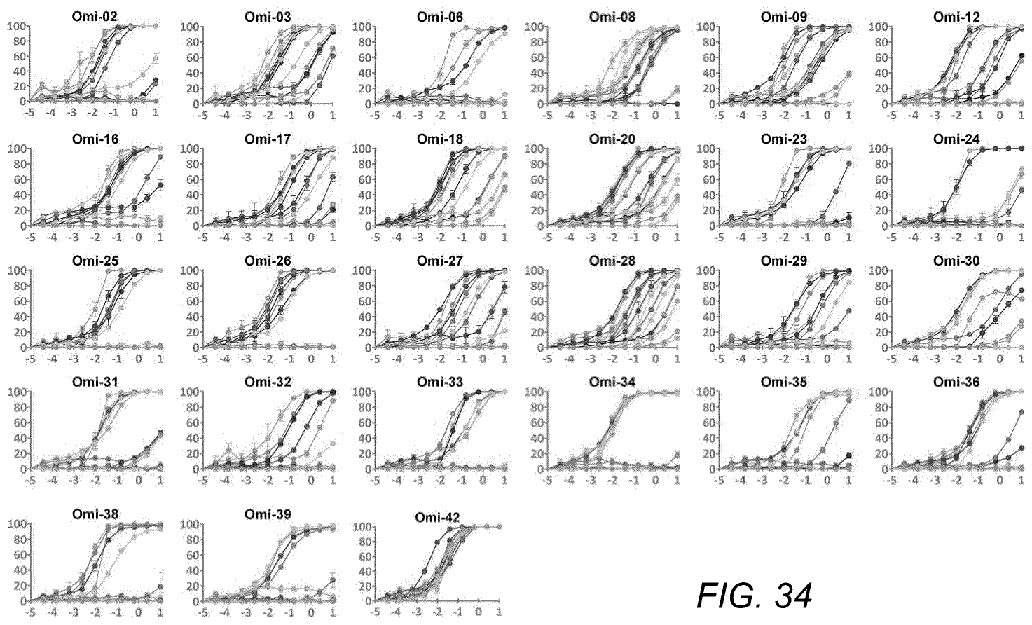

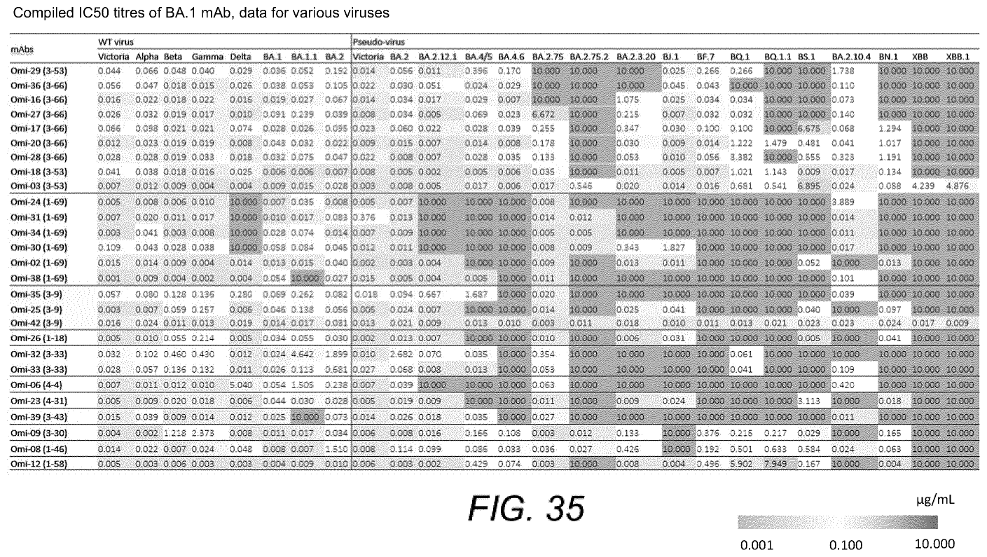

FIG. 5. Neutralization titres on the indicated viruses related to FIG. 3 (A) live viruses (B) pseudoviruses. Geometric mean titres are shown above each column. The Wilcoxon matched-pairs signed rank test was used for the analysis and two-tailed P values were calculated. (C) pseudovirus neutralization curves for selected VH1-58 mAb and control VH3-53 mAb 222 against Victoria and Iota (S477N).

FIG. 6. Structure of BA.1 RBD with Omi-12 Fab. (A) Two ternary complexes of Omi-12 and Beta-54 Fabs with BA.1 (produced by fitting the high-resolution structures of BA.1 RBD, Omi-12 and Beta-54 to the lower-resolution ternary complex density) in the crystal asymmetric unit are compared by overlapping the RBD. Fabs in one complex are in bright colours (cartoon depiction HC red, LC blue) and the other in pale colours. (B) The binding mode of Omi-12. (C) Close-up of the binding differences of Omi-12 with Fab 253 complexed with early pandemic RBD (pale blue) and Beta-47 with Beta RBD (pale cyan). (D) The somatic mutation V53P contributes to re-folding of the H3 loop so that Q493R can be accommodated in Omi-12.

FIG. 7. Pseudoviral neutralization assays of BA.4/5 by vaccine and BA.1 immune serum. IC50 values for the indicated viruses using serum obtained from vaccinees 28 days following their third dose of vaccine (A) AstraZeneca AZD AZD1222 (n=41), (B) 4 weeks after the third dose of Pfizer BNT162b2 (n=20). Serum from volunteers suffering breakthrough BA.1 infection volunteer taken (C) early ≤1 14 (n=12) days from symptom onset (median 13 days) (D) late ≥21 days from symptom onset (median 38 days) n=16. Comparison is made with neutralization titres to Victoria (an early pandemic strain), BA.1, BA.1.1, BA.2 and BA.3. Geometric mean titres are shown above each column. The Wilcoxon matched-pairs signed rank test was used for the analysis and two-tailed P values were calculated.

FIG. 8. Pseudoviral neutralization assays against Omicron and commercial monoclonal antibodies. Neutralization curve for a panel of 28 monoclonal antibodies made from samples taken from vaccinees infected with BA.1. Titration curves for BA.1 are compared with BA.1, BA.1.1, BA.2 and BA.3. mAb proposed to be affected by the L452R and F486L are indicated.

FIG. 9. The Omicron sub-lineage compared to BA.4/5. (A) Comparison of S protein mutations of Omicron BA.1, BA.1.1, BA.2, BA.3 and BA.4/5 with NTD and RBD boundaries indicated. (B) Position of RBD mutations (grey surface with the ACE2 footprint in dark green).

Mutations common to all Omicron lineages are shown in white (Q493R which is reverted in BA.4/5 is shown with a cross), those common to BA.1 and BA.1.1 in cyan, those unique to BA.1.1 in blue and those unique to BA.2 in magenta. Residue 371 (yellow) is mutated in all Omicron viruses but differs between BA.1 and BA.2. The N343 glycan is shown as sticks with a transparent surface FIG. 10. Surface plasmon resonance (SPR) analysis of interaction between BA.2 or BA.4/5 RBD and selected mAbs. (A) Binding of BA.4/5 RBD is severely reduced compared to that of BA.2, so that the binding could not be accurately determined, as shown by a single-injection of 200 nM RBD over sample flow cells containing IgG Omi-31. (B-C; E-I) Sensorgrams (Red: original binding curve; black: fitted curve) showing the interactions between BA.2 or BA.4/5 RBD and selected mAbs, with kinetics data shown. (D) Determination of the affinity of BA.4/5 RBD to Omi-12 using a 1:1 binding equilibrium analysis.

FIG. 11. Interactions between mAb and BA.4/5 mutation sites. Overall structure (left panel) and interactions (≤4 Å) with BA.4/5 mutation sites (right panel) for (A) BA.1-RBD/Omi-31 (PDB 7ZFB), (B) BA.1-RBD/Omi-32 (PDB 7ZFE), (C) BA.1-RBD/Omi-25 (PDB 7ZFD), (D) BA.1-RBD/Omi-42 (PDB7ZR7), (E) Wuhan-RBD/AZD8895 (PDB 7L7D) and (F) BA.1-RBD/Omi-3 (PDB 7ZF3) complexes. In the left panels RBD is shown as surface representation, with BA.4/5 mutation sites highlighted in magenta and the additional two mutation sites of BA.4/5 at 452 and 486 in cyan, and Fab LC as blue and HC as red ribbons. In the right panel, side chains of RBD, Fab HC and LC are drawn as grey, red and blue sticks, respectively. In (B) L452R (green sticks) are modelled to show a salt bridge to D99 of CDR-H3 may be formed (yellow broken sticks). (D) Beta-RBD/Omi-42 complex showing the Fab does not contact any of the two BA.4/5 mutation sites.

FIG. 12. ACE2 RBD affinity. (A)-(D) SPR sensorgrams showing ACE2 binding of BA.4/5 RBD (A) in comparison to binding to ancestral (Wuhan) (B), BA.1 (C) and BA.2 RBD (D). The data for Wuhan, BA.1 and BA.2 have been reported previously in (Nutalai et al., 2022). (E)-(G) Electrostatic surfaces, (E) from left to right, early pandemic, Delta and BA.1 RBD respectively, (F) open book view of BA.2 RBD and ACE2 of the BA.2 RBD/ACE2 complex (PDB 7ZF7), and (G) BA.4/5 RBD (modelled based on the structure of BA.2 RBD). The lozenges on ACE2 and RBD show the interaction areas.

FIG. 13. Antigenic mapping. (A) Neutralization data and model (log titre values) used to calculate antigenic maps in (B). Columns represent sera collected from inoculated volunteers or infected patients. Rows are challenge strains: Victoria, Alpha, Delta, Beta, Gamma, BA.1, BA1.1, BA.2, BA.3 and BA.4/5 in order. Values are colored according to their deviation from the reference value; the reference value is calculated on a serum-type basis as the average of neutralization titres from the row which gives this the highest value. (B) Orthogonal views of the antigenic map showing BA.4/5 in the context of the positions of previous VoC and BA.1, BA.1.1, BA.1 and BA.2, calculated from pseudovirus neutralisation data. Distance between two positions is proportional to the reduction in neutralisation titre when one of the corresponding strains is challenged with serum derived by infection by the other. FIG. 6. ACE2/RBD affinity and antigenic mapping

FIG. 14. Neutralization curves for VH1-58 mAb. Pseudoviral neutralization curves for early pandemic mAb 253 (Dejnirattisai et al., 2021a) and Beta-47 (Liu et al., 2021b) against Victoria and the panel of Omicron lineage constructs.

FIG. 15. Surface plasmon resonance (SPR) analysis of interaction between BA.2 or BA.4/5 RBD and selected mAbs. (A-F) Sensorgrams (Red: original binding curve; black: fitted curve) showing the interactions between BA.2 or BA.4/5 RBD and selected mAbs, with kinetics data shown. (G-K) Binding of BA.4/5 RBD is severely reduced compared to that of BA.2, so that the binding could not be accurately determined, as shown by a single-injection of 200 nM RBD over sample flow cells containing the mAb indicated.

FIG. 16. Sequence changes in BA.2.75 compared to other Omicron sub-lineages. (A) Sequence alignments of BA.2.75 together with Omicron sublineages Omicron BA.1, BA.1.1, BA.2, BA.3 and BA.4/5. Boundaries of the NTD and RBD are marked. (B) Surface representation of mutated residues in BA.2.75 RBD in comparison to BA.2 RBD. Position of BA.2 RBD mutations (grey surface with the ACE2 footprint in dark green) are shown and residues mutated in BA.2.75 are shown in orange and labelled.

FIG. 17. Pseudoviral neutralization assays of BA.2.75 by vaccine and BA.1 and BA.2 immune serum. IC50 values for the indicated viruses using serum obtained from vaccinees 28 days following their third dose of vaccine (A) Pfizer BNT162b2 (n=22). (B) AstraZeneca AZD AZD1222 (n=41). (C, D) Serum from volunteers suffering vaccine breakthrough BA.1 (n=16) or BA.2 (n=23) infections. (EC) IC50 values for single RBD point mutations inserted into the BA.2 pseudovirus using Pfizer BNT162b2 serum (n=22) Geometric mean titres are shown above each column. The Wilcoxon matched-pairs signed rank test was used for the analysis and two-tailed P values were calculated.

FIG. 18. ACE2/RBD affinity. SPR sensorgrams showing ACE2 binding of BA.2.75 RBD using ACE2-Fc (A) or biotinylated ACE2 as ligand (B) in comparison to binding to the RBD of BA.2 (C), BA.4/5 (D), Alpha (E) and BA.2+R493Q (F). The data for BA.2, BA.4/5 and Alpha have been reported previously in Nutalai et al., 2022, Tuekprakhon et al., 2022 and Dejnirattisai et al., 2022, respectively.

FIG. 19. Pseudoviral neutralization assays against monoclonal antibodies. (A) Neutralization curves for a panel of 28 mAb made from samples taken from vaccinees infected with BA.1. Titration curves for BA.2.75 are compared with Victoria, BA.1, BA.1.1, BA.2 and BA.4/5. IC50 titres are shown in Table 22. (B) Pseudoviral neutralization assays with mAbs developed for human use. IC50 titres are shown in Table 23. Data for Victoria, BA.1, BA.1.1 and BA.2 and BA.4/5 are used for comparison and taken from Tuekprakhon et al., 2022

FIG. 20. The Structure of BA.2.75 RBD/ACE2 complex. (A) The overall structure of the BA.2.75 RBD/ACE2 complex. ACE2 is shown as green ribbons and the RBD as surface with mutations common to BA.2 highlighted in magenta and different in orange. (B) BA.2.75 RBD (grey) and ACE2 (green) interface compared with that of BA.2 and ACE2 (both in salmon). Closeups show interactions of Q496R and Q493 (R493 in BA.2) with ACE2.

FIG. 21 Interactions between mAb and BA.75 mutation sites. (A) Front and back views of the binding modes of Omi-3 (PDB, 7ZF3) and Omi-18 (PDB, 7ZFC) complexed with omicron BA.1 RBD by overlapping the RBD. The RBD is shown as grey surface representation with mutations common to both BA.2 and BA.2.75 coloured in magenta, and the four mutations different between the two in cyan. VHs and VLs are shown as ribbons and coloured in red and blue for Omi-3, and light blue and salmon for Omi-18, respectively. (B) Interactions between N460 of the RBD and CDR-H2 of the Fabs. (C) Contacts between R493 of the RBD and CDR-H3 of the Fabs. In (B) and (C) The RBD associated with Omi-3 is in grey and Omi-18 in cyan, and the colours of the Fabs are as in (A). (D) AZD1061 bound with the ancestral SARS-CoV-2 RBD (PDB, 7L7E) and (E) contacts between G446 of the RBD and CDR-L2 of the Fab. (E) AZD8895 bound with the ancestral SARS-CoV-2 spike RBD (PDB, 7L7E) and (F) contacts between Q493 of the RBD and CDR-H2 of the Fab. In (D)-(F), RBD is drawn and coloured as in (A), HC is in red and LC in blue.

FIG. 22. Antigenic mapping. (A) Orthogonal views of the antigenic map showing BA.2.75 in the context of the positions of previous VoC and BA.1, BA.1.1, BA.1 and BA.2, calculated from pseudovirus neutralisation data. Distance between two positions is proportional to the reduction in neutralisation titre when one of the corresponding strains is challenged with serum derived by infection by the other. No scale is provided since the figures are projections of a three-dimensional distribution, however the variation can be calibrated by comparison with (i) BA.1 to BA.2 which is 2.93× reduced and (ii) BA.2 to BA.4/5 which is 3.03× reduced. (B) As (A) but including only Omicron sublineages and early pandemic viruses to allow more accurate projection of this subset into three-dimensions. Note that responses of these viruses against all sera were included in the calculations.

FIG. 23. Pseudoviral neutralization assays against monoclonal antibodies. (A) Neutralization curves for a panel of 28 monoclonal antibodies made from samples taken from vaccinees infected with BA.1. Titration curves for single mutations of BA.2.75 in the BA.2 backbone are compared with BA.2 and BA.2.75. IC50 titres are shown in Table 24.

FIG. 24. Surface plasmon resonance (SPR) analysis of interaction between BA.2 or BA.2.75 RBD and selected mAbs. (A) Binding of Omi-29 (IGHV3-53) to BA.2.75 RBD is severely reduced compared to that of BA.2, as shown by a single-injection of 1 μM Omi-29 Fab over sample flow cells containing biotinylated BA.2 or BA.2.75 RBD. (B) Binding of Omi-36 (IGHV3-66) to BA.2.75 RBD is severely reduced compared to that of BA.2, as shown by a single-injection of 0. μM BA.2 or BA.2.75 RBD over sample flow cells containing Omi-36 in the IgG form. (C-H) Sensorgrams (Red/Coloured: original binding curve; black: fitted curve) showing the interactions between BA.2 or BA.4/5 RBD and selected mAbs, with kinetics data shown.

FIG. 25. Neutralization of BA.2.75 by panels of convalescent serum collected from infection with historic variants. Neutralization titres of the indicated sera against BA.2.75 and the indicated pseudoviruses. Data apart from BA.2.75 has been taken from Tuekprakhon et al., 2022.

FIG. 26. Primers for site-directed PCR mutagenesis of the BA.2.75 RBD Site-directed PCR mutagenesis was performed using the BA.2 Spike construct as the template. D339H, G446S, N460K and R493Q mutations were introduced using the primers shown.

FIG. 27. Characterisation of BA.2.11, BA.2.12.1 and BA.2.13 by pseudoviral neutralization assays, surface plasmon resonance and structural analysis. (a), (b) IC50 values for the indicated viruses using serum obtained 4 weeks after a third dose of vaccine (a) AstraZeneca AZD1222 (n=41), (b) Pfizer BNT162b2 (n=18). (c) Neutralization titres of serum from vaccinated volunteers suffering breakthrough BA.1 infection were taken. Comparison is made with neutralization titers to Victoria, BA.1, BA.1.1, BA.2 and BA.4/5 previously reported in Tuekprakhon et al. (2022). Geometric mean titers are shown above each column. The Wilcoxon matched-pairs signed-rank test was used for the analysis, and two-tailed p values were calculated. (d-g) SPR sensorgrams (red: experimental binding curve; black: fitted curve) showing ACE2 binding of the RBD of BA.2.11 (e), BA.2.12.1 (f), BA.2.13 (g) in comparison with binding to BA.2 RBD (h), with kinetics data shown. The data for BA.2 RBD were reported in Nutalai et al. (2022). (h-m) Crystal structure of BA.2.12.1 RBD/Beta-27/NbC1 complex. (h) Overall structure shown as Cα traces with RBD (grey), Beta-27 HC (red) and LC (blue), and NbC1 (yellow). Cαs of residues L452Q, F486 and Q493R (L, F and R in BA.2, R, V and Q in BA.4/5) are shown as spheres. (i) Comparison of Beta-27 binding modes in the BA.2.12.1 RBD/Beta-27/NbC1 (RBD as surface representation, HC in red and LC in blue), BA.4/5 RBD/Beta-27/NbC1 (cyan, PDB 7ZXU) and Beta RBD/Beta-27 (green, PDB 7PS1) complexes by overlapping the RBDs. Apart from the flexible N- and C-terminal regions of RBD, significant differences occur at N-terminus and CDR-H1 of the Fab HC, α2 helix, 371-375 loop and G446 loop of the RBD. CDR-L3 has double conformations in the BA.4/5 RBD complex, and a single conformation in other two complexes (i). The HC N-terminus and CDR-H1 which contacts residue 486 of the RBD differs to those in both Beta and BA.4/5 RBD complexes, the latter contains F486V mutation. The differences are likely caused by contacts from a symmetry related C1 nanobody shown as grey bonds in (j). (k) The structural difference at G446 loop in the BA.4/5 RBD is also induced by crystal contact. (1) 371-375 loop that carries S371F, S373P and S375F mutations in BA.2.12.1 and BA.4/5 RBDs is stabilized by interactions with CDR-H3 of NbC1. (m) Superimposition of BA.2.12.1 (grey), BA.2 (green, PDB 7ZF9) and BA.4/5 (cyan) RBDs. (n) Mutations at 452 do not introduce significant local structural changes. R452 in BA.4/5 has a double conformation.

FIG. 28. Pseudoviral neutralization assays of BA.4.6 by vaccine. BA.1, BA.2, BA.4.5 immune serum (a-d) and monoclonal antibodies (e-f). IC50 values for the indicated viruses using serum obtained from vaccinees 28 days following their third dose of Pfizer BNT162b2 vaccine (n=22, a). IC50 values for the indicated viruses against serum from volunteers suffering vaccine breakthrough BA.1 (n=14, b), BA.2 (n=23, c) and BA.4/5 (n=11, d) infections. Geometric mean titres are shown above each column. The Wilcoxon matched-pairs signed rank test was used for the analysis and two-tailed P values were calculated. Neutralization curves for a panel of 28 monoclonal antibodies made from samples taken from vaccinees infected with BA.1 (e) against BA.4.6 were compared with Victoria, BA.1, BA.1.1, BA.2, BA.4/5 and BA.2.75 variants. Neutralization curves for a panel of 14 commercial monoclonal antibodies against same variants (e). IC50 values are shown in Table 29A and 29B.

FIG. 29. Pseudoviral neutralization assays. Pseudoviral neutralization assays against Omicron monoclonal antibodies, related to Table 26 where IC50 titers are shown. Neutralization curves for a panel of 27 monoclonal antibodies made from samples taken from vaccinees infected with BA.1. Titration curves for BA.2.11, BA.2.12.1 and BA.2.13 are compared with BA.2.

FIG. 30. Surface plasmon resonance (SPR) analysis of the interaction between BA.2.12.1 or BA.2 RBD and selected mAbs (Omi-6 and Omi-31). (a) Determination of the affinity of BA.2.12.1 RBD to Omi-6 using a 1:1 binding equilibrium analysis. (b), (c), (d) Sensorgrams (red: original binding curve; black: fitted curve) showing the interactions between BA.2.12.1 or BA.2 RBD and selected mAbs, with kinetics data shown.

FIG. 31. Neutralization assays. Neutralization curves using lentivirus pseudotyped with the S gene of indicated BA.2 sub-lineages (A) Omi-mAb, (B) Commercial mAb. See also Table 32. The “BA.4+all” variant is a synthetic variant, designed following the assessment of different mutations that occur in the SARS-CoV-2 Omicron S gene. These mutations were combined and incorporated into an Omicron BA.4 S gene to produce the artificial S gene called “BA.4+all”. This variant was created solely as an experimental tool and does not exist in nature, nor corresponds to the S gene of any circulating SARs-Cov-2 variant.

FIG. 32. Serum neutralization IC50 titres (fold dilution) of lentivirus pseudotyped with the S gene of the indicated BA.2 sub-lineages. (A) Serum obtained 28 days following the third dose of BNT162b2 vaccine or following infection with (B) BA.1 (C) BA.2 or (D) BA.4/5. Geometric mean titres are shown above each column. The Wilcoxon matched-pairs signed rank test (C and D) and Mann-Whitney test (E) were used and two-tailed P values calculated.

FIG. 33. Heatmaps of antibody binding. Heatmap showing the IC50 (μg/ml) of various antibodies against the Victoria and Beta strains in both vaccinated and unvaccinated samples.

FIG. 34. Neutralization assays. Neutralization curves using lentivirus pseudotyped with the S gene of the indicated BA.2 sub-lineages.

FIG. 35. Heat map of IC50 neutralization titres for the panel of BA.1 (Omi) mAb. Pseudoviral neutralization IC50 titres for indicated mAb against a panel of pseudoviruses expressing variant S sequences. Live virus IC50 values against variants found earlier in the pandemic are included for comparison. Data for live virus assays and pseudoviral data for Victoria, BA.2 and BA.4/5 were previously reported in Tuekprakon et al. (2022).

### DETAILED DESCRIPTION OF THE INVENTION

### Antibodies of the Invention

An antibody of the invention specifically binds to the spike protein of SAR-CoV-2.

In particular, it specifically binds to the S1 subunit of the spike protein, such as the receptor binding domain (RBD) or N-terminal domain (NTD).

An antibody of the invention may comprise at least three CDRs of an antibody in Table 3. The antibody may comprise at least four, five, or all six CDRs of an antibody in Table 3. The antibody may comprise a heavy chain variable domain comprising or consisting of an amino acid sequence having at least 80% sequence identity to the heavy chain variable domain of an antibody in Table 3. The antibody may comprise a light chain variable domain comprising or consisting of an amino acid sequence having at least 80% sequence identity to the light chain variable domain of an antibody in Table 3. The antibody may comprise a heavy chain variable domain and a light chain variable domain comprising or consisting of an amino acid sequence having at least 80% identity to the heavy chain variable domain and light chain domain, respectively, of an antibody in Table 3. The antibody may be any one of the antibodies in Table 3.

Table 3 lists 28 individual antibodies that were identified from recovered breakthrough Omicron SARS-CoV-2-infected patients, whom had already been received two doses of the Pfizer vaccine. Table 1 lists 42 individual antibodies that were previously identified from recovered COVID-19 patients [Dejnirattisai, Wanwisa, et al. “The antigenic anatomy of SARS-CoV-2 receptor binding domain.” Cell 184(8) (2021): 2183-2200; Supasa, Piyada, et al. “Reduced neutralization of SARS-CoV-2 B. 1.1.7 variant by convalescent and vaccine sera.” Cell 184(8) (2021): 2201-2211; Zhou, Daming, et al. “Evidence of escape of SARS-CoV-2 variant B.1.351 from natural andvaccine-induced sera.” Cell 184(9) (2021): 2348-2361; Dejnirattisai, Wanwisa, et al. “Antibody evasion by the P.1 strain of SARS-CoV-2.” Cell 184(11) (2021): 2939-2954; Liu, Chang, et al. “Reduced neutralization of SARS-CoV-2 B. 1.617 by vaccine and convalescent serum.” Cell 184(16) (2021): 4220-4236.]. Table 2 lists 28 individual antibodies that were previously identified from recovered Beta SARS-CoV-2 infected patients [Liu, C et al. “The antibody response to SARS-CoV-2 Beta underscores the antigenic distance to other variants”. Cell host & microbe 30(1)(2021): 53-68]. The antibodies in Table 1 are also referred to herein with a pre-fix “COVOX”, e.g. COVOX-222. The antibodies in Table 2 are also referred to with a pre-fix “β”, e.g. “β50”. The antibodies in Table 3 are also referred to with a pre-fix “O”, e.g. “O02”. Tables 1 to 3 list the SEQ ID NOs for the heavy chain variable region and light chain variable region nucleotide and amino acid sequences, and the complementarity determining regions (CDRs) of the variable chains, of each of the antibodies.

The antibody in Table 3 may be selected from the group consisting of Omi03, Omi12, Omi02, Omi39, Omi42, Omi16, Omi18, Omi20, Omi 23, Omi28, Omi08, Omi17, Omi29, Omi36 and Omi38. These antibodies were surprisingly found to retain strong neutralisation of the live SARS-CoV-2 variant strains Victoria, Alpha, Beta, Gamma, Delta and Omicron (e.g. an IC50 of ≤0.1 μg/ml against all live strains tested).

The antibody in Table 3 may be selected from the group consisting of: Omi03, Omi12, Omi02, Omi39, Omi42, Omi16, Omi18, Omi20, Omi 23, Omi28 and Omi 08. These antibodies were surprisingly found to retain strong neutralisation of the live SARS-CoV-2 variant strains Victoria, Alpha, Beta, Gamma, Delta and Omicron (e.g. an IC50 of ≤0.05 μg/ml against all live strains tested).

The antibody in Table 3 may be selected from the group consisting of: Omi03, Omi12, Omi02, Omi39 and Omi42. These antibodies were surprisingly found to retain strong neutralisation of the live SARS-CoV-2 variant strains Victoria, Alpha, Beta, Gamma, Delta and Omicron (e.g. an IC50 of ≤0.02 μg/ml against all live strains tested).

The antibody in Table 3 may be selected from the group consisting of: Omi03 and Omi12. These antibodies were surprisingly found to retain very strong neutralisation of the live SARS-CoV-2 variant strains Victoria, Alpha, Beta, Gamma, Delta and Omicron (e.g. an IC50 of ≤0.01 μg/ml against all live strains tested).

The antibody in Table 3 may be selected from the group consisting of: Omi02, Omi03, Omi12, Omi18, Omi28, Omi39 and Omi42. These antibodies were surprisingly found to retain very strong neutralisation of the live SARS-CoV-2 variant strains Victoria, Alpha, Beta, Gamma, Delta and Omicron.

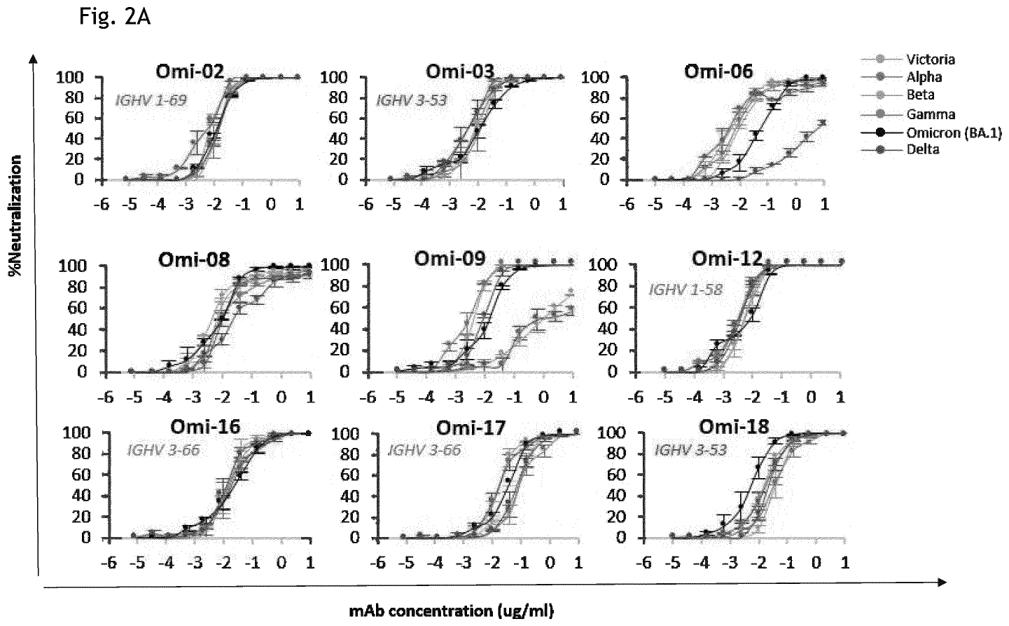

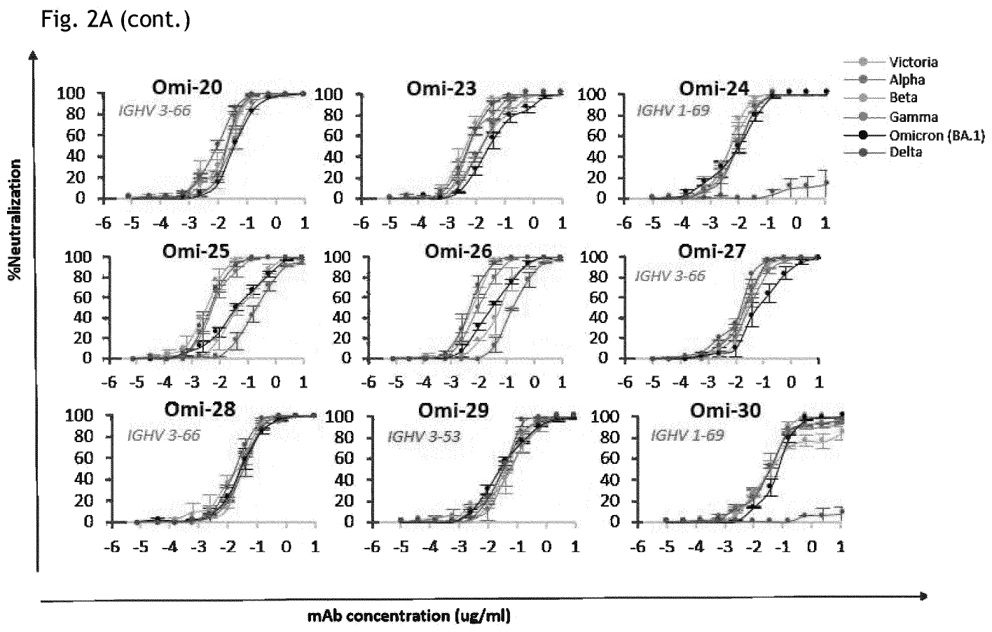

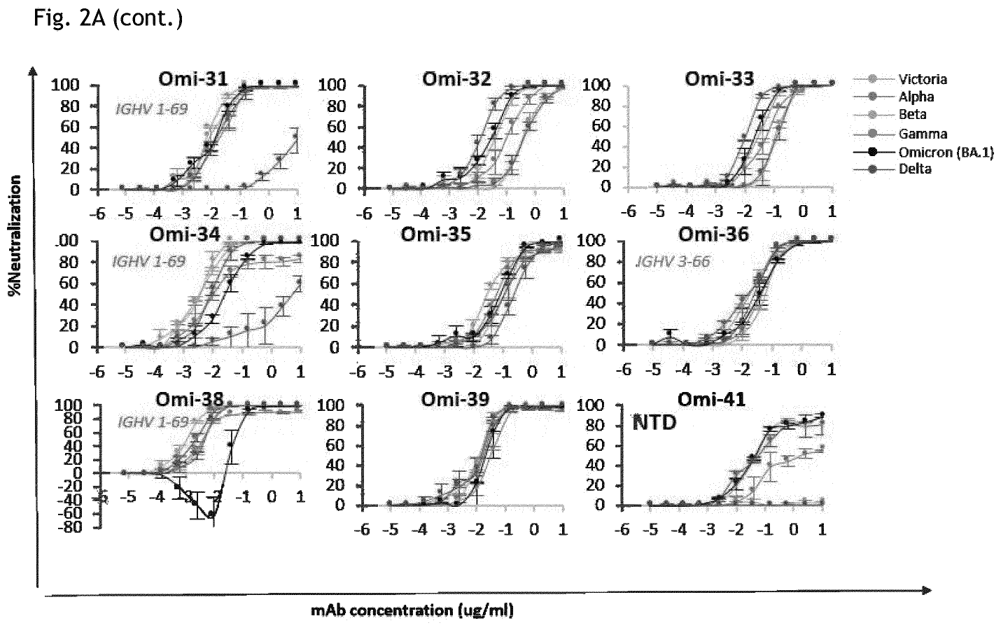

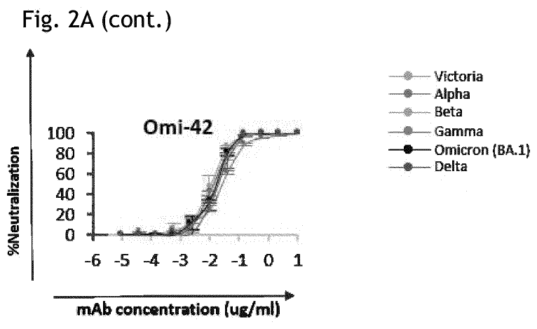

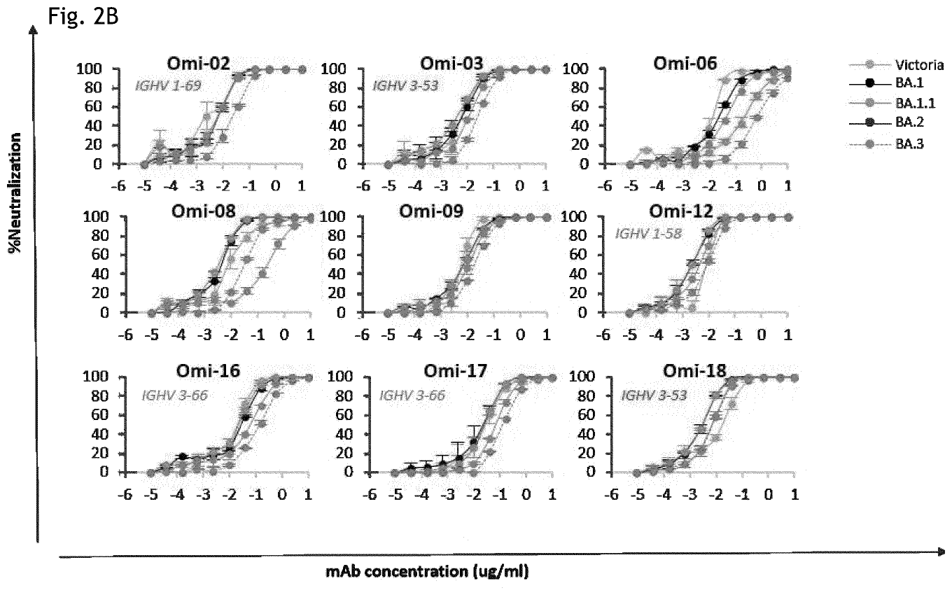

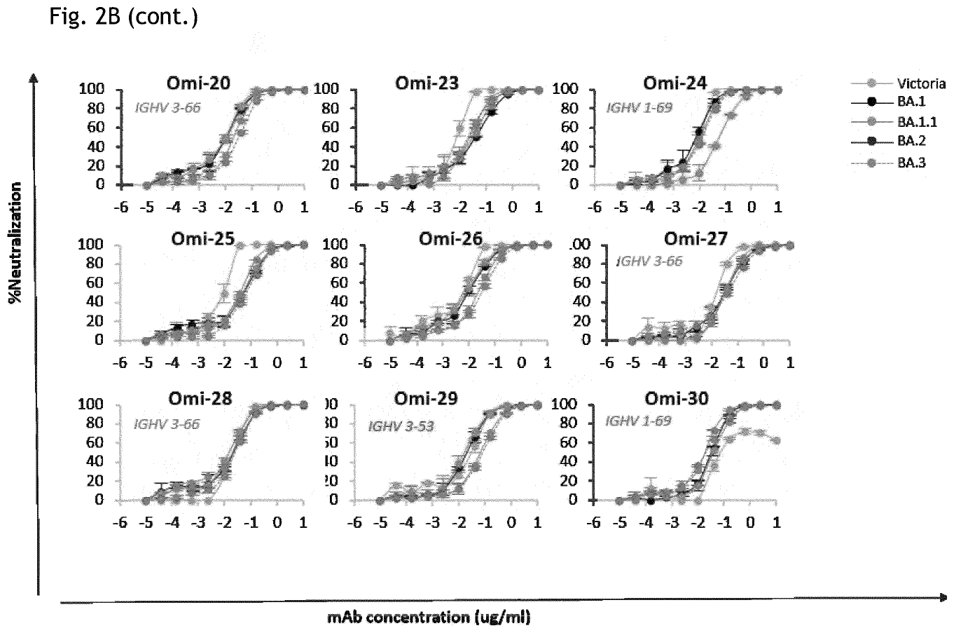

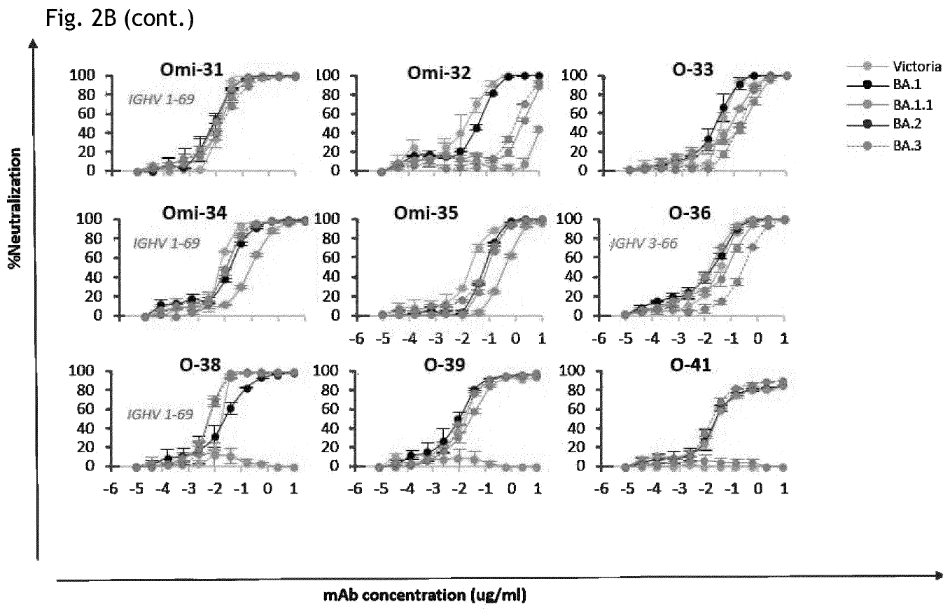

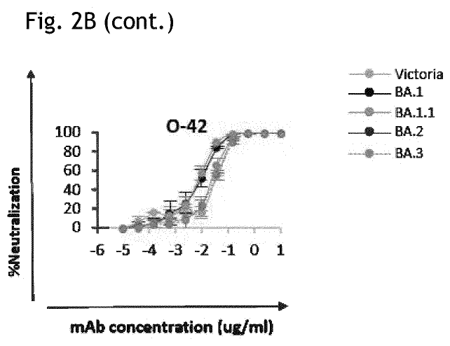

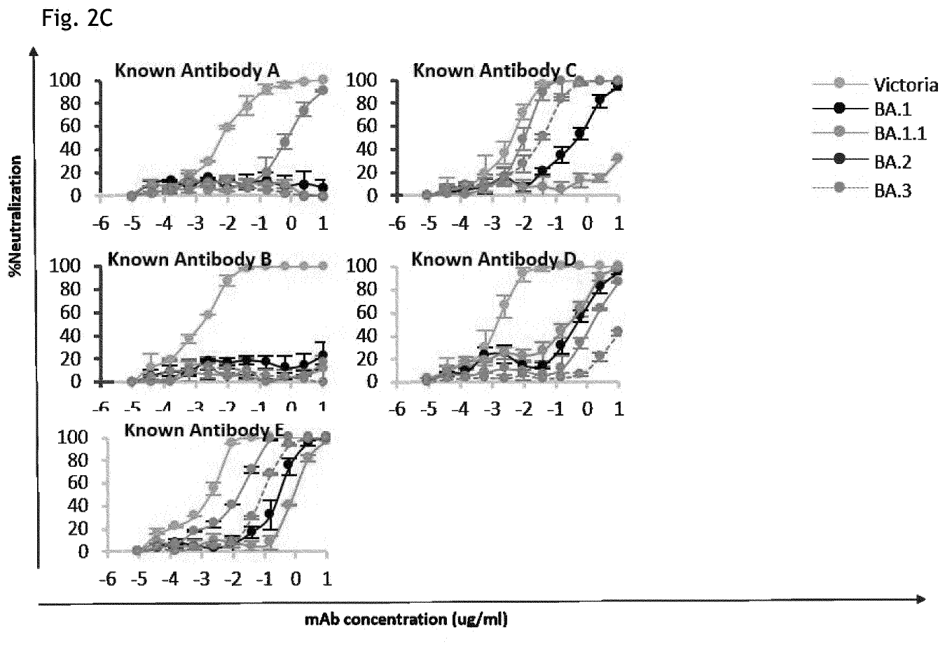

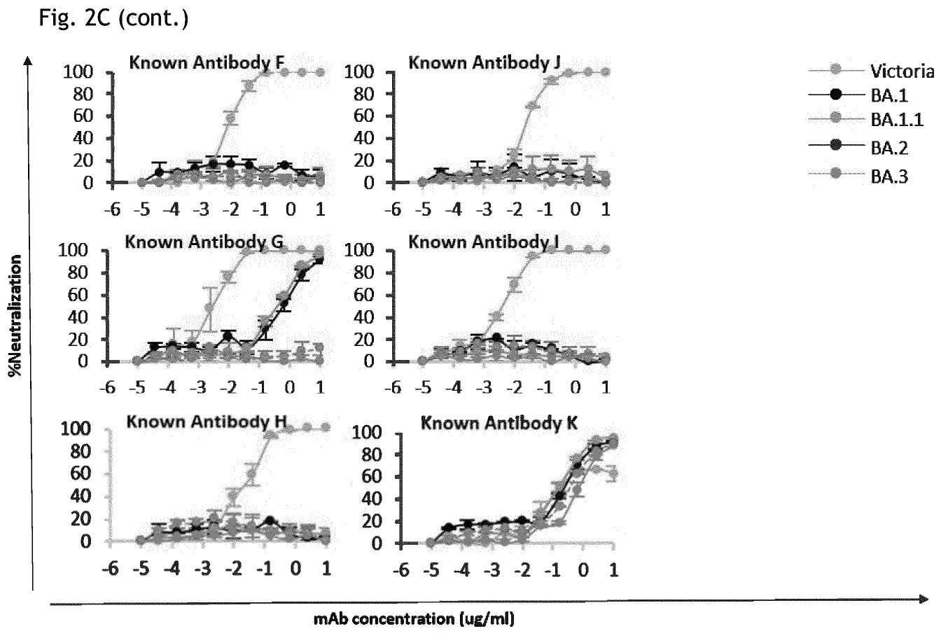

Accordingly, in one embodiment, the antibody in Table 3 may be Omi03. Omi03 was found to neutralise the live SARS-CoV-2 variant strains Victoria, Alpha, Beta, Gamma, Delta and Omicron, and the psuedoviral constructs of Victoria, Omicron BA.1, Omicron BA.1.1, Omicron BA.2 and Omicron BA.3 (see Tables 13 and 14, FIG. 2). In one embodiment, an antibody of the invention may comprise a CDRH1, CDRH2 and CDRH3 having the amino acid sequences specified in SEQ ID NOs: 695, 696 and 697, respectively, and a CDRL1, CDRL2 and CDRL3 having the amino acid sequences specified in SEQ ID NOs: 698, 699 and 700, respectively. In one embodiment, an antibody of the invention may comprise a heavy chain variable domain comprising or consisting of an amino acid sequence having ≥80%, ≥90%, ≥95%, ≥96%, ≥97%, ≥98%, ≥99% or 100% sequence identity to the heavy chain variable domain of antibody Omi03 (i.e. SEQ ID NO: 692). In one embodiment, an antibody of the invention may comprise a light chain variable domain comprising or consisting of an amino acid sequence having ≥80%, ≥90%, ≥95%, ≥96%, ≥97%, ≥98%, ≥99% or 100% sequence identity to the light chain variable domain of antibody Omi03 (i.e. SEQ ID NO: 694). In one embodiment, an antibody of the invention may comprise a heavy chain variable domain and a light chain variable domain comprising or consisting of an amino acid sequence having ≥80%, ≥90%, ≥95%, ≥96%, ≥97%, ≥98%, ≥99%, 100% sequence identity to the heavy chain variable domain and light chain variable domain, respectively, of antibody Omi03 (i.e. SEQ ID NOs: 692 and 694, respectively).

The heavy chain domain of Omi03 is derived from a IGHV3-53 v-region, and the inventors have previously demonstrated that switching of the heavy chains and light chains between antibodies derived from the same v-region results in an antibody that is particularly useful with the invention (explained further below). Hence, an antibody of the invention may comprise the heavy chain of Omi03, and not the light chain of Omi03. For example, the antibody may comprise a CDRH1, CDRH2 and CDRH3 having the amino acid sequences specified in SEQ ID NOs: 695, 696 and 697, respectively. The antibody may comprise a heavy chain variable domain comprising or consisting of an amino acid sequence having ≥80%, ≥90%, ≥95%, ≥96%, ≥97%, ≥98%, ≥99% or 100% sequence identity to the heavy chain variable domain of antibody Omi03 (i.e. SEQ ID NO: 692). The antibody may comprise a heavy chain variable domain comprising or consisting of SEQ ID NO: 692.

Alternatively, in an embodiment of the invention, the antibody may comprise the light chain of Omi03, and not the heavy chain of Omi03. For example, the antibody may comprise a CDRL1, CDRL2 and CDRL3 having the amino acid sequences specified in SEQ ID NOs: 698, 699 and 700, respectively. The antibody may comprise a light chain variable domain comprising or consisting of an amino acid sequence having ≥80%, ≥90%, ≥95%, ≥96%, ≥97%, ≥98%, ≥99% or 100% sequence identity to the light chain variable domain of antibody Omi03 (i.e. SEQ ID NO: 694). The antibody may comprise a light chain variable domain comprising or consisting of SEQ ID NO: 694.

In one embodiment, the antibody in Table 3 may be Omi12. Omi12 was found to neutralise the live SARS-CoV-2 variant strains Victoria, Alpha, Beta, Gamma, Delta and Omicron, and the psuedoviral constructs of Victoria, Omicron BA.1, Omicron BA.1.1, Omicron BA.2 and Omicron BA.3 (see Tables 13 and 14, FIG. 2). In one embodiment, an antibody of the invention may comprise a CDRH1, CDRH2 and CDRH3 having the amino acid sequences specified in SEQ ID NOs: 735, 736 and 737, respectively, and a CDRL1, CDRL2 and CDRL3 having the amino acid sequences specified in SEQ ID NOs: 738, 739 and 740, respectively. In one embodiment, an antibody of the invention may comprise a heavy chain variable domain comprising or consisting of an amino acid sequence having ≥80%, ≥90%, ≥95%, ≥96%, ≥97%, ≥98%, ≥99% or 100% sequence identity to the heavy chain variable domain of antibody Omi12 (i.e. SEQ ID NO: 732). In one embodiment, an antibody of the invention may comprise a light chain variable domain comprising or consisting of an amino acid sequence having ≥80%, ≥90%, ≥95%, ≥96%, ≥97%, ≥98%, ≥99% or 100% sequence identity to the light chain variable domain of antibody Omi12 (i.e. SEQ ID NO: 734). In one embodiment, an antibody of the invention may comprise a heavy chain variable domain and a light chain variable domain comprising or consisting of an amino acid sequence having ≥80%, ≥90%, ≥95%, ≥96%, ≥97%, ≥98%, ≥99%, 100% sequence identity to the heavy chain variable domain and light chain variable domain, respectively, of antibody Omi12 (i.e. SEQ ID NOs: 732 and 734, respectively).

The heavy chain domain of Omi12 is derived from a IGHV1-58 v-region, and the inventors have previously demonstrated that switching of the heavy chains and light chains between antibodies derived from the same v-region results in an antibody that is particularly useful with the invention (explained further below). Hence, an antibody of the invention may comprise the heavy chain of Omi12, and not the light chain of Omi12. For example, the antibody may comprise a CDRH1, CDRH2 and CDRH3 having the amino acid sequences specified in SEQ ID NOs: 735, 736 and 737, respectively. The antibody may comprise a heavy chain variable domain comprising or consisting of an amino acid sequence having ≥80%, ≥90%, ≥95%, ≥96%, ≥97%, ≥98%, ≥99% or 100% sequence identity to the heavy chain variable domain of antibody Omi12 (i.e. SEQ ID NO: 732). The antibody may comprise a heavy chain variable domain comprising or consisting of SEQ ID NO: 732.

Alternatively, in an embodiment of the invention, the antibody may comprise the light chain of Omi12, and not the heavy chain of Omi12. For example, the antibody may comprise a CDRL1, CDRL2 and CDRL3 having the amino acid sequences specified in SEQ ID NOs: 738, 739 and 740, respectively. The antibody may comprise a light chain variable domain comprising or consisting of an amino acid sequence having ≥80%, ≥90%, ≥95%, ≥96%, ≥97%, ≥98%, ≥99% or 100% sequence identity to the light chain variable domain of antibody Omi12 (i.e. SEQ ID NO: 734). The antibody may comprise a light chain variable domain comprising or consisting of SEQ ID NO: 734.

In one embodiment, the antibody in Table 3 may be Omi02. Omi02 was found to neutralise the live SARS-CoV-2 variant strains Victoria, Alpha, Beta, Gamma, Delta and Omicron, and the psuedoviral constructs of Victoria, Omicron BA.1, Omicron BA.1.1, Omicron BA.2 and Omicron BA.3 (see Tables 13 and 14, FIG. 2). In one embodiment, an antibody of the invention may comprise a CDRH1, CDRH2 and CDRH3 having the amino acid sequences specified in SEQ ID NOs: 685, 686 and 687, respectively, and a CDRL1, CDRL2 and CDRL3 having the amino acid sequences specified in SEQ ID NOs: 688, 689 and 690, respectively. In one embodiment, an antibody of the invention may comprise a heavy chain variable domain comprising or consisting of an amino acid sequence having ≥80%, ≥90%, ≥95%, ≥96%, ≥97%, ≥98%, ≥99% or 100% sequence identity to the heavy chain variable domain of antibody Omi02 (i.e. SEQ ID NO: 682). In one embodiment, an antibody of the invention may comprise a light chain variable domain comprising or consisting of an amino acid sequence having ≥80%, ≥90%, ≥95%,≥96%, ≥97%, ≥98%, ≥99% or 100% sequence identity to the light chain variable domain of antibody Omi02 (i.e. SEQ ID NO: 684). In one embodiment, an antibody of the invention may comprise a heavy chain variable domain and a light chain variable domain comprising or consisting of an amino acid sequence having ≥80%, ≥90%, ≥95%, ≥96%, ≥97%, ≥98%, ≥99%, 100% sequence identity to the heavy chain variable domain and light chain variable domain, respectively, of antibody Omi02 (i.e. SEQ ID NOs: 682 and 684, respectively).

The heavy chain domain of Omi02 is derived from a IGHV1-69 v-region, and the inventors have previously demonstrated that switching of the heavy chains and light chains between antibodies derived from the same v-region results in an antibody that is particularly useful with the invention (explained further below). Hence, an antibody of the invention may comprise the heavy chain of Omi02, and not the light chain of Omi02. For example, the antibody may comprise a CDRH1, CDRH2 and CDRH3 having the amino acid sequences specified in SEQ ID NOs: 685, 686 and 687, respectively. The antibody may comprise a heavy chain variable domain comprising or consisting of an amino acid sequence having ≥80%, ≥90%, ≥95%, ≥96%, ≥97%, ≥98%, ≥99% or 100% sequence identity to the heavy chain variable domain of antibody Omi02 (i.e. SEQ ID NO: 682). The antibody may comprise a heavy chain variable domain comprising or consisting of SEQ ID NO: 682.

Alternatively, in an embodiment of the invention, the antibody may comprise the light chain of Omi02, and not the heavy chain of Omi02. For example, the antibody may comprise a CDRL1, CDRL2 and CDRL3 having the amino acid sequences specified in SEQ ID NOs: 688, 689 and 690, respectively. The antibody may comprise a light chain variable domain comprising or consisting of an amino acid sequence having ≥80%, ≥90%, ≥95%, ≥96%, ≥97%, ≥98%, ≥99% or 100% sequence identity to the light chain variable domain of antibody Omi02 (i.e. SEQ ID NO: 684). The antibody may comprise a light chain variable domain comprising or consisting of SEQ ID NO: 684.

In one embodiment, the antibody in Table 3 may be Omi08. Omi08 was found to neutralise the live SARS-CoV-2 variant strains Victoria, Alpha, Beta, Gamma, Delta and Omicron, and the psuedoviral constructs of Victoria, Omicron BA.1 Omicron BA.1, Omicron BA.1.1, Omicron BA.2 and Omicron BA.3 (see Tables 13 and 14, FIG. 2). In one embodiment, an antibody of the invention may comprise a CDRH1, CDRH2 and CDRH3 having the amino acid sequences specified in SEQ ID NOs: 715, 716 and 717, respectively, and a CDRL1, CDRL2 and CDRL3 having the amino acid sequences specified in SEQ ID NOs: 718, 719 and 720, respectively. In one embodiment, an antibody of the invention may comprise a heavy chain variable domain comprising or consisting of an amino acid sequence having ≥80%, ≥90%, ≥95%, ≥96%, ≥97%,≥98%, ≥99% or 100% sequence identity to the heavy chain variable domain of antibody Omi08 (i.e. SEQ ID NO: 712). In one embodiment, an antibody of the invention may comprise a light chain variable domain comprising or consisting of an amino acid sequence having ≥80%, ≥90%, ≥95%, ≥96%, ≥97%, ≥98%, ≥99% or 100% sequence identity to the light chain variable domain of antibody Omi08 (i.e. SEQ ID NO: 714). In one embodiment, an antibody of the invention may comprise a heavy chain variable domain and a light chain variable domain comprising or consisting of an amino acid sequence having ≥80%, ≥90%, ≥95%, ≥96%, ≥97%, ≥98%, ≥99%, 100% sequence identity to the heavy chain variable domain and light chain variable domain, respectively, of antibody Omi08 (i.e. SEQ ID NOs: 712 and 714, respectively).

In one embodiment, the antibody in Table 3 may be Omi42. Omi42 was found to neutralise the live SARS-CoV-2 variant strains Victoria, Alpha, Beta, Gamma, Delta and Omicron, and the psuedoviral constructs of Victoria, Omicron BA.1, Omicron BA.1.1, Omicron BA.2 and Omicron BA.3 (see Tables 13 and 14, FIG. 2). In one embodiment, an antibody of the invention may comprise a CDRH1, CDRH2 and CDRH3 having the amino acid sequences specified in SEQ ID NOs: 955, 956 and 957, respectively, and a CDRL1, CDRL2 and CDRL3 having the amino acid sequences specified in SEQ ID NOs: 958, 959 and 960, respectively. In one embodiment, an antibody of the invention may comprise a heavy chain variable domain comprising or consisting of an amino acid sequence having ≥80%, ≥90%, ≥95%, ≥96%, ≥97%, ≥98%, ≥99% or 100% sequence identity to the heavy chain variable domain of antibody Omi42 (i.e. SEQ ID NO: 952). In one embodiment, an antibody of the invention may comprise a light chain variable domain comprising or consisting of an amino acid sequence having ≥80%, ≥90%, ≥95%,≥96%, ≥97%, ≥98%, ≥99% or 100% sequence identity to the light chain variable domain of antibody Omi42 (i.e. SEQ ID NO: 954). In one embodiment, an antibody of the invention may comprise a heavy chain variable domain and a light chain variable domain comprising or consisting of an amino acid sequence having ≥80%, ≥90%, ≥95%, ≥96%, ≥97%,≥98%, ≥99%, 100% sequence identity to the heavy chain variable domain and light chain variable domain, respectively, of antibody Omi42 (i.e. SEQ ID NOs: 952 and 954, respectively).

The heavy chain domain of Omi42 is derived from a IGHV3-9 v-region, and the inventors have previously demonstrated that switching of the heavy chains and light chains between antibodies derived from the same v-region results in an antibody that is particularly useful with the invention (explained further below). Hence, an antibody of the invention may comprise the heavy chain of Omi42, and not the light chain of Omi42. For example, the antibody may comprise a CDRH1, CDRH2 and CDRH3 having the amino acid sequences specified in SEQ ID NOs: 955, 956 and 957, respectively. The antibody may comprise a heavy chain variable domain comprising or consisting of an amino acid sequence having ≥80%, ≥90%, ≥95%, ≥96%, ≥97%, ≥98%, ≥99% or 100% sequence identity to the heavy chain variable domain of antibody Omi42 (i.e. SEQ ID NO: 952). The antibody may comprise a heavy chain variable domain comprising or consisting of SEQ ID NO: 952.

Alternatively, in an embodiment of the invention, the antibody may comprise the light chain of Omi42, and not the heavy chain of Omi42. For example, the antibody may comprise a CDRL1, CDRL2 and CDRL3 having the amino acid sequences specified in SEQ ID NOs: 958, 959 and 960, respectively. The antibody may comprise a light chain variable domain comprising or consisting of an amino acid sequence having ≥80%, ≥90%, ≥95%, ≥96%, ≥97%, ≥98%, ≥99% or 100% sequence identity to the light chain variable domain of antibody Omi42 (i.e. SEQ ID NO: 954). The antibody may comprise a light chain variable domain comprising or consisting of SEQ ID NO: 954.

In one embodiment, the antibody in Table 3 may be Omi16. Omi16 was found to neutralise the live SARS-CoV-2 variant strains Victoria, Alpha, Beta, Gamma, Delta and Omicron, and the psuedoviral constructs of Victoria, Omicron BA.1, Omicron BA.1, Omicron BA.1.1, Omicron BA.2 and Omicron BA.3 (see Tables 13 and 14, FIG. 2). In one embodiment, an antibody of the invention may comprise a CDRH1, CDRH2 and CDRH3 having the amino acid sequences specified in SEQ ID NOs: 745, 746 and 747, respectively, and a CDRL1, CDRL2 and CDRL3 having the amino acid sequences specified in SEQ ID NOs: 748, 749 and 750, respectively. In one embodiment, an antibody of the invention may comprise a heavy chain variable domain comprising or consisting of an amino acid sequence having ≥80%, ≥90%, ≥95%, ≥96%, ≥97%, ≥98%, ≥99% or 100% sequence identity to the heavy chain variable domain of antibody Omi16 (i.e. SEQ ID NO: 742). In one embodiment, an antibody of the invention may comprise a light chain variable domain comprising or consisting of an amino acid sequence having ≥80%, ≥90%, ≥95%, ≥96%, ≥97%, ≥98%, ≥99% or 100% sequence identity to the light chain variable domain of antibody Omi16 (i.e. SEQ ID NO: 744). In one embodiment, an antibody of the invention may comprise a heavy chain variable domain and a light chain variable domain comprising or consisting of an amino acid sequence having ≥80%, ≥90%, ≥95%, ≥96%, ≥97%, ≥98%, ≥99%, 100% sequence identity to the heavy chain variable domain and light chain variable domain, respectively, of antibody Omi16 (i.e. SEQ ID NOs: 742 and 744, respectively).

The heavy chain domain of Omi16 is derived from a IGHV3-66 v-region, and the inventors have previously demonstrated that switching of the heavy chains and light chains between antibodies derived from the same v-region results in an antibody that is particularly useful with the invention (explained further below). Hence, an antibody of the invention may comprise the heavy chain of Omi16, and not the light chain of Omi16. For example, the antibody may comprise a CDRH1, CDRH2 and CDRH3 having the amino acid sequences specified in SEQ ID NOs: 745, 746 and 747, respectively. The antibody may comprise a heavy chain variable domain comprising or consisting of an amino acid sequence having ≥80%, ≥90%, ≥95%, ≥96%, ≥97%, ≥98%, ≥99% or 100% sequence identity to the heavy chain variable domain of antibody Omi16 (i.e. SEQ ID NO: 742). The antibody may comprise a heavy chain variable domain comprising or consisting of SEQ ID NO: 742.

Alternatively, in an embodiment of the invention, the antibody may comprise the light chain of Omi16, and not the heavy chain of Omi16. For example, the antibody may comprise a CDRL1, CDRL2 and CDRL3 having the amino acid sequences specified in SEQ ID NOs: 748, 749 and 750, respectively. The antibody may comprise a light chain variable domain comprising or consisting of an amino acid sequence having ≥80%,≥90%, ≥95%, ≥96%, ≥97%, ≥98%, ≥99% or 100% sequence identity to the light chain variable domain of antibody Omi16 (i.e. SEQ ID NO: 744). The antibody may comprise a light chain variable domain comprising or consisting of SEQ ID NO: 744.

In one embodiment, the antibody in Table 3 may be Omi18. Omi18 was found to neutralise the live SARS-CoV-2 variant strains Victoria, Alpha, Beta, Gamma, Delta and Omicron, and the psuedoviral constructs of Victoria, Omicron BA.1, Omicron BA.1.1, Omicron BA.2 and Omicron BA.3 (see Tables 13 and 14, FIG. 2). In one embodiment, an antibody of the invention may comprise a CDRH1, CDRH2 and CDRH3 having the amino acid sequences specified in SEQ ID NOs: 765, 766 and 767, respectively, and a CDRL1, CDRL2 and CDRL3 having the amino acid sequences specified in SEQ ID NOs: 768, 769 and 770, respectively. In one embodiment, an antibody of the invention may comprise a heavy chain variable domain comprising or consisting of an amino acid sequence having ≥80%, ≥90%, ≥95%, ≥96%, ≥97%, ≥98%, ≥99% or 100% sequence identity to the heavy chain variable domain of antibody Omi18 (i.e. SEQ ID NO: 762). In one embodiment, an antibody of the invention may comprise a light chain variable domain comprising or consisting of an amino acid sequence having ≥80%, ≥90%, ≥95%, ≥96%, ≥97%, ≥98%, ≥99% or 100% sequence identity to the light chain variable domain of antibody Omi18 (i.e. SEQ ID NO: 764). In one embodiment, an antibody of the invention may comprise a heavy chain variable domain and a light chain variable domain comprising or consisting of an amino acid sequence having ≥80%, ≥90%, ≥95%, ≥96%, ≥97%, ≥98%, ≥99%, 100% sequence identity to the heavy chain variable domain and light chain variable domain, respectively, of antibody Omi18 (i.e. SEQ ID NOs: 762 and 764, respectively).

The heavy chain domain of Omi18 is derived from a IGHV3-53 v-region, and the inventors have previously demonstrated that switching of the heavy chains and light chains between antibodies derived from the same v-region results in an antibody that is particularly useful with the invention (explained further below). Hence, an antibody of the invention may comprise the heavy chain of Omi18, and not the light chain of Omi18. For example, the antibody may comprise a CDRH1, CDRH2 and CDRH3 having the amino acid sequences specified in SEQ ID NOs: 765, 766 and 767, respectively. The antibody may comprise a heavy chain variable domain comprising or consisting of an amino acid sequence having ≥80%, ≥90%, ≥95%, ≥96%, ≥97%, ≥98%, ≥99% or 100% sequence identity to the heavy chain variable domain of antibody Omi18 (i.e. SEQ ID NO: 762).

The antibody may comprise a heavy chain variable domain comprising or consisting of SEQ ID NO: 762.

Alternatively, in an embodiment of the invention, the antibody may comprise the light chain of Omi18, and not the heavy chain of Omi18. For example, the antibody may comprise a CDRL1, CDRL2 and CDRL3 having the amino acid sequences specified in SEQ ID NOs: 768, 769 and 770, respectively. The antibody may comprise a light chain variable domain comprising or consisting of an amino acid sequence having ≥80%, ≥90%, ≥95%, ≥96%, ≥97%, ≥98%, ≥99% or 100% sequence identity to the light chain variable domain of antibody Omi18 (i.e. SEQ ID NO: 764). The antibody may comprise alight chain variable domain comprising or consisting of SEQ ID NO: 764.

In one embodiment, the antibody in Table 3 may be Omi20. Omi20 was found to neutralise the live SARS-CoV-2 variant strains Victoria, Alpha, Beta, Gamma, Delta and Omicron, and the psuedoviral constructs of Victoria, Omicron BA.1, Omicron BA.1.1, Omicron BA.2 and Omicron BA.3 (see Tables 13 and 14, FIG. 2). In one embodiment, an antibody of the invention may comprise a CDRH1, CDRH2 and CDRH3 having the amino acid sequences specified in SEQ ID NOs: 775, 776 and 777, respectively, and a CDRL1, CDRL2 and CDRL3 having the amino acid sequences specified in SEQ ID NOs: 778, 779 and 780, respectively. In one embodiment, an antibody of the invention may comprise a heavy chain variable domain comprising or consisting of an amino acid sequence having ≥80%, ≥90%, ≥95%, ≥96%, ≥97%, ≥98%, ≥99% or 100% sequence identity to the heavy chain variable domain of antibody Omi20 (i.e. SEQ ID NO: 772). In one embodiment, an antibody of the invention may comprise a light chain variable domain comprising or consisting of an amino acid sequence having ≥80%, ≥90%, ≥95%,≥96%, ≥97%, ≥98%, ≥99% or 100% sequence identity to the light chain variable domain of antibody Omi20 (i.e. SEQ ID NO: 774). In one embodiment, an antibody of the invention may comprise a heavy chain variable domain and a light chain variable domain comprising or consisting of an amino acid sequence having ≥80%, ≥90%, ≥95%, ≥96%, ≥97%, ≥98%, ≥99%, 100% sequence identity to the heavy chain variable domain and light chain variable domain, respectively, of antibody Omi20 (i.e. SEQ ID NOs: 772 and 774, respectively).

The heavy chain domain of Omi20 is derived from a IGHV3-66 v-region, and the inventors have previously demonstrated that switching of the heavy chains and light chains between antibodies derived from the same v-region results in an antibody that is particularly useful with the invention (explained further below). Hence, an antibody of the invention may comprise the heavy chain of Omi20, and not the light chain of Omi20. For example, the antibody may comprise a CDRH1, CDRH2 and CDRH3 having the amino acid sequences specified in SEQ ID NOs: 775, 776 and 777, respectively. The antibody may comprise a heavy chain variable domain comprising or consisting of an amino acid sequence having ≥80%, ≥90%, ≥95%, ≥96%, ≥97%, ≥98%, ≥99% or 100% sequence identity to the heavy chain variable domain of antibody Omi20 (i.e. SEQ ID NO: 772). The antibody may comprise a heavy chain variable domain comprising or consisting of SEQ ID NO: 772.

Alternatively, in an embodiment of the invention, the antibody may comprise the light chain of Omi20, and not the heavy chain of Omi20. For example, the antibody may comprise a CDRL1, CDRL2 and CDRL3 having the amino acid sequences specified in SEQ ID NOs: 778, 779 and 780, respectively. The antibody may comprise a light chain variable domain comprising or consisting of an amino acid sequence having ≥80%,≥90%, ≥95%, ≥96%, ≥97%, ≥98%, ≥99% or 100% sequence identity to the light chain variable domain of antibody Omi20 (i.e. SEQ ID NO: 774). The antibody may comprise alight chain variable domain comprising or consisting of SEQ ID NO: 774.

In one embodiment, the antibody in Table 3 may be Omi23. Omi23 was found to neutralise the live SARS-CoV-2 variant strains Victoria, Alpha, Beta, Gamma, Delta and Omicron, and the psuedoviral constructs of Victoria, Omicron BA.1, Omicron BA.1.1, Omicron BA.2 and Omicron BA.3 (see Tables 13 and 14, FIG. 2). In one embodiment, an antibody of the invention may comprise a CDRH1, CDRH2 and CDRH3 having the amino acid sequences specified in SEQ ID NOs: 785, 786 and 787, respectively, and a CDRL1, CDRL2 and CDRL3 having the amino acid sequences specified in SEQ ID NOs: 788, 789 and 790, respectively. In one embodiment, an antibody of the invention may comprise a heavy chain variable domain comprising or consisting of an amino acid sequence having ≥80%, ≥90%, ≥95%, ≥96%, ≥97%, ≥98%, ≥99% or 100% sequence identity to the heavy chain variable domain of antibody Omi23 (i.e. SEQ ID NO: 782). In one embodiment, an antibody of the invention may comprise a light chain variable domain comprising or consisting of an amino acid sequence having ≥80%, ≥90%, ≥95%, ≥96%, ≥97%, ≥98%, ≥99% or 100% sequence identity to the light chain variable domain of antibody Omi23 (i.e. SEQ ID NO: 784). In one embodiment, an antibody of the invention may comprise a heavy chain variable domain and a light chain variable domain comprising or consisting of an amino acid sequence having ≥80%, ≥90%, ≥95%, ≥96%, ≥97%, ≥98%, ≥99%, 100% sequence identity to the heavy chain variable domain and light chain variable domain, respectively, of antibody Omi23 (i.e. SEQ ID NOs: 782 and 784, respectively).

The heavy chain domain of Omi23 is derived from a IGHV4-31 v-region, and the inventors have previously demonstrated that switching of the heavy chains and light chains between antibodies derived from the same v-region results in an antibody that is particularly useful with the invention (explained further below). Hence, an antibody of the invention may comprise the heavy chain of Omi23, and not the light chain of Omi23. For example, the antibody may comprise a CDRH1, CDRH2 and CDRH3 having the amino acid sequences specified in SEQ ID NOs: 785, 786 and 787, respectively. The antibody may comprise a heavy chain variable domain comprising or consisting of an amino acid sequence having ≥80%, ≥90%, ≥95%, ≥96%, ≥97%, ≥98%, ≥99% or 100% sequence identity to the heavy chain variable domain of antibody Omi23 (i.e. SEQ ID NO: 782). The antibody may comprise a heavy chain variable domain comprising or consisting of SEQ ID NO: 782.

Alternatively, in an embodiment of the invention, the antibody may comprise the light chain of Omi23, and not the heavy chain of Omi23. For example, the antibody may comprise a CDRL1, CDRL2 and CDRL3 having the amino acid sequences specified in SEQ ID NOs: 788, 789 and 790, respectively. The antibody may comprise a light chain variable domain comprising or consisting of an amino acid sequence having ≥80%,≥90%, ≥95%, ≥96%, ≥97%, ≥98%, ≥99% or 100% sequence identity to the light chain variable domain of antibody Omi23 (i.e. SEQ ID NO: 784). The antibody may comprise alight chain variable domain comprising or consisting of SEQ ID NO: 784.

In one embodiment, the antibody in Table 3 may be Omi28. Omi28 was found to neutralise the live SARS-CoV-2 variant strains Victoria, Alpha, Beta, Gamma, Delta and Omicron, and the psuedoviral constructs of Victoria, Omicron BA.1, Omicron BA.1.1, Omicron BA.2 and Omicron BA.3 (see Tables 13 and 14, FIG. 2). In one embodiment, an antibody of the invention may comprise a CDRH1, CDRH2 and CDRH3 having the amino acid sequences specified in SEQ ID NOs: 835, 836 and 837, respectively, and a CDRL1, CDRL2 and CDRL3 having the amino acid sequences specified in SEQ ID NOs: 838, 839 and 840, respectively. In one embodiment, an antibody of the invention may comprise a heavy chain variable domain comprising or consisting of an amino acid sequence having ≥80%, ≥90%, ≥95%, ≥96%, ≥97%, ≥98%, ≥99% or 100% sequence identity to the heavy chain variable domain of antibody Omi28 (i.e. SEQ ID NO: 832). In one embodiment, an antibody of the invention may comprise a light chain variable domain comprising or consisting of an amino acid sequence having ≥80%, ≥90%, ≥95%,≥96%, ≥97%, ≥98%, ≥99% or 100% sequence identity to the light chain variable domain of antibody Omi28 (i.e. SEQ ID NO: 834). In one embodiment, an antibody of the invention may comprise a heavy chain variable domain and a light chain variable domain comprising or consisting of an amino acid sequence having ≥80%, ≥90%, ≥95%, ≥96%, ≥97%, ≥98%, ≥99%, 100% sequence identity to the heavy chain variable domain and light chain variable domain, respectively, of antibody Omi28 (i.e. SEQ ID NOs: 832 and 834, respectively). The heavy chain domain of Omi28 is derived from a IGHV3-66 v-region, and the inventors have previously demonstrated that switching of the heavy chains and light chains between antibodies derived from the same v-region results in an antibody that is particularly useful with the invention (explained further below). Hence, an antibody of the invention may comprise the heavy chain of Omi28, and not the light chain of Omi28. For example, the antibody may comprise a CDRH1, CDRH2 and CDRH3 having the amino acid sequences specified in SEQ ID NOs: 835, 836 and 837, respectively. The antibody may comprise a heavy chain variable domain comprising or consisting of an amino acid sequence having ≥80%, ≥90%, ≥95%, ≥96%, ≥97%, ≥98%, ≥99% or 100% sequence 10 identity to the heavy chain variable domain of antibody Omi28 (i.e. SEQ ID NO: 832). The antibody may comprise a heavy chain variable domain comprising or consisting of SEQ ID NO: 832.

Alternatively, in an embodiment of the invention, the antibody may comprise the light chain of Omi28, and not the heavy chain of Omi28. For example, the antibody may
comprise a CDRL1, CDRL2 and CDRL3 having the amino acid sequences specified in SEQ ID NOs: 838, 839 and 840, respectively. The antibody may comprise a light chain variable domain comprising or consisting of an amino acid sequence having ≥80%,≥90%, ≥95%, ≥96%, ≥97%, ≥98%, ≥99% or 100% sequence identity to the light chain variable domain of antibody Omi28 (i.e. SEQ ID NO: 834). The antibody may comprise alight chain variable domain comprising or consisting of SEQ ID NO: 834.

In one embodiment, the antibody in Table 3 may be Omi39. Omi39 was found to neutralise the live SARS-CoV-2 variant strains Victoria, Alpha, Beta, Gamma, Delta and Omicron, and the psuedoviral constructs of Victoria, Omicron BA.1, Omicron BA.1.1, Omicron BA.2 and Omicron BA.3 (see Tables 13 and 14, FIG. 2). In one embodiment, an antibody of the invention may comprise a CDRH1, CDRH2 and CDRH3 having the amino acid sequences specified in SEQ ID NOs: 935, 936 and 937, respectively, and a CDRL1, CDRL2 and CDRL3 having the amino acid sequences specified in SEQ ID NOs: 938, 939 and 940, respectively. In one embodiment, an antibody of the invention may comprise a heavy chain variable domain comprising or consisting of an amino acid sequence having ≥80%, ≥90%, ≥95%, ≥96%, ≥97%, ≥98%, ≥99% or 100% sequence identity to the heavy chain variable domain of antibody Omi39 (i.e. SEQ ID NO: 932). In one embodiment, an antibody of the invention may comprise a light chain variable domain comprising or consisting of an amino acid sequence having ≥80%, ≥90%, ≥95%, ≥96%, ≥97%, ≥98%, ≥99% or 100% sequence identity to the light chain variable domain of antibody Omi39 (i.e. SEQ ID NO: 934). In one embodiment, an antibody of the invention may comprise a heavy chain variable domain and a light chain variable domain comprising or consisting of an amino acid sequence having ≥80%, ≥90%, ≥95%, ≥96%, ≥97%, ≥98%, ≥99%, 100% sequence identity to the heavy chain variable domain and light chain variable domain, respectively, of antibody Omi39 (i.e. SEQ ID NOs: 932 and 934, respectively).

In one embodiment, the antibody in Table 3 may be Omi17. Omi17 was found to neutralise the live SARS-CoV-2 variant strains Victoria, Alpha, Beta, Gamma, Delta and Omicron, and the psuedoviral constructs of Victoria, Omicron BA.1, Omicron BA.1.1, Omicron BA.2 and Omicron BA.3 (see Tables 13 and 14, FIG. 2). In one embodiment, an antibody of the invention may comprise a CDRH1, CDRH2 and CDRH3 having the amino acid sequences specified in SEQ ID NOs: 755, 756 and 757, respectively, and a CDRL1, CDRL2 and CDRL3 having the amino acid sequences specified in SEQ ID NOs: 758, 759 and 760, respectively. In one embodiment, an antibody of the invention may comprise a heavy chain variable domain comprising or consisting of an amino acid sequence having ≥80%, ≥90%, ≥95%, ≥96%, ≥97%, ≥98%, ≥99% or 100% sequence identity to the heavy chain variable domain of antibody Omi17 (i.e. SEQ ID NO: 752). In one embodiment, an antibody of the invention may comprise a light chain variable domain comprising or consisting of an amino acid sequence having ≥80%, ≥90%, ≥95%, ≥96%, ≥97%, ≥98%, ≥99% or 100% sequence identity to the light chain variable domain of antibody Omi17 (i.e. SEQ ID NO: 754). In one embodiment, an antibody of the invention may comprise a heavy chain variable domain and a light chain variable domain comprising or consisting of an amino acid sequence having ≥80%, ≥90%, ≥95%, ≥96%, ≥97%, ≥98%, ≥99%, 100% sequence identity to the heavy chain variable domain and light chain variable domain, respectively, of antibody Omi17 (i.e. SEQ ID NOs: 752 and 754, respectively).

The heavy chain domain of Omi17 is derived from a IGHV3-66 v-region, and the inventors have previously demonstrated that switching of the heavy chains and light chains between antibodies derived from the same v-region results in an antibody that is particularly useful with the invention (explained further below). Hence, an antibody of the invention may comprise the heavy chain of Omi17, and not the light chain of Omi17. For example, the antibody may comprise a CDRH1, CDRH2 and CDRH3 having the amino acid sequences specified in SEQ ID NOs: 755, 756 and 757, respectively. The antibody may comprise a heavy chain variable domain comprising or consisting of an amino acid sequence having ≥80%, ≥90%, ≥95%, ≥96%, ≥97%, ≥98%, ≥99% or 100% sequence identity to the heavy chain variable domain of antibody Omi17 (i.e. SEQ ID NO: 752). The antibody may comprise a heavy chain variable domain comprising or consisting of SEQ ID NO: 752.

Alternatively, in an embodiment of the invention, the antibody may comprise the light chain of Omi17, and not the heavy chain of Omi17. For example, the antibody may comprise a CDRL1, CDRL2 and CDRL3 having the amino acid sequences specified in SEQ ID NOs: 758, 759 and 760, respectively. The antibody may comprise a light chain variable domain comprising or consisting of an amino acid sequence having ≥80%,≥90%, ≥95%, ≥96%, ≥97%, ≥98%, ≥99% or 100% sequence identity to the light chain variable domain of antibody Omi17 (i.e. SEQ ID NO: 754). The antibody may comprise alight chain variable domain comprising or consisting of SEQ ID NO: 754.

In one embodiment, the antibody in Table 3 may be Omi29. Omi29 was found to neutralise the live SARS-CoV-2 variant strains Victoria, Alpha, Beta, Gamma, Delta and Omicron, and the psuedoviral constructs of Victoria Omicron BA.1, Omicron BA.1.1, Omicron BA.2 and Omicron BA.3 (see Tables 13 and 14, FIG. 2). In one embodiment, an antibody of the invention may comprise a CDRH1, CDRH2 and CDRH3 having the amino acid sequences specified in SEQ ID NOs: 845, 846 and 847, respectively, and a CDRL1, CDRL2 and CDRL3 having the amino acid sequences specified in SEQ ID NOs: 848, 849 and 850, respectively. In one embodiment, an antibody of the invention may comprise a heavy chain variable domain comprising or consisting of an amino acid sequence having ≥80%, ≥90%, ≥95%, ≥96%, ≥97%, ≥98%, ≥99% or 100% sequence identity to the heavy chain variable domain of antibody Omi29 (i.e. SEQ ID NO: 842). In one embodiment, an antibody of the invention may comprise a light chain variable domain comprising or consisting of an amino acid sequence having ≥80%, ≥90%, ≥95%, ≥96%, ≥97%, ≥98%, ≥99% or 100% sequence identity to the light chain variable domain of antibody Omi29 (i.e. SEQ ID NO: 844). In one embodiment, an antibody of the invention may comprise a heavy chain variable domain and a light chain variable domain comprising or consisting of an amino acid sequence having ≥80%, ≥90%, ≥95%, ≥96%, ≥97%,≥98%, ≥99%, 100% sequence identity to the heavy chain variable domain and light chain variable domain, respectively, of antibody Omi29 (i.e. SEQ ID NOs: 842 and 844, respectively).

The heavy chain domain of Omi29 is derived from a IGHV3-53 v-region, and the inventors have previously demonstrated that switching of the heavy chains and light chains between antibodies derived from the same v-region results in an antibody that is particularly useful with the invention (explained further below). Hence, an antibody of the invention may comprise the heavy chain of Omi29, and not the light chain of Omi29. For example, the antibody may comprise a CDRH1, CDRH2 and CDRH3 having the amino acid sequences specified in SEQ ID NOs: 845, 846 and 847, respectively. The antibody may comprise a heavy chain variable domain comprising or consisting of an amino acid sequence having ≥80%, ≥90%, ≥95%, ≥96%, ≥97%, ≥98%, ≥99% or 100% sequence identity to the heavy chain variable domain of antibody Omi29 (i.e. SEQ ID NO: 842). The antibody may comprise a heavy chain variable domain comprising or consisting of SEQ ID NO: 842.

Alternatively, in an embodiment of the invention, the antibody may comprise the light chain of Omi29, and not the heavy chain of Omi29. For example, the antibody may comprise a CDRL1, CDRL2 and CDRL3 having the amino acid sequences specified in SEQ ID NOs: 848, 849 and 850, respectively. The antibody may comprise a light chain variable domain comprising or consisting of an amino acid sequence having ≥80%,≥90%, ≥95%, ≥96%, ≥97%, ≥98%, ≥99% or 100% sequence identity to the light chain variable domain of antibody Omi29 (i.e. SEQ ID NO: 844). The antibody may comprise alight chain variable domain comprising or consisting of SEQ ID NO: 844.

In one embodiment, the antibody in Table 3 may be Omi36. Omi36 was found to neutralise the live SARS-CoV-2 variant strains Victoria, Alpha, Beta, Gamma, Delta and Omicron, and the psuedoviral constructs of Victoria, Omicron BA.1, Omicron BA.1.1, Omicron BA.2 and Omicron BA.3 (see Tables 13 and 14, FIG. 2). In one embodiment, an antibody of the invention may comprise a CDRH1, CDRH2 and CDRH3 having the amino acid sequences specified in SEQ ID NOs: 915, 916 and 917, respectively, and a CDRL1, CDRL2 and CDRL3 having the amino acid sequences specified in SEQ ID NOs: 918, 919 and 920, respectively. In one embodiment, an antibody of the invention may comprise a heavy chain variable domain comprising or consisting of an amino acid sequence having ≥80%, ≥90%, ≥95%, ≥96%, ≥97%, ≥98%, ≥99% or 100% sequence identity to the heavy chain variable domain of antibody Omi36 (i.e. SEQ ID NO: 912). In one embodiment, an antibody of the invention may comprise a light chain variable domain comprising or consisting of an amino acid sequence having ≥80%, ≥90%, ≥95%, ≥96%, ≥97%, ≥98%, ≥99% or 100% sequence identity to the light chain variable domain of antibody Omi36 (i.e. SEQ ID NO: 914).

In one embodiment, an antibody of the invention may comprise a heavy chain variable domain and a light chain variable domain comprising or consisting of an amino acid sequence having ≥80%, ≥90%, ≥95%, ≥96%, ≥97%, ≥98%, ≥99%, 100% sequence identity to the heavy chain variable domain and light chain variable domain, respectively, of antibody Omi36 (i.e. SEQ ID NOs: 912 and 914, respectively). The heavy chain domain of Omi36 is derived from a IGHV3-66 v-region, and the inventors have previously demonstrated that switching of the heavy chains and light chains between antibodies derived from the same v-region results in an antibody that is particularly useful with the invention (explained further below). Hence, an antibody of the invention may comprise the heavy chain of Omi36, and not the light chain of Omi36. For example, the antibody may comprise a CDRH1, CDRH2 and CDRH3 having the amino acid sequences specified in SEQ ID NOs: 915, 916 and 917, respectively. The antibody may comprise a heavy chain variable domain comprising or consisting of an amino acid sequence having ≥80%, ≥90%, ≥95%, ≥96%, ≥97%, ≥98%, ≥99% or 100% sequence identity to the heavy chain variable domain of antibody Omi36 (i.e. SEQ ID NO: 912). The antibody may comprise a heavy chain variable domain comprising or consisting of SEQ ID NO: 912.

Alternatively, in an embodiment of the invention, the antibody may comprise the light chain of Omi36, and not the heavy chain of Omi36. For example, the antibody may comprise a CDRL1, CDRL2 and CDRL3 having the amino acid sequences specified in SEQ ID NOs: 918, 919 and 920, respectively. The antibody may comprise a light chain variable domain comprising or consisting of an amino acid sequence having ≥80%,≥90%, ≥95%, ≥96%, ≥97%, ≥98%, ≥99% or 100% sequence identity to the light chain variable domain of antibody Omi36 (i.e. SEQ ID NO: 914). The antibody may comprise alight chain variable domain comprising or consisting of SEQ ID NO: 914.

In one embodiment, the antibody in Table 3 may be Omi38. Omi38 was found to neutralise the live SARS-CoV-2 variant strains Victoria, Alpha, Beta, Gamma, Delta and Omicron, and the psuedoviral constructs of Victoria, Omicron BA.1, Omicron BA.1.1, Omicron BA.2 and Omicron BA.3 (see Tables 13 and 14, FIG. 2). In one embodiment, an antibody of the invention may comprise a CDRH1, CDRH2 and CDRH3 having the amino acid sequences specified in SEQ ID NOs: 925, 926 and 927, respectively, and a CDRL1, CDRL2 and CDRL3 having the amino acid sequences specified in SEQ ID NOs: 928, 929 and 930, respectively. In one embodiment, an antibody of the invention may comprise a heavy chain variable domain comprising or consisting of an amino acid sequence having ≥80%, ≥90%, ≥95%, ≥96%, ≥97%, ≥98%, ≥99% or 100% sequence identity to the heavy chain variable domain of antibody Omi38 (i.e. SEQ ID NO: 922). In one embodiment, an antibody of the invention may comprise a light chain variable domain comprising or consisting of an amino acid sequence having ≥80%, ≥90%, ≥95%, ≥96%, ≥97%, ≥98%, ≥99% or 100% sequence identity to the light chain variable domain of antibody Omi38 (i.e. SEQ ID NO: 924). In one embodiment, an antibody of the invention may comprise a heavy chain variable domain and a light chain variable domain comprising or consisting of an amino acid sequence having ≥80%, ≥90%, ≥95%, ≥96%, ≥97%, ≥98%, ≥99%, 100% sequence identity to the heavy chain variable domain and light chain variable domain, respectively, of antibody Omi38 (i.e. SEQ ID NOs: 922 and 924, respectively).

The heavy chain domain of Omi38 is derived from a IGHV1-69 v-region, and the inventors have previously demonstrated that switching of the heavy chains and light chains between antibodies derived from the same v-region results in an antibody that is particularly useful with the invention (explained further below). Hence, an antibody of the invention may comprise the heavy chain of Omi38, and not the light chain of Omi38. For example, the antibody may comprise a CDRH1, CDRH2 and CDRH3 having the amino acid sequences specified in SEQ ID NOs: 925, 926 and 927, respectively. The antibody may comprise a heavy chain variable domain comprising or consisting of an amino acid sequence having ≥80%, ≥90%, ≥95%, ≥96%, ≥97%, ≥98%, ≥99% or 100% sequence identity to the heavy chain variable domain of antibody Omi38 (i.e. SEQ ID NO: 922). The antibody may comprise a heavy chain variable domain comprising or consisting of SEQ ID NO: 922.

Alternatively, in an embodiment of the invention, the antibody may comprise the light chain of Omi38, and not the heavy chain of Omi38. For example, the antibody may comprise a CDRL1, CDRL2 and CDRL3 having the amino acid sequences specified in SEQ ID NOs: 928, 929 and 930, respectively. The antibody may comprise a light chain variable domain comprising or consisting of an amino acid sequence having ≥80%,≥90%, ≥95%, ≥96%, ≥97%, ≥98%, ≥99% or 100% sequence identity to the light chain variable domain of antibody Om38 (i.e. SEQ ID NO: 924). The antibody may comprise alight chain variable domain comprising or consisting of SEQ ID NO: 924.

### Mixed Chain Antibodies of the Invention

An antibody of the invention may comprise a light chain variable domain comprising CDRL1, CDRL2 and CDRL3 from a first antibody in Table 1, 2 or 3 and a heavy chain variable domain comprising CDRH1, CDRH2 and CDRH3 from a second antibody in Table 1, 2 or 3, with the proviso that the first and second antibodies are different. Such antibodies are referred to as mixed chain antibodies herein.

Examples of the mixed chain antibodies useful with the invention are provided in Tables 4 to 12. Table 4 shows examples of mixed chain antibodies generated from antibodies in Tables 1 to 3 that are derived from the same germline heavy chain IGHV 3-53. Table 5 shows examples of mixed chain antibodies generated from antibodies in Tables 1 to 3 that are derived from the same germline heavy chain IGHV 3-53 and IGHV3-66. Table 6 shows examples of mixed chain antibodies generated from antibodies in Tables 1 to 3 that are derived from the same germline heavy chain IGHV1-58. Table 7 shows examples of mixed chain antibodies generated from antibodies in Tables 2 and 3 that are derived from the same germline heavy chain IGHV1-69. Table 8 shows examples of mixed chain antibodies generated from antibodies in Tables 1 to 3 that are derived from the same germline heavy chain IGHV3-30. Table 9 shows examples of mixed chain antibodies generated from antibodies in Tables 2 and 3 that are derived from the same germline heavy chain IGHV3-33. Table 10 shows examples of mixed chain antibodies generated from antibodies in Tables 1 to 3 that are derived from the same germline heavy chain IGHV1-18. Table 11 shows examples of mixed chain antibodies generated from antibodies in Tables 1 and 3 that are derived from the same germline heavy chain IGHV3-9. Table 12 shows examples of mixed chain antibodies generated from antibodies in Tables 2 and 3 that are derived from the same germline heavy chain IGHV4-31. Examples of mixed chain antibodies that are derived from the same germline heavy chain IGHV1-69 are Omi02H/Beta-49L and Omi38H/Omi24L.

Hence, in one embodiment, an antibody of the invention comprises a heavy chain variable domain comprising CDRH1, CDRH2 and CDRH3 from a first antibody in Table 1, 2 or 3 and a light chain variable domain comprising CDRL1, CDRL2 and CDRL3 from a second antibody in Table 1, 2 or 3, with the proviso that the first and second antibodies are different. The antibody may comprise a heavy chain variable domain amino acid sequence having at least 80% sequence identity to the heavy chain variable domain from a first antibody in Table 1, 2 or 3, and a light chain variable domain amino acid sequence having at least 80% sequence identity to the light chain variable domain from a second antibody in Table 1, 2 or 3, with the proviso that the first and second antibodies are different. For example, the antibody may comprise a heavy chain variable domain comprising or consisting of an amino acid sequence having ≥80%, ≥90%, ≥95%,≥96%, ≥97%, ≥98%, ≥99% or 100% sequence identity to the heavy chain variable domain of an antibody in Table 1, 2 or 3, and a light chain variable domain comprising or consisting of an amino acid sequence having ≥80%, ≥90%, ≥95%, ≥96%, ≥97%, ≥98%, ≥99% or 100% sequence identity to the light chain variable domain of an antibody in Table 1, 2 or 3, with the proviso that the first and second antibodies are different.

The first antibody may be in Table 3 and the second antibody may be in Table 3.

The first antibody may be in Table 3 and the second antibody may be in Table 1. The first antibody may be in Table 3 and the second antibody may be in Table 2. The first antibody may be in Table 1 and the second antibody may be in Table 3. The first antibody may be in Table 2 and the second antibody may be in Table 3. The first antibody may be in Table 1 and the second antibody may be in Table 2. The first antibody may be in Table 2 and the second antibody may be in Table 1. The first antibody may be in Table 2 and the second antibody may be in Table 2. The first antibody may be in Table 1 and the second antibody may be in Table 1.

In one embodiment, at least one of the first and second antibodies is an antibody from Table 3.

In one embodiment, the first and second antibodies are not both in Table 1. In one embodiment, the first and second antibodies are not both in Table 2. In one embodiment, the first and second antibodies are not both selected from an antibody in Table 1 or 2.

In one embodiment, at least one of the heavy chain variable domain and the light chain variable domain are from Table 3.

The antibody in Table 3 may be selected from the group consisting of: Omi02, Omi03, Omi12, Omi18, Omi28, Omi39 and Omi42. The antibody in Table 3 may be selected from the group consisting of: Omi03, Omi12, Omi02, Omi39, Omi42, Omi16, Omi18, Omi20, Omi 23, Omi28, Omi08, Omi17, Omi29, Omi36 and Omi38. For example, the antibody in Table 3 may be selected from the group consisting of Omi03, Omi12, Omi02, Omi39, Omi42, Omi16, Omi18, Omi20, Omi 23, Omi28 and Omi08. The antibody in Table 3 may be selected from the group consisting of Omi03, Omi12, Omi02, Omi39, and Omi42. The antibody in Table 3 may be selected from the group consisting of Omi03 and Omi12.

In one embodiment, the first antibody and the second antibody are both selected from the group consisting of: Omi03, Omi18, Omi29, Beta-27, antibody 150, antibody 158, antibody 175, antibody 222 and antibody 269. The heavy chain variable domain of these antibodies are derived from IGHV3-53. The resulting mixed chain antibodies are set out in Table 4. Hence, the antibody of the invention may comprise all six CDRs (CDRH1-3 and CDRL1-3), and/or a heavy chain variable domain and a light chain variable domain, each comprising or consisting of an amino acid sequence having at least 80% sequence identity to the corresponding variable domain of any one of the mixed chain antibodies as set out in Table 4.

Antibodies derived from IGHV3-53 may be used to produce mixed-chain antibodies with antibodies from IGHV3-66 (e.g. antibodies 40 and 398 in Table 1) (see, e.g. Dejnirattisai, Wanwisa, et al. “The antigenic anatomy of SARS-CoV-2 receptor binding domain.” Cell 184(8) (2021): 2183-2200; Supasa, Piyada, et al. “Reduced neutralization of SARS-CoV-2 B.1.1.7 variant by convalescent and vaccine sera.” Cell 184(8) (2021): 2201-2211; Zhou, Daming, et al. “Evidence of escape of SARS-CoV-2 variant B.1.351 from natural and vaccine-induced sera.” Cell 184(9) (2021): 2348-2361; Dejnirattisai, Wanwisa, et al. “Antibody evasion by the P.1 strain of SARS-CoV-2.” Cell 184(11) (2021): 2939-2954; Liu, Chang, et al. “Reduced neutralization of SARS-CoV-2 B.1.617 by vaccine and convalescent serum.” Cell 184(16) (2021): 4220-4236)). Accordingly, in one embodiment, the first antibody and the second antibody are both selected from the group consisting of: Omi03, Omi18, Omi29, Omi16, Omi17, Omi20, Omi27, Omi36, Beta-27, antibody 150, antibody 158, antibody 175, antibody 222, antibody 269, antibody 40 and antibody 398. The heavy chain variable domain of these antibodies are derived from IGHV3-53 and IGVH3-66. The resulting mixed chain antibodies are set out in Table 5. Hence, the antibody of the invention may comprise all six CDRs (CDRH1-3 and CDRL1-3), and/or a heavy chain variable domain and a light chain variable domain, each comprising or consisting of an amino acid sequence having at least 80% (e.g. ≥80%, ≥90%, ≥95%, ≥96%, ≥97%, ≥98%, ≥99% or 100%) sequence identity to the corresponding variable domain of any one of the mixed chain antibodies as set out in Table 5.

In one embodiment, the first antibody and the second antibody are both selected from the group consisting of: Omi12, Beta-47, Beta-25, antibody 55, antibody 165, antibody 253 and antibody 318. The heavy chain variable domain of these antibodies are derived from IGHV 1-58. The resulting mixed chain antibodies are set out in Table 6. Hence, the antibody of the invention may comprise all six CDRs (CDRH1-3 and CDRL1-3), and/or a heavy chain variable domain and a light chain variable domain, each comprising or consisting of an amino acid sequence having at least 80% (e.g. ≥80%, ≥90%, ≥95%, ≥96%, ≥97%, ≥98%, ≥99% or 100%) sequence identity to the corresponding variable domain of any one of the mixed chain antibodies as set out in Table 6.

In one embodiment, the first antibody and the second antibody are both selected from the group consisting of: Beta-49, Beta-50, Omi02, Omi24, Omi30, Omi31, Omi34 and Omi38. The heavy chain variable domain of these antibodies are derived from IGHV 1-69. The resulting mixed chain antibodies are set out in Table 7. Hence, the antibody of the invention may comprise all six CDRs (CDRH1-3 and CDRL1-3), and/or a heavy chain variable domain and a light chain variable domain, each comprising or consisting of an amino acid sequence having at least 80% (e.g. ≥80%, ≥90%, ≥95%, ≥96%, ≥97%, ≥98%, ≥99% or 100%) sequence identity to the corresponding variable domain of any one of the mixed chain antibodies as set out in Table 7.

In one embodiment, the first antibody and the second antibody are both selected from the group consisting of: Beta-22, Beta-29, antibody 159, and Omi09. The heavy chain variable domain of these antibodies are derived from IGHV 3-30. The resulting mixed chain antibodies are set out in Table 8. Hence, the antibody of the invention may comprise all six CDRs (CDRH1-3 and CDRL1-3), and/or a heavy chain variable domain and a light chain variable domain, each comprising or consisting of an amino acid sequence having at least 80% (e.g. ≥80%, ≥90%, ≥95%, ≥96%, ≥97%, ≥98%, ≥99% or 100%) sequence identity to the corresponding variable domain of any one of the mixed chain antibodies as set out in Table 8.

In one embodiment, the first antibody and the second antibody are both selected from the group consisting of: Beta-20, Beta-43, Omi32 and Omi33. The heavy chain variable domain of these antibodies are derived from IGHV 3-33. The resulting mixed chain antibodies are set out in Table 9. Hence, the antibody of the invention may comprise all six CDRs (CDRH1-3 and CDRL1-3), and/or a heavy chain variable domain and alight chain variable domain, each comprising or consisting of an amino acid sequence having at least 80% (e.g. ≥80%, ≥90%, ≥95%, ≥96%, ≥97%, ≥98%, ≥99% or 100%) sequence identity to the corresponding variable domain of any one of the mixed chain antibodies as set out in Table 9. The CDRL1-3 of Omi32 and Omi33 are identical, meaning that they are, effectively, already exemplary mixed-chain antibodies of the invention.

In one embodiment, the first antibody and the second antibody are both selected from the group consisting of: antibody 278, Beta-44, Omi26 and Omi41. The heavy chain variable domain of these antibodies are derived from IGHV 1-18. The resulting mixed chain antibodies are set out in Table 10. Hence, the antibody of the invention may comprise all six CDRs (CDRH1-3 and CDRL1-3), and/or a heavy chain variable domain and a light chain variable domain, each comprising or consisting of an amino acid sequence having at least 80% (e.g. ≥80%, ≥90%, ≥95%, ≥96%, ≥97%, ≥98%, ≥99% or 100%) sequence identity to the corresponding variable domain of any one of the mixed chain antibodies as set out in Table 10.

In one embodiment, the first antibody and the second antibody are both selected from the group consisting of: antibody 58, Omi25, Omi35 and Omi42. The heavy chain variable domain of these antibodies are derived from IGHV 3-9. The resulting mixed chain antibodies are set out in Table 11. Hence, the antibody of the invention may comprise all six CDRs (CDRH1-3 and CDRL1-3), and/or a heavy chain variable domain and a light chain variable domain, each comprising or consisting of an amino acid sequence having at least 80% (e.g. ≥80%, ≥90%, ≥95%, ≥96%, ≥97%, ≥98%, ≥99% or 100%) sequence identity to the corresponding variable domain of any one of the mixed chain antibodies as set out in Table 11.

In one embodiment, the first antibody and the second antibody are both selected from the group consisting of: Beta-56 and Omi23. The heavy chain variable domain of these antibodies are derived from IGHV 4-31. The resulting mixed chain antibodies are set out in Table 12. Hence, the antibody of the invention may comprise all six CDRs (CDRH1-3 and CDRL1-3), and/or a heavy chain variable domain and a light chain variable domain, each comprising or consisting of an amino acid sequence having at least 80% (e.g. ≥80%, ≥90%, ≥95%, ≥96%, ≥97%, ≥98%, ≥99% or 100%) sequence identity to the corresponding variable domain of any one of the mixed chain antibodies as set out in Table 12.

The constant region domains of an antibody molecule of the invention, if present, may be selected having regard to the proposed function of the antibody molecule, and in particular the effector functions which may be required. For example, the constant region domains may be human IgA, IgD, IgE, IgG or IgM domains. Typically, the constant regions are of human origin. In particular, human IgG (i.e. IgG1, IgG2, IgG3 or IgG4) constant region domains may be used. Typically, the constant region is a human IgG1 constant region.

### Certain Antibodies of the Invention

The invention also provides an antibody which is a full length antibody of any one of the antibodies in Tables 3, 4, 5, 6, 7, 8, 9, 10, 11 or 12. In other words, an antibody of the invention comprises a heavy chain variable domain and a light chain variable domain consisting of the heavy chain variable domain and light chain variable domain, respectively, of any one of the antibodies in Tables 3, 4, 5, 6, 7, 8, 9, 10, 11 or 12, and a IgG (e.g. IgG1) constant region.

For example, the antibody of the invention may be a full length Omi02, Omi03, Omi12, Omi18, Omi28, Omi39 or Omi42 antibody. The antibody of the invention may be a full length Omi03, Omi12, Omi02, Omi39, Omi42, Omi16, Omi18, Omi20, Omi 23, Omi28, Omi08, Omi17, Omi29, Omi36 or Omi38 antibody. These antibodies are all highly potent neutralising mAbs that have been shown to neutralise the Omicron variant of SARS-CoV-2 with an IC50 of ≤0.1 μg/ml. The antibodies also retain neutralisation of inter alia at least the Victoria, Alpha, Beta, Gamma and Delta strains of SARS-CoV-2 with an IC50 of ≤0.1 μg/ml.

The antibody may be derived from germline heavy chain IGHV1-58 and comprises proline at position 53 in the heavy chain variable region (according to absolute numbering). For example, the antibody may comprise a heavy chain variable domain comprising or consisting of an amino acid sequence having ≥80%, ≥90%, ≥95%, ≥96%, ≥97%, ≥98%, ≥99% or 100% sequence identity to the heavy chain variable domain of Omi-12 (SEQ ID NO: 731), Beta-47 (SEQ ID NO: 591), Beta-25 (SEQ ID NO: 461), antibody 55 (SEQ ID NO: 62), antibody 165 (SEQ ID NO: 182), antibody 253 (SEQ ID NO: 262), or antibody 318 (SEQ ID NO: 332), with the proviso that the amino acid at position 53 in the heavy chain variable region is proline (according to absolute numbering). For example, the antibody may comprise the heavy chain variable region and the light chain variable region of Beta-47 (SEQ ID NOs: 591 and 592, respectively), Beta-25 (SEQ ID NOs: 461 and 462, respectively), antibody 55 (SEQ ID NOs: 62 and 61, respectively), antibody 165 (SEQ ID NO: 182 and 181, respectively), antibody 253 (SEQ ID NOs: 262 and 261, respectively), or antibody 318 (SEQ ID NOs: 332 and 331, respectively), except with a V53P mutation in the heavy chain variable region. The inventors found that such antibodies are particularly effective against Omicron strains (e.g. see Example 5).

The position 53 in the heavy chain variable region of the IGHV1-58-derived antibodies Omi-12, Beta-47, Beta-25, antibody 55, antibody 165, antibody 253, and antibody 318 corresponds to position 58 according to IMGT numbering.

Accordingly, the invention also provides an antibody derived from germline heavy chain IGHV1-58, capable of binding to the spike protein of coronavirus SARS-CoV-2, wherein the amino acid at position 58 in the heavy chain variable region according to IMGT numbering is proline or is substituted with proline.

The antibody may comprise a heavy chain variable domain comprising an amino acid sequence having ≥80%, ≥90%, ≥95%, ≥96%, ≥97%, ≥98%, ≥99% or 100% sequence identity to the heavy chain variable domain of an antibody derived from germline heavy chain IGHV1-58, with the proviso that the amino acid at position 58 according to IMGT numbering is proline or is substituted with proline.

The antibody derived from germline heavy chain IGHV1-58 may be AZD8895, Omi-12, Beta-47, Beta-25, antibody 55, antibody 165, antibody 253, or antibody 318. The amino acid sequence of the heavy chain variable domain of Omi-12, Beta-47, Beta-25, antibody 55, antibody 165, antibody 253, or antibody 318 is described herein (e.g. see Tables 1 to 3). The amino acid sequence of the heavy chain variable domain of antibody AZD8895 is provided in SEQ ID NO: 963.

The IGHV1-58 germline V-gene sequence encodes the amino acid sequence:

(SEQ ID NO: 961)
MQLVQSGPEVKKPGTSVKVSCKASGFTFTSSAVQWVRQARGQRLEWIGW
IVVGSGNTNYAQKFQERVTITRDMSTSTAYMELSSLRSEDTAVYYCAA.

Hence, the invention also provides an antibody capable of binding to the spike protein of coronavirus SARS-CoV-2 comprising a heavy chain variable domain comprising an amino acid sequence having ≥60%, ≥70%, ≥80%, ≥90%, ≥95%, ≥96%, ≥97%, ≥98%, ≥99% or 100% sequence identity to SEQ ID NO: 961, with the proviso that the amino acid at position 58 according to IMGT numbering is proline or is substituted with proline.

The antibody may comprise a heavy chain variable domain comprising an amino acid sequence having ≥80%, ≥90%, ≥95%, ≥96%, ≥97%, ≥98%, ≥99% or 100% sequence identity to SEQ ID NO: 731, 591, 461, 62, 182, 262, 332 or 963, with the proviso that the amino acid at position 58 according to IMGT numbering is proline or is substituted with proline. The antibody may comprise a heavy chain variable domain comprising an amino acid sequence having SEQ ID NO: 591 461, 62, 182, 262, or 332, wherein the valine at position 58 according to IMGT numbering is substituted with proline.

The antibody may comprise a heavy chain variable domain comprising an amino acid sequence having SEQ ID NO: 963, wherein the isoleucine at position 58 according to IMGT numbering is substituted with proline.

In some embodiments, the antibody derived from germline heavy chain IGHV1-58, comprises a light chain variable domain derived from IGLV Kappa 3-20. The antibody may comprise a light chain variable domain comprising an amino acid sequence having ≥80%, ≥90%, ≥95%, ≥96%, ≥97%, ≥98%, ≥99% or 100% sequence identity to the light chain variable domain of an antibody derived from germline IGLV Kappa 3-20. The germline IGLV Kappa 3-20V sequence may encode the amino acid sequence: EIVLTQSPGTLSLSPGERATLSCRASQSVSSSYLAWYQQKPGQAPRLLIYGASSRAT GIPDRFSGSGSGTDFTLTISRLEPEDFAVYYCQQYGSSP (SEQ ID NO: 967). Hence, the antibody derived from germline heavy chain IGHV1-58 may comprise a light chain variable domain comprising an amino acid sequence having ≥60%, ≥70%, ≥80%, ≥90%, ≥95%, ≥96%, ≥97%, ≥98%, ≥99% or 100% sequence identity to SEQ ID NO: 967.

The invention also provides an antibody capable of binding to the spike protein of coronavirus SARS-CoV-2 comprising a heavy chain variable domain comprising an amino acid sequence that is a modified version of SEQ ID NO: 961, with the proviso that the amino acid at position 58 according to IMGT numbering is proline or is substituted with proline. The modified version of SEQ ID NO: 961 may comprise a modification as described herein, e.g. a substitution, deletion and/or addition. For example, the modification may comprise ≤50, ≤45, ≤40, ≤35, ≤30, ≤25≤20≤15≤10≤9≤8≤7≤6≤5≤4≤3≤2 or 1 amino acid substitutions and/or deletions from SEQ ID NO: 961. The modification may comprise ≤4, ≤3, ≤2, or 1 amino acid substitutions and/or deletions from SEQ ID NO: 961.

The antibody may comprise a heavy chain variable domain comprising an amino acid sequence that is a modified version of SEQ ID NO: 731, 591, 461, 62, 182, 262, 332 or 963 which comprises ≤10, ≤9, ≤8, ≤7, ≤6, ≤5, ≤4, ≤3, ≤2, or 1 modifications, with the proviso that the amino acid at position 58 according to IMGT numbering is proline or is substituted with proline. The modified version of SEQ ID NO: 731, 591, 461, 62, 182, 262, 332 or 963 may comprise a modification as described herein, e.g. a substitution, deletion and/or addition.

The antibody may comprise a IgG (e.g. IgG1) constant region.

The invention also provides a method of preparing such antibodies. For example, the method may comprise modifying an antibody derived from the germline heavy chain IGHV1-58, capable of binding to the spike protein of coronavirus SARS-CoV-2, by substituting the amino acid at position 58 in the heavy chain variable region (according to IMGT numbering) with proline. The antibody derived from the germline heavy chain IGHV1-58 may be AZD8895, Omi-12, Beta-47, Beta-25, antibody 55, antibody 165, antibody 253, or antibody 318. The amino acid sequence of the heavy chain variable domain of each these antibodies is described herein (e.g. see Tables 1 to 3 and SEQ ID NO: 963). The invention also provides an antibody obtainable or obtained by the method.

### Properties of the Antibodies of the Invention

An antibody of the invention may be or may comprise a modification from the amino acid sequence of an antibody in Tables 1 to 12, whilst maintaining the activity and/or function of the antibody. The modification may a substitution, deletion and/or addition. For example, the modification may comprise 1, 2, 3, 4, 5, up to 10, up to 20, up to 30 or more amino acid substitutions and/or deletions from the amino acid sequence of an antibody in Tables 1 to 12. For example, the modification may comprise an amino acid substituted with an alternative amino acid having similar properties. Some properties of the 20 main amino acids, which can be used to select suitable substituents, are as follows:

| | | | |
|---|---|---|---|
| Ala | aliphatic, hydrophobic, neutral | Met | hydrophobic, neutral |
| Cys | polar, hydrophobic, neutral | Asn | polar, hydrophilic, neutral |
| Asp | polar, hydrophilic, charged (−) | Pro | hydrophobic, neutral |
| Glu | polar, hydrophilic, charged (−) | Gln | polar, hydrophilic, neutral |
| Phe | aromatic, hydrophobic, neutral | Arg | polar, hydrophilic, charged (+) |
| Gly | aliphatic, neutral | Ser | polar, hydrophilic, neutral |
| His | aromatic, polar, hydrophilic, | Thr | polar, hydrophilic, neutral |
| | charged (+) | Val | aliphatic, hydrophobic, neutral |
| Ile | aliphatic, hydrophobic, neutral | Trp | aromatic, hydrophobic, neutral |
| Lys | polar, hydrophilic, charged (+) | Tyr | aromatic, polar, hydrophobic |
| Leu | aliphatic, hydrophobic, neutral | | |

The modification may comprise a derivatised amino acid, e.g. a labelled or non-natural amino acid, providing the function of the antibody is not significantly adversely affected.

Modification of antibodies of the invention as described above may be prepared during synthesis of the antibody or by post-production modification, or when the antibody is in recombinant form using the known techniques of site-directed mutagenesis, random mutagenesis, or enzymatic cleavage and/or ligation of nucleic acids.

Antibodies of the invention may be modified (e.g. as described above) to improve the potency of said antibodies or to adapt said antibodies to new SARS-CoV-2 variants. The modifications may be amino acid substitutions to adapt the antibody to substitutions in a virus variant. For example, the known mode of binding of an antibody to the spike protein (e.g. by crystal structure determination, or modelling) may be used to identify the amino acids of the antibody that interact with the substitution in the virus variant. This information can then be used to identify possible substitutions of the antibody that will compensate for the change in the epitope characteristics. For example, a substitution of a hydrophobic amino acid in the spike protein to a negatively changes amino acid may be compensated by substituting the amino acid from the antibody that interacts with said amino acid in the spike protein to a positively charged amino acid. Methods for identifying residues of an antibody that may be substituted are encompassed by the present disclosure, for example, by determining the structure of antibody-antigen complexes as described herein.

The antibodies of the invention may contain one or more modifications to increase their cross-lineage neutralisation property. For example, E484 of the spike protein, which is a key residue that mediates the interaction with ACE2, is mutated in some SARS-CoV-2 strains (e.g. Victoria strain which contains E484, but P.1 and B.1.351 strains contain E484K) resulting in differing neutralisation effects of the antibodies. Thus, antibodies that bind to E484 can be modified to compensate for the changes in E484 of the spike protein. For example, E484 is mutated from a positively charge to negatively charged amino acid in SAR-CoV-2 strains of B.1.351 or P.1 lineage, when compared to the original strain. The amino acid residues of antibodies that bind to or near E484 may be mutated to compensate for the change in charge. Examples of such amino acid residues may be G104 and/or K108 in SEQ ID NO: 102 of antibody 88, or R52 in SEQ ID NO: 372 of antibody 384.

Antibodies of the invention may be isolated antibodies. An isolated antibody is an antibody which is substantially free of other antibodies having different antigenic specificities.

The term ‘antibody’ as used herein may relate to whole antibodies (i.e. comprising the elements of two heavy chains and two light chains inter-connected by disulphide bonds) as well as antigen-binding fragments thereof. Antibodies typically comprise immunologically active portions of immunoglobulin (Ig) molecules, i.e., molecules that contain an antigen binding site that specifically binds (immunoreacts with) an antigen. By “specifically binds” or “immunoreacts with” is meant that the antibody reacts with one or more antigenic determinants of the desired antigen and does not react with other polypeptides. Each heavy chain is comprised of a heavy chain variable region (abbreviated herein as HCVR or VH) and at least one heavy chain constant region. Each light chain is comprised of a light chain variable region (abbreviated herein as LCVR or VL) and a light chain constant region. The variable regions of the heavy and light chains contain a binding domain that interacts with an antigen. The VH and VL regions can be further subdivided into regions of hypervariability, termed complementarity determining regions (CDR), interspersed with regions that are more conserved, termed framework regions (FR).

Antibodies may include, but are not limited to, polyclonal, monoclonal, chimeric, dAb (domain antibody), single chain, Fab, Fab′ and F(ab′)2 fragments, scFvs, and Fab expression libraries

An antibody of the invention may be a monoclonal antibody. Monoclonal antibodies (mAbs) of the invention may be produced by a variety of techniques, including conventional monoclonal antibody methodology, for example those disclosed in “Monoclonal Antibodies: a manual of techniques” (Zola H, 1987, CRC Press) and in “Monoclonal Hybridoma Antibodies: techniques and applications” (Hurrell J G R, 1982 CRC Press). An antibody of the invention may be multispecific, such as bispecific. A bispecific antibody of the invention binds two different epitopes. The epitopes may be in the same protein (e.g. two epitopes in spike protein of SARS-CoV-2) or different proteins (e.g. one epitope in spike protein and one epitope in another protein (such as coat protein) of SARS-CoV-2).

In one embodiment, a bispecific antibody of the invention may bind to two separate epitopes on the spike protein of SARS-CoV-2. The bispecific antibody may bind to the NTD of the spike protein and to the RBD of the spike protein. The bispecific antibody may bind to two different epitopes in the RBD of the spike protein.

One or more (e.g. two) antibodies of the invention can be coupled to form a multispecific (e.g. bispecific) antibody. Methods to prepare multispecific, e.g. bispecific, antibodies are well known in the art.

An antibody may be selected from the group consisting of single chain antibodies, single chain variable fragments (scFvs), variable fragments (Fvs), fragment antigen-binding regions (Fabs), recombinant antibodies, monoclonal antibodies, fusion proteins comprising the antigen-binding domain of a native antibody or an aptamer, single-domain antibodies (sdAbs), also known as VHH antibodies, nanobodies (Camelid-derived single-domain antibodies), shark IgNAR-derived single-domain antibody fragments called VNAR, diabodies, triabodies, Anticalins, aptamers (DNA or RNA) and active components or fragments thereof.

The constant region domains of an antibody molecule of the invention, if present, may be selected having regard to the proposed function of the antibody molecule, and in particular the effector functions which may be required. For example, the constant region domains may be human IgA, IgD, IgE, IgG or IgM domains. Typically, the constant regions are of human origin. In particular, human IgG (i.e. IgG1, IgG2, IgG3 or IgG4) constant region domains may be used. Typically, the constant region is a human IgG1 constant region.

The light chain constant region may be either lambda or kappa. Antibodies of the invention may be mono-specific or multi-specific (e.g. bi-specific). A multi-specific antibody comprises at least two different variable domains, wherein each variable domain is capable of binding to a separate antigen or to a different epitope on the same antigen.

An antibody of the invention may be a chimeric antibody, a CDR-grafted antibody, a nanobody, a human or humanised antibody. Typically, the antibody is a human antibody. Fully human antibodies are those antibodies in which the variable regions and the constant regions (where present) of both the heavy and the light chains are all of human origin, or substantially identical to sequences of human origin, but not necessarily from the same antibody.

The antibody of the invention may be a full-length antibody.

The antibody of the invention may be an antigen-binding fragment. An antigen-binding fragment of the invention binds to the same epitope of the parent antibody, i.e. the antibody from which the antigen-binding fragment is derived. An antigen-binding fragment of the invention typically retains the parts of the parent antibody that interact with the epitope. The antigen-binding fragment typically comprise the complementarity-determining regions (CDRs) that interact with the antigen, such as one, two, three, four, five or six CDRs. In some embodiments, the antigen-binding fragment further comprises the structural scaffold surrounding the CDRs of the parent antibody, such as the variable region domains of the heavy and/or light chains. Typically, the antigen-binding fragment retains the same or similar binding affinity to the antigen as the parent antibody.

An antigen-binding fragment does not necessarily have an identical sequence to the parent antibody. In one embodiment, the antigen-binding fragment may have ≥70%, ≥80%, ≥90%, ≥95%, ≥96%, ≥97%, ≥98%, ≥99%, 100% sequence identity with the respective CDRs of the parent antibody. In one embodiment, the antigen-binding fragment may have ≥70%, ≥80%, ≥90%, ≥95%, ≥96%, ≥97%, ≥98%, ≥99%, 100% sequence identity with the respective variable region domains of the parent antibody. Typically, the non-identical amino acids of a variable region are not in the CDRs.

The antigen-binding fragments of antibodies of the invention retain the ability to selectively bind to an antigen. Antigen-binding fragments of antibodies include single chain antibodies (i.e. a full-length heavy chain and light chain); Fab, modified Fab, Fab′, modified Fab′, F(ab′)2, Fv, Fab-Fv, Fab-dsFv, single domain antibodies (e.g. VH or VL or VHH), scFv.

An antigen-binding function of an antibody can be performed by fragments of a full-length antibody. The methods for creating and manufacturing these antibody fragments are well known in the art (see for example Verma R et al., 1998, J. Immunol. Methods, 216, 165-181).

Methods for screening antibodies of the invention that do not share 100% amino acid sequence identity with one of the antibodies disclosed herein, that possess the desired specificity, affinity and functional activity include the methods described herein, e.g. enzyme linked immunosorbent assays, biacore, focus reduction neutralisation assay (FRNT), and other techniques known within the art.

With regards to function, an antibody of the invention may be able to neutralise at least one biological activity of SAR-CoV-2 (a neutralising antibody), particularly to neutralise virus infectivity.

Neutralisation may also be determined using IC50 or IC90 values. For example, the antibody may have an IC50 value of ≤0.1 μg/ml, ≤0.05 μg/ml, ≤0.01 μg/ml≤0.005 μg/ml or ≤0.002 μg/ml. In some instances an antibody of the invention may have an IC50 value of between 0.0001 μg/ml and 0.1 μg/ml, sometimes between 0.0001 μg/ml and 0.05 μg/ml or even between 0.0001 μg/ml and 0.001 μg/ml.

For example, the IC50 values of some of the antibodies of Tables 1 to 12 are provided in Tables 13 to 16.

The ability of an antibody to neutralise virus infectivity may be measured using an appropriate assay, particularly using a cell-based neutralisation assay, as shown in the Examples. For example, the neutralisation ability may be measured in a focus reduction neutralisation assay (FRNT) where the reduction in the number of cells (e.g. human cells) infected with the virus (e.g. for 2 hours at 37° C.) in the presence of the antibody is compared to a negative control in which no antibodies were added.

An antibody of the invention may block the interaction between the spike protein of SAR-CoV-2 with the cell surface receptor, angiotensin-converting enzyme 2 (ACE2), of the target cell, e.g. by direct blocking or by disrupting the pre-fusion conformation of the spike protein.

Blocking of the interaction between spike and ACE2 can be total or partial. For example, an antibody of the invention may reduce spike-ACE2 formation by ≥50%, ≥60%, ≥70%, ≥80%, ≥90%, ≥95%, ≥99% or 100%. Blocking of spike-ACE2 formation can be measured by any suitable means known in the art, for example, by ELISA.

Most antibodies showing neutralisation also showed blocking of the interaction between the spike protein and ACE2. Furthermore, a number of non-neutralising antibodies are good ACE2 blockers.

In terms of binding kinetics, an antibody of the invention may have an affinity constant (K_{D}) value for the spike protein of SARS-CoV-2 of ≤5 nM, ≤4 nM, ≤3 nM, ≤2 nM, ≤1 nM, ≤0.5 nM, ≤0.4 nM, ≤0.3 nM, ≤0.2 nM or ≤0.1 nM.

The KD value can be measured by any suitable means known in the art, for example, by ELISA or Surface Plasmon Resonance (Biacore) at 25° C.

Binding affinity (K_{D}) may be quantified by determining the dissociation constant (K_{d}) and association constant (Kₐ) for an antibody and its target. For example, the antibody may have an association constant (Kₐ) of ≥10000 M⁻¹s⁻¹, ≥50000 M⁻¹s⁻¹, ≥100000 M⁻¹s⁻¹, ≥200000 M⁻¹s⁻¹ or ≥500000 M⁻¹s⁻¹, and/or a dissociation constant (K_{d}) of ≤0.001 s⁻¹, ≤0.0005 s⁻¹, ≤0.004 s⁻¹, ≤0.003 s⁻¹, ≤0.002 s⁻¹ or ≤0.0001 s⁻¹.

An antibody of the invention is preferably able to provide in vivo protection in coronavirus (e.g. SARS-CoV-2) infected animals. For example, administration of an antibody of the invention to coronavirus (e.g. SARS-CoV-2) infected animals may result in a survival rate of ≥30%, ≥40%, ≥50%, ≥60%, ≥70%, ≥80%, ≥90%, ≥95% or 100%.

Survival rates may be determined using routine methods.

Antibodies of the invention may have any combination of one or more of the above properties.

Antibodies of the invention may bind to the same epitope as, or compete for binding to SARS-CoV-2 spike protein with, any one of the antibodies described herein (i.e. in particular with antibodies with the heavy and light chain variable regions described above). Methods for identifying antibodies binding to the same epitope, or cross-competing with one another, are used in the Examples and discussed further below.

### Fc Regions

An antibody of the invention may or may not comprise an Fc domain.

The antibodies of the invention may be modified in the Fc region in order to improve their stability. Such modifications are known in the art. Modifications may improve the stability of the antibody during storage of the antibody. The in vivo half-life of the antibody may be improved by modifications of the Fc-region. For example, cysteine residue(s) can be introduced into the Fc region, thereby allowing interchain disulphide bond formation in this region. The homodimeric antibody thus generated can have improved internalization capability and/or increased complement-mediated cell killing and antibody-dependent cellular cytotoxicity (ADCC). (See Caron et al., J. Exp Med., 176: 1191-1195 (1992) and Shopes, J. Immunol., 148: 2918-2922 (1992)).

Alternatively, an antibody can be engineered that has dual Fc regions and can thereby have enhanced complement lysis and ADCC capabilities. (See Stevenson et al., Anti-Cancer Drug Design, 3: 219-230 (1989)).

For example, an antibody of the invention may be modified to promote the interaction of the Fc domain with FcRn. The Fc domain may be modified to improve the stability of the antibody by affecting Fc and FcRn interaction at low pH, such as in the endosome. The M252Y/S254T/T256E (YTE) mutation may be used to improve the half-life of an IgG1 antibody.

The antibody may be modified to affect the interaction of the antibody with other receptors, such as FcγRI, FcγRIIA, FcγRIIB, FcγRIII, and FcαR. Such modifications may be used to affect the effector functions of the antibody.

In one embodiment, an antibody of the invention comprises an altered Fc domain as described herein below. In another preferred embodiment an antibody of the invention comprises an Fc domain, but the sequence of the Fc domain has been altered to modify one or more Fc effector functions.

In one embodiment, an antibody of the invention comprises a “silenced” Fc region. For example, in one embodiment an antibody of the invention does not display the effector function or functions associated with a normal Fc region. An Fc region of an antibody of the invention does not bind to one or more Fe receptors.

In one embodiment, an antibody of the invention does not comprise a CH₂ domain. In one embodiment, an antibody of the invention does not comprise a CH₃ domain. In one embodiment, an antibody of the invention comprises additional CH₂ and/or CH₃ domains.

In one embodiment, an antibody of the invention does not bind Fc receptors. In one embodiment, an antibody of the invention does not bind complement. In an alternative embodiment, an antibody of the invention does not bind FcγR, but does bind complement.

In one embodiment, an antibody of the invention in general may comprise modifications that alter serum half-life of the antibody. Hence, in another embodiment, an antibody of the invention has Fc region modification(s) that alter the half-life of the antibody. Such modifications may be present as well as those that alter Fc functions. In one preferred embodiment, an antibody of the invention has modification(s) that alter the serum half-life of the antibody.

In one embodiment, an antibody of the invention may comprise a human constant region, for instance IgA, IgD, IgE, IgG or IgM domains. In particular, human IgG constant region domains may be used, especially of the IgG1 and IgG3 isotypes when the antibody molecule is intended for therapeutic uses where antibody effector functions are required. Alternatively, IgG2 and IgG4 isotypes may be used when the antibody molecule is intended for therapeutic purposes and antibody effector functions are not required.

In one embodiment, the antibody heavy chain comprises a CH₁ domain and the antibody light chain comprises a CL domain, either kappa or lambda. In one embodiment, the antibody heavy chain comprises a CH₁ domain, a CH₂ domain and a CH₃ domain and the antibody light chain comprises a CL domain, either kappa or lambda.

The four human IgG isotypes bind the activating Fcγ receptors (FcγRI, FcγRIIa, FcγRIIc, FcγRIIIa), the inhibitory FcγRIIb receptor, and the first component of complement (C1q) with different affinities, yielding very different effector functions (Bruhns P. et al., 2009. Specificity and affinity of human Fcγ receptors and their polymorphic variants for human IgG subclasses. Blood. 113(16):3716-25), see also Jeffrey B. Stavenhagen, et al. Cancer Research 2007 Sep. 15; 67(18):8882-90. In one embodiment, an antibody of the invention does not bind to Fc receptors. In another embodiment of the invention, the antibody does bind to one or more type of Fc receptors.

In one embodiment the Fc region employed is mutated, in particular a mutation described herein. In one embodiment the Fc mutation is selected from the group comprising a mutation to remove or enhance binding of the Fc region to an Fc receptor, a mutation to increase or remove an effector function, a mutation to increase or decrease half-life of the antibody and a combination of the same. In one embodiment, where reference is made to the impact of a modification it may be demonstrated by comparison to the equivalent antibody but lacking the modification.

Some antibodies that selectively bind FcRn at pH 6.0, but not pH 7.4, exhibit a higher half-life in a variety of animal models. Several mutations located at the interface between the CH₂ and CH₃ domains, such as T250Q/M428L (Hinton PR. et al., 2004. Engineered human IgG antibodies with longer serum half-lives in primates. J Biol Chem. 279(8):6213-6) and M252Y/S254T/T256E+H433K/N434F (Vaccaro C. et al., 2005. Engineering the Fc region of immunoglobulin G to modulate in vivo antibody levels. Nat Biotechnol. 23(10):1283-8), have been shown to increase the binding affinity to FcRn and the half-life of IgG1 in vivo. Hence, modifications may be present at M252/S254/T256+H44/N434 that alter serum half-life and in particular M252Y/S254T/T256E+H433K/N434F may be present. In one embodiment, it is desired to increase half-life. In another embodiment, it may be actually desired to decrease serum half-life of the antibody and so modifications may be present that decrease serum half-life.

Numerous mutations have been made in the CH₂ domain of human IgG1 and their effect on ADCC and CDC tested in vitro (Idusogie EE. et al., 2001. Engineered antibodies with increased activity to recruit complement. J Immunol. 166(4):2571-5). Notably, alanine substitution at position 333 was reported to increase both ADCC and CDC. Hence, in one embodiment a modification at position 333 may be present, and in particular one that alters ability to recruit complement. Lazar et al. described a triple mutant (S239D/1332E/A330L) with a higher affinity for FcγRIIIa and a lower affinity for FcγRIIb resulting in enhanced ADCC (Lazar GA. et al., 2006). Hence, modifications at S239/I332/A330 may be present, particularly those that alter affinity for Fc receptors and in particular S239D/I332E/A330L. Engineered antibody Fc variants with enhanced effector function. PNAS 103(11): 4005-4010). The same mutations were used to generate an antibody with increased ADCC (Ryan MC. et al., 2007. Antibody targeting of B-cell maturation antigen on malignant plasma cells. Mol. Cancer Ther., 6: 3009-3018). Richards et al. studied a slightly different triple mutant (S239D/I332E/G236A) with improved FcγRIIIa affinity and FcγRIIa/FcγRIIb ratio that mediates enhanced phagocytosis of target cells by macrophages (Richards J O et al 2008. Optimization of antibody binding to Fcgamma RIIa enhances macrophage phagocytosis of tumor cells. Mol Cancer Ther. 7(8):2517-27). In one embodiment, S239D/I332E/G236A modifications may be therefore present.

In another embodiment, an antibody of the invention may have a modified hinge region and/or CH₁ region. Alternatively, the isotype employed may be chosen as it has a particular hinge regions.

### Major Public V Regions

Public V-regions, also described as public V-genes herein, are the V regions of the germline heavy chain and light chain regions that are found in a large proportion of the antibody responses to SARS-CoV-2 found within the population. In this application, the V regions are specific responses to the Beta SARS-CoV-2 variant. That is to say, many individuals utilise the same v-regions from their germline v-region repertoire when generating an immune response to SARS-CoV-2 variants.

As used herein, an antibody “derived” from a specific v-region refers to antibodies that were generated by V(D)J recombination using that germline v-region sequence. For example, the germline IGHV3-53 v-region sequence may undergo somatic recombination and somatic mutation to arrive at an antibody that specifically binds to the spike protein of SARS-CoV-2. The nucleotide sequence encoding the antibody is unlikely to comprise a sequence identical to the IGHV3-53 germline sequence, nevertheless, the antibody is still derived from this v-region. An antibody of the invention typically comprises no more than non-silent mutations in the v-region, when compared to the germline sequence, such as no more than 15, 10, 9, 8, 7, 6, 5, 4, 3, 2 or 1 non-silent mutations. An antibody of the invention typically comprises no between 2-20 non-silent mutations in the v-region, when compared to the germline sequence, such as between 5-15, 6-13 and 7-12 non-silent mutations. Germline v-region sequences are well known in the art, and methods of identifying whether a certain region of an antibody is derived from a particular germline v-region sequence are also well known in the art.

In one embodiment, an antibody of the invention derives from a v-region selected from IGHV3-53, IGHV1-58, IGHV3-66, IGHV1-69, IGHV3-30, IGHV3-33, IGHV1-18, IGHV13-9 or IGHV4-31. The inventors found that the potent neutralising antibodies identified herein comprised relatively few mutations in the CDRs of these v-regions. Thus, in one embodiment, an antibody of the invention encoded by a v-region selected from IGHV3-53, IGHV1-58, IGHV3-66, IGHV1-69, IGHV3-30, IGHV3-33, IGHV1-18, IGHV13-9 or IGHV4-31 and having 2-20 non-silent nucleotide mutations, or 5-15 non-silent mutations, such as 15 or less, 14 or less, 13 or less, 12 or less, 11 or less, 10 or less, 9 or less, 8 or less, 7 or less, 6 or less, 5 or less, 4 or less 3 or less or 2 non-silent mutations when compared to the naturally occurring germline sequence. A silent mutation as defined herein is a change in the nucleotide sequence without a change in the amino acid sequence for which the nucleotide sequence encodes. A non-silent mutation is therefore a mutation that leads to a change in the amino acid sequence encoded by the nucleotide sequence.

The inventors have surprisingly found that the light chain variable region of two antibodies having the same heavy chain v-region may be exchanged to produce a mixed-chain antibody comprising the heavy chain variable region of a first antibody and the light chain variable region of a second antibody. For example, the two antibodies may both comprise a heavy chain variable region derived from IGHV3-53. Preferably, both antibodies also comprise a light chain variable region derived from the same light chain v-region, although this is not essential because, for example, the light chain of antibody 222 may be matched with any heavy chain variable region derived from IGHV3-53 and lead to a potent neutralising antibody. As described above, the two antibodies may comprise a heavy chain variable region derived from IGHV3-53 and/or IGHV3-66.

In one embodiment, an antibody of the invention comprises the CDRs of an heavy chain variable domain of an antibody derived from a major public v-region selected from IGHV3-53, IGHV1-58, IGHV3-66, IGHV4-39, IGHV3-30, IGHV5-51, IGHV1-02 or IGHV3-33, such as antibodies Omi03, Omi18, Omi29, Beta-27, antibody 150, antibody 158, antibody 175, antibody 222 and antibody 269 for IGHV3-53, antibodies Omi16, Omi17, Omi20, Omi27, Omi36, antibody 40 and antibody 398 for IGHV3-66, antibodies Omi12, Beta-47, Beta-25, antibody 55, antibody 165, antibody 253 for IGHV1-58, antibodies Beta-49, Beta-50, Omi02, Omi24, Omi30, Omi31, Omi34 and Omi38 for IGHV1-69, antibodies Beta-22, Beta-29, antibody 159 and Omi09 for IGHV3-30, antibodies Beta-20, Beta-43, Omi32 and Omi 33 for IGHV3-33, antibodies antibody 278, Beta-44, Omi26 and Omi41 for IGHV1-18, antibodies 58, Omi25, Omi35 and Omi42 for IGHV3-9, or antibodies Beta-56 and Omi23 for IGHV4-31. The SEQ ID NOs corresponding to the CDRs of each of these antibodies are shown in Tables 1, 2 and 3.

In one embodiment, an antibody of the invention comprises the heavy chain variable domain of an antibody derived from a major public v-region selected from IGHV3-53, IGHV1-58, IGHV3-66, IGHV4-39, IGHV3-30, IGHV5-51, IGHV1-02 or IGHV3-33, such as antibodies Omi03, Omi18, Omi29, Beta-27, antibody 150, antibody 158, antibody 175, antibody 222 and antibody 269 for IGHV3-53, antibodies Omi16, Omi17, Omi20, Omi27, Omi36, antibody 40 and antibody 398 for IGHV3-66, antibodies Omi12, Beta-47, Beta-25, antibody 55, antibody 165, antibody 253 for IGHV1-58, antibodies Beta-49, Beta-50, Omi02, Omi24, Omi30, Omi31, Omi34 and Omi38 for IGHV1-69, antibodies Beta-22, Beta-29, antibody 159 and Omi09 for IGHV3-30, antibodies Beta-20, Beta-43, Omi32 and Omi 33 for IGHV3-33, antibodies antibody 278, Beta-44, Omi26 and Omi41 for IGHV1-18, antibodies 58, Omi25, Omi35 and Omi42 for IGHV3-9, or antibodies Beta-56 and Omi23 for IGHV4-31. The SEQ ID NOs corresponding to the CDRs of each of these antibodies are shown in Tables 1, 2 and 3.

In one embodiment, the invention provides a method of generating an antibody that binds specifically to the spike protein of SARS-CoV-2 (e.g. a SARS-CoV-2 strain of the Alpha, Beta, Gamma, Delta and/or Omicron lineages), the method comprising identifying two or more antibodies derived from the same light chain and/or heavy chain v-regions, replacing the light chain of a first antibody with the light chain of a second antibody, to thereby generate a mixed-chain antibody comprising the heavy chain of the first antibody and the light chain of the second antibody. In one embodiment, the method further comprises determining the affinity for and/or neutralisation of SARS-CoV-2 of the mixed-chain antibody. The method may further comprise comparing the affinity of the mixed-chain antibody with that of the first and/or second antibodies. The method may further comprise selecting a mixed chain antibody that has the same or greater affinity than the first and/or second antibodies. In some embodiments, the heavy chain v-region is IGHV 1-58 and/or the light chain v-region is IGLV Kappa 3-20.

In another embodiment, the invention provides an antibody that specifically binds to the Omicron variant of SARS-CoV-2, wherein the antibody has a v-region derived from IGHV1-69. It has been surprisingly discovered that antibody responses to infection with the Omicron variant of SARS-CoV-2 is biased towards antibodies with a heavy chain variable region derived from IGHV1-69. In one embodiment, wherein the antibody heavy chain is derived from IGHV1-69, the antibody of the invention comprises the CDRH1, CDRH2 and CDRH3 from Beta-49, Beta-50, Omi02, Omi24, Omi30, Omi31, Omi34 and Omi38.

### Antibody Conjugates

The invention also pertains to immunoconjugates comprising an antibody conjugated to a cytotoxic agent such as a toxin (e.g., an enzymatically active toxin of bacterial, fungal, plant, or animal origin, or fragments thereof), or a radioactive isotope (i.e., a radioconjugate). Conjugates of the antibody and cytotoxic agent may be made using a variety of bifunctional protein-coupling agents known in the art.

An antibody, of the invention may be conjugated to a molecule that modulates or alters serum half-life. An antibody, of the invention may bind to albumin, for example in order to modulate the serum half-life. In one embodiment, an antibody of the invention will also include a binding region specific for albumin. In another embodiment, an antibody of the invention may include a peptide linker which is an albumin binding peptide. Examples of albumin binding peptides are included in WO2015/197772 and WO2007/106120 the entirety of which are incorporated by reference.

### Polynucleotides, Vectors and Host Cells

The invention also provides one or more isolated polynucleotides (e.g. DNA) encoding the antibody of the invention. In one embodiment, the polynucleotide sequence is collectively present on more than one polynucleotide, but collectively together they are able to encode an antibody of the invention. For example, the polynucleotides may encode the heavy and/or light chain variable regions(s) of an antibody of the invention. The polynucleotides may encode the full heavy and/or light chain of an antibody of the invention. Typically, one polynucleotide would encode each of the heavy and light chains.

Polynucleotides which encode an antibody of the invention can be obtained by methods well known to those skilled in the art. For example, DNA sequences coding for part or all of the antibody heavy and light chains may be synthesised as desired from the corresponding amino acid sequences. General methods by which the vectors may be constructed, transfection methods and culture methods are well known to those skilled in the art. In this respect, reference is made to “Current Protocols in Molecular Biology”, 1999, F. M. Ausubel (ed), Wiley Interscience, New York and the Maniatis Manual produced by Cold Spring Harbor Publishing. A polynucleotide of the invention may be provided in the form of an expression cassette, which includes control sequences operably linked to the inserted sequence, thus allowing for expression of the antibody of the invention in vivo. Hence, the invention also provides one or more expression cassettes encoding the one or more polynucleotides that encoding an antibody of the invention. These expression cassettes, in turn, are typically provided within vectors (e.g. plasmids or recombinant viral vectors). Hence, in one embodiment, the invention provides a vector encoding an antibody of the invention. In another embodiment, the invention provides vectors which collectively encode an antibody of the invention. The vectors may be cloning vectors or expression vectors. A suitable vector may be any vector which is capable of carrying a sufficient amount of genetic information, and allowing expression of a polypeptide of the invention. The polynucleotides, expression cassettes or vectors of the invention are introduced into a host cell, e.g. by transfection. Hence, the invention also provides a host cell comprising the one or more polynucleotides, expression cassettes or vectors of the invention. The polynucleotides, expression cassettes or vectors of the invention may be introduced transiently or permanently into the host cell, allowing expression of an antibody from the one or more polynucleotides, expression cassettes or vectors. Such host cells include transient, or preferably stable higher eukaryotic cell lines, such as mammalian cells or insect cells, lower eukaryotic cells, such as yeast, or prokaryotic cells, such as bacteria cells. Particular examples of cells include mammalian HEK293, such as HEK293F, HEK293T, HEK293S or HEK Expi293F, CHO, HeLa, NSO and COS cells, or any other cell line used herein, such as the ones used in the Examples. Preferably the cell line selected will be one which is not only stable, but also allows for mature glycosylation.

The invention also provides a process for the production of an antibody of the invention, comprising culturing a host cell containing one or more vectors of the invention under conditions suitable for the expression of the antibody from the one or more polynucleotides of the invention, and isolating the antibody from said culture.

### Combination of Antibodies

The inventors found that certain Table 3 antibodies are particularly effective when used in combination, and certain combinations of Table 3, Table 2, and Table 1 antibodies, e.g. to minimise loss of activity due to SARS-CoV-2 variants, maximise therapeutic effects and/or increase diagnostic power. Useful combinations include the antibodies that do not cross-compete with one another and/or bind to non-overlapping epitopes.

Thus, the invention provides a combination of the antibodies of the invention, wherein each antibody is capable of binding to the spike protein of coronavirus SARS-CoV-2, wherein at least one antibody comprises at least three CDRs of any one of the 28 antibodies in Table 3.

A combination of the antibodies of the invention may be useful as a therapeutic cocktail. Hence, the invention also provides a pharmaceutical composition comprising a combination of the antibodies of the invention, as explained further below.

A combination of the antibodies of the invention may be useful for diagnosis. Hence, the invention also provides a diagnostic kit comprising a combination of the antibodies of the invention. Also provided herein are methods of diagnosing a disease or complication associated with coronavirus infections in a subject, as explained further below. A fully cross-neutralising antibody, e.g. Omi03, may be used as a reference to confirm the presence and/or amount of any variants of concern (VoC) SARS-CoV-2 in the sample. An antibody that binds to a limited number of VoCs may be used to confirm the presence and/or amount of that VoC in the sample.

For example, if Omi03 exhibits binding to the sample but Omi24 does not exhibit binding to the sample of SARS-CoV-2, then the spike protein may be the spike protein of the Delta VoC. This may be determined by any method known to the skilled person, such as via an immunoassay, e.g. an ELISA or an immunochromatographic assay. Reduced binding may be determined by comparison and/or normalisation to the reference, and/or by comparison to positive/negative control samples or data.

### Pharmaceutical Composition

The invention provides a pharmaceutical composition comprising an antibody of the invention. The composition may comprise a combination (such as two, three or four) of the antibodies of the invention. The pharmaceutical composition may also comprise a pharmaceutically acceptable carrier.

The composition of the invention may include one or more pharmaceutically acceptable salts. A “pharmaceutically acceptable salt” refers to a salt that retains the desired biological activity of the parent compound and does not impart any undesired toxicological effects. Examples of such salts include acid addition salts and base addition salts.

Suitable pharmaceutically acceptable carriers comprise aqueous carriers or diluents. Examples of suitable aqueous carriers include water, buffered water and saline.

Other suitable pharmaceutically acceptable carriers include ethanol, polyols (such as glycerol, propylene glycol, polyethylene glycol, and the like), and suitable mixtures thereof, vegetable oils, such as olive oil, and injectable organic esters, such as ethyl oleate. In many cases, it will be desirable to include isotonic agents, for example, sugars, polyalcohols such as mannitol, sorbitol, or sodium chloride in the composition.

Therapeutic compositions typically must be sterile and stable under the conditions of manufacture and storage. The composition can be formulated as a solution, microemulsion, liposome, or other ordered structure suitable to high drug concentration.

Pharmaceutical compositions of the invention may comprise additional therapeutic agents, for example an anti-viral agent. The anti-viral agent may bind to coronavirus and inhibit viral activity. Alternatively, the anti-viral agent may not bind directly to coronavirus but still affect viral activity/infectivity. The anti-viral agent could be a further anti-coronavirus antibody, which binds somewhere on SARS-CoV-2 other than the spike protein. Examples of an anti-viral agent useful with the invention include Remdesivir, Lopinavir, ritonavir, APN01, and Favilavir.

The additional therapeutic agent may be an anti-inflammatory agent, such as a corticosteroid (e.g. Dexamethasone) or a non-steroidal anti-inflammatory drug (e.g. Tocilizumab).

The additional therapeutic agent may be an anti-coronavirus vaccine. The pharmaceutical composition may be administered subcutaneously, intravenously, intradermally, intramuscularly, intranasally or orally. Also within the scope of the invention are kits comprising antibodies or other compositions of the invention and instructions for use. The kit may further contain one or more additional reagents, such as an additional therapeutic or prophylactic agent as discussed herein.

Methods and uses of the invention The invention further relates to the use of the antibodies, the combinations of the antibodies and the pharmaceutical compositions, described herein, e.g. in a method for treatment of the human or animal body by therapy, or in a diagnostic method. The method of treatment may be therapeutic or prophylactic.

For example, the invention relates to methods of treating coronavirus (e.g. SARS-CoV-2) infections, a disease or complication associated therewith, e.g. COVID-19. The method may comprise administering a therapeutically effective amount of an antibody, a combination of antibodies, or a pharmaceutical composition of the invention. The method may further comprise identifying the presence of coronavirus, or fragments thereof, in a sample, e.g. SARS-CoV-2, from the subject. The invention also relates to an antibody, a combination of antibodies, or a pharmaceutical composition according to the invention for use in a method of treating coronavirus (e.g. SARS-CoV-2) infections, a disease or complication associated therewith, e.g. COVID-19.

The invention also relates to a method of formulating a composition for treating coronavirus (e.g. SARS-CoV-2) infections, a disease or complication associated therewith, e.g. COVID-19, wherein said method comprises mixing an antibody, a combination of antibodies, or a pharmaceutical composition according to the invention with an acceptable carrier to prepare said composition.

The invention also relates to the use of an antibody, a combination of antibodies, or a pharmaceutical composition according to the invention for treating coronavirus (e.g. SARS-CoV-2) infections or a disease or complication associated therewith, e.g. COVID-19.

The invention also relates to the use of an antibody, a combination of antibodies, or a pharmaceutical composition according to the invention for the manufacture of a medicament for treating or preventing coronavirus (e.g. SARS-CoV-2) infections or a disease or complication associated therewith, e.g. COVID-19.

The invention also relates to preventing, treating or diagnosing coronavirus infection caused by any SARS-CoV-2 strain. The coronavirus infection may be caused by any SARS-CoV-2 strain.

The SARS-CoV-2 strain may be the earliest identified Wuhan strain (hCoV-19/Wuhan/WIV04/2019 (WIV04); GISAID accession no. EPI_ISL_402124), and variants thereof. For example, the SARS-CoV-2 strain may be a member of lineage A, A.1, A.2, A.3, A.5, B, B.1, B.1.1, B.2, B.3, B.4, B.1.1.7 (alpha), B.1.351 (beta), P.1 (gamma), delta, kappa, and/or lambda. The SARS-CoV-2 strain may be a member of lineage A.23.1, B.1.1.7 (alpha), B.1.351 (beta), B.1.258, B.1.526.2, B.1.616, B.1.617.1 (kappa), B.1.617.2 (delta), C36.3, C.37 (lambda), P.1 (gamma), B.1.1.529 (omicron), Omicron BA.1, Omicron BA.1.1, Omicron BA.2 and/or Omicron BA.3.

The SARS-CoV-2 strain may comprise one or more mutations, e.g. in the spike protein, relative to the hCoV-19/Wuhan/WIV04/2019 (WIV04) (GISAID accession no. EPI_ISL_402124). In other words, the SARS-CoV-2 strain may be a modified hCoV-19/Wuhan/WIV04/2019 (WIV04) strain comprising one or more modifications, e.g. in the spike protein.

The mutation may be the mutations (e.g. substitutions) observed in the Omicron strain of SARS-CoV-2.

Antibodies Omi02, Omi03, Omi12, Omi18, Omi28, Omi39 and Omi42 are particularly effective in neutralising the Omicron SARS-Cov-2 strain. Hence, the invention may relate to these antibodies for use in treating, prevent, treating or diagnosing coronavirus infection caused by a SARS-Cov-2 strain.

The methods and uses of the invention may comprise inhibiting the disease state (such as COVID-19), e.g. arresting its development; and/or relieving the disease state (such as COVID-19), e.g. causing regression of the disease state until a desired endpoint is reached.

The methods and uses of the invention may comprise the amelioration or the reduction of the severity, duration or frequency of a symptom of the disease state (such as COVID-19) (e.g. lessen the pain or discomfort), and such amelioration may or may not be directly affecting the disease. The symptoms or complications may be fever, headache, fatigue, loss of appetite, myalgia, diarrhoea, vomiting, abdominal pain, dehydration, respiratory tract infections, cytokine storm, acute respiratory distress syndrome (ARDS) sepsis, and/or organ failure (e.g. heart, kidneys, liver, GI, lungs).

The methods and uses of the invention may lead to a decrease in the viral load of coronavirus (e.g. SARS-CoV-2), e.g. by ≥10%, ≥20%, ≥30%, ≥40%, ≥50%, ≥60%, ≥70%, ≥80%, ≥90%, or 100% compared to pre-treatment. Methods of determining viral load are well known in the art, e.g. infection assays.

The methods and uses of the invention may comprise preventing the coronavirus infection from occurring in a subject (e.g. humans), in particular, when such subject is predisposed to complications associated with coronavirus infection.

The invention also relates to identifying subjects that have a coronavirus infection, such as by SARS-CoV-2. For example, the methods and uses of the invention may involve identifying the presence of coronavirus (e.g. SARS-CoV-2), or a protein or a fragment thereof, in a sample. The detection may be carried out in vitro or in vivo. In certain embodiments, the invention relates to population screening.

The invention relates to identifying any SARS-CoV-2 strain, as described herein. The invention may also relate to a method of identifying escape mutants of SARS-CoV-2, comprising contacting a sample with a combination of antibodies of the invention and identifying if each antibody binds to the virus. The term “escape mutants” refers to variants of SARS-CoV-2 comprising non-silent mutations that may affect the efficacy of existing treatments of SARS-CoV-2 infection. Typically, the non-silent mutations is on an epitope recognised by a prior art antibody and/or antibodies described herein that specifically binds to an epitope of SARS-CoV-2, e.g. on the spike protein of SARS-CoV-2. If the antibody does not bind to the target, it may indicate that the target comprises a mutation that may alter the efficacy of existing SARS-CoV-2 treatments.

The methods and uses of the invention may include contacting a sample with an antibody or a combination of the antibodies of the invention, and detecting the presence or absence of an antibody-antigen complex, wherein the presence of the antibody-antigen complex indicates that the subject is infected with SARS-CoV-2.

Methods of determining the presence of an antibody-antigen complex are known in the art. For example, in vitro detection techniques include enzyme linked immunosorbent assays (ELISAs), Western blots, immunoprecipitations, and immunofluorescence. In vivo techniques include introducing into a subject a labelled anti-analyte protein antibody. For example, the antibody can be labelled with a radioactive marker whose presence and location in a subject can be detected by standard imaging techniques. The detection techniques may provide a qualitative or a quantitative readout depending on the assay employed.

Typically, the invention relates to methods and uses for a human subject in need thereof. However, non-human animals such as rats, rabbits, sheep, pigs, cows, cats, or dogs is also contemplated. The subject may be at risk of exposure to coronavirus infection, such as a healthcare worker or a person who has come into contact with an infected individual. A subject may have visited or be planning to visit a country known or suspected of having a coronavirus outbreak. A subject may also be at greater risk, such as an immunocompromised individual, for example an individual receiving immunosuppressive therapy or an individual suffering from human immunodeficiency syndrome (HIV) or acquired immune deficiency syndrome (AIDS). The subject may be asymptomatic or pre-symptomatic.

The subject may be early, middle or late phase of the disease.

The subject may be in hospital or in the community at first presentation, and/or later times in hospital.

The subject may be male or female.

In certain embodiments, the subject is typically male. The subject may not have been infected with coronavirus, such as SARS-CoV-2. The subject may have a predisposition to the more severe symptoms or complications associated with coronavirus infections. The method or use of the invention may comprise a step of identifying whether or not a patient is at risk of developing the more severe symptoms or complications associated with coronavirus.

In embodiments of the invention relating to prevention or treatment, the subject may or may not have been diagnosed to be infected with coronavirus, such as SARS-CoV-2.

The invention relates to analysing samples from subjects. The sample may be tissues, cells and biological fluids isolated from a subject, as well as tissues, cells and fluids present within a subject. The sample may be blood and a fraction or component of blood including blood serum, blood plasma, or lymph. Typically, the sample is from a throat swab, nasal swab, or saliva.

The antibody-antigen complex detection assays may be performed in situ, in which case the sample is a tissue section (fixed and/or frozen) of the tissue obtained from biopsies or resections from a subject.

In the embodiments of the invention where the antibodies pharmaceutical compositions and combinations are administered, they may be administered subcutaneously, intravenously, intradermally, orally, intranasally, intramuscularly or intracranially. Typically, the antibodies pharmaceutical compositions and combinations are administered intravenously or subcutaneously.

The dose of an antibody may vary depending on the age and size of a subject, as well as on the disease, conditions and route of administration. Antibodies may be administered at a dose of about 0.1 mg/kg body weight to a dose of about 100 mg/kg body weight, such as at a dose of about 5 mg/kg to about 10 mg/kg. Antibodies may also be administered at a dose of about 50 mg/kg, 10 mg/kg or about 5 mg/kg body weight.

A combination of the invention may for example be administered at a dose of about 5 mg/kg to about 10 mg/kg for each antibody, or at a dose of about 10 mg/kg or about 5 mg/kg for each antibody. Alternatively, a combination may be administered at a dose of about 5 mg/kg total (e.g. a dose of 1.67 mg/kg of each antibody in a three antibody combination).

The antibody or combination of antibodies of the invention may be administered in a multiple dosage regimen. For example, the initial dose may be followed by administration of a second or plurality of subsequent doses. The second and subsequent doses may be separated by an appropriate time.

As discussed above, the antibodies of the invention are typically used in a single pharmaceutical composition/combination (co-formulated). However, the invention also generally includes the combined use of antibodies of the invention in separate preparations/compositions. The invention also includes combined use of the antibodies with additional therapeutic agents, as described above.

Combined administration of the two or more agents and/or antibodies may be achieved in a number of different ways. In one embodiment, all the components may be administered together in a single composition. In another embodiment, each component may be administered separately as part of a combined therapy.

For example, the antibody of the invention may be administered before, after or concurrently with another antibody, or binding fragment thereof, of the invention. The particularly useful combinations are described above for example.

For example, the antibody of the invention may be administered before, after or concurrently with an anti-viral agent or an anti-inflammatory agent.

In embodiments where the invention relates to detecting the presence of coronavirus, e.g. SARS-CoV-2, or a protein or a fragment thereof, in a sample, the antibody contains a detectable label. Methods of attaching a label to an antibody are known in the art, e.g. by direct labelling of the antibody by coupling (i.e., physically linking) a detectable substance to the antibody. Alternatively, the antibody may be indirect labelled, e.g. by reactivity with another reagent that is directly labelled. Examples of indirect labelling include detection of a primary antibody using a fluorescently-labelled secondary antibody and end-labelling of a DNA probe with biotin such that it can be detected with fluorescently-labelled streptavidin.

The detection may further comprise: (i) an agent known to be useful for detecting the presence of coronavirus, e.g. SARS-CoV-2, or a protein or a fragment thereof, e.g. an antibody against other epitopes of the spike protein, or other proteins of the coronavirus, such as an anti-nucleocapsid antibody; and/or (ii) an agent known to not be capable of detecting the presence of coronavirus, e.g. SARS-CoV-2, or a fragment thereof, i.e. providing a negative control.

In certain embodiments, the antibody is modified to have increased stability. Suitable modifications are explained above.

The invention also encompasses kits for detecting the presence of coronavirus, e.g. SARS-CoV-2, in a sample. For example, the kit may comprise: a labelled antibody or a combination of labelled antibodies of the invention; means for determining the amount of coronavirus, e.g. SARS-CoV-2, in a sample; and means for comparing the amount of coronavirus, e.g. SARS-CoV-2, in the sample with a standard. The labelled antibody or the combination of labelled antibodies can be packaged in a suitable container. The kit can further comprise instructions for using the kit to detect coronavirus, e.g. SARS-CoV-2, in a sample. The kit may further comprise other agents known to be useful for detecting the presence of coronavirus, as discussed above.

For example, the antibodies or combinations of antibodies of the invention are used in a lateral flow test. Typically, the lateral flow test kit is a hand-held device with an absorbent pad, which based on a series of capillary beds, such as pieces of porous paper, microstructured polymer, or sintered polymer. The test runs the liquid sample along the surface of the pad with reactive molecules that show a visual positive or negative result. The test may further comprise using other agents known to be useful for detecting the presence of coronavirus, e.g. SARS-CoV-2, or a fragment thereof, as discussed above, such as anti-an anti-nucleocapsid antibody.

### Other

It is to be understood that different applications of the disclosed antibodies combinations, or pharmaceutical compositions of the invention may be tailored to the specific needs in the art. It is also to be understood that the terminology used herein is for the purpose of describing particular embodiments of the invention only, and is not intended to be limiting. In addition as used in this specification and the appended claims, the singular forms “a”, “an”, and “the” include plural references unless the content clearly dictates otherwise. Thus, for example, reference to “an antibody” includes two or more “antibodies”.

Furthermore, when referring to “≥x” herein, this means equal to or greater than x. When referred to “≤x” herein, this means less than or equal to x.

For the purpose of this invention, in order to determine the percent identity of two sequences (such as two polynucleotide or two polypeptide sequences), the sequences are aligned for optimal comparison purposes (e.g. gaps can be introduced in a first sequence for optimal alignment with a second sequence). The nucleotide or amino acid residues at each position are then compared. When a position in the first sequence is occupied by the same nucleotide or amino acid as the corresponding position in the second sequence, then the nucleotides or amino acids are identical at that position. The percent identity between the two sequences is a function of the number of identical positions shared by the sequences (i.e., % identity=number of identical positions/total number of positions in the reference sequence×100). Typically the sequence comparison is carried out over the length of the reference sequence. For example, if the user wished to determine whether a given (“test”) sequence is 95% identical to SEQ ID NO: 3, SEQ ID NO: 3 would be the reference sequence. To assess whether a sequence is at least 95% identical to SEQ ID NO: 3 (an example of a reference sequence), the skilled person would carry out an alignment over the length of SEQ ID NO: 3, and identify how many positions in the test sequence were identical to those of SEQ ID NO: 3. If at least 95% of the positions are identical, the test sequence is at least 95% identical to SEQ ID NO: 3. If the sequence is shorter than SEQ ID NO: 3, the gaps or missing positions should be considered to be non-identical positions. The skilled person is aware of different computer programs that are available to determine the homology or identity between two sequences. For instance, a comparison of sequences and determination of percent identity between two sequences can be accomplished using a mathematical algorithm. In an embodiment, the percent identity between two amino acid or nucleic acid sequences is determined using the Needleman and Wunsch (1970) algorithm which has been incorporated into the GAP program in the Accelrys GCG software package (available at http://www.accelrys.com/products/gcg/), using either a Blosum 62 matrix or a PAM250 matrix, and a gap weight of 16, 14, 12, 10, 8, 6, or 4 and a length weight of 1, 2, 3, 4, 5, or 6.

The CDRs of the heavy chain (CDRH) and light chain variable domain (CDRL) are located at residues 27-38 (CDR1), residues 56-65 (CDR2) and residues 105-117 (CDR3) of each chain according to the IMGT numbering system (http://www.imgt.org; Lefranc MP, 1997, J, Immunol. Today, 18, 509). This numbering system is used in the present specification except where otherwise indicated.

All publications, patents and patent applications cited herein, whether supra or infra, are hereby incorporated by reference in their entirety.

The following examples illustrate the invention.

### EXAMPLES

### Example 1. Generation of Antibodies Specific Against Early Pandemic SARS-CoV-2 and Beta SARS-CoV-2 Strains

The antibodies in Table 1 relate to a set of mAbs generated against early pandemic strain of SARS-CoV-2. The antibodies in Table 2 relate to a set of mAbs generated against the Beta strain of SARS-CoV-2.

Further details of these antibodies may be found in international application no. PCT/GB2022/050306 & PCT/GB2022/050307. Further information on the generation and properties of these antibodies may be found in the following articles: Dejnirattisai, Wanwisa, et al. “The antigenic anatomy of SARS-CoV-2 receptor binding domain.” Cell 184.8 (2021): 2183-2200. Supasa, Piyada, et al. “Reduced neutralization of SARS-CoV-2 B. 1.1. 7 variant by convalescent and vaccine sera.” Cell 184.8 (2021): 2201-2211.

Liu, Chang, et al. “The antibody response to SARS-CoV-2 Beta underscores the antigenic distance to other variants.” Cell host & microbe (2021).

Zhou, Daming, et al. “Evidence of escape of SARS-CoV-2 variant B. 1.351 from natural and vaccine-induced sera.” Cell 184.9 (2021): 2348-2361.

Dejnirattisai, Wanwisa, et al. “Antibody evasion by the P. 1 strain of SARS-CoV-2.” Cell 184.11 (2021): 2939-2954.

Liu, Chang, et al. “Reduced neutralization of SARS-CoV-2 B. 1.617 by vaccine and convalescent serum.” Cell 184.16 (2021): 4220-4236.

Dejnirattisai, Wanwisa, et al. “SARS-CoV-2 Omicron-B. 1.1. 529 leads to widespread escape from neutralizing antibody responses.” Cell (2022).

### Example 2. Generation of Antibodies Specific Against the Omicron Strains of SARS-CoV-2

### Omicron BA.2 Lineage

Omicron BA.2 was first reported from South Africa on the 17th November 2021, at a similar time that Omicron BA.1 was reported. BA.2 has been increasing relative to BA.1 in a number of countries such as Denmark, India and the UK and now accounts for the majority of Omicron infections in Denmark and evidence is accruing that BA.2 is more transmissible than BA.1, but there is no evidence for increased disease severity.

BA.2 is related to BA.1 sharing 21 amino acid substitutions spread throughout in S, however there are a number of differences BA.1, has an additional 6 amino acid deletions, 3 insertions and 9 substitutions compared to BA.2 and BA.2 has an additional 3 deletions and 7 substitutions compared to BA.1. In the RBD, BA.1 contains unique mutations S371L, G446S and G496S and in some isolates R346K (BA.1.1), while BA.2 carries S371F, T376A, D405N and R408S. All of these residues have the potential to differentially affect antibody binding and could modulate neutralization, particularly BA.1 G446S, G496S, BA.2 D405N, R408S which lie at the edge of the ACE2 binding footprint and for BA.1.1 the R346K change lies close to the N343 glycan and could modulate binding of potent antibodies to this region. BA.3 contains no unique mutations relative to BA.1 and BA.2 and appears to be a fusion of the two, being BA.1 like at the N terminus and switching to become BA.2 like at the C-terminus from the mutation G496S.

### Omicron Lineages BA.4 and BA.5

In early April 2022 two new Omicron lineages were reported from Gauteng in South Africa and designated BA.4 and BA.5. The BA.4 and BA.5 S sequences are identical, and closely related to BA.2. Sequence diversity in Omicron S is shown in FIG. 9. Compared to BA.2, BA.4 has residues 69 and 70 deleted, and contains 2 additional substitutions in the RBD: L452R and F486V. Finally BA.4 lacks the Q493R change seen in BA.1 and BA.2, reverting to Q493 as in the Victoria/Wuhan strain.

The 2 additional mutations in the RBD are of most concern in terms of antibody escape: L452R is a chemically radical change and is one of the pair of changes in Delta RBD (the other, T478K, is already found in the Omicron lineage). Mutation F486L was found in sequences of SARS-CoV-2 isolated from Mink early in the pandemic and is also a site of escape mutations to several mAbs (Gobeil et al., 2021, “Effect of natural mutations of SARS-CoV-2 on spike structure, conformation, and antigenicity”. Science 373, 6555). The change F486V in BA.4/5 is also a reduction in the bulk of the hydrophobic side-chain as in F486L, but more significant. Both residues 452 and 486 lie close to the edge of the ACE2 interaction surface (FIG. 9B) and both, together with the reversion to ancestral sequence Q493 which lies within the ACE2 footprint, have the potential to modulate ACE2 affinity as well as modulate the neutralizing capacity of vaccine or naturally acquired serum. The L452R and F486V mutations are likely to cause more antibody escape, while the reversion at 493 may reduce the escape from responses to earlier viruses.

### The Omicron Lineage BA.2.75

In early May 2022, a new Omicron BA.2 sublineage designated BA.2.75 was reported in India. It has spread to multiple countries, including the UK, US, Australia, Germany and Canada. BA.2.75 contains multiple mutational changes in the S protein compared to BA.2, including four substitutions in the NTD (W152R, F157L, 1210V and G257S) and four in the RBD: D339H, G446S, N460K and R493Q (FIG. 16). The RBD mutations impinge on major epitopes for neutralising antibodies and are likely to modulate ACE2 binding. D339H represents a further evolution of the G339D mutation found in all previous Omicron variants that has been found to impair the binding of certain ‘right-flank’ antibodies belonging to the IGHV1-69 family (e.g. Beta-49 and -50); it also falls in the binding footprint of certain Class 3 antibodies such as S309/sotrovimab (Dejnirattisai et al., 2022; “SARS-CoV-2 Omicron-B.1.1.529 leads to widespread escape from neutralizing antibody responses.” Cell 185, 467-484 e415). G446S was found in BA.1, BA.1.1 and BA.3 but not in BA.2 and other BA.2 subvariants, and is also able to impair binding of certain Class 3 antibodies binding the right shoulder such as REGN10987/imdevimab (Dejnirattisai et al., 2022). The R493Q reversion was also found in BA.4/5, and may make the virus more sensitive to neutralization by a number of class 1 and 2 antibodies binding the neck/left shoulder. This reversion may also increase the affinity for ACE2 (see below).

N460K is a novel mutation not seen in previous VoC or Omicron sublineages, but it was found after in vitro (yeast display) evolution in RBD-62 which has an ultra-high ACE2 affinity (KD=16-18 pM) (Dejnirattisai et al., 2022; Zahradnik et al., 2021 “SARS-CoV-2 variant prediction and antiviral drug design are enabled by RBD in vitro evolution”. Nat Microbiol 6, 1188-1198). Indeed N460K led to substantial increase in affinity for ACE2, second only to the effect of N501Y (Zahradnik et al., 2021). Furthermore, in silico analysis predicts that N460K may affect the binding of certain antibodies belonging to the IGHV3-53 family (e.g. Omi-3) which have been shown to be able to potently neutralise all VoC (Nutalai et al., 2022).

Using neutralization assays, Delta infection in isolation was show to provide no protection (no neutralization) against BA.2.75. The mutations in BA.2.75 lead to a reduction in neutralization titres of vaccine serum compared to BA.2. Individual BA.2.75 mutations can cause greater reduction in neutralization titres compared to the full BA.2.75 S sequence, but these are balanced by the R393Q reversion mutation, which may have been selected to increase affinity to ACE2 and increase the transmissibility of BA.2.75. It seems inevitable that further evolution of the Omicron lineage will occur and there are likely many possible trade-offs between antibody escape and ACE2 affinity, that can and will be made, leading to successive waves of infection.

### Emerging BA.2, BA.4 and BA.5 Sublineages

A number of lineages are growing rapidly from within both the BA.2 and BA.5 branches. Most striking, is the large degree of convergent evolution, particularly at antigenic RBD positions such as 346, 444, 452, 460, 486, 490, 493 and 494. These lineages include examples from the BA.4/5 branches (which contain L452R, F486V and the reversion R493Q), such as BA.4.6 and BF.7 (R346T), BA.4.7 (R346S), BQ.1 (K444T, N460K) and BQ.1.1 (R346T, K444T, N460K); from the BA.2.75 branch (which contains G339H, G446S, N460K and the reversion R493Q), BA.2.75.2 (R346T and F486S and BA.2.75 mutations), BN.1 (aka BA.2.75.5.1 with R346T, K356T, F490S and BA.2.75 mutations), BM.1.1.1 (aka BA.2.75.3.1.1.1 with R346T, F486S, F490S and BA.2.75 mutations). There are also examples of several other second generation BA.2 variant lines such as BJ.1 (aka BA.2.10.1.1; G339H, R346T, L368I, V445P, G446S, V483A and F490V), BA.2.10.4 (G446S, F486P, S494P and the R493Q reversion), BS.1 (aka BA.2.3.2.1; R346T, L452R, N460K, G476S and the Q493R reversion), BA.2.3.20 (K444R, N450D, L452M, N460K, E484R and the Q493R reversion), and finally a BJ.1×BM.1.1.1 (aka BA.2.75.3.1.1.1) recombinant, XBB (which relative to BA.2 contains R346T, L368I, V445P, G446S, N460K, F486S, F490S and the Q493R reversion).

Outside the RBD the degree of convergent evolution is lesser but still present. Many of the second-generation BA.2 variant lineages contain deletions or mutations in the NTD, often similar to those seen in the VoCs, for example Δ˜144 in BJ.1, BS.1, and BA.2.10.4 (previously seen in Alpha and BA.1) and NSP12 G671S in BJ.1, XBB and BA.2.10.4 (previously seen in Delta).

### Potently Neutralizing Antibodies Isolated Following Omicron Infection

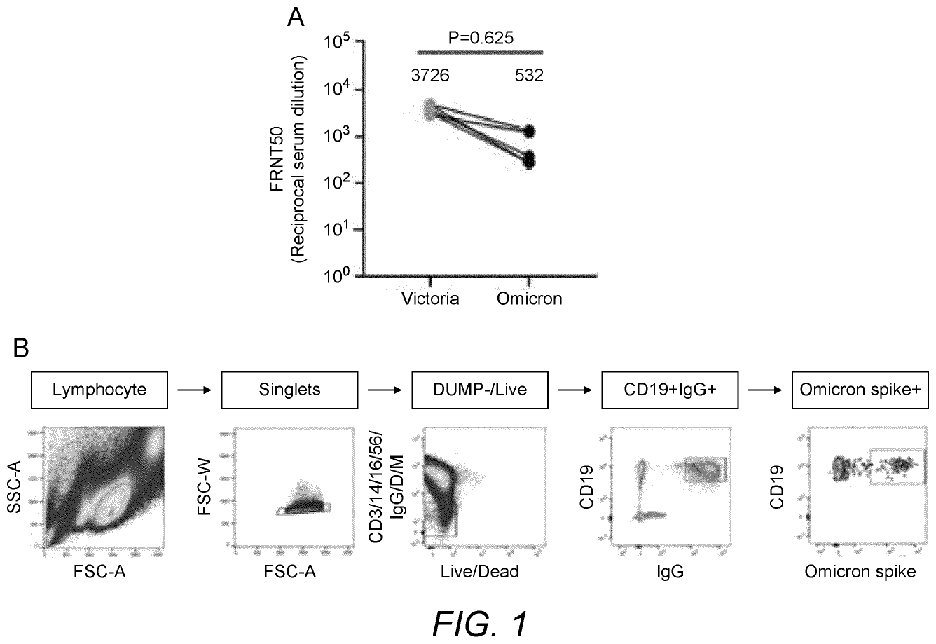

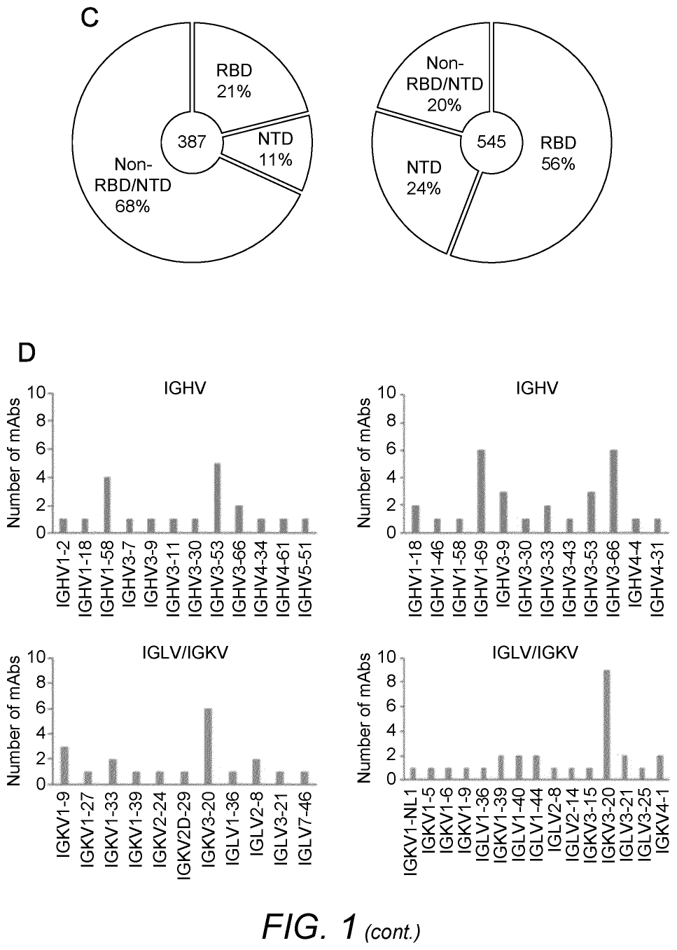

Five volunteers who had recovered from sequence confirmed Omicron infection were recruited and sampled them 10-14 days following symptom onset; all volunteers had received 2 doses of the Pfizer BioNtech vaccine before being infected with Omicron. First, neutralization assays were performed against Omicron BA.1 and Victoria (an early pandemic SARS-CoV-2 isolate containing only a single amino acid substitution in S NTD (S247R) compared to the sequence of the Wuhan stain used in all current vaccines). In all cases the focus reduction neutralization 50% titre (FRNT50) to Omicron was above 100, but at this early time point the titres were considerably below the titres to Victoria (FIG. 1A).

B cells from the five donors were stained with full length BA.1 trimer and single cells sorted by FACS (FIG. 1B). Following a degenerate RT-PCR reaction, heavy and light chain sequences were assembled into expression vectors using the Gibson reaction and the products transfected into 293T cells. Culture supernatants were screened for reactivity to full length BA.1 or wild type S (WT Wuhan) together with BA.1 RBD and NTD. In total 1,122 single cells were sorted and 545 mAb recovered.

All mAbs cross-reacted between WT and BA.1 S by ELISA, suggesting that they could have been generated from memory B cells induced by vaccination. In contrast to a previous panel of monoclonal antibodies were produced from naïve cases infected early during the pandemic (Dejnirattisai, Wanwisa, et al. “The antigenic anatomy of SARS-CoV-2 receptor binding domain.” Cell 184.8 (2021): 2183-2200), a higher proportion the omicron-specific mAbs were found to react to the RBD (56%) when compared to the early pandemic mAbs (21%, p<0.0001) (FIG. 1C). In addition 129 of the 545 isolated mAbs bound the BA.1 NTD.

### Isolation of Potent Omicron mAb

Neutralization assays were performed on all ELISA positive mAb and those showing the highest activity were chosen for further study. The most potent 28 mAbs were selected for full characterization all of which showed BA.1 FRNT50 titres <100 ng/ml. 27/28 bound the RBD (one, Omi-41 bound the NTD) and none cross-reacted with SARS-CoV-1 S protein by ELISA.

Examination of gene usage (FIG. 1D, Table 17) revealed that 9/28 mAbs belong to the VH3-53 and the related VH3-66 gene families. VH3-53 and VH3-66 have been isolated repeatedly in SARS-CoV-2 infection, they form a public antibody response and bind to a site on the neck of the RBD and function to block ACE2 binding. It was previously observed that many VH3-53 and VH3-66 mAbs lose activity on VoCs containing the N501Y mutation, although some VH3-53 antibodies (mAb 222 and Beta-27) were fully resistant to the N501Y change found in Alpha, Beta and Gamma but suffered knock down of activity to Omicron BA.1 or BA.2.

Roughly one half of the gene families observed in the potent early pandemic antibodies (Table 1) are also represented in the Omicron set (FIG. 1C), perhaps the most notable difference is that VH1-69 does not feature in the early antibodies but is found in 6/28 of the potent Omicron set (2, 24, 30, 31, 34 and 38) and we also found it in 2 Beta antibodies, Beta 49 and 50, which bind to a site in proximity to the N343 glycan. Analysis of the Omicron mAb shows much longer CDR3 sequences suggesting a different mode of binding than Beta 49, 50. In the Beta set of mAb, expansion of a public response was found to be mediated through VH 4-39 (6/27 mAb), which bound an epitope around the 501Y mutation, most lost activity against BA.1 and it is noteworthy that none of the current set of Omicron mAb are encoded by VH4-39.

Compared to the early pandemic set of antibodies we found higher levels of somatic mutation in both heavy and light chains compared to the early pandemic set of mAb Omicron (mean of 9.00, 6.00 and early pandemic 4.55, 4.25 for VH and VL respectively). These results would be consistent with the evolution of increased Omicron affinity via somatic mutation of vaccine induced memory B cells.

### Broad Neutralization of VoC by Omicron mAb

Neutralization assays were performed against Victoria and all variants of concern Alpha, Beta, Gamma, Delta and Omicron BA.1, for the panel of 28 potent mAbs (FIG. 2A-C, Tables 13 to 16 and 18). The likely origin of all of these antibodies from vaccine induced memory B cells is apparent in that in almost all cases, FRNT50 titres to Victoria are at the high end of all VoC tested for each mAb (FIG. 2A-C, Tables 13 to 16 and 18). Five of the mAbs neutralize BA.1 with FRNT50 titres <10 ng/ml, mAb Omi-3, 8, 12, 18 and 24 are the most potent with FRNT50 titres of 9, 8, 4, 6, 7 ng/ml and FRNT90 titres of 67, 42, 20, 18, 35 ng/ml respectively.

The data provided in Tables 13, 14 and 16 include some IC50 data obtained using pseudoviral constructs. The data in Table 18 consists of IC50 results obtained exclusively from authentic virus constructs.

17/28 antibodies are cross-reactive against all VoC with <10-fold difference in FRNT50 titres between all viruses. Omi-06, 24, 30, 31, 34 and 41 show reduced or absent activity against Delta, with 3/6 of these belonging to the VH1-69 family, and may have an epitope impinging on the L452R Delta mutation (Delta shares T478K with BA.1). Antibodies Omi-09 and 32 perform poorly on Beta and Gamma and may be sensitive to E484K found in Beta and Gamma, but may tolerate the E484A change in Omicron (Omicron shares N501Y and K417N with Beta whilst Gamma is N501Y, K417T). Finally, although 129 anti-NTD mAbs were isolated only one of these, Omi-41, showed FRNT50 titres <100 ng/ml, Omi-41 showed neutralizing activity against Victoria, Alpha, Beta and Gamma but no activity against Delta, presumably resulting from the unique spectrum of NTD changes found in Delta.

### Neutralization of BA.1 Compared to BA.1.1, BA.2 and BA.3

Lentiviral based reporters were constructed pseudotyped with the S gene sequences for Victoria, BA.1, BA.1.1, BA.2 and BA.3. Neutralization assays against the Omicron mAb are shown in FIG. 2B, Tables 14 and 18, most antibodies show little difference in neutralization of BA.1, BA.1.1, BA.2 and BA.3. However, there were some notable exceptions; BA.2 neutralization was reduced 38, 3 and 158-fold compared to BA.1 for Omi-8, 29 and 32 respectively, while BA.1.1 neutralization was reduced 40.9, 10.8, 7.8 and 6.6-fold compared to BA.1 for Omi-6, 24, 34 and 35 respectively and knocked out for Omi-39 and 40. BA.3 neutralization by the Omi-mAb mirrored that found with BA.2 with the exception of Omi-06 and Omi-36 where BA.3 neutralization titres were considerably lower than either BA.1 or BA.2. For some reason the NTD binding mAb Omi-41 did not neutralize Victoria in the pseudoviral system but did neutralize live virus, this was also found with early pandemic mAb 159 which showed potent activity on live virus but no activity on pseudovirus.

Pseudoviral neutralization curves for panels of mAb isolated from early pandemic cases together with mAb isolated from Beta cases is shown in FIGS. 4A, B, Table 15, in most cases, neutralization titres against BA.1, BA.1.1 and BA.2 are similar, but there are some differences, mAbs 40, 278 and 318 neutralize BA.2>BA.1, whereas 222, Beta 22, 29, 54, 55 and 56 neutralize BA.1 better than BA.2, whilst Beta-53 which binds close to the N343 glycan shows reduced neutralization of BA.1.1.

### Neutralization by Antibodies Developed for Clinical Use.

Finally, neutralization of Victoria, BA.1, BA.1.1, BA.2 and BA.3 strains was tested using mAbs being developed for clinical use where a number of differences were found (FIG. 2C, Tables 16 and 18). Interestingly, activity of Known Antibody A (REGN 10987) was partially restored on BA.2 but still 308-fold reduced compared to Victoria, Activity of Known antibody C (AZD1061) was almost completely restored on BA.2, whilst Known antibody D (AZD8895) was 5.4-fold reduced on BA.2 vs BA.1 and the combination of both Known antibody D (AZD8895) and E, was only reduced 8-fold compared to Victoria. The activity of Known Antibody K (S309) was 6.8-fold reduced on BA.2 compared to BA.1, Finally the activity of Known Antibody G (ADG20) was completely lost on BA.2.

In summary, the neutralization of most Omicron monoclonal antibodies are not affected by the differences between BA.1, BA.1.1, BA.2 or BA.3 mutations. Some monoclonal antibodies do however show differences, in particular Known Antibody A (REGN 10987) and Known antibody C (AZD1061) which neutralize BA.2 more easily than BA.1 and Known Antibody K (S309) which shows reduced neutralization of BA.2 and this may encourage sub-lineage typing before use. The structural explanations for the differences between BA.1, BA.1.1, BA.2 and BA.3 neutralization will be discussed below.

### Neutralization of BA.1, BA.1.1, BA.2 and BA3 by Immune Sera

To determine whether the differences in transmissibility between BA.1 and BA.2 may be due to differential neutralization and also to determine whether there was a possibility that BA.2 could escape the BA.1 antibody response, neutralization assays were performed using sera from a variety of sources. First, neutralization assays were performed on Victoria, BA.1, BA.1.1, BA.2 and BA.3 using sera collected from vaccinees receiving the Oxford/AstraZeneca AZD1222 (n=41) or Pfizer/BioNtech BNT162b2 (n=20) vaccines (FIG. 3A, B).

For AZD1222 samples were taken 4 weeks after the second and third doses of vaccine. Following the third dose of AZD1222 there were small but significant differences between pseudoviral neutralization with reductions of the titres against BA.2 vs BA.1 (1.17-fold p=0.0019) and BA.1.1 vs BA.1 (1.29-fold p=0.0086). For BNT162b2 samples were taken 4 weeks and 6 months following the second dose of vaccine, before the third dose and 4 weeks after the third dose. Following the third vaccine dose, titres against BA.1, BA.2 and BA.1.1 were similar with non-significant differences between them. Next, the neutralization profile of serum collected from cases infected with Omicron were determined. Early samples (n=12) were taken <14 days from symptom onset (median 13 days), later samples (n=17) were taken >21 days following symptom onset (median 38 days). All cases had received at least 2 doses of vaccine and a number of the late convalescent cases received a third dose of vaccine following Omicron infection. Neutralization against Victoria, Alpha, Beta, Gamma, Delta and Omicron was tested using live virus neutralization assays (FIG. 3C). At early time points, all vaccinated cases had high titres to Victoria with geometric mean FRNT50 close to 1/3000 and exhibited broad neutralization of VoC with FRNT50 >1/1000 for all viruses except Omicron (FRNT50=558). At the later time point titres against Victoria were unchanged whilst there were increases in titres to the VoC and Omicron (3-fold p=0.0123). Pairwise comparison of early and late samples taken from the same individuals confirmed the broad boosting of the response following Omicron infection (FIG. 5A) Neutralization of Victoria, BA.1, BA.1.1, BA.2 and BA.3 was assayed by pseudoviral neutralization. BA.1 neutralization titres were higher at later time points. However, all of the sera were obtained from BA.1 infected cases and there were small but significant reductions in the neutralization titres of BA.2 vs BA.1 (1.7 and 1.5-fold p=0.0034 and 0.0067 at <14 and >21 days respectively), the titres of BA.1.1 vs BA.1 were not significantly reduced while at >21 days the titre against BA.3 vs BA.1 was reduced 1.7-fold (p=0.0012) (FIG. 3D, 5B).

In summary, following three doses of vaccine, particularly BNT162b2, good neutralizing titres of antibody against Omicron BA.1 BA.1.1, BA.2 and BA.3 are induced, with only minor differences between the titre against BA.1 BA.1.1, BA.2 and BA.3. This may indicate that the increased transmissibility of BA.2 is not due to increased vaccine escape. Following break through Omicron infection, in previously vaccinated individuals, there is boosting of a broad antibody response to variants of concern and the generation of strong responses to Omicron. Since there are only small differences in the neutralization between BA.1 and BA.2, BA.2 superinfection of BA.1 exposed and vaccinated cases is unlikely, at least in the short term.

### Neutralization of BA.4 Compared to BA.1, BA.1.1, BA.2 and BA.3

Neutralization of BA.4/5 was also assessed in comparison to Omicron sub-lineages BA.1, BA.1.1, BA.2, BA.3 and the early pandemic Victoria strain. BA.4/5 was shown to have a more extreme antibody escape phenotype than BA.1 and BA.2, and serum from triple vaccinated donors had ˜2-3-fold reduction in neutralization titres compared to the neutralization of BA.1 and BA.2. Additionally, serum from breakthrough BA.1 infections in vaccinees showed ˜2-3-fold reduction in neutralization titres to BA.4/5 compared to BA.1 and BA.2. This suggests that currently approved vaccines and mAbs may be less effective at preventing BA.4/5 transmission. New monoclonals and combinations may therefore be needed to plug the gap to protect the extremely vulnerable and those unable to mount adequate vaccine responses.

### Neutralization of BA.4 by Vaccine Serum

A panel of pseudotyped lentiviruses (Di Genova et al., 2020, “Production, titration, neutralisation and storage of SARS-CoV-2 lentiviral pseudotypes”. Figshare preprint.) expressing the S gene from the Omicron sub-lineages BA.1, BA.1.1, BA.2, BA.3 and BA.4/5 was contructed, together with early pandemic Wuhan related strain, Victoria, used as a control.

Neutralization assays were performed using serum obtained 28 days following a third dose of the Oxford-AstraZeneca vaccine ADZ1222 (n=41)) (Flaxman et al, 2021, “Reactogenicity and immunogenicity after a late second dose or a third dose of ChAdOx1 nCoV-19 in the UK: a substudy of two randomised controlled trials (COV001 and COV002)”. Lancet 398, 981-990.) or the Pfizer-BioNtech vaccine BNT162b2) (Cele et al., 2021, “Omicron extensively but incompletely escapes Pfizer BNT162b2 neutralization”. Nature 602, 654-666) (n=20) (FIG. 7 A,B). For AZD1222 neutralization titres for BA.4 were reduced 2.1-fold compared to BA.1 (p=0.0001) and 1.8-fold compared to BA.2 (p=0.0001). For BNT162b2 neutralization titres were reduced 3.2-fold (p=0.0001) and 3.1-fold (p=0.0001) compared to BA.1 and BA.2 respectively. These reductions in titre are likely to reduce vaccine effectiveness particularly at longer time points as antibody titres naturally wane.

Neutralization of BA.4/5 by Serum from Breakthrough BA.1 Infection

At the onset of the Omicron outbreak, vaccinated volunteers who had suffered breakthrough Omicron infections were recruited. Samples were first taken ≤14 days from symptom onset (median 13 days), while late samples were taken ≥21 days from symptom onset (median 38 days) n=16. Pseudoviral neutralization assays were performed against the panel of pseudoviruses representing variants of concern and the Omicron Sub-lineages (FIG. 7 C, D).

BA.1 infection following vaccination leads to a broad neutralizing response, with high titres to all the VoC, which is boosted at later time points (Nutalai et al. 2022, “Potent cross-reactive antibodies following Omicron breakthrough in vaccines”. Cell (in press)). Neutralization titres against BA.4 were significantly less than BA.1 and BA.2, at the early time point BA.4/5 titres were reduced 1.9-fold (p=0.0001) and 1.5-fold (p=0.0015) compared to BA.1 and BA.2 respectively. At the later point BA.4/5 titres were reduced 3.4-fold (p=0.0001) and 2-fold (p=0.0017) compared to BA.1 and BA.2 respectively.

Thus, BA.4/5 shows a degree of immune escape from the vaccine/BA.1 response when compared with BA.1 and BA.2. These samples were all taken reasonably close to the time of infection meaning that further waning in the intervening months may render individuals susceptible to reinfection with BA.4/5.

Escape from Monoclonal Antibodies by BA.4/5

Sensitivity to L452R: It has previously been reported that Omi-24, 30, 31, 34 and 41 show complete knock out of neutralizing activity against Delta, with Omi-06 showing severe knock-down of activity (Nutalai et al., 2022). Since BA.1 and BA.2 harbour only one (T478K) of the 2 Delta RBD mutations, whilst BA.4/5 also harbour L452R, it is expected that all five of these L452 directed mAbs to be knocked out on BA.4/5. This is indeed observed (FIG. 8A, Table 20). Omi-41 also fails to neutralize, which is attributed to the differences in mutations in the NTD (FIG. 9A).

To confirm that the neutralization effects observed are directly attributable to alterations in RBD interactions, binding analyses of selected antibodies to BA.4/5 and BA.2 RBDs by surface plasmon resonance (SPR) were also performed (FIGS. 10, 15). Omi-31 was chosen as representative of the set of L452R sensitive antibodies, and as expected the binding is severely affected (FIG. 10A).

Since detailed information on the interaction of several Omicron responsive antibodies with the RBD is available, the BA.4/5 RBD mutations were modelled in the context of known structures for Omicron Fabs complexed with BA.1 or Delta RBDs (Dejnirattisai et al., 2022, “SARS-CoV-2 Omicron-B.1.1.529 leads to widespread escape from neutralizing antibody responses”. Cell 185, 467-484 e415; Nutalai et al., 2022), (FIG. 11). The Omi-31 complex is shown in FIG. 11A and shows L452 tucked neatly into a hydrophobic pocket, which is unable to accommodate the larger positively charged arginine in BA.4/5 and Delta.

L452R enhancement of binding: Omi-32 shows 77-fold enhanced neutralization of BA.4/5 compared to BA.2. Kinetic analysis of Fab binding to the RBDs suggests that this is mainly achieved by a 5-fold increase in the on-rate of binding (FIG. 10B, C). This is largely explained by the favorable interaction of the arginine at 452 making a salt bridge to residue 99 of the heavy chain (HC) CDR3 (FIG. 11B), perhaps assisted by removal of slightly unfavourable charge interactions at residue 493. It is possible that these electrostatic changes enhance on-rate by electrostatic steering of the incoming antibody.

Sensitivity to F486V: Extending the logic used to understand Delta sensitivity, the remaining antibodies affected by BA.4/5>BA.2, but which retain activity against Delta, are likely sensitive to the F486V change, namely Omi-02, 09, 12, 23, 25, 26, 29. The binding sensitivity was confirmed by SPR analysis of Omi-12 (FIG. 10D, E) which showed an almost 1,000-fold reduction in affinity. An example of the structural basis of sensitivity is provided by the Omi-25 complex (FIG. 11C), which shows that the phenylalanine side chain acts as a binding hot-spot, nestled in a hydrophobic cavity making favorable ring-stacking interactions with Y106 of the HC CDR3.

### Activity of Commercial Antibodies Against BA.4 and BA.5

A panel of antibodies that have been developed for therapeutic/prophylactic use was tested against BA.4/5 (FIG. 8B, Table 21). Many of these antibodies have already suffered severe reductions or knock out of activity against BA.1, BA.1.1 or BA.2. For AstraZeneca AZD1061, activity to BA.4/5 was similar to BA.2 (<2-fold reduction), whilst for AZD8895 residual activity against BA.2 was knocked out. The activity of the combination of both antibodies in AZD7442 (Dong et al., 2021, “Genetic and structural basis for recognition of SARS-CoV-2 spike protein by a two-antibody cocktail”. Nature Microbiol. 6, 1233-1244) was reduced 8.1-fold compared with BA.2. The residual activity of REG10987 (Weinreich et al., 2021, “REGN-COV2, a Neutralizing Antibody Cocktail, in Outpatients with Covid-19”. N Engl J Med 384, 238-251) against BA.2 was further reduced on BA.4/5, likewise residual BA.1 neutralizing activity was knocked out for ADG20 (Yuan et al., 2022, “A broad and potent neutralization epitope in SARS-related coronaviruses”. bioRxiv. https://doi.org/10.1101/2022.03.13.484037) on BA.4/5. For S309 (VIR-7831/7832) (Sun and Ho, 2020, “Emerging antibody-based therapeutics against SARS-CoV-2 during the global pandemic”. Antib Ther 3, 246-256.), activity against BA.4/5 was 1.6 fold reduced compared to BA.2.

These effects can be rationalized by reference to the way the antibodies interact with the RBD, for instance in the case of AZD8895 (an IGHV1-58 genotype mAb, FIG. 11E), F486 forms a hydrophobic interaction hotspot which will be abrogated by the mutation to a much smaller valine sidechain. Antibody residues involved in the interactions with F486 are highly conserved among this genotype of mAbs, including Omi-12, 253 and Beta-47 (Nutalai et al., 2022, “Potent cross-reactive antibodies following Omicron breakthrough in vaccines”. Cell (in press); Dejnirattisai et al., 2021, “The antigenic anatomy of SARS-CoV-2 receptor binding domain”. Cell 184, 2183-2200 e2122; Liu et al., 2021, “The Beta mAb response underscores the antigenic distance to other SARS-CoV-2 variants”. Cell, Host and Microbe 30, 53-68), explaining the severe effect of the F486V mutation on neutralization of these mAbs (FIGS. 8A, 13).

### Neutralisation of BA.2.75 by Vaccine Serum

A panel of pseudotyped lentiviruses was constructed as above (Di Genova et al., 2020) expressing the S gene from the Omicron sub-lineages BA.1, BA.1.1, BA.2, BA.2.12.1, BA.4/5, BA.2.75, together with Victoria, an early pandemic Wuhan related strain, used as a control. D339H, G446S, N460K and R493Q were also included as single mutations on the BA.2 background. Neutralization assays were performed using serum obtained 28 days following a third dose of the Oxford-AstraZeneca vaccine AZD1222 (n=41) (Flaxman et al., 2021 “Reactogenicity and immunogenicity after a late second dose or a third dose of ChAdOx1 nCoV-19 in the UK: a substudy of two randomised controlled trials (COV001 and COV002).” Lancet 398, 981-990) or of Pfizer-BioNtech vaccine BNT162b2 (n=22) (Cele et al., 2021; “Omicron extensively but incompletely escapes Pfizer BNT162b2 neutralization”. Nature 602, 654-666e) (FIG. 17). For AZD1222, neutralization of BA.2.75 was reduced 1.2-fold compared to BA.2 (p=0.0182) and 1.1-fold compared to BA.2.12.1 (p=0.0065), but increased 1.5-fold compared to BA.4/5 (p<0.0001) (FIG. 17B). Overall, there are reductions in BA.2.75 neutralization titres of vaccine serum compared to BA.2 but not to the level seen with BA.4/5.

Neutralization of BA.2.75 by Serum from Vaccine Breakthrough BA.1 or BA.2 Infections

Breakthrough BA.1 serum samples were taken from vaccinated volunteers ≥28 days from symptom onset (median 38 days; n=16). Pseudoviral neutralization assays were performed against the panel of pseudoviruses described above (FIG. 17C). Neutralisation titres for BA.2.75 were similar to BA.2, and 1.4-fold (p=0.0052) and 2.0-fold (p=0.0001) higher than BA.2.12.1 and BA.4/5 respectively, suggesting that BA.2.75 might be less likely to cause reinfections in individuals who have suffered BA.1 breakthrough infections than BA.2.12.1 or BA.4/5.

Breakthrough BA.2 serum samples were taken from vaccinated volunteers ≥12 days from symptom onset (median 29 days; n=23). Pseudoviral neutralization assays were performed against the panel of pseudoviruses Victoria, BA.1, BA.1.1, BA.2, BA.2.12.1, BA.4/5 and BA.2.75 (FIG. 17D). Here, neutralization titres against BA.2.75 were significantly reduced compared to BA.2 (1.4-fold; P=0.0021), similar to BA.2.12.1, but still higher than BA.4/5 (1.4-fold; P=0.0123). Taken together, BA.2.75 shows a degree of escape from humoral response induced by BA.2 breakthrough infection but not BA.1 infection.

Individual BA.2.75 Mutation have Differential Effects on Neutralization

To understand the effects of the individual mutations in the BA.2.75 RBD, these mutations were introduced individually into the pseudovirus BA.2 background and their neutralization was assayed using triple vaccinated Pfizer BNT162b2 serum (FIG. 17E). Neutralization titres for BA.2 were reduced for 3/4 single mutation variants of BA.2, with the greatest decrease for N460K (3.1-fold, p<0.0001), followed by D339H (1.3-fold, p=0.0006), then by G446S (1.2-fold, p=0.2312), however neutralization titres were increased 1.5-fold by the R493Q reversion mutation (p<0.0001). Q493 is present in all vaccines thus explaining the increase in activity of vaccine serum to this reversion mutation.

Escape from Monoclonal Antibodies by BA.2.75

To dissect how BA.2.75 might affect neutralising antibody activity, pseudoviral assays were used to test a recently reported panel of potent human mAbs generated from cases of Omicron breakthrough infection (BA.1 IC50 titres <0.1 g/ml) (Nutalai et al., 2022.) (FIG. 19A, Table 22). Among the 27 RBD-specific mAbs, those belonging to the IGHV3-53/66 families are most severely affected. Three (Omi-16, Omi-29 and Omi-36) showed a complete knock out of BA.2.75 neutralization; an additional four (Omi-18, Omi-20, Omi-27 and Omi-28) showed >5-fold reduction compared to BA.2, which is in line with the observation that N460 interacts with highly conserved GGS/T motif of CDR-H2 in the structures of RBD/IGHV-3/66 complexes (FIG. 21B) (Dejnirattisai et al. 2021, Liu et al. 2021, Nutalai et al. 2022).

Like BA.2 and BA.4/5, BA.2.75 is not neutralised by the anti-NTD mAb Omi-41, which only interacts with the NTD of BA.1, BA.1.1 and BA.3.

The Omi mAbs were also tested against the pseudoviruses encoding single point mutations in the BA.2 RBD described above (FIG. 23, Table 24). The VH3-53/66 mAbs that lost neutralization to BA.2.75 were also impacted by the N460K mutation, confirming the prediction that this residue was critical for the binding of a number of members of this public gene family. Interestingly, The BA.2+N460K mutation in isolation shows a larger impact than BA.2.75 on the activity of several mAbs: the neutralisation titre of Omi-03 (IGHV3-53) was reduced 50-fold for BA.2+N460K but only 2-fold for BA.2.75; Omi-17 (IGHV3-66) was completely knocked out on BA.2+N460K but only reduced 4-fold for BA.2.75; and Omi-33 (IGHV3-33) was reduced 7-fold for BA.2+N460K but there was no change observed for BA.2.75. Thus, other mutations in BA.2.75 might have mitigated the effect of the N460K mutation, particularly the R493Q mutation.

Interestingly, BA.2.75 is more sensitive to Omi-32 (IGHV-3-33) than BA.2, with an 8-fold increase in neutralisation titre. The enhancement in activity by Omi-32 is likely due to a stronger interaction of the antibody with the RBD through the G446S mutation (FIG. 19A, Table 22).

To confirm that the change in neutralising activities observed are associated with alterations in RBD interaction, binding analyses of selected antibodies to BA.2.75 and BA.2 RBDs were performed by surface plasmon resonance (SPR) (FIG. 24). Binding of Omi-29 (IGHV3-53) and Omi-36 (IGHV3-66) to BA.2.75 was severely impaired, and Omi-18 and Omi-20 showed 8-fold reductions compared to BA.2. On the other hand, a 2-fold increase in binding affinity of Omi-32 was seen for BA.2.75 in comparison with BA.2, in line with the enhanced neutralisation titre observed.

Escape from Commercial Monoclonals Against BA.2.75

The sensitivity of a panel of mAbs that have been developed as therapeutics against BA.2.75 (FIG. 19B, Table 23) was evaluated. The neutralisation profiles are in general similar between BA.2.75 and BA.2; however, further to the 6/12 mAbs (REGN10933, ADG10, ADG20, ADG30, Ly-CoV555, Ly-CoV16) which have already suffered complete loss of neutralising activity for BA.2, the residual activity of REG10987 (Weinreich et al., 2021, “REGN-COV2, a Neutralizing Antibody Cocktail, in Outpatients with Covid-19.” N Engl J Med 384, 238-251) against BA.2 was further knocked out for BA.2.75 due to the G446S mutation (Dejnirattisai et al, 2022). For AstraZeneca AZD1061, activity against BA.2.75 was similar to that against BA.2 (<3-fold reduction); whilst the AZD8895 titre was restored to 0.008 g/ml for BA.2.75 from 1.333 g/ml for BA.2, a 167-fold increase in activity. As a result, AZD7442 (a combination of AZD8895 and AZD1061) (Dong et al., 2021, “Genetic and structural basis for recognition of SARS-CoV-2 spike protein by a two-antibody cocktail”. Nature Microbiol. 6, 1233-1244) showed similar activity against BA.2.75 and BA.2 (2-fold reduction). The results can be explained by the structure of the ternary complex of the ancestral SARS-CoV-2 RBD/AZD1061/AZD8895 (Dong et al., 2021). G446 has contacts with CDR-L2 Y55 and W56 of AZD1061, G446S mutation will induce steric clashes (FIGS. 21D, E). While CDR-H2 of AZD8895 sits above and makes a hydrogen bond to Q493 of the RBD, an arginine at 493 will severely clash with CDR-H2 of the mAb (FIGS. 21F, G). The activity of S309 (Sun and Ho, 2020, “Emerging antibody-based therapeutics against SARS-CoV-2 during the global pandemic.” Antib Ther 3, 246-256) is increased 3-fold for BA.2.75 compared to BA.2, suggesting that the D339H mutation in BA.2.75 reduces the impact of the preceding G339D mutation in BA.2 on the activity of S309. LY-CoV 1404 (bebtelovimab) (Westendorf et al., 2022, “LY-CoV1404 (bebtelovimab) potently neutralizes SARS-CoV-2 variants.” Cell Rep 39, 110812) is the only mAb where neutralization is fully retained on all Omicron sublineages.

Escape from Monoclonal Antibodies by BA.2, BA.4 and BA.5 Sublineages

Attrition of mAb activity was also observed with the new BA.2, BA.4 and BA.5 sublineages (including BA.4.6, BA.2.75, BA.2.75.2, BA.2.3.20, BJ.1, BQ.1, BQ.1.1, XBB, XBB.1 and XBB.1.5) (FIG. 34), with XBB leading to the most extreme escape. Activity of all 9 IGHV3-53/66 mAbs was reduced >100-fold with complete knock out of activity in 5/9 by BA.2.75.2. Only a single mAb, Omi-42 was unaffected by all variants. Omi-42 is unusual as it binds at the back of the left shoulder of the RBD (Nutalai et al., 2022) in a region that has not yet been targeted for mutation by the set of newly emerging BA.2 variants, perhaps because of the relative rarity of antibodies binding in this region.

Further data can be found in Nutalai, et al. (2022) “Potent cross-reactive antibodies following Omicron breakthrough in vaccinees”, Cell 185(12), 2116-2131; Huo et al. (2022) “Humoral responses against SARS-CoV-2 Omicron BA.2.11, BA.2.12.1 and BA.2.13 from vaccine and BA.1 serum”, Cell discovery 8, 119; and Huo, et al. (2022) “A delicate balance between antibody evasion and ACE2 affinity for Omicron BA.2.75” Cell Reports, 42(1).2023.

### Neutralisation of BA.2 Subvariants BA.2.11, BA.2.12 and BA.2.13 by Vaccine Serum

The receptor binding capacity of the BA.2 subvariants BA.2.11, BA.2.12 and BA.2.13 was also evaluated. A high-resolution crystal structure of BA.2.12.1 RBD was generated, showing differential sensitivity of new BA.2 subvariants BA.2.11, BA.2.12 and BA.2.13 to serum samples and monoclonal antibodies (mAbs) compared to BA.2.

Considering the physico-chemical properties of the side chain of residue 452, BA.2.13 would be expected to be a relatively modest change; L to M will increase the size of the side chain but it remains hydrophobic. L to Q in BA.2.12.1 introduces some polar character, whilst BA.2.11 is the most radical with L to R introducing a large basic amino acid.

### Neutralisation of BA.2 Subvariants BA.2.11, BA.2.12 and BA.2.13 by Vaccine Serum

To evaluate the susceptibility of the BA.2 subvariants to neutralisation by immune sera, neutralization assays were performed on pseudotyped lentiviruses expressing the Spike gene of BA.2.11, BA.2.12 and BA.2.13, using a series of serum samples.

Firstly, the neutralisation profile with sera collected 4 weeks following a third dose of the Oxford-AstraZeneca vaccine AZD1222 (n=41) or Pfizer-BioNtech vaccine BNT162b2 (n=18) was observed. No significant loss in neutralisation titre was seen compared to BA.2. In fact, BA.2.13 showed a significant increase (1.6-fold, p<0.0001) for AZD1222 vaccinees (FIG. 27a, b). This contrasts with a recent report (Cao, Y., et al., “BA.2.12.1, BA.4 and BA.5 escape antibodies elicited by Omicron infection”. Nature, 2022), where sera collected from triple-dose vaccinees (4 weeks following a third dose of the inactivated vaccine CoronaVac or a ZF2001 booster after two doses of CoronaVac) showed significant reductions in neutralisation titre for both BA.2.12.1 and BA.2.13 (BA.2.11 was not tested).

Neutralisation of BA.2 Subvariants BA.2.11, BA.2.12 and BA.2.13 by Serum from Vaccine Breakthrough BA.1 or BA.2 Infections

Next, the neutralisation profile for serum samples collected from vaccinees infected with BA.1 were examined. Samples (n=14) were taken ≥28 days following symptom onset (median 38 days); all convalescent individuals had received at least 2 doses of vaccine, 3 of them received a third dose of vaccine following Omicron infection. There were significant reductions in neutralisation titre for all three variants compared to BA.2, with the greatest decrease for BA.2.11 (1.6-fold, P=0.0067), followed by BA.2.12.1 (1.4-fold, P=0.0085) and BA.2.13 (1.2-fold, P=0.0085) (FIG. 27c). Together, these observations suggest that, in comparison with BA.2, its subvariants are not showing stronger humoral immune escape in individuals vaccinated with three doses of AZD1222 or BNT162b2. However, for vaccinees who had a BA.1 breakthrough infection, regardless of the type of vaccine they had received, the BA.2 variants are more capable of evading the humoral response, although a broad neutralizing antibody response, with high titres to all the variants of concern, is induced (Nutalai, R., et al., “Potent cross-reactive antibodies following Omicron breakthrough in vaccines”. Cell, 2022. 185(12): p. 2116-2131 e18). This may indicate the different selective pressure on BA.2 and its subvariants on a high background of breakthrough infection. As antibody titres naturally wane at longer time points, people with BA.1 breakthrough infections are expected to be more susceptible to reinfection with the BA.2 subvariants.

To further elucidate the differential responses between BA.2 and its subvariants, pseudoviral assays were performed on a panel of potent human monoclonal antibodies (mAbs) generated from cases of BA.1 breakthrough infection (Nutalai, et al., 2022). (FIG. 29). In line with the structural observation and neutralisation results, the greatest reduction of neutralisation titre was seen for BA.2.11, followed by BA.2.12.1 and BA.2.13, neutralization of BA.2.11 was completely knocked out for 5/27 mAbs (Omi-06, Omi-24, Omi-30, Omi-31 and Omi-34). The neutralising activity against BA.2.12.1 was also reduced to varying degrees for the same set of mAbs, whilst the profiles were largely unchanged against BA.2.13. Among them, Omi-06 belongs to the IGVH4-4 family, and the other four mAbs belong to the IGVH1-69 family. Indeed, previous structural studies predicted Omi-06 and Omi-31 to be sensitive to the L452R mutation in Delta (Nutalai, et al., 2022). To confirm that the differential neutralization effects observed are directly attributable to the changes in RBD binding, surface plasmon resonance (SPR) was used to compare the binding behaviour of BA.2 and BA.2.12.1 RBD, using Omi-06 and Omi-31 as examples. As expected the affinities were reduced, for Omi-06, BA.2.12.1 RBD was 15-fold weaker binding than BA.2 and, strikingly, the binding of BA.2.12.1 RBD to Omi-31 was about 1300-fold weaker (FIG. 30).

The spike mutations in the BA.2.11, BA.2.12 and BA.2.13 variants could render it slightly more transmissible than BA.2. However, compared to BA.2, they do not appear to have acquired greater humoral immune escape in healthy vaccinees who have received three doses of the Oxford-AstraZeneca or Pfizer-BioNtech BNT162b2 vaccine. This result differs from that of vaccinees who have received the triple-dose CoronaVac vaccine, for whom significant reductions in neutralisation titres were observed (Cao, Y., et al., BA.2.12.1, BA.4 and BA.5 escape antibodies elicited by Omicron infection. Nature, 2022). Nevertheless, significant reductions in neutralisation titres were seen in vaccinees who had experienced BA.1 breakthrough infections, no matter which type of vaccine was received, perhaps partly due to partial or complete knock-out of neutralising activity of antibodies belonging to the IGVH1-69 family, many of which are sensitive the mutation at leucine 452 of the Spike RBD. This suggests that the continuously evolving Omicron sublineages are able to gain evasion from the humoral immune responses mounted by BA.1, thus implying that BA.1 Spike or RBD might not be a substantially better immunogen than that of the ancestral Wuhan strain for the development of the next-generation SARS-CoV-2 vaccine.

### BA.2.12.1 Crystal Structures

The crystal structure of BA.2.12.1 RBD was determined at 2.38 Å as a ternary complex with a neutralizing Fab and nanobody (FIG. 27h-m), demonstrating that structural differences are essentially restricted to the side-chain of residue 452.

Neutralisation of BA.2.75.2 by mAbs Made Following BA.1 Infection

Neutralisation of BA.2.75.2 by a panel of mAbs made following BA.1 infection (Nutalai et al., 2022) was investigated. Attrition of mAb activity was observed against BA.2.75.2 (Table 32a). Activity of all 9 IGVH3-53/66 mAbs was reduced >100-fold with complete knock out of activity in 4/9 by BA.2.75.2. Only a single mAb, Omi-42 was unaffected by all variants showing neutralization of the BA.4+14 mutations described with IC50 of 11 ng/ml. Omi-42 is unusual as it binds at the back of the left shoulder of the RBD (Nutalai et al., 2022) in a region that has not yet been targeted for mutation, perhaps because of the relative rarity of antibodies binding in this region.

A panel of mAb that have been developed for clinical use was also tested (Dong et al., 2021; Sun and Ho, 2020; Weinreich et al., 2021; Yuan et al., 2022). Many of these were severely impacted by a number of variants. Activity of all mAbs apart from S309 was knocked out by one or more variants including Ly-CoV1404 (Westendorf et al., 2022) (see Table 32b).

### Pfizer BNT162b2 Vaccine Serum Neutralization Titres for BA.2.75.2 and BA.2.3.20.

Neutralization on serum collected 28 days following a third dose of Pfizer BNT162b2 vaccine (Polack et al., 2020) and in cases infected with BA.1, BA.2 of BA.4/5 the characteristics of these subjects are described in the methods.

Using serum obtained 28 days following BNT162b, infection titres to BA.2.75.2 showed large reductions compared to BA.2 and BA.4, and were the lowest of all variants tested compared to the ancestral strain Victoria. The reduction in titres to BA.2.75.2 were in contrast to BA.2.75 which showed only a modest reduction compared to BA.2. There were also large reductions in titres to BA.2.3.20.

Using serum obtained following BA.1, BA.2 and BA.4/5 infection, there were similar large reductions in the neutralization titres of BA.2.75.2 and BA.2.3.20 compared to BA.4/5. Neutralization of the BA.4+14 RBD mutations described above were also reduced compared to BA.2 and BA.4/5 but not a great deal more than BA.2.75.2 indicating a dominant effect of mutations in BA.2.75.2

Neutralisation of BA4.6 by Serum from Vaccine Breakthrough BA.1 or BA.2 Infections

Here, we study the neutralisation profile of BA.4.6 using: Pfizer-BioNtech vaccine serum, BA.1, BA.2 and BA.4/5 vaccine breakthrough immune serum, as well as panels of monoclonal antibodies. Remarkably, we show further antibody evasion of BA.4.6, providing guidance for vaccine design and the use of therapeutic monoclonals.

To evaluate the antibody evasion capacity of BA.4.6, we constructed a panel of pseudotyped lentiviruses (Di Genova, C., et al., Production, titration, neutralisation and storage of SARS-CoV-2 lentiviral pseudotypes. figshare, 2020.) expressing the S gene from BA.4.6 and other SARS-CoV-2 variants together with early pandemic Wuhan related strain, Victoria, used as a control. Firstly, the neutralisation profile was examined with sera collected 4 weeks following a third dose of the Pfizer-BioNtech vaccine BNT162b2 (n=22). Compared to BA.4/5, neutralisation titres against BA.4.6 were reduced 2-fold (p<0.0001) for BNT162b2 sera (FIG. 28a).

The neutralisation profile for serum samples collected from vaccinees infected with BA.1 were assated. Samples (n=16) were taken ≥28 days following symptom onset. BA.2 samples (n=23) were taken ≥12 days following symptom onset or BA.4/5. Samples (n=11; all but one vaccinated) were taken ≥23 days following symptom onset (FIG. 28b-d). Neutralization titres against BA.4.6 were significantly reduced compared to BA.4/5 for both breakthrough BA.1 (1.5-fold; P=0.0006) and BA.2 (1.2-fold; P=0.0384) serum samples. Notably, BA.4.6 was able to effectively escape neutralisation by serum samples from BA.1 breakthrough infections, showing substantial reduction in titres compared to BA.1 (4.4-fold; p=0.0001), BA.2 (3-fold; p=0.0009) and BA.4/5 (1.5-fold; p=0.0006). A small non-significant increase in neutralisation titres against BA.4.6 was observed in the BA.4/5 breakthrough cohort compared to BA.4/5.

To further characterise the antigenic escape properties of BA.4.6, pseudoviral assays were performed on a panel of potent human mAbs generated from BA.1 breakthrough convalescents (Nutalai et al., 2022) (FIG. 28e). In general, the neutralisation profiles of BA.4.6 were similar to those of BA.4/5. However, the residual activity of Omi-35 (IC50=1.687 μg/mL) was further knocked out for BA.4.6, and the potency of Omi-32 and Omi-33 against BA.4/5 (IC50=0.035 and 0.013 μg/mL, respectively) was completely impaired for BA.4.6. The loss in activity of Omi-32 could be explained by the disrupted interaction between H1 and R346 as illustrated by previous structural analysis (Nutalai et al., 2022).

Neutralisation of BA.4.6, by mAbs in Clinical Use

Finally, neutralisation activities of a number of mAbs in clinical use was evaluated (FIG. 28f). The potency of AZ1061/cilgavimab against BA.4/5 was completely knocked out against BA.4.6, leading to a total loss in activity of AZ7742/Evusheld (a combination of AZ1061/cilgavimab and AZ8895/tixagevimab which is already inactive against BA.4/5). The activity of S309/sotrovimab (no longer authorized by the U.S. food and drug administration FDA for COVID-19 treatment since April 2022 due to its inefficacy against BA.2) was further reduced compared to BA.2 and BA.4/5. This therefore leaves Ly-Cov1404/bebtelovimab the only option for treatment of BA.4.6.

In summary, BA.4.6 showed further reduction in neutralisation by serum from triple dose Pfizer vaccinees, as well as from BA.1 and BA.2 vaccine breakthrough convalescents compared to BA.4/5. Notably, BA.4.6 does not seem to more resistant to neutralisation by serum from BA.4/5 breakthrough infection compared to other variants. This altogether suggests that there is a strong likelihood of infection or breakthrough infection by BA.4.6 unless one has been triply vaccinated and recovered from BA.4/5 infections, which seems to provide some protection against BA.4.6.

As of September 2022, bivalent booster vaccination, combining the ancestral strain with Omicron BA.1 is being rolled out in the UK, and has been recently authorised by FDA. It remains to be seen how effective these bivalent boosters are at preventing BA.4.6 infection. Finally, BA.4.6 has further impaired the activity of Evusheld which remained active against BA.4/5; as a result, now only LY-CoV1404/bebtelovimab retains potency against all circulating SARS-CoV-2 variants.

Systematic themes in mAb interactions Both Omi-3 (a representative of the IGVH3-53 gene family) and AZD8895 (IGVH1-58) make contacts with F486. Whilst the F486V mutation has little effect on Omi-3 (FIG. 10F, G, 11F), it seriously reduces the neutralization of AZD8895 and other IGVH1-58 mAbs e.g. Omi-12 (FIG. 10D, E, 11E). It is notable that whereas the numerous Omi series antibodies belonging to the closely related IGVH3-53 and IGVH3-66 gene families (9/28 in total FIG. 8A Table 21) are almost entirely resilient to the BA.4/5 changes, the large majority of antibodies from these gene families elicited against earlier variants are knocked out on BA.1 and BA.2 (Nutalai et al., 2022), consistent with selection of a subset of antibodies by breakthrough Omicron infection that are insensitive to the further BA.4/5 mutations.

The effects on antibodies with broadly similar epitopes can vary dramatically, and this is equally true for antibodies which have 452 or 486 central to their binding footprint. Thus Omi-31 (IGVH1-69) and Omi-32 (IGVH3-33), both bind in front of the right shoulder with their CDR-H3 positioned close to 452, whilst the activity of Omi-31 is abolished by L452R (as detailed above), Omi-32 is markedly enhanced (FIG. 8A, 11A, B). Similarly, Omi-25 and Omi-42 both belong to the IGVH3-9 gene family and their footprints are in the 486 region (FIG. 11C, D). Omi-25 contacts F486 thus neutralization of BA.4/5 is abolished. In contrast Omi-42 does not contact either of the mutation sites and neutralization is fully retained for BA.4/5 (FIG. 10H, I, 11D).

### Fine Mapping of RBD Antibody Binding Using Competition Measurements.

A matrix of pairwise BLI measurements were used to map the potent RBD binding Omicron mAbs and several pre-pandemic mAbs of known binding position.

The method yielded a consistent prediction. The mAbs segregate into a restricted set of epitopes, which appear to be subset of the epitopes observed for the early pandemic virus, and are quite distinct from the focus seen for Beta. Essentially the antibodies cluster in two regions, one which includes the VH3-53 and VH3-66 type antibodies is towards the back of the neck/left shoulder, extending up to the top of the left shoulder, whilst the other is on the front of the neck right shoulder region, spilling towards the S309 known antibody binding site. This region is occupied by the VH1-69 family antibodies, with the exception of Omi-2 which is sited within the other cluster. mAb Omi-09 which shows reduced neutralization of Beta and Gamma positions close to residue 484 which is mutated from Glu to Lys in Beta/Gamma and Ala in Omicron. VH1-69 mAb Omi-24, 30, 31 and 34, which show reduced neutralization of Delta are placed close to residue 452 which is mutated from Leu to Arg in Delta.

### Structures of Anti-Omicron Fab RBD Complexes

Structural analyses of selected potent Omicron mAbs were performed. Crystal structures were determined for complexes of Omicron BA.1 RBD with 3 different Fabs: Omi-3, 9 and 12. The complex of Omi-12 was at low resolution (5.5 Å) and so the structure of the Fab alone was determined at high resolution and rigid-body fitted to obtain the complex structure.

Omi-3 belongs to the VH3-53 gene family and demonstrates how this gene family can be adapted to be broadly neutralising against all major SARS-CoV-2 variants (but, like all the potent Omicron antibodies it does not bind SARS-CoV-1 RBD). A fundamental problem for these antibodies is that most VoC harbour mutation N501Y, which introduces a steric clash with the LC CDR1 (L1) capable of abrogating the binding of the large majority of VH3-53 containing antibodies. However, two mechanisms for displacing L1 to avoid this clash have been previously reported (Dejnirattisai et al., 2021b; Liu et al., 2021b). In mAb-222, isolated from individuals infected with early pandemic strains, a proline is inserted at residue 30 which can pack against Tyr-501 without clashes (Dejnirattisai et al., 2021b), allowing it to effectively neutralize Alpha, Beta and Gamma variants. Beta-27 uses an alternative mechanism, lengthening the HC CDR3 (H3) loop to 11 residues from the usual 9, displacing L1 to produce enough space to allow 501Y to be stabilised by main chain interactions conferring similar cross-reactivity (Liu et al., 2021b).

Omi-3 uses the same mechanism as Beta-27 for accommodating the N501Y mutation, although the Omi-3 H3 is one residue longer again. Other VH3-53 Omicron antibodies (Omi-18 and Omi-29) have H3s very similar to Beta-27 and presumably use the same mechanism. This L1 configuration is also compatible with the Y505H mutation in Omicron. However, neither 222 nor Beta-27 can effectively neutralize Omicron and this may be due to specific features of the H3 loop which makes close contact with the Q493R Omicron mutation.

Omi-9 is a one of three VH3-30 mAbs and binds across the left shoulder of the RBD. Omi-9 shows relatively weak neutralization of Beta and Gamma (FIG. 2). Other antibodies with a high degree of sequence similarity bind similarly, with H3 contacting residue 484. Although the Omi-9/BA.1 complex is lower resolution (4.2 Å), it is clear that H3 contacts residue 484 explaining the sensitivity to E484K in Beta and Gamma whilst E484A in Omicron is tolerated.

Omi-12 belongs to the VH1-58 gene family (it is the only member of this family amongst the 28 potent Omicron antibodies). Like Omi-12, several members of this gene family have a glycosylation site at residue 102 of the heavy chain CDR3, the role of which is unclear. VH1-58 antibodies elicited during early pandemic or beta virus infection show reduced ability to neutralize Omicron e.g. mAb 253, Beta-47 and Known antibody D (AZD8895) show a reduction in activity of Omicron BA.1 vs Victoria respectively.

In contrast, Omi-12 has adapted and can potently neutralize Omicron and all VoCs (FIG. 2 A, B). VH1-58 antibodies bind a left shoulder epitope, H3 contacts S477N but a mutation at this position in Iota had no effect on VH1-58 mAb neutralization using a pseudovirus assay. Additionally, mAb 253 is still able to neutralize Delta despite the T478K mutation. BA.2 with early pandemic mAb 150 (VH3-53). Detectable residual activity was observed with BA.1, BA.1.1 and BA.2 (BA.3 not tested). Two complex structures were obtained in different space groups which were very similar and provided 3 independent views of the complex. mAb 150 binds in a pose similar to that observed previously for early pandemic virus however it is translated and forms looser interactions, consistent with almost complete loss of neutralization activity. This shows the dramatic impact of the accommodating mutations found in Omi-3.

Interestingly, in BA.2 the three serine residues mutated in BA.1 RBD: S371L, S373P and S375F in the loop adjacent to the lipid binding pocket are also muted in BA.2 but the mutation at 371 is to a Phe, which means that this is likely a single point mutation from early pandemic, whereas the S317L mutation in BA.1 requires two mutations. BA.2 may therefore have features common to earlier versions of the Omicron lineage. In addition, the various views provided of this part of the structure show that it adopts a range of different conformations. This is likely due to different crystal contacts and reflects flexibility in this loop region. This is likely to have a biological function since the Ser mutation required a double codon change and may possibly affect the presentation of the RBDs. Since we have multiple views of this loop in early pandemic virus, VoC, Omicron BA.1 and BA.2 we can see that flexibility is maintained across all variants.

### Modelling of Effects on Selected Commercial Known Antibodies, Early Pandemic and Beta mAb for BA.1, BA.1.1 and BA.2 Changes

Known Antibody A (REGN10987) and B (10933): Known Antibody B (REGN 10933) binds the back of the left shoulder and REGN 10987 the right shoulder. Activity of both is knocked out by the Omicron lineage apart from Known Antibody A (REGN 10987) with BA.2. Known Antibody B (REGN 10933) H2 contacts residue 493 and since Q493R is present in all Omicron strains, neutralizing activity to Omicron is universally lost. Known Antibody A (REGN 10987) H2 contacts residue 446. BA.2 uniquely lacks the G446S mutation thus regn10987 retains some neutralization capability.

Known Antibody C (AZD1061) & D (AZD8895): Known antibody C (AZD1061) and D (AZD8895) bind the back of the left shoulder and the front of the right shoulder respectively both show reduced neutralization. Known antibody C (AZD1061) is still able to neutralize BA.2 and BA.3 (˜10-fold reduction) but neutralization of BA.1 is reduced >100-fold compared to Victoria and BA.1.1 >1000-fold compared to Victoria. Known antibody C (AZD1061) is affected due to contacts with G446S (absent in BA.2 and BA.3) and R346K (BA.1.1) mutations (contacted by L2 and H3). Known antibody D (AZD8895) is a VH1-58 antibody and contacts residues 477 (H3) & 493 (H2) and is compromised by the S477N and Q493R mutations universally present in the Omicron lineage. Known Antibody E (AZD7442) (a combination of C and D) maintains some neutralizing activity against Omicron strains as the sum of its components.

Known Antibodies F, G and H: All of Known Antibodies F, G and H suffer considerable loss of activity against Omicron. Activity of Known Antibodies F and H are completely lost whilst the activity of Known Antibody G (ADG20) on Omicron is reduced 276-fold

Known Antibodies I and J: Activity of both antibodies on the entire Omicron lineage is knocked out. Known Antibody J (Ly-CoV16) (VH3-53) makes extensive interactions with N501 and Y505 via L1 and L3 making it sensitive to mutations at these residues. Known Antibody I (Ly-CoV-555) is vulnerable to the E484K mutation in delta but likely tolerates E484A however, it also contacts residue 493, thus the universal Omicron Q493R mutation will abrogate binding across the board.

Known Antibody K (S309): Known Antibody K (S309) retains reasonable activity across the Omicron lineage. S309 binds on the right flank with H3 contacting G339 and N343 glycans the latter close to the Serine 371, 373 and 375 mutations. The S371F mutation in BA.2 as opposed to S371L) may affect binding resulting in the slightly weaker activity with this virus.

### Structure of BA.2 RBD and ACE2 Affinity

The affinity of Omicron BA.1, BA.1.1, BA.2 and BA.3 RBDs for ACE2 was measured by SPR and BLI. The affinity of BA.1 was on a par with that of the early virus, 8 nM and 7 nM respectively (binding affinities for Omicron RBDs shown in Tables 14 and 18), implying that the increased affinity imparted by S477N, Q498R and N501Y is counter balanced by other mutations in the ACE2 footprint. The affinity of BA.2 was slightly increased compared to early virus (˜1.5-fold x and Y nM respectively). On the basis of earlier measurements of the contributions of individual mutations to binding affinity G496S and the triple-mutation S371L, S373P and S375F reduce binding by 2-fold and 2.2-fold respectively whereas BA.2 lacks G496S and has S371F. This may account for some of the difference but more likely the mutations in BA.2 on the edge of the ACE2 footprint may enhance binding. This is confirmed by the structure of the BA.2/ACE2.

### BA.4/5 RBD and ACE2 Affinity

The affinity of BA.4/5 RBD for ACE2 was also measured by SPR (FIG. 12A-D). The affinity of BA.4/5 RBD was increased compared to the ancestral virus (Wuhan), BA.1 and BA.2 (approximately 3-fold, 3-fold and 2-fold, respectively (BA.4/5/ACE2 K_{D}=2.4 nM) (Dejnirattisai et al., 2022; Nutalai et al., 2022), which is mainly attributed to an increase in binding half-life. Modelling of the ACE2/RBD complex suggests that the bulk of this effect comes from the electrostatic complementary between ACE2 and the RBD contributed by the L452R mutation (FIG. 12E-G).

### BA.2.75 RBD and ACE2 Affinity

Surface plasmon resonance (SPR) was also used to characterise the interaction between ACE2 and the BA.2.75 RBD. The off-rate was very slow, leading to a sub-nanomolar affinity (BA.2.75/ACE2 KD=0.45 nM) (FIGS. 18A, B). This represents a considerable increase in affinity compared to BA.2 (9-fold) (FIG. 18C), and even tighter than BA.4/5 (5-fold) (FIG. 18D), which binds to ACE2 with higher affinity than BA.2 (Tuekprakhon et al., 2022). BA.2.75 was found to be the strongest ACE2 binder amongst all SARS-CoV-2 VoC, including Alpha (Alpha/ACE2 KD=1.5 nM; FIG. 18E), and the first SARS-CoV-2 VoC to have a sub-nanomolar affinity.

BA.2+N460K RBD could not be expressed, but the binding affinity of BA.2+R493Q RBD to ACE2 (FIG. 18F) was also measured (KD=0.55 nM). This confirms that the R493Q reversion mutation contributes to the high affinity of BA.2.75 RBD.

### Impact of Mutations in BA.2.75

The constellation of mutations in BA.2.75 compared to BA.2 have opposing effects on neutralization. The reversion mutation R493Q makes the virus easier to neutralize using vaccine serum (the vaccine contains Q493), whilst N460K reduces neutralization titres to a greater extent when expressed in isolation compared to the combination of mutations seen in BA.2.75. N460K is a novel substitution that has not appeared in preceding variants of SARS-CoV-2. This mutation was introduced into the BA.2 backbone and its impact on neutralisation by BNT162b2 serum was evaluated. Strikingly, BA.2+N460K titres were reduced 3.1-fold compared to BA.2, greater than the reduction seen with BA.2.75, and on a par with the reduction seen for BA.4/5.

Using a panel of potent mAbs derived from vaccinated individuals who suffered BA.1 vaccine breakthrough infection, it was shown that the activity of a number of mAbs belonging to the IGHV3-53/66 family are reduced or knocked out against BA.2.75. IGHV3-53/66 are the most frequently isolated mAbs in SARS-CoV-2, and bind an epitope on the ‘neck’. IGHV53/66 thus forms a major public antibody response and it is no surprise that the virus has evolved to escape this response.

Although BA.2+N460K RBD could not be expressed, a previous study using yeast display showed N460K can enhance RBD binding for ACE2, an effect similar to that seen with the N501Y mutation first described in Alpha (Zahradnik et al., 2021). Thus, N460K can both enhance antibody escape and increase receptor binding affinity.

Interestingly, BA.2.75 has also acquired the R493Q reversion (Q493R was acquired in BA.1 and present in all other Omicron sublineages except BA.4/5). BA.2.75 RBD was able to bind ACE2 with 9-fold higher affinity than BA.2 and more tightly than BA.4/5 (Dejnirattisai et al., 2022; Tuekprakhon et al., 2022). This is partly contributed by the R493Q mutation. BA.2.75 RBD has the highest receptor binding affinity among all SARS-CoV-2 variants measured to date.

These data suggest there may be a fine balance between antibody escape and ACE2 receptor affinity. Mutations in BA.2.75 lead to a reduction in neutralization titres of vaccine serum compared to BA.2. Individual BA.2.75 mutations can cause greater reduction in neutralization titres compared to the full BA.2.75 S sequence, but these are balanced by the R393Q reversion mutation, which may have been selected to increase affinity to ACE2 and increase the transmissibility of BA.2.75.

### BA.2.11, BA.2.12 and BA.2.13 RBD and ACE2 Affinity

To evaluate the possible change in transmissibility of the BA.2 subvariants SPR experiments were performed to analyse their RBD binding to ACE2 (FIG. 27d-g). The three RBD variants have an affinity of approximately 3 nM for ACE2, slightly higher than that of BA.2 RBD (KD=4 nM) as previously reported (Nutalai et al., 2022). Modelling of the ACE2/RBD complex suggests that this increase in affinity may result from slightly improved complementarity between ACE2 and the RBD contributed by the mutation at leucine 452. Therefore, these variants might have a subtle advantage in transmission over BA.2.

### Antigenic Cartography of BA.3 and BA.4/5

The neutralization data above has been used to place BA.3 and BA.4/5 on an antigenic map. The method used for analysis of the Delta and Omicron variants was repeated (Liu et al., 2021, “Reduced neutralization of SARS-CoV-2 B.1.617 by vaccine and convalescent serum”. Cell 184, 4220-4236 e4213), where individual viruses were independently modelled allowing for serum specific scaling of the responses. The measured and modelled responses are shown in FIG. 13A (with 1551 observations and 340 parameters the residual error is 23%). The results are best visualized in three dimensions (see 2D projections in FIG. 13B). This shows, as expected, that the Omicron sub-lineages are clustered together but well separated from early pandemic virus and earlier VoC. Amongst the Omicron cluster BA.4/5 is the most distant from the pre-Omicron viruses.

### Antigenic Cartography of BA.2.75

Neutralization of BA.2.75 was tested using serum from individuals previously infected during the course of the pandemic. These included serum obtained early in the pandemic (before the emergence of Alpha) together with serum obtained following Alpha, Beta, Gamma, Delta, BA.1 and BA.2 infection (FIG. 25). As expected, BA.2.75 neutralization titres were lower than the homologous infecting strain (e.g. Alpha serum on Alpha virus). Most striking however was the complete loss of BA.2.75 neutralization using Delta serum (zero samples achieved 50% neutralization at 1/20 dilution). However, titres to BA.2.75 were much higher in cases who had been vaccinated before or after Delta infection.

These data were used to place BA.2.75 onto a three dimensional antigenic map using the method previously reported in Tuekprakhon et al., 2022 (FIGS. 22A, B). Initially all VoC were included (FIG. 22A); this showed that BA.2.75 was grouped with the other Omicron viruses, which segregated into one hemisphere of the 3D plot. BA.2.75 appeared well separated from other Omicron sub-lineages and especially from BA.4/5. It is also notable that BA.2.75 and Delta are diametrically opposed in the diagram, emphasizing the antigenic distance between these two viruses. Since the data are higher dimensional, the 3D projection is likely to distort the true distances and so were calculated for only the Omicron and early pandemic viruses (but retaining the full serology information for each of these). The results are shown in FIG. 22B and recapitulate the major features of the full plot, but allow the Omicron sublineages to distribute more broadly in 3D space. Remarkably, if the clustered early pandemic and BA.2/BA.3 pairs are merged then the points are distributed as a trigonal bi-pyramid maximising their separation, consistent with antigenic escape being a significant factor in their evolution.

### Example 3. Examples of Antibodies that May be Created b Swapping the Light Chain Between Antibodies Derived from the Same Heavy Chain V-Gene

As discussed in the detailed description above, antibodies derived from the same heavy chain V-gene may swap light chains to result in an antibody comprising the heavy chain variable region of a first antibody and a light chain variable region of a second antibody, and such new antibodies may have improved neutralisation and/or other characteristics when compared to the ‘parent’ antibodies.

Tables 4 to 12 provide examples of such antibodies that may be creased by swapping the light chain between antibodies derived from the same heavy chain V-gene. Table 17 provides information as to the heavy chain and light chain V-genes from which the 28 Omicron-specific mAbs are derived, together with their specificity to the RBD or NTD of the spike protein of SARS-CoV-2.

### Example 4. Materials and Methods

### Viral Stocks

SARS-CoV-2/human/AUS/VIC01/2020 (Caly et al, 2020), Alpha and Beta were provided by Public Health England, Gamma cultured from a throat swab from Brazil, Delta was a gift from Wendy Barclay and Thushan de Silva, from the UK G2P genotype to phenotype consortium and Omicron was grown from a positive throat swab (IRAS Project ID: 269573, Ethics Ref: 19/NW/0730. Briefly, VeroE6/TMPRSS2 cells (NIBSC) were maintained in Dulbecco's Modified Eagle Medium (DMEM) high glucose supplemented with 1% fetal bovine serum, 2 mM Glutamax, 100 IU/ml penicillin-streptomycin and 2.5 ug/ml amphotericin B, at 37° C. in the presence of 5% CO2 before inoculation with 200 ul of swab fluid. Cells were further maintained at 37° C. with daily observations for cytopathic effect (CPE). Virus containing supernatant were clarified at 80% CPE by centrifugation at 3,000 r.p.m. at 4° C. before being stored at −80° C. in single-use aliquots. Viral titres were determined by a focus-forming assay on Vero CCL-81 cells (ATCC).

Sequencing of the Omicron isolate shows the expected consensus S gene changes (A67V, Δ69-70, T95I, G142D/Δ143-145, A211/L2121, ins214EPE, G339D, S371L, S373P, S375F, K417N, N440K, G446S, S477N, T478K, E484A, Q493R, G496S, Q498R, N501Y, Y505H, T547K, D614G, H655Y, N679K, P681H, N764K, D796Y, N856K, Q954H, N969K, L981F), an intact furin cleavage site and a single additional mutation A701V.

Cells were infected with the SARS-CoV-2 virus using an MOI of 0.0001.

Virus containing supernatant were harvested at 80% CPE and spun at 3000 rpm at 4° C. before storage at −80° C. Viral titres were determined by a focus-forming assay on Vero cells. Victoria passage 5, Alpha passage 2 and Beta passage 4 stocks Gamma passage 1, Delta passage 3 and Omicron passage 1 were sequenced to verify that they contained the expected spike protein sequence and no changes to the furin cleavage sites.

### Bacterial Strains and Cell Culture

Vero (ATCC CCL-81) and VeroE6/TMPRSS2 cells were cultured at 37° C. in Dulbecco's Modified Eagle medium (DMEM) high glucose (Sigma-Aldrich) supplemented with 10% fetal bovine serum (FBS), 2 mM GlutaMAX (Gibco, 35050061) and 100 U/ml of penicillin-streptomycin. Human mAbs were expressed in HEK293T cells cultured in UltraDOMA PF Protein-free Medium (Cat #12-727F, LONZA) at 37° C. with 5% CO2. HEK293T (ATCC CRL-11268) cells were cultured in DMEM high glucose (Sigma-Aldrich) supplemented with 10% FBS, 1% 100× Mem Neaa (Gibco) and 1% 100× L-Glutamine (Gibco) at 37° C. with 5% CO2. To express RBD, RBD variants and ACE2, HEK293T cells were cultured in DMEM high glucose (Sigma) supplemented with 2% FBS, 1% 100× Mem Neaa and 1% 100× L-Glutamine at 37° C. for transfection. Omicron RBD and human mAbs were also expressed in HEK293T (ATCC CRL-11268) cells cultured in FreeStyle 293 Expression Medium (ThermoFisher, 12338018) at 37° C. with 5% CO2. E. coli DH5a bacteria were used for transformation and large-scale preparation of plasmids. A single colony was picked and cultured in LB broth at 37° C. at 200 rpm in a shaker overnight.

Sera from Pfizer Vaccinees

Pfizer vaccine serum was obtained from volunteers who had received either one or two doses of the BNT162b2 vaccine. Vaccinees were Health Care Workers, based at Oxford University Hospitals NHS Foundation Trust, not known to have prior infection with SARS-CoV-2 and were enrolled in the OPTIC Study as part of the Oxford Translational Gastrointestinal Unit GI Biobank Study 16/YH/0247 [research ethics committee (REC) at Yorkshire & The Humber—Sheffield] which has been amended for this purpose on 8 Jun. 2020. The study was conducted according to the principles of the Declaration of Helsinki (2008) and the International Conference on Harmonization (ICH) Good Clinical Practice (GCP) guidelines. Written informed consent was obtained for all participants enrolled in the study. Participants were studied after receiving two doses of, and were sampled approximately 28 days (range 25-38), 180 days (range 178-221) and 270 days (range 243-273) after receiving two doses of Pfizer/BioNtech BNT162b2 mRNA Vaccine, 30 micrograms, administered intramuscularly after dilution (0.3 mL each), 17-28 days apart, then approximately 28 days (range 25-56) after receiving a third “booster dose of BNT162B2 vaccine. The mean age of vaccinees was 37 years (range 22-66), 21 male and 35 female.

Plasma from Early Pandemic and Alpha Cases

Participants from the first wave of SARS-CoV2 in the U.K. and those sequence confirmed with B.1.1.7 lineage in December 2020 and February 2021 were recruited through three studies: Sepsis Immunomics [Oxford REC C, reference:19/SC/0296]), ISARIC/WHO Clinical Characterisation Protocol for Severe Emerging Infections [Oxford REC C, reference 13/SC/0149] and the Gastro-intestinal illness in Oxford: COVID sub study [Sheffield REC, reference: 16/YH/0247]. Diagnosis was confirmed through reporting of symptoms consistent with COVID-19 and a test positive for SARS-CoV-2 using reverse transcriptase polymerase chain reaction (RT-PCR) from an upper respiratory tract (nose/throat) swab tested in accredited laboratories. A blood sample was taken following consent at least 14 days after symptom onset. Clinical information including severity of disease (mild, severe or critical infection according to recommendations from the World Health Organisation) and times between symptom onset and sampling and age of participant was captured for all individuals at the time of sampling. Following heat inactivation of plasma/serum samples they were aliquoted so that no more than 3 freeze thaw cycles were performed for data generation.

Sera from Beta, Gamma and Delta and BA.1 Infected Cases

Beta and Delta samples from UK infected cases were collected under the “Innate and adaptive immunity against SARS-CoV-2 in healthcare worker family and household members” protocol affiliated to the Gastro-intestinal illness in Oxford: COVID sub study discussed above and approved by the University of Oxford Central University Research Ethics Committee. All individuals had sequence confirmed Beta/Delta infection or PCR-confirmed symptomatic disease occurring whilst in isolation and in direct contact with Beta/Delta sequence-confirmed cases. Additional Beta infected serum (sequence confirmed) was obtained from South Africa. At the time of swab collection patients signed an informed consent to consent for the collection of data and serial blood samples. The study was approved by the Human Research Ethics Committee of the University of the Witwatersrand (reference number 200313) and conducted in accordance with Good Clinical Practice guidelines. Gamma samples were provided by the International Reference Laboratory for Coronavirus at FIOCRUZ (WHO) as part of the national surveillance for coronavirus and had the approval of the FIOCRUZ ethical committee (CEP 4.128.241) to continuously receive and analyse samples of COVID-19 suspected cases for virological surveillance. Clinical samples were shared with Oxford University, UK under the MTA IOC FIOCRUZ 21-02.

Sera from BA.1 Infected Cases, Study Subjects

Following informed consent, individuals with omicron BA.1 were co-enrolled into the ISARIC/WHO Clinical Characterisation Protocol for Severe Emerging Infections [Oxford REC C, reference 13/SC/0149] and the “Innate and adaptive immunity against SARS-CoV-2 in healthcare worker family and household members” protocol affiliated to the Gastro-intestinal illness in Oxford: COVID sub study [Sheffield REC, reference: 16/YH/0247] further approved by the University of Oxford Central University Research Ethics Committee. Diagnosis was confirmed through reporting of symptoms consistent with COVID-19 or a positive contact of a known Omicron case, and a test positive for SARS-CoV-2 using reverse transcriptase polymerase chain reaction (RT-PCR) from an upper respiratory tract (nose/throat) swab tested in accredited laboratories and lineage sequence confirmed through national reference laboratories. A blood sample was taken following consent at least 10 days after PCR test confirmation. Clinical information including severity of disease (mild, severe or critical infection according to recommendations from the World Health Organisation) and times between symptom onset and sampling and age of participant was captured for all individuals at the time of sampling.

### AstraZeneca-Oxford Vaccine Study Procedures and Sample Processing

Full details of the randomized controlled trial of ChAdOx1 nCoV-19 (AZD1222), were previously published (PMID: 33220855/PMID: 32702298). These studies were registered at ISRCTN (U.S. Pat. Nos. 15,281,137 and 89,951,424) and ClinicalTrials.gov (NCT04324606 and NCT04400838). A copy of the protocols was included in previous publications (Folegatti et al., 2020, Lancet 396, 467-478.).

Data from vaccinated volunteers who received two vaccinations are included in the Examples. Vaccine doses were either 5×10¹⁰ viral particles (standard dose; SD/SD cohort n=21) or half dose as their first dose (low dose) and a standard dose as their second dose (LD/SD cohort n=4). The interval between first and second dose was in the range of 8-14 weeks. Blood samples were collected and serum separated on the day of vaccination and on pre-specified days after vaccination e.g. 14 and 28 days after boost.

### Focus Reduction Neutralization Assay (FRNT)

The neutralization potential of Ab was measured using a Focus Reduction Neutralization Test (FRNT), where the reduction in the number of the infected foci is compared to a negative control well without antibody. Briefly, serially diluted Ab or plasma was mixed with SARS-CoV-2 strains and incubated for 1 hr at 37° C. The mixtures were then transferred to 96-well, cell culture-treated, flat-bottom microplates containing confluent Vero cell monolayers in duplicate and incubated for a further 2 hrs followed by the addition of 1.5% semi-solid carboxymethyl cellulose (CMC) overlay medium to each well to limit virus diffusion. A focus forming assay was then performed by staining Vero cells with human anti-NP mAb (mAb206) followed by peroxidase-conjugated goat anti-human IgG (A0170; Sigma). Finally, the foci (infected cells) approximately 100 per well in the absence of antibodies, were visualized by adding TrueBlue Peroxidase Substrate. Virus-infected cell foci were counted on the classic AID EliSpot reader using AID ELISpot software. The percentage of focus reduction was calculated and IC50 was determined using the probit program from the SPSS package.

Plasmid construction and pseudotyped lentiviral particles production Pseudotyped lentivirus expressing SARS-CoV-2 S proteins from ancestral strain (Victoria, S247R), BA.1, BA.1.1, and BA.2 were constructed as described before (Nie, Jianhui, et al. “Establishment and validation of a pseudovirus neutralization assay for SARS-CoV-2.” Emerging microbes & infections 9.1 (2020): 680-686., Liu, Chang, et al. “Reduced neutralization of SARS-CoV-2 B. 1.617 by vaccine and convalescent serum.” Cell 184.16 (2021): 4220-4236.), with some modifications. Briefly, synthetic codon-optimized SARS-CoV-2 BA.1 and BA.2 were custom synthesized by GeneArt (Thermo Fisher Scientific GENEART). The insert fragments and pcDNA3.1 vector were cloned by using Gibson assembly. Victoria (S247R) construct is as previously described in Liu, Chang, et al. “Reduced neutralization of SARS-CoV-2 B. 1.617 by vaccine and convalescent serum.” Cell 184.16 (2021): 4220-4236. To construct BA.1.1, mutagenic primers of R346K (R346K_F 5′-GTGTTCAATGCCACCAAATTCGCCAGCGTGTAC-3′ and R346K_R5′-GTACACGCTGGCGAATTTGGTGGCATTGAACAC-3′) were PCR amplified by using BA.1 construct as a template, together with two primers of pcDNA3.1 vector (pcDNA3.1_BamHI_F 5′-GGATCCATGTTCCTGCTGACCACCAAGAG-3′ and pcDNA3.1_Tag_S_EcoRI_R5′-GAATTCTCACTTCTCGAACTGAGGGTGGC-3′), purified by using QIAquick Gel Extraction Kit (QIAGEN) and followed by Gibson assembly. All constructs were verified by Sanger sequencing after plasmid isolation using QIAGEN Miniprep kit (QIAGEN).

A similar strategy was applied for BA.3 and BA.4/5, briefly, BA.3 mutations were constructed using the combination fragments from BA.1 and BA.2. The resulting mutations are as follows. The resulting mutations are as follows, A67V, A69-70, T95I, G142D, A143-145, A211/L2121, G339D, S371F, S373P, S375F, D405N, K417N, N440K, G446S, S477N, T478K, E484A, Q493R, Q498R, N501Y, Y505H, D614G, H655Y, N679K, P681H, N764K, D796Y, Q954H, and N969K. Although BA.4/5 S protein shared some amino acid mutations with BA.2 (Nutalai et al., 2022), to generate BA.4/5 mutations were added A69-70, L452R, F486V, and R498Q. The resulting S gene-carrying pcDNA3.1 was used for generating pseudoviral particles together with the lentiviral packaging vector and transfer vector encoding luciferase reporter. Integrity of contructs was sequence confirmed.

The same method was also used to construct BA.2.12.1, and BA.2.75, by adding more mutations into the BA.2 construct. To generate BA.2.75, K147E, W152R, F157L, 1210V, G275S, G446S and N460K were added into a BA.2 backbone. 339D was also changed in BA.2 S into 339H, and 493R was reversed in BA.2 to 493Q as in the ancestral strain. To test single mutation impact, D339H, G446S, N460K and R493Q were introduced individually into a BA.2 backbone. The resulting pcDNA3.1 plasmid carrying S gene was used for generating pseudoviral particles together with the lentiviral packaging vector and transfer vector encoding a

### Pseudoviral Neutralization Test

The details of pseudoviral neutralization test were described previously (Liu, Chang, et al. “Reduced neutralization of SARS-CoV-2 B. 1.617 by vaccine and convalescent serum.” Cell 184.16 (2021): 4220-4236) with some modifications. Briefly, the neutralizing activity of potent monoclonal antibodies (mAbs) generated from donors who had recovered from Omicron- and Beta-infection as well as those who were infected during the early pandemic in UK were performed against Victoria, Omicron-BA.1, BA.1.1, BA.2, BA.2.11, BA.2.12.1, BA.2.13, BA.3, BA.4.6, BA.4/5, BA.2.75 and BA.2+N460K. A four-fold serial dilution of each mAb was incubated with pseudoviral particles at 37° C., 5% CO2 for 1 hr. The stable HEK293T/17 cells expressing humanACE2 were then added to the mixture at 1.5×104 cells/well. At 48 hr. post transduction, culture supernatants were removed and 50 μL of 1:2 Bright-Glo™ Luciferase assay system (Promega, USA) in 1×PBS was added to each well. The reaction was incubated at room temperature for 5 mins and the firefly luciferase activity was measured using CLARIOstar® (BMG Labtech, Ortenberg, Germany). The percentage of mAb neutralization was calculated relative to the control. Probit analysis was used to estimate the value of dilution that inhibits half of the maximum pseudotyped lentivirus infection (PVNT50).

To determine the neutralizing activity of convalescent plasma/serum samples or vaccine sera, 3-fold serial dilutions of samples were incubated with the pseudoviral particles for 1 hr and the same strategy as mAb was applied.

### DNA Manipulations

Cloning was done by using a restriction-free approach (Peleg and Unger, 2014). Mutagenic megaprimers were PCR amplified (KAPA HiFi HotStart ReadyMix, Roche, Switzerland, cat. KK3605), purified by using NucleoSpin® Gel and PCR Clean-up kit (Nacherey-Nagel, Germany, REF 740609.50) and cloned into pJYDC1 (Adgene ID: 162458) (Zahradnik et al., 2021a). Parental pJYDC1 molecules were cleaved by DpnI treatment (1 h, NEB, USA, cat. R0176) and the reaction mixture was electroporated into E. coli Cloni® 10G cells (Lucigen, USA). The correctness of mutagenesis was verified by sequencing.

### Cloning of Spike and RBD

Expression plasmids of wild-type and Omicron BA.1 spike and RBD of BA.1 and BA.2 were constructed encoding for human codon-optimized sequences from BA.1 (EPI_ISL_6640917) and BA.2 (EPI_ISL_6795834.2). The constructs of Wild-type and BA.1 Spike and RBD plasmids are the same as previously described (Dejnirattisai, Wanwisa, et al. “The antigenic anatomy of SARS-CoV-2 receptor binding domain.” Cell 184.8 (2021): 2183-2200). A synthetic codon-optimized RBD fragment of BA.2 was used as a template and construct was amplified by PCR and cloned into pNEO vector as previously described (Dejnirattisai et al., 2021a; Supasa et al., 2021; Zhou et al., 2021). The construct was verified by Sanger sequencing.

To generate His-tagged constructs of BA.4/5 RBD, site-directed PCR mutagenesis was performed using the BA.2 RBD construct as the template (Nutalai et al., 2022), with the introduction of L452R. F486V and R493Q mutations. The gene fragment was amplified with pNeoRBD3330miIF (5′-GGTTGCGTAGCTGAAACCGGTCATCACCATCACCATCACACC AATCTGTGCCCTTTCGAC-3′) and pNeoRBD333_R (5′-GTGATGGTGGTGCTTGGTACCT TATTACTTCT TGCCGCACACGGTAGC-3′), and cloned into the pNeo vector (Supasa et al., 2021, “Reduced neutralization of SARS-CoV-2 B.1.1.7 variant by convalescent and vaccine sera”. Cell 184, 2201-2211 e2207). To generate the BA.4/5 RBD construct containing a BAP-His tag, the gene fragment was amplified with RBD333 F (5′-GCGTAGCTGAAACCGGCACCAATCTGTGC CCTTTCGAC-3′) and RBD333_BAP_R (5′-GTCATTCAGCAAGCTCTTCTTGCCGCACACGG TAGC-3′), and cloned into the pOPINTTGneo-BAP vector (Huo et al., 2020, “Neutralizing nanobodies bind SARS-CoV-2 spike RBD and block interaction with ACE2”. Nature structural & molecular biology 27, 846-854.). Cloning was performed using the ClonExpress II One Step Cloning Kit (Vazyme). The Constructs were verified by Sanger sequencing after plasmid isolation using QIAGEN Minprep kit (QIAGEN).

To generate the BA.2.75 RBD construct, site-directed PCR mutagenesis was performed using the BA.2 Spike construct as the template (Nutalai et al., 2022), with the introduction of D339H, G446S, N460K and R493Q mutations suing primers listed in FIG. 26; the gene fragment was amplified with D339_pNeoF and RBD 333_BAP_R (FIG. 26), and cloned into the pOPINTTGneo-BAP vector (Huo et al., 2020 “Neutralizing nanobodies bind SARS-CoV-2 spike RBD and block interaction with ACE2.” Nature structural & molecular biology 27, 846-854). To generate the BA.2+R493Q RBD construct, site-directed PCR mutagenesis was performed using the BA.2 Spike construct as the template, with the introduction of the R493Q mutation using primers listed in FIG. 26; the gene fragment was amplified with pNeoRBD333Omi_F and BD333_BAP_R, and cloned into the pNeo vector (Supasa et al., 2021 “Reduced neutralization of SARS-CoV-2 B.1.1.7 variant by convalescent and vaccine sera.” Cell 184, 2201-2211 e2207). Cloning was performed using the ClonExpress 11 One Step Cloning Kit (Vazyme). The Constructs were verified by Sanger sequencing after plasmid isolation using QIAGEN Miniprep kit (QIAGEN).

### Production of RBDs

Plasmids encoding RBDs were transfected into Expi293F™ Cells (ThermoFisher) by PEI, cultured in FreeStyle™ 293 Expression Medium (ThermoFisher) at 30° C. with 8% CO2 for 4 days. To express biotinylated RBDs, the RBD-BAP plasmid was co-transfected with pDisplay-BirA-ER (Addgene plasmid 20856; coding for an ER-localized biotin ligase), in the presence of 0.8 mM D-biotin (Sigma-Aldrich).

### Production of BA.2.75 RBDs

Plasmids encoding RBDs were transfected into Expi293F™ Cells (ThermoFisher) by PEI, cultured in FreeStyle™ 293 Expression Medium (ThermoFisher) at 37° C. for 1 day followed by 30° C. for 3 days with 8% CO2. To express biotinylated RBDs, the RBD-BAP plasmid was co-transfected with pDisplay-BirA-ER (Addgene plasmid 20856; coding for an ER-localized biotin ligase), in the presence of 0.8 mM D-biotin (Sigma-Aldrich). The conditioned medium was diluted 1:2 into binding buffer (50 mM sodium phosphate, 500 mM sodium chloride, pH 8.0). RBDs were purified with a 5 mL HisTrap nickel column (GE Healthcare) through His-tag binding, followed by a Superdex 75 10/300 GL gel filtration column (GE Healthcare) in 10 mM HEPES and 150 mM sodium chloride.

### Protein Production

Protein expression and purification were conducted as described previously (Dejnirattisai et al., 2021a; Zhou et al., 2020). Briefly, plasmids encoding proteins were transiently expressed in HEK293T (ATCC CRL-11268) cells. The conditioned medium was concentrated using a QuixStand benchtop system. His-tagged Omicron RBD were purified with a 5 mL HisTrap nickel column (GE Healthcare) and further polished using a Superdex 75 HiLoad 16/60 gel filtration column (GE Healthcare). Twin-strep tagged Omicron spike was purified with Strep-Tactin XT resin (IBA lifesciences). ˜4 mg of ACE2 was mixed with homemade His-tagged 3C protease and DTT (final concentration 1 mM). After incubated at 4° C. for one day, the sample was flown through a 5 mL HisTrap nickel column (GE Healthcare). His-tagged proteins were removed by the nickel column and purified ACE2 was harvested and concentrated.

IgG mAbs and Fab Purification

To purify full length IgG mAbs, supernatants of mAb expression were collected and filtered by a vacuum filter system and loaded on protein A/G beads over night at 4° C. Beads were washed with PBS three times and 0.1 M glycine pH 2.7 was used to elute IgG. The eluate was neutralized with Tris-HCl pH 8 buffer to make the final pH=7. The IgG concentration was determined by spectro-photometry and buffered exchanged into PBS. To express and purify Fabs 158 and EY6A, heavy chain and light chain expression plasmids of Fab were co-transfected into HEK293T cells by PEI. After cells cultured for 5 days at 37° C. with 5% CO2, culture supernatant was harvested and filtered using a 0.22 mm polyethersulfone filter. Fab 158 was purified using Strep-Tactin XT resin (IBA lifesciences) and Fab EY6A was purified with Ni-NTA column (GE HealthCare) and a Superdex 75 HiLoad 16/60 gel filtration column (GE Healthcare). AstraZeneca and Regeneron antibodies were provided by AstraZeneca, Vir, Lilly and Adagio antibodies were provided by Adagio. For the antibodies heavy and light chains of the indicated antibodies were transiently transfected into 293Y cells and antibody purified from supernatant on protein A. Fab fragments of 58 and beta-55 were digested from purified IgGs with papain using a Pierce Fab Preparation Kit (Thermo Fisher), following the manufacturer's protocol.

### Surface Plasmon Resonance

The surface plasmon resonance experiments were performed using a Biacore T200 (GE Healthcare). All assays were performed with a running buffer of HBS-EP (Cytiva) at 25° C.

To determine the binding kinetics between the SARS-CoV-2 RBDs and ACE2/monoclonal antibody (mAb), a Protein A sensor chip (Cytiva) was used. ACE2-Fc or mAb was immobilised onto the sample flow cell of the sensor chip. The reference flow cell was left blank. RBD was injected over the two flow cells at a range of five concentrations prepared by serial twofold dilutions, at a flow rate of 30 μl min⁻¹ using a single-cycle kinetics programme. Running buffer was also injected using the same programme for background subtraction. All data were fitted to a 1:1 binding model using Biacore T200 Evaluation Software 3.1.

To determine the binding kinetics between the SARS-CoV-2 Spikes and ACE2, a CM5 sensor chip was used. The sensor chip was firstly activated by an injection of equal volume mix of EDC and NHS (Cytiva) at 20 uL/min for 300 s, followed by an injection of Spike sample at 20 μg/mL in 10 mM sodium acetate pH 5.0 (Cytiva) onto the sample flow cell of the sensor chip at 10 uL/min, and finally with an injection of 1.0 M Ethanolamine-HCl, pH 8.5 (Cytiva) at 20 uL/min for 180 s. The reference flow cell was left blank. ACE2 was injected over the two flow cells at a range of five concentrations prepared by serial twofold dilutions, at a flow rate of 30 μl min⁻¹ using a single-cycle kinetics programme. Running buffer was also injected using the same programme for background subtraction.

All data were fitted to a 1:1 binding model using Biacore T200 Evaluation Software 3.1. To determine the binding kinetics between the RBDs and mAb Omi-32/Omi-42, a Biotin CAPture Kit (Cytiva) was used. Biotinylated RBD was immobilised onto the sample flow cell of the sensor chip. The reference flow cell was left blank. The mAb Fab was injected over the two flow cells at a range of five concentrations prepared by serial two-fold dilutions, at a flow rate of 30 μl min−1 using a single-cycle kinetics programme. Running buffer was also injected using the same programme for background subtraction. All data were fitted to a 1:1 binding model using Biacore T200 Evaluation Software 3.1.

To determine the binding affinity of BA.4/5 RBD and mAb Omi-12, a Protein A sensor chip (Cytiva) was used. The Ig Omi-12 was immobilised onto the sample flow cell of the sensor chip. The reference flow cell was left blank. RBD was injected over the two flow cells at a range of seven concentrations prepared by serial twofold dilutions, at a flow rate of 30 μl min−1. Running buffer was also injected using the same programme for background subtraction. All data were fitted to a 1:1 binding model using Prism9 (GraphPad).

To compare the binding profiles between BA.2 and BA.4/5 RBD for mAb Omi-06/Omi-25/Omi-26, a Protein A sensor chip (Cytiva) was used. mAb in the IgG form was immobilised onto the sample flow cell of the sensor chip to a similar level (˜350 RU). The reference flow cell was left blank. A single injection of RBD was performed over the two flow cells at 200 nM, at a flow rate of 30 μl min−1. Running buffer was also injected using the same programme for background subtraction. The sensorgrams were plotted using Prism9 (GraphPad).

To compare the binding profiles between BA.2 and BA.4/5 RBD for mAb Omi-02/Omi-23/Omi-31, a Biotin CAPture Kit (Cytiva) was used. Biotinylated BA.2 and BA.4/5 RBD was immobilised onto the sample flow cell of the sensor chip to a similar level (˜120 RU). The reference flow cell was left blank. A single injection of mAb Fab was performed over the two flow cells at 200 nM, at a flow rate of 30 μl min−1. Running buffer was also injected using the same programme for background subtraction. The sensorgrams were plotted using Prism9 (GraphPad).

To determine the binding kinetics between BA.2.75 or BA.2+R493Q RBD and ACE2, a Protein A sensor chip (Cytiva) was used. ACE2-Fc was immobilised onto the sample flow cell of the sensor chip. The reference flow cell was left blank. RBD was injected over the two flow cells at a range of five concentrations prepared by serial two-fold dilutions, at a flow rate of 30 μl min−1 using a single-cycle kinetics programme. Running buffer was also injected using the same programme for background subtraction. All data were fitted to a 1:1 binding model using Biacore T200 Evaluation Software 3.1.

To confirm the binding kinetics between the BA.2.75 RBD and ACE2, a Biotin CAPture Kit (Cytiva) was used. Biotinylated ACE2 (bio-ACE2) was immobilised onto the sample flow cell of the sensor chip. The reference flow cell was left blank. The BA.2.75 RBD was injected over the two flow cells at a range of five concentrations prepared by serial two-fold dilutions, at a flow rate of 30 μl min⁻¹ using a single-cycle kinetics programme. Running buffer was also injected using the same programme for background subtraction. All data were fitted to a 1:1 binding model using Biacore T200 Evaluation Software 3.1.

To determine the binding kinetics between the BA.2.75 or BA.2 RBD and mAbs, a Biotin CAPture Kit (Cytiva) was used. Biotinylated RBD was immobilised onto the sample flow cell of the sensor chip. The reference flow cell was left blank. The Fab of Omi-18 or Omi-32 was injected over the two flow cells at a range of five concentrations prepared by serial two-fold dilutions, at a flow rate of 30 μl min⁻¹ using a single-cycle kinetics programme. For the binding of Omi-20 for bio-BA.2 RBD, the Fab of Omi-20 was injected over the two flow cells at a range of five concentrations prepared by serial two-fold dilutions, at a flow rate of 30 μl min⁻¹ using a single-cycle kinetics programme. For the binding of Omi-20 for bio-BA.2.75 RBD, the Fab of Omi-20 was injected over the two flow cells at a range of eight concentrations prepared by serial twofold dilutions, at a flow rate of 30 μl min⁻¹. Running buffer was also injected using the same programme for background subtraction. All data were fitted to a 1:1 binding model using Biacore T200 Evaluation Software 3.1.

To compare the binding profiles between BA.2 and BA.2.75 RBD for mAb Omi-29, a Biotin CAPture Kit (Cytiva) was used. Biotinylated BA.2 and BA.2.75 RBD was immobilised onto the sample flow cell of the sensor chip to a similar level (˜110 RU). The reference flow cell was left blank. A single injection of mAb Fab was performed over the two flow cells at 1 μM, at a flow rate of 30 μl min⁻¹. Running buffer was also injected using the same programme for background subtraction. The sensorgrams were plotted using Prism9 (GraphPad).

To compare the binding profiles between BA.2 and BA.2.75 RBD for mAb Omi-36, a sensor chip Protein A (Cytiva) was used. mAb Omi-36 in the IgG form was immobilised onto the sample flow cell of the sensor chip. The reference flow cell was left blank. A single injection of RBD was performed over the two flow cells at 200 nM, at a flow rate of 30 μl min⁻¹. Running buffer was also injected using the same programme for background subtraction. The sensorgrams were plotted using Prism9 (GraphPad).

IgG mAbs and Fabs Production

AstraZeneca and Regeneron antibodies were provided by AstraZeneca, Vir, Lilly and Adagio antibodies were provided by Adagio, LY-CoV1404 was provided by LifeArc. For the in-house antibodies, heavy and light chains of the indicated antibodies were transiently transfected into 293Y or 293T cells and antibody purified from supernatant on protein A as previously described (Nutalai et al., 2022). Fabs were digested from purified IgGs with papain using a Pierce Fab Preparation Kit (Thermo Fisher), following the manufacturer's protocol.

### Quantification and Statistical Analysis

Statistical analyses are reported in the results and figure legends. Neutralization was measured by FRNT. The percentage of focus reduction was calculated and IC50 (FRNT50) was determined using the probit program from the SPSS package. The Wilcoxon matched-pairs signed rank test was used for the analysis and two-tailed P values were calculated on geometric mean values.

### Crystallization

RBD proteins were deglycosylated with Endoglycosidase F1 before used for crystallization. Omicron BA.1-RBD was mixed with Omi-12 and beta-54 Fabs, separately, in a 1:1:1 molar ratio, with a final concentration of 7 mg ml-1. These complexes were separately incubated at room temperature for 30 min. Initial screening of crystals was set up in Crystalquick 96-well X plates (Greiner Bio-One) with a Cartesian Robot using the nanoliter sitting-drop vapor-diffusion method, with 100 nL of protein plus 100 nL of reservoir in each drop, as previously described (Walter et al., 2003, Journal of Applied Crystallography 36, 308-314).

Crystals of BA.1-RBD/Omi-12/beta-54 were formed in Hampton Research PEGRx condition 1-46, containing 0.1 M Sodium citrate tribasic dihydrate pH 5.0 and 18% (w/v) PEG 20000. Complex of BA.1-RBD/Omi-12/beta-54 was screen in Hampton Research Ammonium sulphate screen C2, containing 2.4 M (NH₄)₂SO₄ and 0.1 M citric acid pH 5.0, but only crystals of Fab Omi-12 alone were formed in this condition.

### Crystallization of BA.2.75 RBD

Purified BA.2.75 RBD was deglycosylated with Endoglycosidase H1 and mixed with ACE2 in a 1:1 molar ratio, with a final concentration of 13.0 mg ml-1. Initial screening of crystals was set up in Crystalquick 96-well X plates (Greiner Bio-One) with a Cartesian Robot using the nanoliter sitting-drop vapor-diffusion method, with 100 nL of protein plus 100 nL of reservoir in each drop, as previously described (Walter et al., 2003). Crystals of BA.2.75 RBD-ACE2 complex were formed in Hampton Research PEGRx condition 2-25, containing 0.1% (w/v) n-Octyl-b-D-glucoside, 0.1 M Sodium citrate tribasic dihydrate pH 5.5 and 22% (w/v) PEG 3350. Diffraction data were collected at 100 K at beamline 103 of Diamond Light Source, UK, using the automated queue system that allows unattended automated data collection (https://www.diamond.ac.uk/Instruments/Mx/I03/I03-Manual/Unattended-Data-Collections.html).

### X-Ray Data Collection, Structure Determination and Refinement

Diffraction data were collected at 100 K at beamline 103 of Diamond Light Source, UK. All data were collected as part of an automated queue system allowing unattended automated data collection (https://www.diamond.ac.uk/Instruments/Mx/I03/I03-Manual/Unattended-Data-Collections.html). Crystals were pre-frozen by mounting in loops and soaked for a second in cryo-protectant containing 25% glycerol and 75% mother liquor. Diffraction images of 0.1° rotation were recorded on an Eiger2 XE 16M detector (exposure time from 0.018 s per image, beam size 80×20 m, 10% beam transmission and wavelength of 0.9762 Å). Data were indexed, integrated and scaled with the automated data processing program Xia2-dials (Winter, 2010, Journal of applied crystallography 43, 186-190; Winter et al., 2018, Acta Crystallogr D Struct Biol 74, 85-97). 360° of data was collected from a single crystal for each of the data sets.

Structures were determined by molecular replacement with PHASER (McCoy et al., 2007, J Appl Crystallogr 40, 658-674). VhVl and ChCl domains which have the most sequence similarity to previously determined SARS-CoV-2 RBD/Fab structures (Dejnirattisai et al., 2021, Cell 184, 2183-2200 e2122; Dejnirattisai et al., 2021, Cell 184, 2939-2954 e2939; Huo et al., 2020, Cell Host Microbe 28, 445-454; Liu et al., 2021, Cell 184, 4220-4236 e4213; Supasa et al., 2021, Cell 184, 2201-2211 e2207; Zhou et al., 2021, Cell 184, 2348-2361 e2346; Zhou et al., 2020, Nature structural & molecular biology 27, 950-958) were used as search models for each of the current structure determination.

Model rebuilding with COOT (Emsley et al., 2010, Biological Crystallography 66, 486-501) and refinement with Phenix (Liebschner et al., 2019, Acta Crystallogr D Struct Biol 75, 861-877) were used for all the structures. Due to the lower resolution, only rigid-body and group B-factor refinement were performed for structures of BA.1-RBD/O-12/Beta-54 complex.

Data collection and structure refinement statistics are given in Tables 19 and 25. Structural comparisons used SHP (Stuart et al., 1979, J Mol Biol 134, 109-142), residues forming the RBD/Fab interface were identified with PISA (Krissinel and Henrick, 2007, J Mol Biol 372, 774-797) and figures were prepared with PyMOL (The PyMOL Molecular Graphics System, Version 1.2r3pre, Schrödinger, LLC).

### Example 5

### Antibody Structure

The structure of the BA.1 RBD/Fab Omi-12/Fab Beta-54 ternary complex was determined to 5.5 Å resolution (Table 19, FIG. 6A). A slight clash was observed between the two Fabs despite the BLI experiment showing no significant competition for binding between them. A high-resolution structure of un-complexed Omi-12 fab (2.1 Å resolution, Table 19) has been modelled into the electron density for the complex (FIG. 6B, 6C). Superimposing Fab 253 onto Fab Omi-12 suggests that Q493R would clash with the H2 loop of Fab 253, whereas in Omi-12, H2 adopts a slightly flattened structure. This structural change is attributable to antibody maturation via the somatic mutation V53P in the heavy chain variable region of Omi-12 which forms a stacking interaction with Y489 (FIG. 6D).

Omi-12 and Antibody 253 are both derived from the germline heavy chain IGHV1-58. Interestingly, similar to antibody 253, other antibodies derived from the germline heavy chain IGHV1-58 described herein, i.e. Beta-47, Beta-25, antibody 55, antibody 165 and antibody 318, also has a valine (V) at position 53 in the heavy chain variable region, i.e. valine (V) at position 53 in SEQ ID NO: 262 (antibody 253), SEQ ID NO: 591 (Beta-47), SEQ ID NO: 461 (Beta-25), SEQ ID NO: 62 (antibody 55), SEQ ID NO: 182 (antibody 165) and SEQ ID NO: 332 (antibody 318). Position 53 in these sequences corresponds to position 58 according to IMGT numbering. Based on the data, modification of any of these antibodies by substitution of valine at position 53 with proline (i.e. V53P absolute numbering, or V58P according to IMGT numbering) would result in an antibody that would be effective against Omicron.

Furthermore, antibody AZD8895 (heavy chain variable region amino acid sequences provided in SEQ ID NO: 963 and light chain variable region amino acid sequence provided in SEQ ID NO: 965) is also derived from the germline heavy chain IGHV1-58 (e.g. see Dong et al. Nat Microbiol 6, 1233-1244 (2021)). AZD8895 has an isoleucine (I) at position 53 in the heavy chain variable region, which corresponds to position 58 according to IMGT numbering. Based on the data herein, modification of the heavy chain variable region AZD8895 (SEQ ID NO: 963) by substitution of isoleucine at position 53 with proline (i.e. 153P) using absolute numbering, or I58P using IMGT numbering, would result in an antibody that would be effective against Omicron.

Hence, the data indicate that the modification of VH1-58 antibodies such that a proline is present at position 53 (corresponding to position 58 according to IMGT numbering) in the heavy chain variable region would make them particularly effective against Omicron.

### ACE2 BA.2. 75 RBD Structure

To elucidate the molecular mechanism for high affinity, the structure of the BA.2.75 RBD with ACE2 was determined by crystallography (according to the methods described in Example 4). As expected the binding mode was essentially indistinguishable from that observed before (FIG. 20A), although there were significant rearrangements outside of the ACE2 footprint, with the flexible RBD 371-375 loop re-arranging and part of the C-terminal 6×His tag becoming ordered. FIG. 20B shows a close-up of the binding interface, compared with the ACE2/BA.2 RBD complex. In other complexes (with either R or Q at RBD 493) K31 of ACE2 tends to be disordered, whereas it is well ordered in the BA.2.75 complex, allowing K31 to form a potential hydrogen bond with the glutamine sidechain possibly increasing the affinity of ACE2.

### Tables

TABLE 1
SEQ ID NOs of antibodies raised against early pandemic strains

| Antibody | Heavy Chain | Heavy Chain | Light Chain | Light Chain | | | | | | |
|---|---|---|---|---|---|---|---|---|---|---|
| number | protein sequence | nucleotide [symbol] | protein sequence | nucleotid [symbol] | CDRH1 | CDRH2 | CDRH3 | CDRL1 | CDRL2 | CDRL3 |
| 2 | 2 | 1 | 4 | 3 | 5 | 6 | 7 | 8 | 9 | 10 |
| 22 | 12 | 11 | 14 | 13 | 15 | 16 | 17 | 18 | 19 | 20 |
| 40 | 22 | 21 | 24 | 23 | 25 | 26 | 27 | 28 | 29 | 30 |
| 44 | 32 | 31 | 34 | 33 | 35 | 36 | 37 | 38 | 39 | 40 |
| 45 | 42 | 41 | 44 | 43 | 45 | 46 | 47 | 48 | 49 | 50 |
| 54 | 52 | 51 | 54 | 53 | 55 | 56 | 57 | 58 | 59 | 60 |
| 55 | 62 | 61 | 64 | 63 | 65 | 66 | 67 | 68 | 69 | 70 |
| 58 | 72 | 71 | 74 | 73 | 75 | 76 | 77 | 78 | 79 | 80 |
| 61 | 82 | 81 | 84 | 83 | 85 | 86 | 87 | 88 | 89 | 90 |
| 75 | 92 | 91 | 94 | 93 | 95 | 96 | 97 | 98 | 99 | 100 |
| 88 | 102 | 101 | 104 | 103 | 105 | 106 | 107 | 108 | 109 | 110 |
| 111 | 112 | 111 | 114 | 113 | 115 | 116 | 117 | 118 | 119 | 120 |
| 132 | 122 | 121 | 124 | 123 | 125 | 126 | 127 | 128 | 129 | 130 |
| 140 | 132 | 131 | 134 | 133 | 135 | 136 | 137 | 138 | 139 | 140 |
| 148 | 142 | 141 | 144 | 143 | 145 | 146 | 147 | 148 | 149 | 150 |
| 150 | 152 | 151 | 154 | 153 | 155 | 156 | 157 | 158 | 159 | 160 |
| 158 | 162 | 161 | 164 | 163 | 165 | 166 | 167 | 168 | 169 | 170 |
| 159 | 172 | 171 | 174 | 173 | 175 | 176 | 177 | 178 | 179 | 180 |
| 165 | 182 | 181 | 184 | 183 | 185 | 186 | 187 | 188 | 189 | 190 |
| 170 | 192 | 191 | 194 | 193 | 195 | 196 | 197 | 198 | 199 | 200 |
| 175 | 202 | 201 | 204 | 203 | 205 | 206 | 207 | 208 | 209 | 210 |
| 177 | 212 | 211 | 214 | 213 | 215 | 216 | 217 | 218 | 219 | 220 |
| 181 | 222 | 221 | 224 | 223 | 225 | 226 | 227 | 228 | 229 | 230 |
| 182 | 232 | 231 | 234 | 233 | 235 | 236 | 237 | 238 | 239 | 240 |
| 183 | 242 | 241 | 244 | 243 | 245 | 246 | 247 | 248 | 249 | 250 |
| 222 | 252 | 251 | 254 | 253 | 255 | 256 | 257 | 258 | 259 | 260 |
| 253 | 262 | 261 | 264 | 263 | 265 | 266 | 267 | 268 | 269 | 270 |
| 253H55L | 262 | 261 | 64 | 63 | 265 | 266 | 267 | 68 | 69 | 70 |
| 253H165L | 262 | 261 | 184 | 183 | 265 | 266 | 267 | 188 | 189 | 190 |
| 269 | 272 | 271 | 274 | 273 | 275 | 276 | 277 | 278 | 279 | 280 |
| 278 | 282 | 281 | 284 | 283 | 285 | 286 | 287 | 288 | 289 | 290 |
| 281 | 292 | 291 | 294 | 293 | 295 | 296 | 297 | 298 | 299 | 300 |
| 282 | 302 | 301 | 304 | 303 | 305 | 306 | 307 | 308 | 309 | 310 |
| 285 | 312 | 311 | 314 | 313 | 315 | 316 | 317 | 318 | 319 | 320 |
| 316 | 322 | 321 | 324 | 323 | 325 | 326 | 327 | 328 | 329 | 330 |
| 318 | 332 | 331 | 334 | 333 | 335 | 336 | 337 | 338 | 339 | 340 |
| 334 | 342 | 341 | 344 | 343 | 345 | 346 | 347 | 348 | 349 | 350 |
| 361 | 352 | 351 | 354 | 353 | 355 | 356 | 357 | 358 | 359 | 360 |
| 382 | 362 | 361 | 364 | 363 | 365 | 366 | 367 | 368 | 369 | 370 |
| 384 | 372 | 371 | 374 | 373 | 375 | 376 | 377 | 378 | 379 | 380 |
| 394 | 382 | 381 | 384 | 383 | 385 | 386 | 387 | 388 | 389 | 390 |
| 398 | 392 | 391 | 394 | 393 | 395 | 396 | 397 | 398 | 399 | 400 |

[symbol]

indicates data missing or illegible when filed

TABLE 2
SEQ ID NOs of antibodies raised against the Beta strain

| | Heavy Chain | Heavy Chain | Light Chain | Light Chain | | | | | | |
|---|---|---|---|---|---|---|---|---|---|---|
| Antibody | nucleotide | protein | nucleotide | protein | | | | | | |
| number | sequence | [symbol] | sequence | sequence | CDRH1 | CDRH2 | CDRH3 | CDRL1 | CDRL2 | CDRL3 |
| Beta-06 | 401 | 402 | 403 | 404 | 405 | 406 | 407 | 408 | 409 | 410 |
| Beta-10 | 411 | 412 | 413 | 414 | 415 | 416 | 417 | 418 | 419 | 420 |
| Beta-20 | 421 | 422 | 423 | 424 | 425 | 426 | 427 | 428 | 429 | 430 |
| Beta-22 | 431 | 432 | 433 | 434 | 435 | 436 | 437 | 438 | 439 | 440 |
| Beta-23 | 441 | 442 | 443 | 444 | 445 | 446 | 447 | 448 | 449 | 450 |
| Beta-24 | 451 | 452 | 453 | 454 | 455 | 456 | 457 | 458 | 459 | 460 |
| Beta-25 | 461 | 462 | 463 | 464 | 465 | 466 | 467 | 468 | 469 | 470 |
| Beta-26 | 471 | 472 | 473 | 474 | 475 | 476 | 477 | 478 | 479 | 480 |
| Beta-27 | 481 | 482 | 483 | 484 | 485 | 486 | 487 | 488 | 489 | 490 |
| Beta-29 | 491 | 492 | 493 | 494 | 495 | 496 | 497 | 498 | 499 | 500 |
| Beta-30 | 501 | 502 | 503 | 504 | 505 | 506 | 507 | 508 | 509 | 510 |
| Beta-32 | 511 | 512 | 513 | 514 | 515 | 516 | 517 | 518 | 519 | 520 |
| Beta-33 | 521 | 522 | 523 | 524 | 525 | 526 | 527 | 528 | 529 | 530 |
| Beta-34 | 531 | 532 | 533 | 534 | 535 | 536 | 537 | 538 | 539 | 540 |
| Beta-38 | 541 | 542 | 543 | 544 | 545 | 546 | 547 | 548 | 549 | 550 |
| Beta-40 | 551 | 552 | 553 | 554 | 555 | 556 | 557 | 558 | 559 | 560 |
| Beta-43 | 561 | 562 | 563 | 564 | 565 | 566 | 567 | 568 | 569 | 570 |
| Beta-44 | 571 | 572 | 573 | 574 | 575 | 576 | 577 | 578 | 579 | 580 |
| Beta-45 | 581 | 582 | 583 | 584 | 585 | 586 | 587 | 588 | 589 | 590 |
| Beta-47 | 591 | 592 | 593 | 594 | 595 | 596 | 597 | 598 | 599 | 600 |
| Beta-48 | 601 | 602 | 603 | 604 | 605 | 606 | 607 | 608 | 609 | 610 |
| Beta-49 | 611 | 612 | 613 | 614 | 615 | 616 | 617 | 618 | 619 | 620 |
| Beta-50 | 621 | 622 | 623 | 624 | 625 | 626 | 627 | 628 | 629 | 630 |
| Beta-51 | 631 | 632 | 633 | 634 | 635 | 636 | 637 | 638 | 639 | 640 |
| Beta-53 | 641 | 642 | 643 | 644 | 645 | 646 | 647 | 648 | 649 | 650 |
| Beta-54 | 651 | 652 | 653 | 654 | 655 | 656 | 657 | 658 | 659 | 660 |
| Beta-55 | 661 | 662 | 663 | 664 | 665 | 666 | 667 | 668 | 669 | 670 |
| Beta-56 | 671 | 672 | 673 | 674 | 675 | 676 | 677 | 678 | 679 | 680 |

[symbol]

indicates data missing or illegible when filed

TABLE 3
SEQ ID NOs of antibodies raised against the Omicron strain

| | | Heavy | | Light | | | | | | |
|---|---|---|---|---|---|---|---|---|---|---|
| | Heavy | Chain | Light | Chain | | | | | | |
| Antibody | Chain | protein | Chain | protein | | | | | | |
| number | nucleotide | sequence | nucleotide | sequence | CDRH1 | CDRH2 | CDRH3 | CDRL1 | CDRL2 | CDRL3 |
| Omi02 | 681 | 682 | 683 | 684 | 685 | 686 | 687 | 688 | 689 | 690 |
| Omi03 | 691 | 692 | 693 | 694 | 695 | 696 | 697 | 698 | 699 | 700 |
| Omi06 | 701 | 702 | 703 | 704 | 705 | 706 | 707 | 708 | 709 | 710 |
| Omi08 | 711 | 712 | 713 | 714 | 715 | 716 | 717 | 718 | 719 | 720 |
| Omi09 | 721 | 722 | 723 | 724 | 725 | 726 | 727 | 728 | 729 | 730 |
| Omi12 | 731 | 732 | 733 | 734 | 735 | 736 | 737 | 738 | 739 | 740 |
| Omi16 | 741 | 742 | 743 | 744 | 745 | 746 | 747 | 748 | 749 | 750 |
| Omi17 | 751 | 752 | 753 | 754 | 755 | 756 | 757 | 758 | 759 | 760 |
| Omi18 | 761 | 762 | 763 | 764 | 765 | 766 | 767 | 768 | 769 | 770 |
| Omi20 | 771 | 772 | 773 | 774 | 775 | 776 | 777 | 778 | 779 | 780 |
| Omi23 | 781 | 782 | 783 | 784 | 785 | 786 | 787 | 788 | 789 | 790 |
| Omi24 | 791 | 792 | 793 | 794 | 795 | 796 | 797 | 798 | 799 | 800 |
| Omi25 | 801 | 802 | 803 | 804 | 805 | 806 | 807 | 808 | 809 | 810 |
| Omi26 | 811 | 812 | 813 | 814 | 815 | 816 | 817 | 818 | 819 | 820 |
| Omi27 | 821 | 822 | 823 | 824 | 825 | 826 | 827 | 828 | 829 | 830 |
| Omi28 | 831 | 832 | 833 | 834 | 835 | 836 | 837 | 838 | 839 | 840 |
| Omi29 | 841 | 842 | 843 | 844 | 845 | 846 | 847 | 848 | 849 | 850 |
| Omi30 | 851 | 852 | 853 | 854 | 855 | 856 | 857 | 858 | 859 | 860 |
| Omi31 | 861 | 862 | 863 | 864 | 865 | 866 | 867 | 868 | 869 | 870 |
| Omi32 | 871 | 872 | 873 | 874 | 875 | 876 | 877 | 878 | 879 | 880 |
| Omi33 | 881 | 882 | 883 | 884 | 885 | 886 | 887 | 888 | 889 | 890 |
| Omi34 | 891 | 892 | 893 | 894 | 895 | 896 | 897 | 898 | 899 | 900 |
| Omi35 | 901 | 902 | 903 | 904 | 905 | 906 | 907 | 908 | 909 | 910 |
| Omi36 | 911 | 912 | 913 | 914 | 915 | 916 | 917 | 918 | 919 | 920 |
| Omi38 | 921 | 922 | 923 | 924 | 925 | 926 | 927 | 928 | 929 | 930 |
| Omi39 | 931 | 932 | 933 | 934 | 935 | 936 | 937 | 938 | 939 | 940 |
| Omi41 | 941 | 942 | 943 | 944 | 945 | 946 | 947 | 948 | 949 | 950 |
| Omi42 | 951 | 952 | 953 | 954 | 955 | 956 | 957 | 958 | 959 | 960 |

TABLE 4
Examples of the mixed chain antibodies generated from antibodies derived from the same germline heavy chain IGHV3-53

| Heavy | | | | | | | | | |
|---|---|---|---|---|---|---|---|---|---|
| chain | | | | | | | | | |
| (H)/light | | | | | | | | | |
| chain (L) of | | | | | | | | | |
| antibody | Omi03H | Omi18H | Omi29H | Beta-27H | 150H | 158H | 175H | 222H | 269H |
| Omi03L | — | Omi18H | Omi29H | Beta-27H | 150H | 158H | 175H | 222H | 269H |
| | | Omi03L | Omi03L | Omi03L | Omi03L | Omi03L | Omi03L | Omi03L | Omi03L |
| Omi18L | Omi03H | — | Omi29H | Beta-27H | 150H | 158H | 175H | 222H | 269H |
| | Omi18L | | Omi18L | Omi18L | Omi18L | Omi18L | Omi18L | Omi18L | Omi18L |
| Omi29L | Omi03H | Omi18H | — | Beta-27H | 150H | 158H | 175H | 222H | 269H |
| | Omi29L | Omi29L | | Omi29L | Omi29L | Omi29L | Omi29L | Omi29L | Omi29L |
| Beta-27L | Omi03H | Omi18H | Omi29H | — | 150H | 158H | 175H | 222H | 269H |
| | Beta- | Beta- | Beta- | | Beta- | Beta- | Beta- | Beta- | Beta- |
| | 27L | 27L | 27L | | 27L | 27L | 27L | 27L | 27L |
| 150L | Omi03H | Omi18H | Omi29H | Beta-27H | — | 158H | 175H | 222H | 269H |
| | 150L | 150L | 150L | 150L | | 150L | 150L | 150L | 150L |
| 158L | Omi03H | Omi18H | Omi29H | Beta-27H | 150H | — | 175H | 222H | 269H |
| | 158L | 158L | 158L | 158L | 158L | | 158L | 158L | 158L |
| 175L | Omi03H | Omi18H | Omi29H | Beta-27H | 150H | 158H | — | 222H | 269H |
| | 175L | 175L | 175L | 175L | 175L | 175L | | 175L | 175L |
| 222L | Omi03H | Omi18H | Omi29H | Beta-27H | 150H | 158H | 175H | — | 269H |
| | 222L | 222L | 222L | 222L | 222L | 222L | 222L | | 222L |
| 269L | Omi03H | Omi18H | Omi29H | Beta-27H | 150H | 158H | 175H | 222H | — |
| | 269L | 269L | 269L | 269L | 269L | 269L | 269L | 269L | |

TABLE 5
Examples of the mixed chain antibodies generated from antibodies derived from the same germline
heavy chain IGHV3-53 + IGHV3-66

| Heavy | | | | | | | | | | | |
|---|---|---|---|---|---|---|---|---|---|---|---|
| chain | | | | | | | | | | | |
| (H)/light | | | | | | | | | | | |
| chain | | | | | | | | | | | |
| (L) of | | | | | | | | | | | |
| antibody | Omi03H | Omi18H | Omi29H | Omi16H | Omi17H | Omi20H | Omi27H | Omi28H | Omi36H | Beta-27H | 150H |
| Omi03L | — | Omi18H | Omi29H | Omi16H | Omi17H | Omi20H | Omi27H | Omi28H | Omi36H | Beta-27H | 150H |
| | | Omi03L | Omi03L | Omi03L | Omi03L | Omi03L | Omi03L | Omi03L | Omi03L | Omi03L | Omi03L |
| Omi18L | Omi03H | — | Omi29H | Omi16H | Omi17H | Omi20H | Omi27H | Omi28H | Omi36H | Beta-27H | 150H |
| | Omi18L | | Omi18L | Omi18L | Omi18L | Omi18L | Omi18L | Omi18L | Omi18L | Omi18L | Omi18L |
| Omi29L | Omi03H | Omi18H | — | Omi16H | Omi17H | Omi20H | Omi27H | Omi28H | Omi36H | Beta-27H | 150H |
| | Omi29L | Omi29L | | Omi29L | Omi29L | Omi29L | Omi29L | Omi29L | Omi29L | Omi29L | Omi29L |
| Omi16H | Omi03H | Omi18H | Omi29H | — | Omi17H | Omi20H | Omi27H | Omi28H | Omi36H | Beta-27H | 150H |
| | Omi16L | Omi16L | Omi16L | | Omi16L | Omi16L | Omi16L | Omi16L | Omi16L | Omi16L | Omi16L |
| Omi17H | Omi03H | Omi18H | Omi29H | Omi16H | — | Omi20H | Omi27H | Omi28H | Omi36H | Beta-27H | 150H |
| | Omi17L | Omi17L | Omi17L | Omi17L | | Omi17L | Omi17L | Omi17L | Omi17L | Omi17L | Omi17L |
| Omi20H | Omi03H | Omi18H | Omi29H | Omi16H | Omi17H | — | Omi27H | Omi28H | Omi36H | Beta-27H | 150H |
| | Omi20L | Omi20L | Omi20L | Omi20L | Omi20L | | Omi20L | Omi20L | Omi20L | Omi20L | Omi20L |
| Omi27H | Omi03H | Omi18H | Omi29H | Omi16H | Omi17H | Omi20H | — | Omi28H | Omi36H | Beta-27H | 150H |
| | Omi27L | Omi27L | Omi27L | Omi27L | Omi27L | Omi27L | | Omi27L | Omi27L | Omi27L | Omi27L |
| Omi28H | Omi03H | Omi18H | Omi29H | Omi16H | Omi17H | Omi20H | Omi27H | — | Omi36H | Beta-27H | 150H |
| | Omi28L | Omi28L | Omi28L | Omi28L | Omi28L | Omi28L | Omi28L | | Omi28L | Omi28L | Omi28L |
| Omi36H | Omi03H | Omi18H | Omi29H | Omi16H | Omi17H | Omi20H | Omi27H | Omi28H | — | Beta-27H | 150H |
| | Omi36L | Omi36L | Omi36L | Omi36L | Omi36L | Omi36L | Omi36L | Omi36L | | Omi36L | Omi36L |
| Beta-27L | Omi03H | Omi18H | Omi29H | Omi16H | Omi17H | Omi20H | Omi27H | Omi28H | Omi36H | — | 150H |
| | Beta-27L | Beta-27L | Beta-27L | Beta-27L | Beta-27L | Beta-27L | Beta-27L | Beta-27L | Beta-27L | | Beta-27L |
| 150L | Omi03H | Omi18H | Omi29H | Omi16H | Omi17H | Omi20H | Omi27H | Omi28H | Omi36H | Beta-27H | — |
| | 150L | 150L | 150L | 150L | 150L | 150L | 150L | 150L | 150L | 150L | |
| 158L | Omi0H3 | Omi18H | Omi29H | Omi16H | Omi17H | Omi20H | Omi27H | Omi28H | Omi36H | Beta-27H | 150H |
| | 158L | 158L | 158L | 158L | 158L | 158L | 158L | 158L | 158L | 158L | 158L |
| 175L | Omi0H3 | Omi18H | Omi29H | Omi16H | Omi17H | Omi20H | Omi27H | Omi28H | Omi3H6 | Beta-27H | 150H |
| | 175L | 175L | 175L | 175L | 175L | 175L | 175L | 175L | 175L | 175L | 175L |
| 222L | Omi03H | Omi18H | Omi29H | Omi16H | Omi17H | Omi20H | Omi27H | Omi28H | Omi36H | Beta-27H | 150H |
| | 222L | 222L | 222L | 222L | 222L | 222L | 222L | 222L | 222L | 222L | 222L |
| 269L | Omi03H | Omi18H | Omi29H | Omi16H | Omi17H | Omi20H | Omi27H | Omi28H | Omi36H | Beta-27H | 150H |
| | 269L | 269L | 269L | 269L | 269L | 269L | 269L | 269L | 269L | 269L | 269L |
| 40L | Omi03H | Omi18H | Omi29H | Omi16H | Omi17H | Omi20H | Omi27H | Omi28H | Omi36H | Beta-27H | 150H |
| | 40L | 40L | 40L | 40L | 40L | 40L | 40L | 40L | 40L | 40L | 40L |
| 398L | Omi03H | Omi18H | Omi29H | Omi16H | Omi17H | Omi20H | Omi27H | Omi28H | Omi36H | Beta-27H | 150H |
| | 398L | 398L | 398L | 398L | 398L | 398L | 398L | 398L | 398L | 398L | 398L |

| Heavy | | | | | | |
|---|---|---|---|---|---|---|
| chain | | | | | | |
| (H)/light | | | | | | |
| chain | | | | | | |
| (L) of | | | | | | |
| antibody | 158H | 175H | 222H | 269H | 40H | 398H |
| Omi03L | 158H | 175H | 222H | 269H | 40H | 398H |
| | Omi03L | Omi03L | Omi03L | Omi03L | Omi03L | Omi03L |
| Omi18L | 158H | 175H | 222H | 269H | 40H | 398H |
| | Omi18L | Omi18L | Omi18L | Omi18L | Omi18L | Omi18L |
| Omi29L | 158H | 175H | 222H | 269H | 40H | 398H |
| | Omi29L | Omi29L | Omi29L | Omi29L | Omi29L | Omi29L |
| Omi16H | 158H | 175H | 222H | 269H | 40H | 398H |
| | Omi16L | Omi16L | Omi16L | Omi16L | Omi16L | Omi16L |
| Omi17H | 158H | 175H | 222H | 269H | 40H | 398H |
| | Omi17L | Omi17L | Omi17L | Omi17L | Omi17L | Omi17L |
| Omi20H | 158H | 175H | 222H | 269H | 40H | 398H |
| | Omi20L | Omi20L | Omi20L | Omi20L | Omi20L | Omi20L |
| Omi27H | 158H | 175H | 222H | 269H | 40H | 398H |
| | Omi27L | Omi27L | Omi27L | Omi27L | Omi27L | Omi27L |
| Omi28H | 158H | 175H | 222H | 269H | 40H | 398H |
| | Omi28L | Omi28L | Omi28L | Omi28L | Omi28L | Omi28L |
| Omi36H | 158H | 175H | 222H | 269H | 40H | 398H |
| | Omi36L | Omi36L | Omi36L | Omi36L | Omi36L | Omi36L |
| Beta-27L | 158H | 175H | 222H | 269H | 40H | 398H |
| | Beta-27L | Beta-27L | Beta-27L | Beta-27L | Beta- 27L | Beta-27L |
| 150L | 158H | 175H | 222H | 269H | 40H 150L | 398H |
| | 150L | 150L | 150L | 150L | | 150L |
| 158L | — | 175H | 222H | 269H | 40H 158L | 398H |
| | | 158L | 158L | 158L | | 158L |
| 175L | 158H | — | 222H | 269H | 40H 175L | 398H |
| | 175L | | 175L | 175L | | 175L |
| 222L | 158H | 175H | — | 269H | 40H 222L | 398H |
| | 222L | 222L | | 222L | | 222L |
| 269L | 158H | 175H | 222H | — | 40H 269L | 398H |
| | 269L | 269L | 269L | | | 269L |
| 40L | 158H | 175H | 222H | 269H | — | 398H 40L |
| | 40L | 40L | 40L | 40L | | |
| 398L | 158H | 175H | 222H | 269H | 40H 398L | — |
| | 398L | 398L | 398L | 398L | | |

TABLE 6
Examples of the mixed chain antibodies generated from antibodies derived from the
same germline heavy chain IGHV1-58

| Heavy chain | | | | | | | |
|---|---|---|---|---|---|---|---|
| (H)/light chain | | | | | | | |
| (L) of antibody | Omi12H | Beta-47H | Beta-25H | 55H | 165H | 253H | 318H |
| Omi12L | — | Beta-47H | Beta-25H | 55H | 165H | 253H | 318H |
| | | Omi12L | Omi12L | Omi12L | Omi12L | Omi12L | Omi12L |
| Beta-47L | Omi12H | — | Beta-25H | 55H | 165H | 253H | 318H |
| | Beta-47L | | Beta-47L | Beta-47L | Beta-47L | Beta-47L | Beta-47L |
| Beta-25L | Omi12H | Beta-47H | — | 55H | 165H | 253H | 318H |
| | Beta- | Beta-25L | | Beta 25L | Beta 25L | Beta 25L | Beta 25L |
| | 25L | | | | | | |
| 55L | Omi12H | Beta-47H | Beta-25H | — | 165H | 253H | 318H |
| | 55L | 55L | 55L | | 55L | 55L | 55L |
| 165L | Omi12 | Beta-47H | Beta-25H | 55H | — | 253H | 318H |
| | H165L | 165L | 165L | 165L | | 165L | 165L |
| 253L | Omi12 | Beta-47H | Beta-25H | 55H | 165H | — | 318H |
| | H253L | 253L | 253L | 253L | 253L | | 253L |
| 318L | Omi12 | Beta-47H | Beta-25H | 55H | 165H | 253H | — |
| | H318L | 318L | 318L | 318L | 318L | 318L | |

TABLE 7
Examples of the mixed chain antibodies generated from antibodies derived from the same germline heavy chain IGHV1-69

| Heavy chain | | | | | | | | |
|---|---|---|---|---|---|---|---|---|
| (H)/light chain | | | | | | | | |
| (L) of antibody | Beta-49H | Beta-50H | Omi02H | Omi24H | Omi30H | Omi31H | Omi34H | Omi38H |
| Beta-49L | — | Beta-50H | Omi02H | Omi24H | Omi30H | Omi31H | Omi34H | Omi38H |
| | | Beta-49L | Beta-49L | Beta-49L | Beta-49L | Beta-49L | Beta-49L | Beta-49L |
| Beta-50L | Beta-49H | — | Omi02H | Omi24H | Omi30H | Omi31H | Omi34H | Omi38H |
| | Beta-50L | | Beta-50L | Beta-50L | Beta-50L | Beta-50L | Beta-50L | Beta-50L |
| Omi02L | Beta-49H | Beta-50H | — | Omi24H | Omi30H | Omi31H | Omi34H | Omi38H |
| | Omi02L | Omi02L | | Omi02L | Omi02L | Omi02L | Omi02L | Omi02L |
| Omi24L | Beta-49H | Beta-50H | Omi02H | — | Omi30H | Omi31H | Omi34H | Omi38H |
| | Omi24L | Omi24L | Omi24L | | Omi24L | Omi24L | Omi24L | Omi24L |
| Omi30L | Beta-49H | Beta-50H | Omi02H | Omi24H | — | Omi31H | Omi34H | Omi38H |
| | Omi30L | Omi30L | Omi30L | Omi30L | | Omi30L | Omi30L | Omi30L |
| Omi31L | Beta-49H | Beta-50H | Omi02H | Omi24H | Omi30H | — | Omi34H | Omi38H |
| | Omi31L | Omi31L | Omi31L | Omi31L | Omi31L | | Omi31L | Omi31L |
| Omi34H | Beta-49H | Beta-50H | Omi02H | Omi24H | Omi30H | Omi31H | — | Omi38H |
| | Omi34L | Omi34L | Omi34L | Omi34L | Omi34L | Omi34L | | Omi34L |
| Omi38H | Beta-49H | Beta-50H | Omi02H | Omi24H | Omi30H | Omi31H | Omi34H | — |
| | Omi38L | Omi38L | Omi38L | Omi38L | Omi38L | Omi38L | Omi38L | |

TABLE 8
Examples of the mixed chain antibodies generated from antibodies
derived from the same germline heavy chain IGHV3-30

| Heavy chain | | | | |
|---|---|---|---|---|
| (H)/light chain | | | | |
| (L) of antibody | Beta-22H | Beta-29H | 159H | Omi09H |
| Beta-22L | — | Beta- | 159H | Omi09H |
| | | 29H | Beta- 22L | Beta- |
| | | Beta-22L | | 22L |
| Beta-29L | Beta-22H | — | 159H | Omi09H |
| | Beta 29L | | Beta-29L | Beta |
| | | | | 29L |
| 159L | Beta-22H | Beta-29H | — | Omi09 |
| | 159L | 159L | | H159L |
| Omi09L | Beta-22H | Beta-29H | 159H | — |
| | Omi09L | Omi09L | Omi09L | |

TABLE 9
Examples of the mixed chain antibodies generated from
antibodies derived from the same germline heavy chain IGHV3-33

| Heavy chain | | | | |
|---|---|---|---|---|
| (H)/light chain | | | | |
| (L) of antibody | Beta-20H | Beta-43H | Omi32H | Omi33H |
| Beta-20L | — | Beta- | Omi32H | Omi33H |
| | | 43H | Beta- 20L | Beta- 20L |
| | | Beta-20L | | |
| Beta-43L | Beta- | — | Omi32H | Omi33H |
| | 20H | | Beta- 43L | Beta- 43L |
| | Beta-43L | | | |
| Omi32L | Beta-20H | Beta-43H | — | Omi33H |
| | Omi32L | Omi32L | | Omi32L |
| Omi33L | Beta-20H | Beta-43H | Omi32H | — |
| | Omi33L | Omi33L | Omi33L | |

TABLE 10
Examples of the mixed chain antibodies generated from
antibodies derived from the same germline heavy chain IGHV1-18

| Heavy chain | | | | |
|---|---|---|---|---|
| (H)/lightchain | | | | |
| (L) of antibody | 278H | Beta-44H | Omi26H | Omi41H |
| 278L | — | Beta-44H | Omi26 | Omi41 |
| | | 278L | H278L | H278L |
| Beta-44L | 278H | — | Omi26H | Omi41H |
| | Beta- 44L | | Beta- | Beta- |
| | | | 44L | 44L |
| Omi26L | 278H | Beta-44H | — | Omi41H |
| | Omi26L | Omi26L | | Omi26L |
| Omi41L | 278H | Beta-44H | Omi26H | — |
| | Omi41L | Omi41L | Omi41L | |

TABLE 11
Examples of the mixed chain antibodies generated from
antibodies derived from the same germline heavy chain IGHV3-9

| Heavy chain | | | | |
|---|---|---|---|---|
| (H)/light chain | | | | |
| (L) of antibody | 58H | Omi25H | Omi35H | Omi42H |
| 58L | — | Omi25 | Omi35 | Omi42 |
| | | H58L | H58L | H58L |
| Omi25L | 58H | — | Omi35H | Omi42H |
| | Omi25L | | Omi25L | Omi25L |
| Omi35L | 58H | Omi25H | — | Omi42H |
| | Omi35L | Omi35L | | Omi35L |
| Omi42L | 58H | Omi25H | Omi35H | — |
| | Omi42L | Omi42L | Omi42L | |

TABLE 12
Examples of the mixed chain antibodies generated from
antibodies derived from the same germline heavy chain IGHV4-31

| Heavy chain | | |
|---|---|---|
| (H)/light chain | | |
| (L) of | | |
| antibody | Beta-56H | Omi23H |
| Beta-56L | — | Omi23H |
| | | Beta-56L |
| Omi23L | Beta-56HOmi23L | — |

TABLE 13
IC50 titres of 22 Omicron SARS-COV-2-specific human mAbs against live virus strains
Victoria, Alpha, Beta. Gamma, Delta and Omicron (BA.1).

| | Authentic virus-IC50 (μg/ml) | | | | | |
|---|---|---|---|---|---|---|
| | | | | | | Omicron |
| | Victoria | Alpha | Beta | Gamma | Delta | (BA.1) |
| Omi-02 | 0.015 ± | 0.014 ± | 0.009 ± | 0.004 ± | 0.014 ± | 0.013 ± |
| | 0.001 | 0.005 | 0.000 | 0.000 | 0.003 | 0.001 |
| Omi-03 | 0.007 ± | 0.012 ± | 0.009 ± | 0.004 ± | 0.004 ± | 0.009 ± |
| | 0.000 | 0.007 | 0.001 | 0.000 | 0.000 | 0.002 |
| Omi-06 | 0.007 ± | 0.011 ± | 0.012 ± | 0.010 ± | 5.040 ± | 0.054 ± |
| | 0.001 | 0.002 | 0.000 | 0.003 | 0.747 | 0.005 |
| Omi-08 | 0.014 ± | 0.022 ± | 0.007 ± | 0.024 ± | 0.048 ± | 0.008 ± |
| | 0.007 | 0.002 | 0.000 | 0.007 | 0.012 | 0.004 |
| Omi-09 | 0.004 ± | 0.002 ± | 1.218 ± | 2.373 ± | 0.008 ± | 0.011 ± |
| | 0.001 | 0.000 | 0.324 | 1.008 | 0.002 | 0.005 |
| Omi-12 | 0.005 ± | 0.003 ± | 0.006 ± | 0.003 ± | 0.003 ± | 0.004 ± |
| | 0.000 | 0.001 | 0.001 | 0.000 | 0.000 | 0.001 |
| Omi-16 | 0.016 ± | 0.022 ± | 0.018 ± | 0.022 ± | 0.016 ± | 0.019 ± |
| | 0.002 | 0.009 | 0.004 | 0.007 | 0.002 | 0.003 |
| Omi-17 | 0.066 ± | 0.098 ± | 0.021 ± | 0.021 ± | 0.074 ± | 0.028 ± |
| | 0.015 | 0.027 | 0.007 | 0.007 | 0.019 | 0.005 |
| Omi-18 | 0.041 ± | 0.038 ± | 0.018 ± | 0.016 ± | 0.025 ± | 0.006 ± |
| | 0.005 | 0.008 | 0.006 | 0.004 | 0.000 | 0.003 |
| Omi-20 | 0.012 ± | 0.023 ± | 0.019 ± | 0.019 ± | 0.008 ± | 0.043 ± |
| | 0.002 | 0.004 | 0.009 | 0.006 | 0.001 | 0.012 |
| Omi-23 | 0.005 ± | 0.009 ± | 0.020 ± | 0.018 ± | 0.006 ± | 0.044 ± |
| | 0.002 | 0.004 | 0.005 | 0.006 | 0.002 | 0.013 |
| Omi-24 | 0.005 ± | 0.008 ± | 0.006 ± | 0.010 ± | >10 | 0.007 ± |
| | 0.001 | 0.003 | 0.001 | 0.005 | | 0.001 |
| Omi-25 | 0.003 ± | 0.007 ± | 0.059 ± | 0.257 ± | 0.006 ± | 0.046 ± |
| | 0.001 | 0.001 | 0.007 | 0.079 | 0.002 | 0.015 |
| Omi-26 | 0.005 ± | 0.010 ± | 0.055 ± | 0.214 ± | 0.005 ± | 0.034 ± |
| | 0.000 | 0.003 | 0.020 | 0.046 | 0.001 | 0.000 |
| Omi-27 | 0.026 ± | 0.032 ± | 0.019 ± | 0.017 ± | 0.010 ± | 0.091 ± |
| | 0.001 | 0.012 | 0.006 | 0.006 | 0.001 | 0.050 |
| Omi-28 | 0.028 ± | 0.028 ± | 0.019 ± | 0.033 ± | 0.018 ± | 0.032 ± |
| | 0.004 | 0.001 | 0.010 | 0.008 | 0.002 | 0.009 |
| Omi-29 | 0.044 ± | 0.066 ± | 0.048 ± | 0.040 ± | 0.029 ± | 0.036 ± |
| | 0.002 | 0.034 | 0.020 | 0.007 | 0.004 | 0.003 |
| Omi-30 | 0.109 ± | 0.043 ± | 0.028 ± | 0.038 ± | >10 | 0.058 ± |
| | 0.035 | 0.016 | 0.009 | 0.004 | | 0.008 |
| Omi-31 | 0.007 ± | 0.020 ± | 0.011 ± | 0.017 ± | >10 | 0.010 ± |
| | 0.001 | 0.003 | 0.005 | 0.006 | | 0.002 |
| Omi-32 | 0.032 ± | 0.102 ± | 0.460 ± | 0.430 ± | 0.012 ± | 0.024 ± |
| | 0.016 | 0.041 | 0.092 | 0.012 | 0.002 | 0.011 |
| Omi-33 | 0.028 ± | 0.057 ± | 0.136 ± | 0.132 ± | 0.011 ± | 0.026 ± |
| | 0.005 | 0.017 | 0.002 | 0.037 | 0.001 | 0.008 |
| Omi-34 | 0.003 ± | 0.041 ± | 0.003 ± | 0.008 ± | >10 | 0.028 ± |
| | 0.001 | 0.027 | 0.000 | 0.002 | | 0.009 |
| Omi-35 | 0.057 ± | 0.080 ± | 0.128 ± | 0.136 ± | 0.280 ± | 0.069 ± |
| | 0.003 | 0.030 | 0.058 | 0.024 | 0.059 | 0.032 |
| Omi-36 | 0.056 ± | 0.047 ± | 0.018 ± | 0.015 ± | 0.026 ± | 0.038 ± |
| | 0.008 | 0.009 | 0.001 | 0.000 | 0.003 | 0.006 |
| Omi-38 | 0.001 ± | 0.009 ± | 0.004 ± | 0.002 ± | 0.004 ± | 0.054 ± |
| | 0.000 | 0.001 | 0.000 | 0.000 | 0.001 | 0.028 |
| Omi-39 | 0.015 ± | 0.039 ± | 0.009 ± | 0.014 ± | 0.012 ± | 0.025 ± |
| | 0.006 | 0.007 | 0.000 | 0.001 | 0.007 | 0.004 |
| Omi-41 | 0.090 ± | 2.262 ± | >10 | 0.126 ± | >10 | 0.081 ± |
| | 0.013 | 1.199 | | 0.059 | | 0.004 |
| Omi-42 | 0.016 ± | 0.024 ± | 0.011± | 0.013 ± | 0.019 ± | 0.014 ± |
| | 0.003 | 0.001 | 0.004 | 0.003 | 0.001 | 0.002 |

TABLE 14
IC50 titres of 22 Omicron SARS-CoV-2-specific human mAbs
against pseudovirus strains Victoria, Omicron BA.1,
Omicron BA.1.1, Omicron BA.2 and Omicron BA.3.

| | Pseudovirus - IC50 (μg/ml) | | | | |
|---|---|---|---|---|---|
| | Victoria | OmicronBA.1 | OmicronBA.1.1 | OmicronBA.2 | OmicronBA.3 |
| Omi-02 | 0.002 ± 0.001 | 0.004 ± 0.001 | 0.004 ± 0.001 | 0.003 ± 0.001 | 0.019 ± 0.007 |
| Omi-03 | 0.003 ± 0.000 | 0.005 ± 0.002 | 0.003 ± 0.001 | 0.008 ± 0.001 | 0.022 ± 0.003 |
| Omi-06 | 0.007 ± 0.000 | 0.017 ± 0.003 | 0.139 ± 0.033 | 0.039 ± 0.008 | 0.696 ± 0.106 |
| Omi-08 | 0.008 ± 0.004 | 0.003 ± 0.000 | 0.002 ± 0.000 | 0.114 ± 0.045 | 0.032 ± 0.001 |
| Omi-09 | 0.006 ± 0.002 | 0.005 ± 0.000 | 0.005 ± 0.002 | 0.008 ± 0.002 | 0.017 ± 0.002 |
| Omi-12 | 0.006 ± 0.002 | 0.002 ± 0.000 | 0.002 ± 0.001 | 0.003 ± 0.001 | 0.006 ± 0.001 |
| Omi-16 | 0.014 ± 0.003 | 0.012 ± 0.002 | 0.011 ± 0.003 | 0.034 ± 0.012 | 0.111 ± 0.008 |
| Omi-17 | 0.023 ± 0.011 | 0.018 ± 0.012 | 0.022 ± 0.009 | 0.060 ± 0.004 | 0.123 ± 0.002 |
| Omi-18 | 0.008 ± 0.003 | 0.002 ± 0.000 | 0.002 ± 0.000 | 0.005 ± 0.000 | 0.006 ± 0.002 |
| Omi-20 | 0.009 ± 0.002 | 0.006 ± 0.001 | 0.005 ± 0.001 | 0.015 ± 0.003 | 0.020 ± 0.004 |
| Omi-23 | 0.005 ± 0.002 | 0.029 ± 0.006 | 0.023 ± 0.12 | 0.019 ± 0.005 | 0.011 ± 0.000 |
| Omi-24 | 0.005 ± 0.000 | 0.006 ± 0.002 | 0.054 ± 0.015 | 0.007 ± 0.001 | 0.009 ± 0.002 |
| Omi-25 | 0.005 ± 0.001 | 0.023 ± 0.005 | 0.027 ± 0.005 | 0.024 ± 0.004 | 0.050 ± 0.004 |
| Omi-26 | 0.002 ± 0.001 | 0.006 ± 0.002 | 0.005 ± 0.001 | 0.013 ± 0.001 | 0.018 ± 0.002 |
| Omi-27 | 0.008 ± 0.003 | 0.026 ± 0.006 | 0.034 ± 0.009 | 0.034 ± 0.005 | 0.026 ± 0.007 |
| Omi-28 | 0.022 ± 0.000 | 0.011 ± 0.004 | 0.009 ± 0.002 | 0.008 ± 0.000 | 0.019 ± 0.000 |
| Omi-29 | 0.014 ± 0.006 | 0.017 ± 0.003 | 0.016 ± 0.009 | 0.056 ± 0.014 | 0.064 ± 0.017 |
| Omi-30 | 0.012 ± 0.002 | 0.008 ± 0.003 | 0.008 ± 0.004 | 0.011 ± 0.002 | 0.015 ± 0.003 |
| Omi-31 | 0.376 ± 0.090 | 0.029 ± 0.002 | 0.031 ± 0.012 | 0.013 ± 0.002 | 0.013 ± 0.004 |
| Omi-32 | 0.010 ± 0.006 | 0.017 ± 0.000 | >10 | 2.682 ± 0.553 | 1.018 ± 0.139 |
| Omi-33 | 0.027 ± 0.011 | 0.014 ± 0.005 | 0.042 ± 0.018 | 0.068 ± 0.022 | 0.133 ± 0.021 |
| Omi-34 | 0.007 ± 0.004 | 0.008 ± 0.001 | 0.062 ± 0.004 | 0.009 ± 0.003 | 0.014 ± 0.000 |
| Omi-35 | 0.018 ± 0.004 | 0.058 ± 0.009 | 0.381 ± 0.086 | 0.093 ± 0.005 | 0.044 ± 0.018 |
| Omi-36 | 0.022 ± 0.004 | 0.009 ± 0.003 | 0.009 ± 0.003 | 0.030 ± 0.014 | 0.178 ± 0.048 |
| Omi-38 | 0.015 ± 0.004 | 0.024 ± 0.015 | >10 | 0.005 ± 0.000 | 0.008 ± 0.002 |
| Omi-39 | 0.014 ± 0.002 | 0.009 ± 0.004 | >10 | 0.026 ± 0.011 | 0.014 ± 0.001 |
| Omi-41 | >10 | 0.053 ± 0.028 | 0.037 ± 0.002 | >10 | 0.032 ± 0.007 |
| Omi-42 | 0.013 ± 0.004 | 0.007 ± 0.004 | 0.006 ± 0.002 | 0.021 ± 0.011 | 0.025 ± 0.012 |

TABLE 15
IC50 titres of early pandemic SARS-CoV-2-specific human mAbs and
Beta SARS-CoV-2 specific human mAbs against pseudovirus strains
Victoria, Omicron BA.1, Omicron BA.1.1 and Omicron BA.2.

| Early | IC50 (ug/ml) | | | |
|---|---|---|---|---|
| pandemic | | | Omicron | Omicron |
| mAbs | Victoria | Omicron BA.1 | BA.1.1 | BA.2 |
| 40 | 0.006 ± 0.002 | 1.705 ± 0.840 | 0.544 ± 0.007 | 0.100 ± 0.007 |
| 55 | 0.006 ± 0.002 | >10 | >10 | >10 |
| 58 | 0.019 ± 0.004 | 0.060 ± 0.041 | 0.876 ± 0.135 | 0.043 ± 0.007 |
| 88 | 0.005 ± 0.002 | >10 | >10 | >10 |
| 132 | 0.012 ± 0.004 | >10 | >10 | >10 |
| 150 | 0.008 ± 0.004 | >10 | 3.500 ± 0.712 | >10 |
| 158 | 0.021 ± 0.006 | >10 | 2.843 ± 0.733 | 4.249 ± 0.694 |
| 159 | >10 | >10 | >10 | >10 |
| 165 | 0.007 ± 0.005 | >10 | >10 | >10 |
| 170 | 0.006 ± 0.001 | >10 | >10 | >10 |
| 175 | 0.012 ± 0.004 | >10 | >10 | >10 |
| 222 | 0.006 ± 0.000 | 0.021 ± 0.002 | 0.023 ± 0.001 | 0.249 ± 0.082 |
| 253 | 0.021 ± 0.009 | 0.875 ± 0.373 | 0.415 ± 0.161 | 1.100 ± 0.049 |
| 269 | 0.008 ± 0.004 | >10 | >10 | >10 |
| 278 | 0.001 ± 0.000 | >10 | >10 | 0.326 ± 0.011 |
| 281 | 0.001 ± 0.000 | >10 | >10 | >10 |
| 316 | 0.001 ± 0.000 | >10 | >10 | >10 |
| 318 | 0.012 ± 0.003 | 9.490 ± 4.540 | >10 | 0.303 ± 0.190 |
| 384 | 0.001 ± 0.000 | >10 | >10 | >10 |
| 398 | 0.072 ± 0.065 | >10 | >10 | >10 |
| 253 + 55 | 0.001 ± 0.000 | 0.638 ± 0.315 | 0.451 ± 0.014 | >10 |
| 253 + 165 | 0.001 ± 0.000 | >10 | 6.591 ± 0.799 | >10 |
| Beta | IC50 (ug/ml) | | | |
| mAbs | Beta | Omicron BA.1 | Omicron BA.1.1 | Omicron BA.2 |
| β06 | 0.005 ± 0.001 | >10 | >10 | >10 |
| β10 | 0.021 ± 0.008 | >10 | >10 | >10 |
| β20 | 0.006 ± 0.002 | 5.679 ± 0.452 | 1.836 ± 0.780 | >10 |
| β22 | 0.041 ± 0.014 | 0.479 ± 0.029 | 0.130 ± 0.005 | >10 |
| β23 | 0.005 ± 0.001 | >10 | >10 | >10 |
| β24 | 0.002 ± 0.000 | >10 | >10 | >10 |
| β26 | 0.004 ± 0.001 | >10 | >10 | >10 |
| β27 | 0.003 ± 0.001 | 0.766 ± 0.043 | 0.274 ± 0.095 | 0.348 ± 0.030 |
| β29 | 0.009 ± 0.000 | 0.095 ± 0.029 | 0.066 ± 0.002 | 4.029 ± 0.402 |
| β30 | 0.002 ± 0.000 | >10 | >10 | >10 |
| β32 | 0.023 ± 0.001 | >10 | >10 | >10 |
| β33 | 0.020 ± 0.002 | >10 | >10 | >10 |
| β34 | 0.030 ± 0.004 | >10 | >10 | >10 |
| β38 | 0.004 ± 0.001 | >10 | >10 | >10 |
| β40 | 0.001 ± 0.000 | 0.005 ± 0.001 | 0.002 ± 0.000 | 0.008 ± 0.002 |
| β43 | 0.014 ± 0.003 | >10 | >10 | >10 |
| β44 | 0.008 ± 0.001 | >10 | >10 | >10 |
| β45 | 0.010 ± 0.001 | >10 | >10 | >10 |
| β47 | 0.002 ± 0.000 | 0.018 ± 0.009 | 0.011 ± 0.002 | 0.044 ± 0.006 |
| β48 | 0.003 ± 0.001 | 5.706 ± 0.676 | 0.752 ± 0.052 | 5.042 ± 0.650 |
| β49 | 0.014 ± 0.004 | >10 | >10 | >10 |
| β50 | 0.008 ± 0.001 | >10 | >10 | >10 |
| β51 | 0.003 ± 0.000 | >10 | >10 | >10 |
| β53 | 0.007 ± 0.001 | 0.141 ± 0.026 | 5.849 ± 0.036 | 0.170 ± 0.073 |
| β54 | 0.002 ± 0.000 | 0.003 ± 0.001 | 0.001 ± 0.000 | 0.076 ± 0.029 |
| β55 | 0.009 ± 0.002 | 0.033 ± 0.008 | 0.009 ± 0.001 | 0.069 ± 0.008 |

TABLE 16
IC50 titres of commercial mAbs against pseudovirus strains Victoria,
Omicron BA.1, Omicron BA.1.1, Omicron BA.2 and Omicron BA.3.

| | IC50 (ug/ml) | | | | |
|---|---|---|---|---|---|
| Commercial | | Omicron | Omicron | Omicron | Omicron |
| mAbs | Victoria | BA.1 | BA.1.1 | BA.2 | BA.3 |
| Known Antibody | 0.002 ± 0.001 | >10 | >10 | 0.616 ± | >10 |
| A (REGN 10987) | | | | 0.347 | |
| Known Antibody | 0.001 ± 0.002 | >10 | >10 | >10 | >10 |
| B (REGN 10933) | | | | | |
| Known Antibody | 0.002 ± 0.001 | 0.308 ± | >10 | 0.008 ± | 0.019 |
| C (AZD1061) | | 0.058 | | 0.003 | |
| Known Antibody | 0.001 ± 0.000 | 0.246 ± | 0.100 ± | 1.333 ± | >10 |
| D (AZD8895) | | 0.027 | 0.053 | 0.317 | |
| Known Antibody | 0.001 ± 0.000 | 0.232 ± | 0.806 ± | 0.008 ± | 0.065 ± |
| E (AZD7442) | | 0.113 | 0.093 | 0.001 | 0.011 |
| Known Antibody | 0.007 ± 0.002 | >10 | >10 | >10 | >10 |
| F (ADG10) | | | | | |
| Known Antibody | 0.003 ± 0.002 | 0.348 ± | 0.253 ± | >10 | >10 |
| G (ADG20) | | 0.169 | 0.070 | | |
| Known Antibody | 0.014 ± 0.006 | >10 | >10 | >10 | >10 |
| H (ADG30) | | | | | |
| Known Antibody | 0.002 ± 0.000 | >10 | >10 | >10 | >10 |
| I (Ly-CoV-555) | | | | | |
| Known Antibody | 0.014 ± 0.010 | >10 | >10 | >10 | >10 |
| J (Ly-CoV16) | | | | | |
| Known Antibody | 0.587 ± 0.286 | 0.094 ± | 0.138 ± | 0.638 ± | 0.228 ± |
| K (S309) | | 0.008 | 0.020 | 0.107 | 0.009 |

TABLE 17
Properties of omicron antibodies

| | Heavy Chain | | | |
|---|---|---|---|---|
| | | | | #Amino |
| | | | | acid |
| mAbs | V-GENE | J-GENE | D-GENE | substitutions |
| Omi-02 | 1-69*01, | 2*01 | 2-21*02 | 7 |
| | or 1-69D*01 | | | |
| Omi-03 | 3-53*01 | 4*02 | 1-26*01 | 5 |
| Omi-06 | 4-4*07 | 3*02 | 3-16*02 | 4 |
| Omi-08 | 1-46*01, | 4*02 | 6-13*01 | 12 |
| | or 1-46*03 | | | |
| Omi-09 | 3-30*01 | 3*02 | 4-17*01 | 6 |
| Omi-12 | 1-58*02 | 3*02 | 2-2*01 | 12 |
| Omi-16 | 3-66*02 | 4*02 | 2-15*01 | 9 |
| Omi-17 | 3-66*02 | 4*02 | 6-19*01 | 7 |
| Omi-18 | 3-53*01 | 6*02 | 4-11*01 | 11 |
| Omi-20 | 3-66*02 | 6*02 | 5-12*01 | 11 |
| Omi-23 | 4-31*03 | 4*02 | 3-22*01 | 6 |
| Omi-24 | 1-69*06 | 4*02 | 3-16*02 | 9 |
| Omi-25 | 3-9*01 | 6*02 | 3-16*01 | 6 |
| Omi-26 | 1-18*01 | 4*02 | 1-26*01 | 12 |
| Omi-27 | 3-66*01, | 6*02 | 6-19*01 | 8 |
| | or 3-66*04 | | | |
| Omi-28 | 3-66*01, | 4*02 | 3-16*01 | 4 |
| | or 3-66*04 | | | |
| Omi-29 | 3-53*04 | 6*02 | 2-15*01 | 11 |
| Omi-30 | 1-69*06 | 6*02 | 2-15*01 | 10 |
| Omi-31 | 1-69*06 | 6*02 | 3-16*01 | 11 |
| Omi-32 | 3-33*01, | 4*02 | 2-21*02 | 6 |
| | or 3-33*06 | | | |
| Omi-33 | 3-33*01, | 4*02 | 2-21*02 | 10 |
| | or 3-33*06 | | | |
| Omi-34 | 1-69*06, | 4*02 | 2-2*01 | 10 |
| | or 1-69*14 | | | |
| Omi-35 | 3-9*01 | 6*02 | 2-2*02 | 5 |
| Omi-36 | 3-66*02 | 4*02 | 2-15*01 | 9 |
| Omi-38 | 1-69*09 | 3*01 | 1-26*01 | 16 |
| Omi-39 | 3-43*01 | 6*03 | 2-2*01 | 8 |
| Omi-41 | 1-18*04 | 4*02 | 3-9*01 | 11 |
| Omi-42 | 3-9*01 | 6*02 | 6-19*01 | 7 |
| | Light Chain | | | |
| | | | | #Amino |
| | | | | acid |
| mAbs | K/λ | V-GENE | J-GENE | substitutions |
| Omi-02 | K | 3-20*01 | 5*01 | 9 |
| Omi-03 | K | 3-20*01 | 2*01 | 10 |
| Omi-06 | K | 1-39*01, or | 4*01 | 9 |
| | | 1D-39*01 | | |
| Omi-08 | λ | 1-40*02 | 1*01 | 13 |
| Omi-09 | λ | 3-25*02 | 2*01, or | 14 |
| | | | 3*01 | |
| Omi-12 | K | 3-20*01 | 1*01 | 9 |
| Omi-16 | K | 3-20*01 | 2*01 | 10 |
| Omi-17 | K | 3-20*01 | 2*01 | 10 |
| Omi-18 | λ | 3-21*02 | 1*01 | 10 |
| Omi-20 | K | 1-9*01 | 4*02 ( ) | 9 |
| Omi-23 | K | 1-NL1*01 | 1*01 | 10 |
| Omi-24 | K | 3-15*01 | 1*01 | 10 |
| Omi-25 | K | 1-39*01, or | 2*01 | 9 |
| | | 1D-39*01 | | |
| Omi-26 | λ | 1-36*01 | 3*02 | 11 |
| Omi-27 | K | 1-6*01 | 2*01 | 9 |
| Omi-28 | K | 3-20*01 | 1*01 | 9 |
| Omi-29 | λ | 2-14*01, or 2- | 3*02 | 10 |
| | | 14*03 | | |
| Omi-30 | λ | 1-44*01 | 3*02 | 11 |
| Omi-31 | λ | 1-44*01 | 3*02 | 11 |
| Omi-32 | K | 3-20*01 | 4*01 | 10 |
| Omi-33 | K | 3-20*01 | 4*01 | 4 |
| Omi-34 | λ | 1-40*01 | 1*01 | 12 |
| Omi-35 | λ | 3-21*02 | 2*01, or | 11 |
| | | | 3*01 | |
| Omi-36 | K | 3-20*01 | 2*01 | 5 |
| Omi-38 | K | 1-5*01 | 5*01 | 6 |
| Omi-39 | K | 4-1*01 | 3*01 | 5 |
| Omi-41 | K | 4-1*01 | 2*02 ( ) | 5 |
| Omi-42 | λ | 2-8*01 | 2*01, or | 8 |
| | | | 3*01 or | |
| | | | 3*02 | |

TABLE 18
IC50 titres of 22 Omicron SARS-COV-2-specific human mAbs or commercial mAbs
against various SARS-COV-2 strains.

| | IC50 (ug/ml) | | | | | | | |
|---|---|---|---|---|---|---|---|---|
| mAbs | Victoria | Alpha | Beta | Gamma | Delta | BA.1 | BA.1.1 | BA.2 |
| Omi-02 | 0.015 ± | 0.014 ± | 0.009 ± | 0.004 ± | 0.014 ± | 0.013 ± | 0.015 ± | 0.040 ± |
| | 0.001 | 0.005 | 0.000 | 0.000 | 0.003 | 0.001 | 0.001 | 0.021 |
| Omi-03 | 0.007 ± | 0.012 ± | 0.009 ± | 0.004 ± | 0.004 ± | 0.009 ± | 0.015 ± | 0.028 ± |
| | 0.000 | 0.007 | 0.001 | 0.000 | 0.000 | 0.002 | 0.000 | 0.002 |
| Omi-06 | 0.007 ± | 0.011 ± | 0.012 ± | 0.010 ± | 5.040 ± | 0.054 ± | 1.505 ± | 0.238 ± |
| | 0.001 | 0,002 | 0.000 | 0.003 | 0.747 | 0.005 | 0.341 | 0.007 |
| Omi-08 | 0.014 ± | 0.022 ± | 0.007 ± | 0.024 ± | 0.048 ± | 0.008 ± | 0.007 ± | 1.510 ± |
| | 0.007 | 0.002 | 0.000 | 0.007 | 0.012 | 0.004 | 0.001 | 0.683 |
| Omi-09 | 0.004 ± | 0.002 ± | 1.218 ± | 2.373 ± | 0.008 ± | 0.011 ± | 0.017 ± | 0.034 ± |
| | 0.001 | 0.000 | 0.324 | 1.008 | 0.002 | 0.005 | 0.003 | 0.010 |
| Omi-12 | 0.005 ± | 0.003 ± | 0.006 ± | 0.003 ± | 0.003 ± | 0.004 ± | 0.009 ± | 0.010 ± |
| | 0.000 | 0.001 | 0.001 | 0.000 | 0.000 | 0.001 | 0.001 | 0.001 |
| Omi-16 | 0.016 ± | 0.022 ± | 0.018 ± | 0.022 ± | 0.016 ± | 0.019 ± | 0.027 ± | 0.067 ± |
| | 0.002 | 0.009 | 0.004 | 0.007 | 0.002 | 0.003 | 0.007 | 0.021 |
| Omi-17 | 0.066 ± | 0.098 ± | 0.021 ± | 0.021 ± | 0.074 ± | 0.028 ± | 0.026 ± | 0.095 ± |
| | 0.015 | 0.027 | 0.007 | 0.007 | 0.019 | 0.005 | 0.001 | 0.008 |
| Omi-18 | 0.041 ± | 0.038 ± | 0.018 ± | 0.016 ± | 0.025 ± | 0.006 ± | 0.006 ± | 0.007 ± |
| | 0.005 | 0.008 | 0.006 | 0.004 | 0.000 | 0.003 | 0.001 | 0.001 |
| Omi-20 | 0.012 ± | 0.023 ± | 0.019 ± | 0.019 ± | 0.008 ± | 0.043 ± | 0.032 ± | 0.022 ± |
| | 0.002 | 0.004 | 0.009 | 0.006 | 0.001 | 0.012 | 0.002 | 0.005 |
| Omi-23 | 0.005 ± | 0.009 ± | 0.020 ± | 0.018 ± | 0.006 ± | 0.044 ± | 0.03 ± | 0.028 ± |
| | 0.002 | 0.004 | 0.005 | 0.006 | 0.002 | 0.013 | 0.001 | 0.001 |
| Omi-24 | 0.005 ± | 0.008 ± | 0.006 ± | 0.010 ± | >10 | 0.007 ± | 0.035 ± | 0.008 ± |
| | 0.001 | 0.003 | 0.001 | 0.005 | | 0.001 | 0.010 | 0002 |
| Omi-25 | 0.003 ± | 0.007 ± | 0.059 ± | 0.257 ± | 0.006 ± | 0.046 ± | 0.138 ± | 0.056 ± |
| | 0.001 | 0.001 | 0.007 | 0.079 | 0.002 | 0.015 | 0.046 | 0.030 |
| Omi-26 | 0.005 ± | 0.010 ± | 0.055 ± | 0.214 ± | 0.005 ± | 0.034 ± | 0.055 ± | 0.03 ± |
| | 0.000 | 0.003 | 0.020 | 0.046 | 0.001 | 0.000 | 0.030 | 0.011 |
| Omi-27 | 0.026 ± | 0.032 ± | 0.019 ± | 0.017 ± | 0.010 ± | 0.091 ± | 0.239 ± | 0.039 ± |
| | 0.001 | 0.012 | 0.006 | 0.006 | 0.001 | 0.050 | 0.052 | 0.006 |
| Omi-28 | 0.028 ± | 0.028 ± | 0.019 ± | 0.033 ± | 0.018 ± | 0.032 ± | 0.075 ± | 0.047 ± |
| | 0.004 | 0.001 | 0.010 | 0.008 | 0.002 | 0.009 | 0.032 | 0.010 |
| Omi-29 | 0.044 ± | 0.066 ± | 0.048 ± | 0.040 ± | 0.029 ± | 0.036 ± | 0.052 ± | 0.192 ± |
| | 0.002 | 0.034 | 0.020 | 0.007 | 0.004 | 0.003 | 0.004 | 0.021 |
| Omi-30 | 0.109 ± | 0.043 ± | 0.028 ± | 0.038 ± | >10 | 0.058 ± | 0.084 ± | 0.045 ± |
| | 0.035 | 0.016 | 0.009 | 0.004 | | 0.008 | 0.021 | 0.010 |
| Omi-31 | 0.007 ± | 0.020 ± | 0.011 ± | 0.017 ± | >10 | 0.010 ± | 0.017 ± | 0.083 ± |
| | 0.001 | 0.003 | 0.005 | 0.006 | | 0.002 | 0.009 | 0.040 |
| Omi-32 | 0.032 ± | 0.102 ± | 0.460 ± | 0.430 ± | 0.012 ± | 0.024 ± | 4.642 ± | 1.899 ± |
| | 0.016 | 0.041 | 0.092 | 0.012 | 0.002 | 0.011 | 0.283 | 0.280 |
| Omi-33 | 0.028 ± | 0.057 ± | 0.136 ± | 0.132 ± | 0.011 ± | 0.026 ± | 0.113 ± | 0.681 ± |
| | 0.005 | 0.017 | 0.002 | 0.037 | 0.001 | 0.008 | 0.035 | 0.0170 |
| Omi-34 | 0.003 ± | 0.041 ± | 0.003 ± | 0.008 ± | >10 | 0.028 ± | 0.074 ± | 0.014 ± |
| | 0.001 | 0.027 | 0.000 | 0.002 | | 0.009 | 0.016 | 0.003 |
| Omi-35 | 0.057 ± | 0.080 ± | 0.128 ± | 0.136 ± | 0.280 ± | 0.069 ± | 0.262 ± | 0.082 ± |
| | 0.003 | 0.030 | 0.058 | 0.024 | 0.059 | 0.032 | 0.086 | 0.043 |
| Omi-36 | 0.056 ± | 0.047 ± | 0.018 ± | 0.015 ± | 0.026 ± | 0.038 ± | 0.053 ± | 0.105 ± |
| | 0.008 | 0.009 | 0.001 | 0.000 | 0.003 | 0.006 | 0.022 | 0.023 |
| Omi-38 | 0.001 ± | 0.009 ± | 0.004 ± | 0.002 ± | 0.004 ± | 0.054 ± | >10 | 0.027 ± |
| | 0.000 | 0.001 | 0.000 | 0.000 | 0.001 | 0.028 | | 0.001 |
| Omi-39 | 0.015 ± | 0.039 ± | 0.009 ± | 0.014 ± | 0.012 ± | 0.025 ± | >10 | 0.073 ± |
| | 0.006 | 0.007 | 0.000 | 0.001 | 0.007 | 0.004 | | 0.014 |
| Omi-41 | 0.090 ± | 2.262 ± | >10 | 0.126 ± | >10 | 0.081 ± | 0.191 ± | >10 |
| | 0.013 | 1.199 | | 0.059 | | 0.004 | 0.014 | |
| Omi-42 | 0.016 ± | 0.024 ± | 0.011 ± | 0.013 ± | 0.019 ± | 0.014 ± | 0.017 ± | 0.031 ± |
| | 0.003 | 0.001 | 0.004 | 0.003 | 0.001 | 0.002 | 0.004 | 0.008 |
| REGN10987 | 0.032 ± | 0.028 ± | 0.007 ± | 0.013 ± | 0.017 ± | >10 | >10 | 1.847 ± |
| | 0.007 | 0.003 | 0.001 | 0.002 | 0.009 | | | 1.231 |
| REGN10933 | 0.004 ± | 0.014 ± | 3.284 ± | 6.177 ± | 0.003 ± | >10 | >10 | >10 |
| | 0.002 | 0.002 | 2.014 | 1.914 | 0.001 | | | |
| AZD1061 | 0.013 ± | 0.012 ± | 0.014 ± | 0.007 ± | 0.038 ± | 3.488 ± | >10 | 0.028 ± |
| | 0.003 | 0.002 | 0.002 | 0.002 | 0.006 | 2.085 | | 0.014 |
| AZD8895 | 0.005 ± | 0.011 ± | 0.046 ± | 0.046 ± | 0.003 ± | 1.152 ± | 6.078 ± | 7.702 ± |
| | 0.001 | 0.002 | 0.031 | 0.016 | 0.000 | 0.170 | 1.558 | 2.224 |
| AZD7442 | 0.009 ± | 0.007 ± | 0.012 ± | 0.006 ± | 0.005 ± | 0.273 ± | 3.816 ± | 0.052 ± |
| | 0.000 | 0.001 | 0.001 | 0.003 | 0.000 | 0.062 | 0.138 | 0.004 |
| ADG10 | 0.006 ± | 0.010 ± | 0.011 ± | 0.003 ± | 0.026 ± | >10 | >10 | >10 |
| | 0.000 | 0.001 | 0.001 | 0.000 | 0.005 | | | |
| ADG20 | 0.004 ± | 0.006 ± | 0.01 ± | 0.009 ± | 0.006 ± | 1.104 ± | 1.269 ± | >10 |
| | 0.001 | 0.000 | 0.001 | 0,000 | 0.001 | 0.509 | 0.223 | |
| ADG30 | 0.007 ± | 0.016 ± | 0.029 ± | 0.002 ± | 0.033 ± | >10 | >10 | >10 |
| | 0.002 | 0.001 | 0.003 | 0.001 | 0.007 | | | |
| Ly-CoV-555 | 0.006 ± | 0.009 ± | >10 | >10 | 8.311 ± | >10 | >10 | >10 |
| | 0.002 | 0.000 | | | 4.059 | | | |
| Ly-CoV16 | 0.034 ± | 3.225 ± | >10 | >10 | 0.012 ± | >10 | >10 | >10 |
| | 0.007 | 1.030 | | | 0.002 | | | |
| S309 | 0.040 ± | 0.078 ± | 0.082 ± | 0.076 ± | 0.113 ± | 0.256 ± | 1.119 ± | 5.035 ± |
| | 0.005 | 0.069 | 0.002 | 0.014 | 0.028 | 0.034 | 0.119 | 0.244 |

TABLE 19
X-ray data collection and structure refinement statistics
for BA.1 RBD/Omi-12-Beta54 and Omi-12 Fab

| | BA.1 RBD/Omi-12- | | | |
|---|---|---|---|---|
| Structure | Beta54^{a} | | Omi-12 Fab^{a} | |
| Data collection | | | | |
| Space group | P2¹ | | C222¹ | |
| Cell dimensions | | | | |
| a, b, c (Å) | 95.7, 156.3, 122.4 | | 65.0, 210.1, 85.9 | |
| α, β, γ (°) | 90, 90.3, 90 | | 90, 90, 90 | |
| Resolution (Å) | 78-5.50 | (5.60-5.50) | 53-2.08 | (2.12-2.08) |
| R^{merge} | 0.641 | (—) | 0.179 | (—) |
| R^{pim} | 0.259 | (0.919) | 0.052 | (1.151) |
| I/_{σ}(I) | 2.1 | (0.4) | 6.2 | (0.2) |
| CC^{1/2} | 0.849 | (0.332) | 0.994 | (0.255) |
| Completeness (%) | 100 | (98.2) | 93.3 | (62.9) |
| Redundancy | 7.1 | (7.4) | 12.1 | (6.8) |

| | | |
|---|---|---|
| Refinement | | |
| Resolution (Å) | 78-5.50^{c} | 53-2.08 |
| No. reflections | 11051/615 | 29710/1547 |
| R^{work}/R^{free} | 0.285/0.285 | 0.241/0.267 |
| No. atoms | | |
| Protein | 16328 | 3320 |
| Ligand/ion/water | | 133 |
| B factors (Å²) | | |
| Protein | 248 | 59 |
| Ligand/ion/water | | 74 |
| r.m.s. deviations | | |
| Bond lengths (Å) | 0.010 | 0.002 |
| Bond angles (°) | 0.7 | 0.6 |

^{a}Omi12 is glycosylated at N102 of the heavy chain.
^{b}Values in parentheses are for highest-resolution shell.
^{c}Rigid body and group B-factor refinement only.

TABLE 20
Pseudoviral assays comparing BA.4 neutralization with neutralization of BA.1,
BA.1.1, BA.2 and BA.3

| | IC50 (μg/mL) | | | | | |
|---|---|---|---|---|---|---|
| | Pseudovirus | | | | | |
| | Victoria | BA.1 | BA.1.1 | BA.2 | BA.3 | BA.4 |
| Omi-02 | 0.002 ± 0.001 | 0.004 ± 0.001 | 0.004 ± 0.001 | 0.003 ± 0.001 | 0.019 ± 0.007 | >10 |
| Omi-03 | 0.003 ± 0.000 | 0.005 ± 0.002 | 0.003 ± 0.001 | 0.008 ± 0.001 | 0.022 ± 0.003 | 0.017 ± 0.005 |
| Omi-06 | 0.007 ± 0.000 | 0.017 ± 0.003 | 0.139 ± 0.033 | 0.039 ± 0.008 | 0.696 ± 0.106 | >10 |
| Omi-08 | 0.008 ± 0.004 | 0.003 ± 0.000 | 0.002 ± 0.000 | 0.114 ± 0.045 | 0.032 ± 0.001 | 0.086 ± 0.005 |
| Omi-09 | 0.006 ± 0.002 | 0.005 ± 0.000 | 0.005 ± 0.002 | 0.008 ± 0.002 | 0.017 ± 0.002 | 0.166 ± 0.007 |
| Omi-12 | 0.006 ± 0.002 | 0.002 ± 0.000 | 0.002 ± 0.001 | 0.003 ± 0.001 | 0.006 ± 0.001 | 0.429 ± 0.060 |
| Omi-16 | 0.014 ± 0.003 | 0.012 ± 0.002 | 0.011 ± 0.003 | 0.034 ± 0.012 | 0.111 ± 0.008 | 0.029 ± 0.007 |
| Omi-17 | 0.023 ± 0.011 | 0.018 ± 0.012 | 0.022 ± 0.009 | 0.060 ± 0.004 | 0.123 ± 0.002 | 0.028 ± 0.001 |
| Omi-18 | 0.008 ± 0.003 | 0.002 ± 0.000 | 0.002 ± 0.000 | 0.005 ± 0.000 | 0.006 ± 0.002 | 0.005 ± 0.001 |
| Omi-20 | 0.009 ± 0.002 | 0.006 ± 0.001 | 0.005 ± 0.001 | 0.015 ± 0.003 | 0.020 ± 0.004 | 0.014 ± 0.006 |
| Omi-23 | 0.005 ± 0.002 | 0.029 ± 0.006 | 0.023 ± 0.12 | 0.019 ± 0.005 | 0.011 ± 0.000 | >10 |
| Omi-24 | 0.005 ± 0.000 | 0.006 ± 0.002 | 0.054 ± 0.015 | 0.007 ± 0.001 | 0.009 ± 0.002 | >10 |
| Omi-25 | 0.005 ± 0.001 | 0.023 ± 0.005 | 0.027 ± 0.005 | 0.024 ± 0.004 | 0.050 ± 0.004 | >10 |
| Omi-26 | 0.002 ± 0.001 | 0.006 ± 0.002 | 0.005 ± 0.001 | 0.013 ± 0.001 | 0.018 ± 0.002 | >10 |
| Omi-27 | 0.008 ± 0.003 | 0.026 ± 0.006 | 0.034 ± 0.009 | 0.034 ± 0.005 | 0.026 ± 0.007 | 0.069 ± 0.023 |
| Omi-28 | 0.022 ± 0.000 | 0.011 ± 0.004 | 0.009 ± 0.002 | 0.008 ± 0.000 | 0.019 ± 0.000 | 0.028 ± 0.009 |
| Omi-29 | 0.014 ± 0.006 | 0.017 ± 0.003 | 0.016 ± 0.009 | 0.056 ± 0.014 | 0.064 ± 0.017 | 0.396 ± 0.007 |
| Omi-30 | 0.053 ± 0.010 | 0.029 ± 0.002 | 0.031 ± 0.012 | 0.013 ± 0.002 | 0.015 ± 0.003 | >10 |
| Omi-31 | 0.012 ± 0.002 | 0.008 ± 0.003 | 0.008 ± 0.004 | 0.011 ± 0.002 | 0.013 ± 0.004 | >10 |
| Omi-32 | 0.010 ± 0.006 | 0.017 ± 0.000 | >10 | 2.682 ± 0.553 | 1.018 ± 0.139 | 0.035 ± 0.016 |
| Omi-33 | 0.027 ± 0.011 | 0.014 ± 0.005 | 0.042 ± 0.018 | 0.068 ± 0.022 | 0.133 ± 0.021 | 0.013 ± 0.004 |
| Omi-34 | 0.007 ± 0.004 | 0.008 ± 0.001 | 0.062 ± 0.004 | 0.009 ± 0.003 | 0.014 ± 0.000 | >10 |
| Omi-35 | 0.021 ± 0.003 | 0.058 ± 0.006 | 0.381 ± 0.061 | 0.094 ± 0.004 | 0.044 ± 0.018 | 1.687 ± 0.441 |
| Omi-36 | 0.022 ± 0.004 | 0.009 ± 0.003 | 0.009 ± 0.003 | 0.030 ± 0.014 | 0.178 ± 0.048 | 0.024 ± 0.006 |
| Omi-38 | 0.015 ± 0.004 | 0.024 ± 0.015 | >10 | 0.005 ± 0.000 | 0.008 ± 0.002 | 0.005 ± 0.001 |
| Omi-39 | 0.014 ± 0.002 | 0.009 ± 0.004 | >10 | 0.026 ± 0.011 | 0.014 ± 0.001 | 0.035 ± 0.003 |
| Omi-41 | >10 | 0.053 ± 0.028 | 0.037 ± 0.002 | >10 | 0.032 ± 0.007 | >10 |
| Omi-42 | 0.013 ± 0.004 | 0.007 ± 0.004 | 0.006 ± 0.002 | 0.021 ± 0.011 | 0.025 ± 0.012 | 0.013 ± 0.001 |

TABLE 21
Activity of commercial antibodies against BA.4 and BA.5

| | IC50 (μg/mL) | | | | | |
|---|---|---|---|---|---|---|
| | Pseudovirus | | | | | |
| | Victoria | BA.1 | BA.1.1 | BA.2 | BA.3 | BA.4 |
| REGN10987 | 0.002 ± 0.001 | >10 | >10 | 0.616 ± 0.347 | >10 | >10 |
| REGN10933 | 0.001 ± 0.002 | >10 | >10 | >10 | >10 | >10 |
| AZD1061 | 0.002 ± 0.001 | 0.308 ± 0.058 | >10 | 0.008 ± 0.003 | 0.019 ± 0.007 | 0.015 ± 0.004 |
| AZD8895 | 0.001 ± 0.000 | 0.246 ± 0.027 | 0.100 ± 0.053 | 1.333 ± 0.317 | >10 | >10 |
| AZD7442 | 0.001 ± 0.000 | 0.232 ± 0.113 | 0.806 ± 0.093 | 0.008 ± 0.001 | 0.065 ± 0.011 | 0.065 ± 0.007 |
| ADG10 | 0.007 ± 0.002 | >10 | >10 | >10 | >10 | >10 |
| ADG20 | 0.003 ± 0.002 | 0.348 ± 0.169 | 0.253 ± 0.070 | >10 | >10 | >10 |
| ADG30 | 0.014 ± 0.006 | >10 | >10 | >10 | >10 | >10 |
| Ly-CoV-555 | 0.002 ± 0.000 | >10 | >10 | >10 | >10 | >10 |
| Ly-CoV16 | 0.014 ± 0.010 | >10 | >10 | >10 | >10 | >10 |
| S309 | 0.130 ± 0.030 | 0.094 ± 0.008 | 0.138± 0.020 | 0.638 ± 0.107 | 0.228 ± 0.009 | 1.041 ± 0.072 |

TABLE 22
IC50 of BA. 1 mAbs against PV BA.2.75 and BA.2 + N460K

| mAbs | Victoria | BA.1 | BA.1.1 | BA.2 | BA.3 | BA.4/5 | BA.2.75 | BA.2 + N460K |
|---|---|---|---|---|---|---|---|---|
| Omi-02 | 0.002 ± 0.001 | 0.004 ± 0.001 | 0.004 ± 0.001 | 0.003 ± 0.001 | 0.019 ± 0.007 | >10 | 0.009 ± 0.002 | 0.025 ± 0.003 |
| Omi-03 | 0.003 ± 0.000 | 0.005 ± 0.002 | 0.003 ± 0.001 | 0.008 ± 0.001 | 0.022 ± 0.003 | 0.017 ± 0.005 | 0.017 ± 0.000 | 0.401 ± 0.026 |
| (3-53) | | | | | | | | |
| Omi-06 | 0.007 ± 0.000 | 0.017 ± 0.003 | 0.139 ± 0.033 | 0.039 ± 0.008 | 0.696 ± 0.106 | >10 | 0.063 ± 0.005 | 0.026 ± 0.002 |
| Omi-08 | 0.008 ± 0.004 | 0.003 ± 0.000 | 0.002 ± 0.000 | 0.114 ± 0.045 | 0.032 ± 0.001 | 0.086 ± 0.005 | 0.036 ± 0.002 | 0.552 ± 0.090 |
| Omi-09 | 0.006 ± 0.002 | 0.005 ± 0.000 | 0.005 ± 0.002 | 0.008 ± 0.002 | 0.017 ± 0.002 | 0.166 ± 0.007 | 0.003 ± 0.000 | 0.010 ± 0.002 |
| Omi-12 | 0.006 ± 0.002 | 0.002 ± 0.000 | 0.002 ± 0.001 | 0.003 ± 0.001 | 0.006 ± 0.001 | 0.429 ± 0.060 | 0.003 ± 0.001 | 0.011 ± 0.002 |
| Omi-16 | 0.014 ± 0.003 | 0.012 ± 0.002 | 0.011 ± 0.003 | 0.034 ± 0.012 | 0.111 ± 0.008 | 0.029 ± 0.007 | >10 | >10 |
| (3-66) | | | | | | | | |
| Omi-17 | 0.023 ± 0.011 | 0.018 ± 0.012 | 0.022 ± 0.009 | 0.060 ± 0.004 | 0.123 ± 0.002 | 0.028 ± 0.001 | 0.255 ± 0.169 | >10 |
| (3-66) | | | | | | | | |
| Omi-18 | 0.008 ± 0.003 | 0.002 ± 0.000 | 0.002 ± 0.000 | 0.005 ± 0.000 | 0.006 ± 0.002 | 0.005 ± 0.001 | 0.035 ± 0.007 | 0.014 ± 0.002 |
| (3-53) | | | | | | | | |
| Omi-20 | 0.009 ± 0.002 | 0.006 ± 0.001 | 0.005 ± 0.001 | 0.015 ± 0.003 | 0.020 ± 0.004 | 0.014 ± 0.006 | 0.178 ± 0.075 | 0.315 ± 0.142 |
| (3-66) | | | | | | | | |
| Omi-23 | 0.005 ± 0.002 | 0.029 ± 0.006 | 0.023 ± 0.12 | 0.019 ± 0.005 | 0.011 ± 0.000 | >10 | 0.011 ± 0.006 | 0.022 ± 0.005 |
| Omi-24 | 0.005 ± 0.000 | 0.006 ± 0.002 | 0.054 ± 0.015 | 0.007 ± 0.001 | 0.009 ± 0.002 | >10 | 0.008 ± 0.004 | 0.014 ± 0.000 |
| Omi-25 | 0.005 ± 0.001 | 0.023 ± 0.005 | 0.027 ± 0.005 | 0.024 ± 0.004 | 0.050 ± 0.004 | >10 | 0.014 ± 0.005 | 0.050 ± 0.010 |
| Omi-26 | 0.002 ± 0.001 | 0.006 ± 0.002 | 0.005 ± 0.001 | 0.013 ± 0.001 | 0.018 ± 0.002 | >10 | 0.010 ± 0.004 | 0.010 ± 0.000 |
| Omi-27 | 0.008 ± 0.003 | 0.026 ± 0.006 | 0.034 ± 0.009 | 0.034 ± 0.005 | 0.026 ± 0.007 | 0.069 ± 0.023 | 6.672 ± 4.466 | >10 |
| (3-66) | | | | | | | | |
| Omi-28 | 0.022 ± 0.000 | 0.011 ± 0.004 | 0.009 ± 0.002 | 0.008 ± 0.000 | 0.019 ± 0.000 | 0.028 ± 0.009 | 0.133 ± 0.082 | 0.103 ± 0.048 |
| (3-66) | | | | | | | | |
| Omi-29 | 0.014 ± 0.006 | 0.017 ± 0.003 | 0.016 ± 0.009 | 0.056 ± 0.014 | 0.064 ± 0.017 | 0.396 ± 0.007 | >10 | >10 |
| (3-53) | | | | | | | | |
| Omi-30 | 0.012 ± 0.002 | 0.008 ± 0.003 | 0.008 ± 0.004 | 0.011 ± 0.002 | 0.015 ± 0.003 | >10 | 0.008 ± 0.002 | 0.018 ± 0.001 |
| Omi-31 | 0.376 ± 0.090 | 0.029 ± 0.002 | 0.031 ± 0.012 | 0.013 ± 0.002 | 0.013 ± 0.004 | >10 | 0.014 ± 0.008 | 0.015 ± 0.001 |
| Omi-32 | 0.010 ± 0.006 | 0.017 ± 0.000 | >10 | 2.682 ± 0.553 | 1.018 ± 0.139 | 0.035 ± 0.016 | 0.354 ± 0.064 | 2.341 ± 0.282 |
| Omi-33 | 0.027 ± 0.011 | 0.014 ± 0.005 | 0.042 ± 0.018 | 0.068 ± 0.022 | 0.133 ± 0.021 | 0.013 ± 0.004 | 0.053 ± 0.006 | 0.490 ± 0.156 |
| Omi-34 | 0.007 ± 0.004 | 0.008 ± 0.001 | 0.062 ± 0.004 | 0.009 ± 0.003 | 0.014 ± 0.000 | >10 | 0.005 ± 0.000 | 0.020 ± 0.001 |
| Omi-35 | 0.018 ± 0.004 | 0.058 ± 0.006 | 0.381 ± 0.061 | 0.094 ± 0.004 | 0.044 ± 0.018 | 1.687 ± 0.441 | 0.020 ± 0.000 | 0.056 ± 0.012 |
| Omi-36 | 0.022 ± 0.004 | 0.009 ± 0.003 | 0.009 ± 0.003 | 0.030 ± 0.014 | 0.178 ± 0.048 | 0.024 ± 0.006 | >10 | >10 |
| (3-66) | | | | | | | | |
| Omi-38 | 0.015 ± 0.004 | 0.024 ± 0.015 | >10 | 0.005 ± 0.000 | 0.008 ± 0.002 | 0.005 ± 0.001 | 0.011 ± 0.005 | 0.010 ± 0.001 |
| Omi-39 | 0.014 ± 0.002 | 0.009 ± 0.004 | >10 | 0.026 ± 0.011 | 0.014 ± 0.001 | 0.035 ± 0.003 | 0.027 ± 0.009 | 0.045 ± 0.017 |
| Omi-41 | >10 | 0.053 ± 0.028 | 0.037 ± 0.002 | >10 | 0.032 ± 0.007 | >10 | >10 | >10 |
| Omi-42 | 0.013 ± 0.004 | 0.007 ± 0.004 | 0.006 ± 0.002 | 0.021 ± 0.011 | 0.025 ± 0.012 | 0.013 ± 0.001 | 0.003 ± 0.000 | 0.007 ± 0.002 |

TABLE 23
IC50 of commercial mAbs against PV BA.2.75

| | IC50 (μg/mL) | | | | | | |
|---|---|---|---|---|---|---|---|
| | Pseudovirus | | | | | | |
| | Victoria | BA.1 | BA.1.1 | BA.2 | BA.3 | BA.4/5 | BA.2.75 |
| REGN10987 | 0.002 ± 0.001 | >10 | >10 | 0.616 ± 0.347 | >10 | >10 | >10 |
| REGN10933 | 0.001 ± 0.002 | >10 | >10 | >10 | >10 | >10 | >10 |
| AZD1061 | 0.002 ± 0.001 | 0.308 ± 0.058 | >10 | 0.008 ± 0.003 | 0.019 ± 0.007 | 0.015 ± 0.004 | 0.021 ± 0.002 |
| AZD8895 | 0.001 ± 0.000 | 0.246 ± 0.027 | 0.100 ± 0.053 | 1.989 ± 0.517 | >10 | >10 | 0.008 ± 0.000 |
| AZD7442 | 0.001 ± 0.000 | 0.232 ± 0.119 | 0.806 ± 0.093 | 0.008 ± 0.001 | 0.065 ± 0.011 | 0.065 ± 0.007 | 0.017 ± 0.003 |
| ADG10 | 0.007 ± 0.002 | >10 | >10 | >10 | >10 | >10 | >10 |
| ADG20 | 0.003 ± 0.002 | 0.348 ± 0.169 | 0.253 ± 0.070 | >10 | >10 | >10 | >10 |
| ADG30 | 0.014 ± 0.006 | >10 | >10 | >10 | >10 | >10 | >10 |
| Ly-CoV555 | 0.002 ± 0.000 | >10 | >10 | >10 | >10 | >10 | >10 |
| Ly-CoV16 | 0.014 ± 0.010 | >10 | >10 | >10 | >10 | >10 | >10 |
| Ly-CoV1404 | 0.001 ± 0.000 | 0.002 ± 0.000 | 0.001 ± 0.000 | 0.001 ± 0.000 | 0.002 ± 0.000 | 0.002 ± 0.000 | 0.002 ± 0.000 |
| S309 | 0.079 ± 0.027 | 0.113 ± 0.006 | 0.142 ± 0.012 | 0.638 ± 0.15 | 0.311 ± 0.023 | 0.689 ± 0.041 | 0.202 ± 0.017 |

TABLE 24
IC50 (μg/ml)

| mAbs | BA.2 | BA.2 + D339H | BA.2 + R493Q | BA.2 + G446S | BA.2. + N460K | BA.2.75 |
|---|---|---|---|---|---|---|
| Omi02 | 0.003 ± 0.000 | 0.007 ± 0.003 | 0.003 ± 0.000 | 0.007 ± 0.002 | 0.025 ± 0.003 | 0.009 ± 0.002 |
| Omi03 | 0.008 ± 0.001 | 0.006 ± 0.000 | 0.002 ± 0.001 | 0.005 ± 0.001 | 0.401 ± 0.026 | 0.017 ± 0.000 |
| Omi06 | 0.039 ± 0.008 | 0.012 ± 0.002 | 0.023 ± 0.010 | 0.087 ± 0.002 | 0.026 ± 0.002 | 0.063 ± 0.005 |
| Omi08 | 0.114 ± 0.045 | 0.250 ± 0.009 | 0.194 ± 0.020 | 0.017 ± 0.001 | 0.552 ± 0.090 | 0.036 ± 0.002 |
| Omi09 | 0.008 ± 0.002 | 0.005 ± 0.001 | 0.003 ± 0.000 | 0.006 ± 0.001 | 0.010 ± 0.002 | 0.003 ± 0.000 |
| Omi12 | 0.003 ± 0.001 | 0.003 ± 0.001 | 0.001 ± 0.000 | 0.003 ± 0.001 | 0.011 ± 0.002 | 0.003 ± 0.001 |
| Omi16 | 0.034 ± 0.012 | 0.014 ± 0.004 | 0.008 ± 0.003 | 0.018 ± 0.004 | >10 | >10 |
| Omi17 | 0.060 ± 0.004 | 0.036 ± 0.015 | 0.013 ± 0.001 | 0.038 ± 0.002 | >10 | 0.255 ± 0.169 |
| Omi18 | 0.005 ± 0.000 | 0.003 ± 0.000 | 0.004 ± 0.000 | 0.003 ± 0.000 | 0.014 ± 0.002 | 0.035 ± 0.007 |
| Omi20 | 0.015 ± 0.003 | 0.007 ± 0.000 | 0.005 ± 0.001 | 0.005 ± 0.001 | 0.315 ± 0.142 | 0.178 ± 0.075 |
| Omi23 | 0.019 ± 0.005 | 0.006 ± 0.000 | 0.007 ± 0.000 | 0.010 ± 0.002 | 0.022 ± 0.005 | 0.011 ± 0.006 |
| Omi24 | 0.007 ± 0.001 | 0.005 ± 0.001 | 0.004 ± 0.000 | 0.005 ± 0.000 | 0.014 ± 0.000 | 0.008 ± 0.004 |
| Omi25 | 0.024 ± 0.004 | 0.016 ± 0.003 | 0.007 ± 0.002 | 0.022 ± 0.000 | 0.050 ± 0.010 | 0.014 ± 0.005 |
| Omi26 | 0.013 ± 0.001 | 0.007 ± 0.002 | 0.008 ± 0.001 | 0.008 ± 0.002 | 0.010 ± 0.000 | 0.010 ± 0.004 |
| Omi27 | 0.034 ± 0.006 | 0.007 ± 0.001 | 0.007 ± 0.001 | 0.011 ± 0.001 | >10 | 6.672 ± 4.466 |
| Omi28 | 0.008 ± 0.000 | 0.009 ± 0.001 | 0.010 ± 0.001 | 0.014 ± 0.000 | 0.103 ± 0.048 | 0.133 ± 0.082 |
| Omi29 | 0.056 ± 0.014 | 0.018 ± 0.006 | 0.042 ± 0.012 | 0.024 ± 0.002 | >10 | >10 |
| Omi30 | 0.013 ± 0.002 | 0.006 ± 0.001 | 0.002 ± 0.000 | 0.003 ± 0.000 | 0.018 ± 0.001 | 0.008 ± 0.002 |
| Omi31 | 0.011 ± 0.002 | 0.005 ± 0.001 | 0.003 ± 0.000 | 0.005 ± 0.001 | 0.015 ± 0.001 | 0.014 ± 0.008 |
| Omi32 | 2.614 ± 0.533 | 0.683 ± 0.179 | 0.312 ± 0.008 | 0.330 ± 0.010 | 2.341 ± 0.282 | 0.354 ± 0.064 |
| Omi33 | 0.070 ± 0.024 | 0.177 ± 0.035 | 0.063 ± 0.008 | 0.043 ± 0.016 | 0.490 ± 0.156 | 0.053 ± 0.006 |
| Omi34 | 0.009 ± 0.003 | 0.004 ± 0.000 | 0.002 ± 0.000 | 0.002 ± 0.000 | 0.020 ± 0.001 | 0.005 ± 0.000 |
| Omi35 | 0.092 ± 0.004 | 0.012 ± 0.003 | 0.017 ± 0.011 | 0.014 ± 0.006 | 0.056 ± 0.012 | 0.020 ± 0.000 |
| Omi36 | 0.030 ± 0.014 | 0.036 ± 0.002 | 0.013 ± 0.003 | 0.067 ± 0.015 | >10 | >10 |
| Omi38 | 0.005 ± 0.000 | 0.011 ± 0.000 | 0.003 ± 0.001 | 0.010 ± 0.000 | 0.010 ± 0.001 | 0.011 ± 0.005 |
| Omi39 | 0.026 ± 0.011 | 0.012 ± 0.002 | 0.021 ± 0.007 | 0.009 ± 0.002 | 0.045 ± 0.017 | 0.027 ± 0.009 |
| Omi41 | >10 | >10 | >10 | >10 | >10 | >10 |
| Omi42 | 0.021 ± 0.011 | 0.011 ± 0.002 | 0.006 ± 0.001 | 0.016 ± 0.002 | 0.007 ± 0.002 | 0.003 ± 0.000 |

TABLE 25
X-ray data collection and structure
refinement statistics for
BA.2.75 RBD/ACE2

| Structure | BA.2.75 RBD/ACE2 |
|---|---|
| Data collection | |
| Space group | P4₁2₁2 |
| Cell dimensions | |
| a, b, c (Å) | 105.3, 105.3, 220.8 |
| a, b, g (°) | 90, 90, 90 |
| Resolution (Å) | 76-2.85 (2.80-2.85)^{a} |
| R_{merge} | 0.443 (—) |
| Rₚᵢₘ | 0.086 (1.401) |
| I/s(I) | 7.6 (0.4) |
| CC_{1/2} | 0.971 (0.279) |
| Completeness (%) | 99.8 (96.9) |
| Redundancy | 26.8 (25.7) |
| Refinement | |
| Resolution (Å) | 76-2.85 |
| No. reflections | 2089/1439 |
| R_{work}/R_{free} | 0.217/0.265 |
| No. atoms | |
| Protein | 6464 |
| Ligand/ion/water | 167 |
| B factors (Å²) | |
| Protein | 86 |
| Ligand/ion/water | 108 |
| r.m.s. deviations | |
| Bond lengths (Å) | 0.002 |
| Bond angles (°) | 0.4 |

^{a}Values in parentheses are for highest-resolution shell.

TABLE 26
IC50 values for Omicron mAbs

| | IC50 (μg/ml) | | | |
|---|---|---|---|---|
| mAbs | BA.2 | BA.2.11 | BA.2.12.1 | BA.2.13 |
| Omi02 | 0.003 ± 0.000 | 0.004 ± 0.001 | 0.005 ± 0.001 | 0.004 ± 0.000 |
| Omi03 | 0.008 ± 0.001 | 0.005 ± 0.002 | 0.003 ± 0.001 | 0.007 ± 0.005 |
| Omi06 | 0.039 ± 0.008 | 0.000 ± 0.000 | 0.616 ± 0.123 | 0.046 ± 0.024 |
| Omi08 | 0.114 ± 0.045 | 0.099 ± 0.020 | 0.358 ± 0.076 | 0.117 ± 0.009 |
| Omi09 | 0.008 ± 0.002 | 0.016 ± 0.005 | 0.015 ± 0.003 | 0.022 ± 0.002 |
| Omi12 | 0.003 ± 0.001 | 0.002 ± 0.000 | 0.001 ± 0.000 | 0.003 ± 0.000 |
| Omi16 | 0.034 ± 0.012 | 0.017 ± 0.004 | 0.011 ± 0.005 | 0.008 ± 0.000 |
| Omi17 | 0.060 ± 0.004 | 0.022 ± 0.008 | 0.034 ± 0.001 | 0.016 ± 0.001 |
| Omi18 | 0.005 ± 0.000 | 0.002 ± 0.000 | 0.002 ± 0.000 | 0.002 ± 0.001 |
| Omi20 | 0.015 ± 0.003 | 0.007 ± 0.004 | 0.007 ± 0.000 | 0.006 ± 0.000 |
| Omi23 | 0.019 ± 0.005 | 0.009 ± 0.003 | 0.006 ± 0.002 | 0.005 ± 0.001 |
| Omi24 | 0.007 ± 0.001 | 0.000 ± 0.000 | 0.450 ± 0.140 | 0.008 ± 0.000 |
| Omi25 | 0.024 ± 0.004 | 0.007 ± 0.001 | 0.009 ± 0.002 | 0.010 ± 0.000 |
| Omi26 | 0.013 ± 0.001 | 0.007 ± 0.003 | 0.002 ± 0.000 | 0.006 ± 0.000 |
| Omi27 | 0.034 ± 0.006 | 0.005 ± 0.001 | 0.003 ± 0.001 | 0.006 ± 0.000 |
| Omi28 | 0.008 ± 0.000 | 0.007 ± 0.000 | 0.005 ± 0.000 | 0.009 ± 0.001 |
| Omi29 | 0.056 ± 0.014 | 0.011 ± 0.001 | 0.007 ± 0.001 | 0.012 ± 0.001 |
| Omi30 | 0.013 ± 0.002 | 10 | 0.086 ± 0.026 | 0.020 ± 0.002 |
| Omi31 | 0.011 ± 0.002 | 10 | 0.089 ± 0.035 | 0.008 ± 0.004 |
| Omi32 | 2.614 ± 0.53 | 0.070 ± 0.008 | 10 | 0.503 ± 0.080 |
| Omi33 | 0.070 ± 0.024 | 0.008 ± 0.002 | 0.086 ± 0.045 | 0.055 ± 0.007 |
| Omi34 | 0.009 ± 0.003 | 10 | 0.408 ± 0.140 | 0.003 ± 0.001 |
| Omi35 | 0.092 ± 0.003 | 0.667 ± 0.104 | 0.188 ± 0.074 | 0.016 ± 0.004 |
| Omi36 | 0.030 ± 0.014 | 0.051 ± 0.027 | 0.026 ± 0.011 | 0.020 ± 0.004 |
| Omi38 | 0.005 ± 0.000 | 0.004 ± 0.001 | 0.003 ± 0.000 | 0.003 ± 0.001 |
| Omi39 | 0.026 ± 0.011 | 0.018 ± 0.003 | 0.068 ± 0.008 | 0.025 ± 0.007 |
| Omi42 | 0.021 ± 0.011 | 0.009 ± 0.003 | 0.012 ± 0.001 | 0.009 ± 0.001 |

TABLE 27
Primer sequences used to generate
pseudoviruses. Related to Plasmid
construction and pseudotyped
lentiviral partic les production.

| Primer | Sequence (5′ to 3′) |
|---|---|
| BA.2.11 | |
| L452R_F | GGAGGCAATTACAAT |
| | TACCGGTACAGACTG |
| | TTCAGAAAG |
| L452R_R | CTTTCTGAACAGTCT |
| | GTACCGGTAATTGTA |
| | ATTGCCTCC |
| BA.2.12.1 | |
| L452Q_R | CTTTCTGAACAGTCT |
| | GTACTGGTAATTGTA |
| | ATTGCCTCC |
| L452Q_F | GGAGGCAATTACAAT |
| | TACCAGTACAGACTG |
| | TTCAGAAAG |
| S704L_F | GAGCCTGGGCGCCGA |
| | GAATCTAGTGGCCTA |
| | CAGCAATAATAG |
| S704L_R | CTATTATTGCTGTAG |
| | GCCACTAGATTCTCG |
| | GCGCCCAGGCTC |
| BA.2.13 | |
| L452M_F | GTTGGAGGCAATTAC |
| | AATTACATGTACAGA |
| | CTGTTCAGAAAGA |
| L452M_R | TCTTTCTGAACAGTC |
| | TGTACATGTAATTGT |
| | AATTGCCTCCAAC |

TABLE 28
X-ray data collection and structure refinement statistics a
Values in parentheses are for highest-resolution shell.

| Structure | BA.2.12.1 RBD/Beta-27/NbC1 | |
|---|---|---|
| Data collection | | |
| Space group | C2 | |
| Cell dimensions | | |
| a, b, c (Å) | 186.8, 100.0, 56.5 | |
| α, β, γ (°) | 90, 104.1, 90 | |
| Resolution (Å) | 55-2.38 | (2.42-2.38)^{a} |
| R_{merge} | 0.240 | (—) |
| Rₚᵢₘ | 0.071 | (1.366) |
| I/σ(I) | 6.3 | (0.3) |
| CC_{1/2} | 0.988 | (0.13) |
| Completeness (%) | 94.8 | (67.7) |
| Redundancy | 11.2 | (4.4) |

| | |
|---|---|
| Refinement | |
| Resolution (Å) | 55-2.38 |
| No. reflections | 35221/1842 |
| R_{work}/R_{free} | 0.186/0.233 |
| No. atoms | |
| Protein | 5723 |
| Ligand/ion/water | 259 |
| B factors (Å²) | |
| Protein | 58 |
| Ligand/ion/water | 60 |
| r.m.s. deviations | |
| Bond lengths (Å) | 0.002 |
| Bond angles (°) | 0.5 |

TABLE 29
IC50 values for Omicron mAbs and commercial monoclonals
a

| mAbs | Victoria | BA.1 | BA.1.1 | BA.2 | BA.4/5 | BA.4.6 |
|---|---|---|---|---|---|---|
| Omi-02 | 0.002 ± 0.001 | 0.004 ± 0.001 | 0.004 ± 0.001 | 0.003 ± 0.001 | >10 | >10 |
| Omi-03 (3-53) | 0.003 ± 0.000 | 0.005 ± 0.002 | 0.003 ± 0.001 | 0.008 ± 0.001 | 0.017 ± 0.005 | 0.006 ± 0.002 |
| Omi-06 | 0.007 ± 0.000 | 0.017 ± 0.003 | 0.139 ± 0.033 | 0.039 ± 0.008 | >10 | >10 |
| Omi-08 | 0.008 ± 0.004 | 0.003 ± 0.000 | 0.002 ± 0.000 | 0.114 ± 0.045 | 0.086 ± 0.005 | 0.033 ± 0.002 |
| Omi-09 | 0.006 ± 0.002 | 0.005 ± 0.000 | 0.005 ± 0.002 | 0.008 ± 0.002 | 0.166 ± 0.007 | 0.108 ± 0.009 |
| Omi-12 | 0.006 ± 0.002 | 0.002 ± 0.000 | 0.002 ± 0.001 | 0.003 ± 0.001 | 0.429 ± 0.060 | 0.074 ± 0.018 |
| Omi-16 (3-66) | 0.014 ± 0.003 | 0.012 ± 0.002 | 0.011 ± 0.003 | 0.034 ± 0.012 | 0.029 ± 0.007 | 0.007 ± 0.001 |
| Omi-17 (3-66) | 0.023 ± 0.011 | 0.018 ± 0.012 | 0.022 ± 0.009 | 0.060 ± 0.004 | 0.028 ± 0.001 | 0.039 ± 0.008 |
| Omi-18 (3-53) | 0.008 ± 0.003 | 0.002 ± 0.000 | 0.002 ± 0.000 | 0.005 ± 0.000 | 0.005 ± 0.001 | 0.006 ± 0.001 |
| Omi-20 (3-66) | 0.009 ± 0.002 | 0.006 ± 0.001 | 0.005 ± 0.001 | 0.015 ± 0.003 | 0.014 ± 0.006 | 0.008 ± 0.003 |
| Omi-23 | 0.005 ± 0.002 | 0.029 ± 0.006 | 0.023 ± 0.12 | 0.019 ± 0.005 | >10 | >10 |
| Omi-24 | 0.005 ± 0.000 | 0.006 ± 0.002 | 0.054 ± 0.015 | 0.007 ± 0.001 | >10 | >10 |
| Omi-25 | 0.005 ± 0.001 | 0.023 ± 0.005 | 0.027 ± 0.005 | 0.024 ± 0.004 | >10 | >10 |
| Omi-26 | 0.002 ± 0.001 | 0.006 ± 0.002 | 0.005 ± 0.001 | 0.013 ± 0.001 | >10 | >10 |
| Omi-27 (3-66) | 0.008 ± 0.003 | 0.026 ± 0.006 | 0.034 ± 0.009 | 0.034 ± 0.005 | 0.069 ± 0.023 | 0.023 ± 0.002 |
| Omi-28 (3-66) | 0.022 ± 0.000 | 0.011 ± 0.004 | 0.009 ± 0.002 | 0.008 ± 0.000 | 0.028 ± 0.009 | 0.035 ± 0.011 |
| Omi-29 (3-53) | 0.014 ± 0.006 | 0.017 ± 0.003 | 0.016 ± 0.009 | 0.056 ± 0.014 | 0.396 ± 0.007 | 0.170 ± 0.030 |
| Omi-30 | 0.012 ± 0.002 | 0.008 ± 0.003 | 0.008 ± 0.004 | 0.011 ± 0.002 | >10 | >10 |
| Omi-31 | 0.376 ± 0.090 | 0.029 ± 0.002 | 0.031 ± 0.012 | 0.013 ± 0.002 | >10 | >10 |
| Omi-32 | 0.010 ± 0.006 | 0.017 ± 0.000 | >10 | 2.682 ± 0.553 | 0.035 ± 0.016 | >10 |
| Omi-33 | 0.027 ± 0.011 | 0.014 ± 0.005 | 0.042 ± 0.018 | 0.068 ± 0.022 | 0.013 ± 0.004 | >10 |
| Omi-34 | 0.007 ± 0.004 | 0.008 ± 0.001 | 0.062 ± 0.004 | 0.009 ± 0.003 | >10 | >10 |
| Omi-35 | 0.018 ± 0.004 | 0.058 ± 0.006 | 0.381 ± 0.061 | 0.094 ± 0.004 | 1.687 ± 0.441 | >10 |
| Omi-36 (3-66) | 0.022 ± 0.004 | 0.009 ± 0.003 | 0.009 ± 0.003 | 0.030 ± 0.014 | 0.024 ± 0.006 | 0.029 ± 0.001 |
| Omi-38 | 0.015 ± 0.004 | 0.024 ± 0.015 | >10 | 0.005 ± 0.000 | 0.005 ± 0.001 | >10 |
| Omi-39 | 0.014 ± 0.002 | 0.009 ± 0.004 | >10 | 0.026 ± 0.011 | 0.035 ± 0.003 | >10 |
| Omi-41 | >10 | 0.053 ± 0.028 | 0.037 ± 0.002 | >10 | >10 | >10 |
| Omi-42 | 0.013 ± 0.004 | 0.007 ± 0.004 | 0.006 ± 0.002 | 0.021 ± 0.011 | 0.013 ± 0.001 | 0.010 ± 0.001 |

b

| | IC50 (μg/mL) | | | | | | |
|---|---|---|---|---|---|---|---|
| | Pseudovirus | | | | | | |
| | Victoria | BA.1 | BA.1.1 | BA.2 | BA.3 | BA.4 | BA.4.6 |
| AZD1061 | 0.002 ± 0.001 | 0.308 ± 0.058 | >10 | 0.008 ± 0.003 | 0.019 ± 0.007 | 0.015 ± 0.004 | >10 |
| AZD8895 | 0.001 ± 0.000 | 0.246 ± 0.027 | 0.100 ± 0.053 | 1.333 ± 0.317 | >10 | >10 | >10 |
| AZD7442 | 0.001 ± 0.000 | 0.232 ± 0.113 | 0.806 ± 0.093 | 0.008 ± 0.001 | 0.065 ± 0.011 | 0.065 ± 0.007 | >10 |
| REGN10987 | 0.002 ± 0.001 | >10 | >10 | 0.616 ± 0.347 | >10 | >10 | >10 |
| REGN10933 | 0.001 ± 0.002 | >10 | >10 | >10 | >10 | >10 | >10 |
| ADG10 | 0.007 ± 0.002 | >10 | >10 | >10 | >10 | >10 | >10 |
| ADG20 | 0.003 ± 0.002 | 0.348 ± 0.169 | 0.253 ± 0.070 | >10 | >10 | >10 | >10 |
| ADG30 | 0.014 ± 0.006 | >10 | >10 | >10 | >10 | >10 | >10 |
| Ly-CoVS55 | 0.002 ± 0.000 | >10 | >10 | >10 | >10 | >10 | >10 |
| Ly-CoV16 | 0.014 ± 0.010 | >10 | >10 | >10 | >10 | >10 | >10 |
| Ly-CoV1404 | 0.001 ± 0.000 | 0.002 ± 0.000 | 0.001 ± 0.000 | 0.001 ± 0.000 | 0.002 ± 0.000 | 0.002 ± 0.000 | 0.001 ± 0.000 |
| S309 | 0.079 ± 0.027 | 0.113 ± 0.006 | 0.142 ± 0.012 | 0.638 ± 0.154 | 0.311 ± 0.023 | 0.689 ± 0.041 | 1.029 ± 0.058 |

TABLE 30
Primer sequences used to generate
pseudoviruses. Related to Plasmid
construction and pseudotyped
lentiviral particle production.

| | Sequence |
|---|---|
| Primer | (5′ to 3′) |
| pcDNA3.1_BamHI_F | GGATCCATGTTCCTG |
| | CTGACCACCAAGAG |
| pcDNA3.1_Tag_S_ | GAATTCTCACTTCTC |
| EcoRI_R | GAACTGAGGGTGGC |
| pcDNA3.1_Tag_S_ | GCCACCCTCAGTTCG |
| EcoRI_F | AGAAGTGAGAATTC |
| pcDNA3.1_BamHI_R | CTCTTGGTGGTCAGC |
| | AGGAACATGGATCC |
| BA.4 + R346T_F | GTGTTCAATGCCACC |
| | ACGTTCGCCAGCGTG |
| | TACG |
| BA.4 + R346T_R | CGTACACGCTGGCGA |
| | ACGTGGTGGCATTGA |
| | ACAC |
| BA4 + N658S_F | CGGCGCCGAGTACGT |
| | GAATAGTAGCTACGA |
| | GTGCG |
| BA.4 + N658S_R | CGCACTCGTAGCTAC |
| | TATTCACGTACTCCG |
| | GCGCCG |

TABLE 31
Table of SARS-COV-2 lineages and genomic mutations

| | | | | Country/ | | |
|---|---|---|---|---|---|---|
| | | | | Region | Date of | |
| | | Example early | | of earliest | earliest | Pango issue, |
| Lineage | Defining RBD mutations | genome | Submitting scientist, laboratory | sequences | sequences | contributor |
| BA.4.6 | BA.4/5 + R346T | EPI_ISL_12475182 | Oliver et al, HOSPITAL | Europe/ | April 2022 | #741, ryhisner |
| | | | UNIVERSITARIO | South | | |
| | | | SON ESPASES | Africa | | |
| BA.4.7 | BA.4/5 + R346S | EPI_ISL_12644817 | Iranzadeh et al. NHLS/UCT | South | April 2022 | #777, FedeGueli |
| | | | | Africa/ | | |
| | | | | Israel | | |
| BF.7 | BA.4/5 R346T | EPI_ISL_12810243 | Coppens et al. LaboKlinische | Belgium | May 2022 | #827, ryhisner |
| (BA.5.2.1.7) | | | Biologie, UZA | | | |
| BQ.1 | BA.4/5 K444T, N460K | EPI_ISL_14294806 | Howard et al., Centers for | Nigeria | July 2022 | #993, FedeGueli |
| (BA.5.3.1.1.1.1.1) | | | Disease Control and Prevention | | | |
| | | | Division of Viral Diseases | | | |
| | | | Pathogen Discovery | | | |
| BQ.1.1 | BQ.1 + R346T | EPI_ISL_14752457 | Christensen et al., Houston | USA | August 2022 | #993, FedeGueli |
| (BA.5.3.1.1.1.1.1.1) | | | Methodist Hospital | | | |
| BA.2.75 | BA.2 + G446S, N460K, | EPI_ISL_13302209 | Khaimar et al. CSIR-NEERI, | India | April 2022 | #773, Silcn |
| | R495Q* | | Nagpur Covid-19 Testing Lab | | | |
| BA.2.75.2 | BA.2.75 + R346T, F486S | EPI_ISL_14290506 | Gupta et al. ILBS/INSACOG | India | July 2022 | #966, agamedilab |
| BN.1 | BA.2.75 + R346T, K356I, | EPI_ISL_14601644 | Sima et al, Lifebrain Covid | India | July 2022 | #994, |
| (BA.2.75.5.1) | F490S | | Labor GmbH | | | corneliusroemer |
| BJ.1 | BA.2 + R346T, L368I, | EPI_ISL_14166909 | Maitra eta l. National Institute | India | June 2022 | #915, Silcn |
| (BA.2.10.1.1) | V445P, G446S, V483A | | of Biomedical Genomics- | | | |
| | F490V | | INSACOG | | | |
| BA.2.10.4 | BA.2 + G446S, F486P, | EPI_ISL_13929780 | Karyakarte et al. Center for | India | June 2022 | #898, Silcn |
| | R493Q*, S494P | | Genomics, Department of | | | |
| | | | Microbiology, BJ Government | | | |
| | | | Medical College and Sassoon | | | |
| | | | Hospitals | | | |
| BS.1 | BA.2 + R346T, L452R, | EPI_ISL_14853710 | Sekizuka et al. Pathogen | Japan ex | August 2022 | #1052, Takaken6 |
| (BA.2.3.2.1) | N460K, G476S | | Genomics Center, National | Vietnam | | |
| | | | Institute of Infectious Diseases | | | |
| BA.2.3.20 | K444R, N450D, L452M, | EPI_ISL_14723265 | Selway et al, SA Pathology | USA/ | August 2022 | #1013, ryhisner |
| | N460K, E484R, R495Q* | | | Singapore/ | | |
| | | | | Australia | | |
| XBB | BA.2 + R346T, L368I, | EPI_ISL_14917761 | Ngan et al, National Public | India | August 2022 | #1058, |
| | V445P, G446S, N460K, | | Health Laboratory, National | | | corneliusroemer |
| | F486S, F490S | | Centre for Infectious Diseases | | | |

TABLE 32
IC50 values for BA.1 mAbs and commercial mAbs
a

| mAbs | Victoria | BA.2 | BA.4/5 | BA.4.6 | BA.2.75 | BA.2.75.2 | BA.2.3.20 | BJ.1 | BA.4 ± all |
|---|---|---|---|---|---|---|---|---|---|
| Omi-02 | 0.002 ± 0.001 | 0.003 ± 0.001 | >10 | >10 | 0.009 ± 0.002 | >10 | 0.013 ± 0.001 | 0.011 ± 0.001 | >10 |
| Omi-03 (3-53) | 0.003 ± 0.000 | 0.008 ± 0.001 | 0.017 ± 0.005 | 0.006 ± 0.002 | 0.017 ± 0.000 | 0.546 ± 0.166 | 0.020 ± 0.007 | 0.014 ± 0.000 | 0.432 ± 0.106 |
| Omi-06 | 0.007 ± 0.000 | 0.039 ± 0.008 | >10 | >10 | 0.063 ± 0.005 | >10 | >10 | >10 | >10 |
| Omi-08 | 0.008 ± 0.004 | 0.114 ± 0.045 | 0.086 ± 0.005 | 0.033 ± 0.002 | 0.036 ± 0.002 | 0.027 ± 0.012 | 0.426 ± 0.024 | >10 | >10 |
| Omi-09 | 0.006 ± 0.002 | 0.008 ± 0.002 | 0.166 ± 0.007 | 0.108 ± 0.009 | 0.003 ± 0.000 | 0.012 ± 0.000 | 0.133 ± 0.003 | >10 | >10 |
| Omi-12 | 0.006 ± 0.002 | 0.003 ± 0.001 | 0.429 ± 0.060 | 0.074 ± 0.018 | 0.003 ± 0.001 | >10 | 0.008 ± 0.001 | 0.004 ± 0.000 | >10 |
| Omi-16 (3-66) | 0.014 ± 0.003 | 0.034 ± 0.012 | 0.029 ± 0.007 | 0.007 ± 0.001 | 8.666 ± 4.596 | >10 | 1.075 ± 0.241 | 0.025 ± 0.000 | >10 |
| Omi-17 (3-66) | 0.023 ± 0.011 | 0.060 ± 0.004 | 0.028 ± 0.001 | 0.039 ± 0.008 | 0.255 ± 0.169 | >10 | 0.347 ± 0.123 | 0.030 ± 0.006 | >10 |
| Omi-18 (3-53) | 0.008 ± 0.003 | 0.005 ± 0.000 | 0.005 ± 0.001 | 0.006 ± 0.001 | 0.035 ± 0.007 | 4.800 ± 0.568 | 0.011 ± 0.001 | 0.005 ± 0.002 | 3.607 ± 0.807 |
| Omi-20 (3-66) | 0.009 ± 0.002 | 0.015 ± 0.003 | 0.014 ± 0.006 | 0.008 ± 0.003 | 0.178 ± 0.075 | 8.948 ± 3.561 | 0.030 ± 0.005 | 0.009 ± 0.002 | >10 |
| Omi-23 | 0.005 ± 0.002 | 0.019 ± 0.005 | >10 | >10 | 0.011 ± 0.006 | >10 | 0.009 ± 0.003 | 0.024 ± 0.001 | >10 |
| Omi-24 | 0.005 ± 0.000 | 0.007 ± 0.001 | >10 | >10 | 0.008 ± 0.004 | 4.681 ± 1.859 | >10 | >10 | >10 |
| Omi-25 | 0.005 ± 0.001 | 0.024 ± 0.004 | >10 | >10 | 0.014 ± 0.005 | >10 | 0.025 ± 0.004 | 0.041 ± 0.028 | >10 |
| Omi-26 | 0.002 ± 0.001 | 0.013 ± 0.001 | >10 | >10 | 0.010 ± 0.004 | >10 | 0.006 ± 0.001 | 0.031 ± 0.015 | >10 |
| Omi-27 (3-66) | 0.008 ± 0.003 | 0.034 ± 0.005 | 0.069 ± 0.023 | 0.023 ± 0.002 | 6.672 ± 4.466 | >10 | 0.215 ± 0.111 | 0.007 ± 0.000 | >10 |
| Omi-28 (3-66) | 0.022 ± 0.000 | 0.008 ± 0.000 | 0.028 ± 0.009 | 0.035 ± 0.011 | 0.133 ± 0.082 | 7.592 ± 0.028 | 0.053 ± 0.013 | 0.010 ± 0.001 | >10 |
| Omi-29 (3-53) | 0.014 ± 0.006 | 0.056 ± 0.014 | 0.396 ± 0.007 | 0.170 ± 0.030 | >10 | >10 | >10 | 0.025 ± 0.012 | >10 |
| Omi-30 | 0.085 ± 0.008 | 0.011 ± 0.002 | >10 | >10 | 0.008 ± 0.002 | 0.009 ± 0.001 | 0.343 ± 0.023 | 1.827 ± 0.436 | >10 |
| Omi-31 | 0.014 ± 0.001 | 0.013 ± 0.002 | >10 | >10 | 0.014 ± 0.008 | 0.012 ± 0.001 | >10 | >10 | >10 |
| Omi-32 | 0.010 ± 0.006 | 2.682 ± 0.553 | 0.035 ± 0.016 | >10 | 0.354 ± 0.064 | >10 | >10 | >10 | >10 |
| Omi-33 | 0.027 ± 0.011 | 0.068 ± 0.022 | 0.013 ± 0.004 | >10 | 0.053 ± 0.006 | >10 | >10 | >10 | >10 |
| Omi-34 | 0.007 ± 0.004 | 0.009 ± 0.003 | >10 | >10 | 0.005 ± 0.000 | 0.005 ± 0.001 | >10 | >10 | >10 |
| Omi-35 | 0.018 ± 0.004 | 0.094 ± 0.004 | 1.687 ± 0.441 | >10 | 0.020 ± 0.000 | >10 | >10 | >10 | >10 |
| Omi-36 (3-66) | 0.022 ± 0.004 | 0.030 ± 0.014 | 0.024 ± 0.006 | 0.029 ± 0.001 | >10 | 3.815 ± 0.064 | >10 | 0.045 ± 0.005 | >10 |
| Omi-38 | 0.015 ± 0.004 | 0.005 ± 0.000 | 0.005 ± 0.001 | >10 | 0.011 ± 0.005 | >10 | >10 | >10 | >10 |
| Omi-39 | 0.014 ± 0.002 | 0.026 ± 0.011 | 0.035 ± 0.003 | >10 | 0.027 ± 0.009 | >10 | >10 | >10 | >10 |
| Omi-41 | >10 | >10 | >10 | >10 | >10 | >10 | >10 | >10 | >10 |
| Omi-42 | 0.013 ± 0.004 | 0.021 ± 0.011 | 0.013 ± 0.001 | 0.010 ± 0.001 | 0.003 ± 0.000 | 0.011 ± 0.005 | 0.018 ± 0.002 | 0.010 ± 0.001 | 0.008 ± 0.001 |

b

| | Victoria | BA.2 | BA.4/5 | BA.2.75 | BA.4.6 | BA.2.75.2 | BA.2.3.20 | BJ.1 | BA.4 + all |
|---|---|---|---|---|---|---|---|---|---|
| AZD1061 | 0.002 ± 0.001 | 0.008 ± 0.003 | 0.015 ± 0.004 | 0.021 ± 0.002 | >10 | >10 | >10 | >10 | >10 |
| AZ08895 | 0.001 ± 0.000 | 1.333 ± 0.517 | >10 | 0.008 ± 0.000 | >10 | >10 | 0.007 ± 0.001 | 0.114 ± 0.013 | >10 |
| AZD7442 | 0.001 ± 0.000 | 0.008 ± 0.001 | 0.065 ± 0.007 | 0.017 ± 0.005 | >10 | >10 | 0.025 ± 0.001 | 2.735 ± 0.579 | >10 |
| REGN10987 | 0.002 ± 0.001 | 0.615 ± 0.347 | >10 | >10 | >10 | >10 | >10 | >10 | >10 |
| REGN10933 | 0.001 ± 0.002 | >10 | >10 | >10 | >10 | >10 | 8.624 ± 0.019 | >10 | >10 |
| ADG20 | 0.000 ± 0.002 | >10 | >10 | >10 | >10 | >10 | >10 | >10 | >10 |
| Ly-COV555 | 0.002 ± 0.000 | >10 | >10 | >10 | >10 | >10 | >10 | >10 | >10 |
| Ly-Cov16 | 0.014 ± 0.010 | | >10 | >10 | >10 | >10 | >10 | >10 | >10 |
| Ly-COV1404 | 0.001 ± 0.000 | 0.001 ± 0.000 | 002 ± 0.000 | 0.002 ± 0.000 | 0.001 ± 0.000 | 0.002 ± 0.001 | 0.018 ± 0.005 | >10 | >10 |
| S309 | 0.079 ± 0.027 | 0.638 ± 0.154 | 0.689 ± 0.041 | 0.202 ± 0.017 | 1.029 ± 0.058 | 0.465 ± 0.135 | 0.977 ± 0.107 | 0.431 ± 0.010 | 0.584 ± 0.072 |

1. An antibody capable of binding to the spike protein of coronavirus SARS-CoV-2, wherein the antibody comprises at least three CDRs of antibody Omi12, or of any one of the 27 antibodies in Table 3.

2. The antibody according to claim 1, comprising:
(a) at least four, five, or all six CDRs of an antibody in Table 3;
(b) a heavy chain variable domain comprising or consisting of an amino acid sequence having at least 80% sequence identity to the heavy chain variable domain of an antibody in Table 3;
(c) a light chain variable domain comprising or consisting of an amino acid sequence having at least 80% sequence identity to the light chain variable domain of an antibody in Table 3; and/or
(d) a heavy chain variable domain and a light chain variable domain comprising or consisting of an amino acid sequence having at least 80% identity to the heavy chain variable domain and light chain domain, respectively, of an antibody in Table 3.

3. The antibody of claim 1 or claim 2, wherein the antibody in Table 3 is selected from the group consisting of:
(a) Omi02, Omi03, Omi12, Omi18, Omi28, Omi39 and Omi42;
(b) Omi03 and Omi12;
(c) Omi03, Omi12, Omi02, Omi39, and Omi42;
(d) Omi03, Omi12, Omi02, Omi39, Omi42, Omi16, Omi18, Omi20, Omi23, Omi28 and Omi08; or
(e) Omi03, Omi12, Omi02, Omi39, Omi42, Omi16, Omi18, Omi20, Omi 23, Omi28, Omi08, Omi17, Omi29, Omi36 and Omi38.

4. The antibody according to claim 1, comprising CDRH1, CDRH2 and CDRH3, from a first antibody in any one of Tables 1 to 3, and CDRL1, CDRL2 and CDRL3 from a second antibody in any one of Tables 1 to 3, with the proviso that the first antibody and the second antibody are different.

5. The antibody according to claim 4, comprising a heavy chain variable domain amino acid sequence having at least 80% sequence identity to the heavy chain variable domain from a first antibody in any one of Tables 1 to 3, and a light chain variable domain amino acid sequence having at least 80% sequence identity to the light chain variable domain from a second antibody in any one of Tables 1 to 3.

6. The antibody according to claim 4 or claim 5, wherein the first and second antibodies derive from the same germline heavy chain v-region, optionally wherein the heavy chain v-region is IGHV1-69, IGHV3-53, IGHV1-58, IGHV3-66, IGHV3-30, IGHV3-33, IGHV1-18, IGHV3-9, or IGHV4-31.

7. The antibody according to any one of claims 4 to 6, wherein the first antibody and the second antibody are both selected from the group consisting of:
(a) Omi03, Omi18, Omi29, Beta-27, antibody 150, antibody 158, antibody 175, antibody 222 and antibody 269; optionally wherein the antibody comprises heavy chain variable domain and light chain variable domain of one of the antibodies as set out in Table 4;
(b) Omi03, Omi18, Omi29, Omi16, Omi17, Omi20, Omi27, Omi36, Beta-27, antibody 150, antibody 158, antibody 175, antibody 222, antibody 269, antibody 40 and antibody 398; optionally wherein the antibody comprises heavy chain variable domain and light chain variable domain of one of the antibodies as set out in Table 5;
(c) Omi12, Beta-47, Beta-25, antibody 55, antibody 165, antibody 253 and antibody 318; optionally wherein the antibody comprises heavy chain variable domain and light chain variable domain of one of the antibodies as set out in Table 6;
(d) Beta-49, Beta-50, Omi02, Omi24, Omi30, Omi31, Omi34 and Omi38; optionally wherein the antibody comprises heavy chain variable domain and light chain variable domain of one of the antibodies as set out in Table 7;
(e) Beta-22, Beta-29, antibody 159 and Omi09; optionally wherein the antibody comprises heavy chain variable domain and light chain variable domain of one of the antibodies as set out in Table 8;
(f) Beta-20, Beta-43, Omi32 and Omi33; optionally wherein the antibody comprises heavy chain variable domain and light chain variable domain of one of the antibodies as set out in Table 9;
(g) antibody 278, Beta-44, Omi26 and Omi41; optionally wherein the antibody comprises heavy chain variable domain and light chain variable domain of one of the antibodies as set out in Table 10;
(h) antibody 58, Omi25, Omi35 and Omi42; optionally wherein the antibody comprises heavy chain variable domain and light chain variable domain of one of the antibodies as set out in Table 11; or
(i) Beta-56 and Omi23; optionally wherein the antibody comprises heavy chain variable domain and light chain variable domain of one of the antibodies as set out in Table 12.

8. The antibody of any one of the preceding claims, wherein the antibody in Tables 3 to 12 is selected from the group consisting of:
(a) Omi02, Omi03, Omi12, Omi18, Omi28, Omi39 and Omi42;
(b) Omi03 and Omi12;
(c) Omi03, Omi12, Omi02, Omi39, and Omi42;
(d) Omi03, Omi12, Omi02, Omi39, Omi42, Omi16, Omi18, Omi20, Omi23, Omi28 and Omi08; or
(e) Omi03, Omi12, Omi02, Omi39, Omi42, Omi16, Omi18, Omi20, Omi 23, Omi28, Omi08, Omi17, Omi29, Omi36 and Omi38.

9. The antibody according to any one of the preceding claims, which is a full-length antibody, e.g. comprising a IgG1 constant region, or comprises an Fc region comprising at least one modification such that serum half-life is extended.

10. The antibody according to any one of the preceding claims, wherein the antibody is derived from germline heavy chain IGHV1-58, such as Omi-12, Beta-47, Beta-25, antibody 55, antibody 165, antibody 253, and antibody 318, and comprises proline at position 53 in the heavy chain variable region.

11. A combination of antibodies comprising two or more antibodies according to any one of the preceding claims.

12. A combination of antibodies comprising:
(a) the antibody according to any one of claims 1 to 10; and
(b) an antibody comprising at least three CDRs of an antibody in Table 1 or Table 2, for example, the antibody comprises:
(i) at least four, five, or all six CDRs of an antibody in Table 1 or Table 2;
(ii) a heavy chain variable domain comprising or consist of an amino acid sequence having at least 80% sequence identity to the heavy chain variable domain of an antibody in Table 1 or Table 2;
(iii) a light chain variable domain comprising or consist of an amino acid sequence having at least 80% sequence identity to the light chain variable domain of an antibody in Table 1 or Table 2; and/or
(iv) a heavy chain variable domain and a light chain variable domain comprising or consist of an amino acid sequence having at least 80% identity to the heavy chain variable domain and light chain domain, respectively, of an antibody in Table 1 or Table 2.

13. The combination of antibodies according to claim 11 or claim 12, comprising two, three or four antibodies according to any one of claims 1 to 10.

14. One or more polynucleotides encoding the antibody according to any one of claims 1 to 10, one or more vectors comprising said polynucleotides, or a host cell comprising said vectors.

15. A method for producing an antibody that is capable of binding to the spike protein of coronavirus SARS-CoV-2, comprising culturing the host cell of claim 14 and isolating the antibody from said culture.

16. A pharmaceutical composition comprising: (a) the antibody according to any one of claims 1 to 12, or the combination of antibodies according to any one of claims 11 to 13, and (b) at least one pharmaceutically acceptable diluent or carrier.

17. The antibody according to any one of claims 1 to 10, the combination according to any one of claims 11 to 13, or the pharmaceutical composition according to claim 16, for use in a method for treatment of the human or animal body by therapy.

18. The antibody according to any one of claims 1 to 10, the combination according to any one of claims 11 to 13, or the pharmaceutical composition according to claim 16, for use in a method of treating or preventing coronavirus infection, or a disease or complication associated with coronavirus infection.

19. A method of treating or preventing coronavirus infection, or a disease or complication associated with coronavirus infection in a subject, comprising administering a therapeutically effective amount of the antibody according to any one of claims 1 to 10, the combination according to any one of claims 11 to 13, or the pharmaceutical composition according to claim 16, to said subject.

20. The method according to claim 19 or 20, wherein the method is for treating SARS-CoV-2 infection, or a disease or complication associated therewith, such as COVID-19.

21. A method of identifying the presence of coronavirus, or a protein fragment thereof, in a sample, comprising:
(i) contacting the sample with the antibody according to any one of claims 1 to 10, or the combination according to any one of claims 11 to 13, and
(ii) detecting the presence or absence of an antibody-antigen complex,
wherein the presence of the antibody-antigen complex indicates the presence of coronavirus, or a fragment thereof, in the sample.

22. A method of treating or preventing coronavirus infection, or a disease or complication associated therewith, in a subject, comprising identifying the presence of coronavirus according to the method of claim 21 in a sample, and treating the subject with the antibody or combination according to any one of claims 1 to 13, an anti-viral drug or an anti-inflammatory agent.

23. Use of the antibody according to any one of claims 1 to 10, the combination according to any one of claims 11 to 13, or the pharmaceutical composition according to claim 16, for preventing, treating and/or diagnosing coronavirus infection, or a disease or complication associated therewith.

24. Use of the antibody according to any one of claims 1 to 10, the combination according to any one of claims 11 to 13, or the pharmaceutical composition according to claim 16, for the manufacture of a medicament for treating or preventing coronavirus infection, or a disease or complication associated therewith.

25. The antibody for use according to claim 17 or 18, the method according to any one of claims 19 to 22 and the use according to claim 23 or 24, wherein the coronavirus infection is caused by a SARS-CoV-2 strain of the lineage alpha, beta, gamma, delta or omicron, optionally wherein the lineage of the omicron strain is omicron BA.1, omicron BA1.1, omicron BA.2 or omicron BA.3.

26. An antibody derived from germline heavy chain IGHV1-58, capable of binding to the spike protein of coronavirus SARS-CoV-2, wherein the amino acid at position 58 in the heavy chain variable region according to IMGT numbering is proline or is substituted with proline.

27. The antibody of claim 26, wherein the antibody derived from germline heavy chain IGHV1-58 is AZD8895, Omi-12, Beta-47, Beta-25, antibody 55, antibody 165, antibody 253, or antibody 318.

28. An antibody capable of binding to the spike protein of coronavirus SARS-CoV-2 comprising a heavy chain variable domain comprising an amino acid sequence having ≥60%, ≥70%, ≥80%, ≥90%, ≥95%, ≥96%, ≥97%, ≥98%, ≥99% or 100% sequence identity to SEQ ID NO: 961, with the proviso that the amino acid at position 58 according to IMGT numbering is proline or is substituted with proline.

29. The antibody of any one of claims 26 to 28, wherein the antibody comprises a heavy chain variable domain comprising an amino acid sequence having ≥80%, ≥90%, ≥95%, ≥96%, ≥97%, ≥98%, ≥99% or 100% sequence identity to SEQ ID NO: 731, 591, 461, 62, 182, 262, 332 or 963, with the proviso that the amino acid at position 58 according to IMGT numbering is proline or is substituted with proline.

30. The antibody of any one of claims 26 to 29, wherein the antibody comprises a heavy chain variable domain comprising an amino acid sequence having SEQ ID NO: 591 461, 62, 182, 262, or 332, wherein the valine at position 58 according to IMGT numbering is substituted with a proline.

31. The antibody of any one of claims 26 to 29, wherein the antibody comprises a heavy chain variable domain comprising an amino acid sequence having SEQ ID NO: 961, wherein the isoleucine at position 58 according to IMGT numbering is substituted with a proline.
